SEQUENCE LISTING (653 sequences)

SEQ ID NO: 1 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 2 - protein, 292 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFV

SEQ ID NO: 3 - protein, 305 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 4 - protein, 311 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLN

SEQ ID NO: 5 - protein, 323 aa, synthetic construct
MWPLVAALLLGSACCGSAQLLFNKTKSVEFTFCNDTVVIPCFVTNMEAQNTTEVYVKWKFKGRDIYTFDGALNKCTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 6 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNACGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 7 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYCVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 8 - protein, 323 aa, synthetic construct
MWPLVAALLLGSACCGSAQLLFNKTKSVEFTFCNDTVVIPCFVTNMEAQNTTEVYVKWKFKGRDIYTFDGALNKCTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNACGLGLIVTSTGILILLHYCVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 9 - protein, 323 aa, synthetic construct; identical to SEQ ID NO: 8

SEQ ID NO: 10 - protein, 276 aa, synthetic construct
MGSAQLMFNVTDVVQKTDCNKTVILPCHVINLNENNPNVMFVTWKRQGKIIFSFDGGRKEFFRDSTVPSANLVSQVDLPKGIASLRLNSEEADVGNYSCEVTESNREGETRVELRKYSGSWFLLVERAIIIALLFLIVILCLAQLILVALKYEIGPEKRMGIIVTAVISIVVAVVGTVLFVQDGYTAQNQTGLGLIVVPAVIFVPLQYFMFGIVFGSLPQATFALIGLQILGYVIAVVGFALCVSACPPIHGSVVIAGLAILAIANLLSLGYVFIM

SEQ ID NO: 11 - protein, 290 aa, synthetic construct
MGSAQLIFSVTDVVERTDCNKTVILPCYVTNLKEDNADVMFVTWKKQGKIIFSFDGAKRQFFRDAAVPSANLVSQADLPKGIASLMLKSAEADAGNYSCEVTESNREGETRVELRNHSGSWFLLVERAIIIALLFLVIILCSAQLSVIALKYEIVPQKKMGMIVAVTIFTVAAVVGTVLFVQDGYTAQNQAGLGLIVVPAVILVPLQYFVFGIVFDSLPQATFALIGLHILGYVIAVVGFALCVSACPPLHGSVLIAGLAIMAIANLLSLAYMFIMGSRMKDHPRPGKAV

SEQ ID NO: 12 - protein, 275 aa, synthetic construct
MGSAQLALTTTDVETTNCNKTVILPCYVTDLHENNEKTMIVLWALNEREIFSYEGANRTVFRDNAFPSANLLSPGDLLKGVASLTLSSAEAEVGNYTCQVTESNREGKIRVELKSRVGSWFPLVERAIIIVLLFLIIVLCSAQLSVVAFKYEIVPQKKVSIAVAGFIFTVAAVIGTVLYVQDGFTAQNQAGLGLIVIPAVSMVPLQYFIFRIAFDSLPQATFALIGLHILGYVIAVVGFALCVSACPPSHGSILIAGLAIMAITNLLSLAYVFIM

SEQ ID NO: 13 - protein, 309 aa, synthetic construct
MWPLVGALLLGSACCGSAQITFNITKSAEFTVCNTTVVIPCFATNVETKLLTDLYVKWKFKGKDIFTYDGAQGQSTPSSGFKSAKITPTELLHGIASLKMKKSDAELGNYTCEVTELTREGETVIELKYRVVSWFSPKENILIIIFPVLAILLSWGQFGVVTLKYASDPMKGKTILLFVAGLLLTIVVIVGAILFIPGEYSTKNASGLGLIVIPTVILILLQYFVLMTAFGMTSFAIAILILQVLGFVLAVVGLSLCIPECTPVQGPLLISGLGIIALAELLGLVSMKVVASSQGTIQPPRKAVAFKED

SEQ ID NO: 14 - protein, 277 aa, synthetic construct
MGSAQLILSGTSVIEKSDCNKTVVLPCHVTNLRQKNENAMFVIWKKQGDTIFSYRGGNKKFNIDPSFSSARFLHPENLTDGDASLVLNSKQATVGNYSCEVTESNREGELRMELKNSSGSWFLLVERAVIISLVILLVILCAAQLIFIGLKYEIESQRKVCIIVALVIFAVVAGVGAALFIQDGYTVQNQAGLGLMVIPAVILVPLQYVMFGIVFDSQLQATLALIGLKLLGFIIAVVGFALCVPACPPLHGSVLIAGLAIMAIASLLSLAYVFIMG

SEQ ID NO: 15 - protein, 277 aa, synthetic construct
MGSAQLILIGTSVIEKSGCNETVVLPCHVTNLRQKNENAMFVIWKKEGDTIFSYRGGNKKYNVDNSFSSAKFLHPENLTDGDASLVLSSKQAVVGNYSCEVTESNREGELRMELRNGSGSWFLLVERAVIVSLVILLVILCAAQLTFIGLKYEIESQRKVCIIVALVIFAVVAAVGAALFIQDGYTVQNQAGLGLMVIPAVILVPLQYVMFGIVFDSQLQATLALIGLKLLGFIIAVVGFALCVPACPPLHGSVLIAGIAIMAIASLLSLAYVFIMG

SEQ ID NO: 16 - protein, 277 aa, synthetic construct
MGSAQLILSGTSVIEKSDCNKTVVLPCHVTNLRQKNENAMFVIWKKEGDTIFSYRGGNKKFNIDPSFSSARFLHPENLTDGDASLVLDSKQATVGNYSCEVTESNREGELRMELKNSSGSWFLLVERAVIISLVILLVILCAAQLIFIGLKYEIESQRKVCIIVALVIFAVVAGVGAALFIQDGYTVQNQAGLGLMVIPAVILVPLQYVMFGIVFDSQLQATLALIGLKLLGFIIAVVGFALCVPGKQPLHGSVLLSLLGKCAFYSCLHLIFVYFMG

SEQ ID NO: 17 - protein, 330 aa, synthetic construct
MWLLAASVLLGALRAGSAELTLEGGGIIEKNDCNETVVLPCYVTNLEEKNENALFVTWKKQETPIFSYRGATKKFTIDPSFSSARFLSQGKLIDGDASLVLNSAQATVGNYSCKVTESNREGEIKMELKSSSVVSCGDETPEDKRGSWFLLVERAVIISLVCLLVILCAAQLSVIGLKYEIESQRKVCIIVALVIFAVVAGVGAALFIQDGYTAQNQAGLGLIVIPAVILVPLQYVMFGIVFDSLLQATLALIGLKLLGFIIAVVGFALCVPACPPLHGSVLIAGLAIMAIASLLSLAYVFFMVSRMKDHPRPGAVIIVPLAQAAGLSHL

SEQ ID NO: 18 - protein, 304 aa, synthetic construct
MWLLAASVLLAALRAGSAQLSLYGTSVVEKNDCNKTVVLPCYVTDLKQNNENVMSVTWKRQGVTIFFYHGGNKKFNIDPSFSSARFLSKAELIKGVASLVIDSAQATVGNYSCKVTESNSEGEIKMELKNSSGSWFLLMERVGIVSLMCLLVILCVAQLCVIGLKYEIESQRLVYMIVALVIFAVVAGVGAALFIPDGYTVRNQAGLGLIVIPAVILVPLQYFMFGIVFDSLLRATLALIGLKLLGFIIAVVGFALCVSACPPLHVSVLIAGLAIMAFASLCSLVYVFFMGSRMKDHPRPGKAV

SEQ ID NO: 19 - protein, 309 aa, synthetic construct
MWPLVAAHLLSSASSGSAQITFNTTKFVEITVCNETVVLPCFANNVETKNLRELYLKWKFRGKDIFIYDGAQDLATPSSNFSSAEITPSELLKGNASLKMKKSDAVLGNYTCEVTELSREGETVVELKYRVVSWFSPKENILIIIFPILAILLSWGQFGIVTLKYKSNRTNEKTILMFVAGLLLTIVIIVGAILFIPGEYSIKNASGLGLIVIPTVILILLQYCAFMTAFGMTSFAIAILILQVLGYVLAVVGLSLCISECTPVQGPLLISGLGIIALAELLGLVYMKFVASNQKTIQPPRKAVAFKED

SEQ ID NO: 20 - protein, 318 aa, synthetic construct
MWLLAVSVLLGALRAGSAQLSLSGASVIEKNDCNDTVVLPCYVTNLKQNSENAMFVTWKKEGVTIFSYHGGNKKFNRDPSFLSARFLSQPDLIKGVASLVLGSEQATVGNYSCKVTESNREGEIKMELKNSSVVSCGDETPEEKCGSWFLLVERAVIISLVCLLVILCAAQLSFIGLKYEIESQRKVCIIVALVIFAVVAGIGAALFIQDGYTVQNQAGLGLIVIPAVILVPLQYVMFGIVFDSLLQATLALIGLKLLGFIIAVVGFALCVPACPPLHGSVLIAGLAIMAIASLLSLAYVFIMGSRMKDHPRPGKAVE

SEQ ID NO: 21 - protein, 277 aa, synthetic construct
MGSAQLILSGTSIVEKNDCNETVVLPCYVTNLIQNNENVMFVTWTSQGVIIFSYHGGTKKVNTHPSFSSARLLSPPELIKGVASLVLNNAQATVGNYSCKVTESNREGKIRMQLITSSGSWFLLVERAVIVSLVCLLVILCAAQLTVIGLKYEIESQRKVCIIVALVIFAVVAGVGAALFIQDGYTVQNQAGLGLIVIPAVILVPLQYVMFGIVFDSLLQATLALIGLKLLGFIIAVVGFALCVPACPPLHGSVLIAGSAIMAIASLLSLAYVFIMG

SEQ ID NO: 22 - protein, 276 aa, synthetic construct
MGSAQLTLVGTSIIERNDCNATVVLPCYVTNLEQNNEKVMFVTWKKQGNTIFSYRGETKKTNTDPAFSSARFLSQVDLVKGVASLVLSSAQATVGNYSCEVTESNREGEVKMELRNSSGSWFLLVERAVMISLVCSLVILCAAQLTVIGLKYEIESQRKVCIIVALVIFAVVAGVGAALFIQDGYTVQNQAGLGLMVIPAVILVPLQYVMFGIVFDSLMQATLALIGLKLLGFIIAVVGFALCVPACPPLHGSVLIAGLAIMAIASLLSLAYVNGL

SEQ ID NO: 23 - protein, 275 aa, synthetic construct
MGSAQLSLVGTGVIEKNDCNETVVLPCYVTNLKENNENLMLVTWKKQGDIIFSYHGGKKEFNINPSFSSARLLSQPDLIKGVASLVLSSAQATVGNYSCEVIESNSVGEIKMELRNSSGSWFLLVERAVIISLMSLLVVLCAAQLSVIGLKYEIESQRKVCSIVALVIFAVVAGVGAALFVQDGYTVQSQAGLGLTVIPAVILVPLQYVMFGIFDSLPQATLALIGLKLLGYIIAVVGFALCVPACPQLHGSVLIAGLAIMAIASLLSLAYVFIM

SEQ ID NO: 24 - protein, 276 aa, synthetic construct
MGSAQLILTGTDVVEKNDCNETVILPCYVTNLKENNENVMFVTWKKEGKVIFSFRGGKKEFYKDPAFSSAKLLSQADLTKGVASLVLNTAQATVGNYSCEVTESNREGETRMELRQSSGSWFLLVERAIIISLLFLVIILCAAQLSVIALKYEIESQRKVCIIVAVVILAVVAGVGTALFVQDGYTAQNQAGLGLIVVPAVILVPLQYFMFGIVFDSLPQTTLALIGLKILGYIIAVVGFALCVPACPPLHGSVLIAGLAIMAIASLLSLAYVFIM

SEQ ID NO: 25 - protein, 290 aa, synthetic construct
CGSAKLNLTGTDVVEKNDCNKTVILPCYVTNLMKNNETLMFVKWKKEGKVIFSFHGGRNEFYRDGDFSSAKLLSRADLTKGVASLVLTTRQATVGNYSCEVTESNTEGDTKMELRQSSGSWFLLVERAIIISLLCIVIILCAAQLTVIALKYDIEFQRKVCIIVAVVILAVVAGVGTALFVQDGYTALNQAGLGLIVVPAGILVPLQYFMFGIVFDSLPHTTLALIGLKILGYIIAVVGFALCVPACPPLHASVLIAGLAIMATASLLSLVYVFIMGSRMKDHPRPGKAV

SEQ ID NO: 26 - protein, 317 aa, synthetic construct
MWLLPAWVLLSAVGAGSAQLIFSGTDVVERTDCNKTVILPCYVTNLKDNNPNVTFVTWKKQRKIIFSFDGKNQEFFRDPEFQSADLVSRADLPKGIASLMLSGAEATVGNYSCEVTESNREGETRIELRNHSGSWFLLMERAIIIALLFLVITLCAAQSSVIVLKYETVPQKKTGIIVAVIIFVVAAVVGTVLFVQDGYTAQNQAGLGLIVVPAVILVPLQYVMFGIVFDSLPQATFALIGLHILGYVIAVVGFALCVSACPPLHGSVVIAGLAIMAIANLLSLAYVFIMGSRMKDHPRPGKAVEEPLNDLQSNVIC

SEQ ID NO: 27 - protein, 304 aa, synthetic construct
MWALGAWVLLGAVCAGSAQLQFNVIKSVEQTICNKTVILPCIVTNLAQNNTKVMFVKWKLEKKEFFSFDGSTEKIHRNENFSSAKLESLQNLKLGIASLTISKTQAVPGNYSCEVTESNREGETVIELIYRIVSWFTPVENILIILFMVLAILFYWIQLAVTVSKFETTLLKIGLFIAGVVITVFAVVGFSLLISDEFKPENQIGLGLIVLPAVILVPSQYFLLQIVCENNLPSFAIGVVVLESLGYLIAVIGFGLCITACRPKNGAVVIAGLAIMALTALVGLIYAILMGSSKKDQPPRKAVD

SEQ ID NO: 28 - protein, 304 aa, synthetic construct
MWALGAWVLLGAVCAGSAQLQFNAIKSVEQTICNKTVILPCIVTNLAQKNIKVMFVKWKLEKKEFFSFDGSTEKTARNNNFSSAVLESVQNLSLGIASLSISKTQAVPGNYSCEVTESNREGETVIELIYRIEPWFPQVESILIILFLVLAILFYWIQLGVTVSKGTTFQEKIGLIIAGIIITVVAVVGFILLVPDGFEPKNQAGLGLIVLPAVILVPLQYFLLKIVFKNNLPPFAILVVVLESLGYLIAVIGFALCITACPPKNGAVVIAGLAIMALTAVVGLIYVIALGSSKKDQPPRKAVD

SEQ ID NO: 29 - protein, 323 aa, synthetic construct
MWPLVLLLLLGSACCGSAQLIFNVIKSVEYTVCNKTVIIPCFVTNVKATNISDMFVKWKFKGKDIFIYDGQNQNQMSPSSEGFKSTEITPSELLKGVASLKMDKSDACVGNYTCEVTELSREGETIIELKYRVVSWFSPKENILIVIFPILAILLSWGQFGIVTLKYKSSDTKKKIIFIFVVGLVLTIDVIVGAILFVPGEFSTKNACGLGLLVIPTAALILLQYCVFMISFRMTAFTIAILILQGLGFVLAVDGLSLCITECTPVHGPLLISGLGIIALAELLGLIYMKLAASNQTTIQPPRKAVEEPLNAFKESKGMMNDE

SEQ ID NO: 30 - protein, 305 aa, synthetic construct
MWALRVWVLLGTLGAGSAQLLFEKVRSVEMDSCNSTVIIPCIVTNLKLNNTKAMFVKWKLQGKEFFSYDGFEDKFHRNSSFQSANLLNLSNLPNGIASLSLSRKEAIPGNYTCEVTESNREGETIVELRYSSASWFQPVENFLIIATMILAVILYWSQLGVVAIKFDMTLQKKIGLIIAGLIVTIVAVIGAILLVPDGYTASNQAGLGLIVVPAVILVPLLYFLFQSVFEKPPLFAIILVVLKALGYIIAVAGFALCVSACPPKQGSVVIAGLAIIDLVAVIGLIYIIVIGSNLKDHQPPRKAVD

SEQ ID NO: 31 - protein, 311 aa, synthetic construct
MWPLLVLLLLSSACRGSAQLIFNITKFAEYTTCNKTVVIPCLVNNVGAQSLKEMFVKWKFRDKYIFFYDGDKNESSPNSNFSSAFIVPEALLSGIASLKMDKSDAVPGNYTCEVTELTREGETIVELKYRVVSWFSPNENILIVIFPMLAVLLFWGQFGIVTLKYNASVTKEKTILLFVAGLVLTTFVIVGAILFVPGEYSTKRASGLGLIVVPTVILILLQYSVFMIAPAIGMSSFSITILILQVLGFVLSVVGLSLCVSECTPMHGPLLISGLGIIALAELLGLVYMKFVASNQRAIQPPRKAVAFKED

SEQ ID NO: 32 - protein, 305 aa, synthetic construct
MWPLLVLLLLGLASCGKTQLLFTTIESVEYTICNETLLIPCLVTNVGAKSLKEMFVKWRFRDKQIYFYDGDKNESNPTSDFSSAYIVPDALLTGNASLMMDKSDAVPGNYTCEVTELTREGETTIELKYRIALWFSANDYVLTIIFPMLAVLLFWGSDIIVTLKYNSNITKEKIIILSVAGLVLSILVIVGAVFFIPGEYSTKRASGLGLIVASTVILILLQISMFMIAPAKRKLPISIILLIVQVLGLLISVVGLSFCVSECIPVHGSLLISGLGITTLGALLGLVYMIFVASKQNTIHPTTET

SEQ ID NO: 33 - protein, 304 aa, synthetic construct
MWPLLVLVLLGLASCGKTQLLFTTIESVDYTICNETLLIPCLVTNVGAMSLKEMFVKWRLGDKQILFYDGDKNETNPISDFPSAYIIPNALLTGDASLMMDKSDAVPGNYTCEVTELTREGETTIELKYHIALWFSANDYVLTIIFPMLAVLLFWGSDIVTLKYNSNITKEKIIILSVAGLVLSILVIVGAVFFIPGEYSTKRASGLGLIVASTVILILLQISMFLIAPAKRKLPISVILLIVQVLGLLISVVGLSFCVSECIPVHGSLLISGLGITTLGALLGLVYMIFVASKQNTIHPQTDK

SEQ ID NO: 34 - protein, 311 aa, synthetic construct
MWPLLVLLLLGSASRGSAQLIFNITKFVDYTTCNQTVVIPCIVNNVEAKSLREMFVKWKFRDIYFFSYNGEKNESKPNSNFSSAYIVPEALLSGIASLKMDKSQAVPGNYTCEVTELTREGETTIELKYRVVSWFSPNENILIVIFPMLAVLLFWGQFGIVTLKYNASVTKEKTILLFVAGLVLTTFVIVGAILFVPGEYSTKRASGLGLIVVPTVILILLQYSVFMIAPAIGMSSFSITILILQVLGFVLSVVGLSLCVSECTPMHGPLLISGLGIIALAELLGLVYMKFVASNQRAIQPPRKAVAFKED

SEQ ID NO: 35 - protein, 311 aa, synthetic construct
HSPSAAVHLASKACCGSAQLTFNAVKSVEYTACNDTVIIPCFVNNVEADNLKEMYLKWKFGKSWIFIYDGDRDVSNHSEGYSSAEINKLGLLKGIAALKMKRKDAVPGNYTCEVTELSREGETIIELKYREVSWFSTNENILIIIFPMLAVLLFWGQFGIVTLKYSSSVSREKSIFLFIAGIAITAFVVVGAILLVPGEYSTKKASGLGLIVVPTVILILLQYSVFMISPAVGLSSFSIAILVIQLLGFVLSVVGLSLCVSACSPVHGPLLISGLGVVALAELLGLVYMKFNASDQRTIQPPRKAVAFKED

SEQ ID NO: 36 - protein, 311 aa, synthetic construct
MWPLVVLLLLSSACCGSAQLTFNPVKSIEYTTCNETVVIPCFVTNVEADSLREMYLKWKFGKDWIFIYDGDRDFSNHSRGYSSANIVKFALLKGIASLRMNKKDAVPGNYTCEVTELSREGETYIELKYREVSWFSTNENILIIIFPMLAVLLFWGQFGIVTLKYSSSVPKEKSIFLFVAGIVLTAFVIIGAILLVPGEYSAKKASGLGLIVVPTVILILLQYSMFMISPAVGLSSFSIAILVIQLLGFVLSVVGLSLCVSACSPVHGPLLISGLGVVALAELLGLVYMKFNASDQRTIQPPRSSVANKTT

SEQ ID NO: 37 - protein, 311 aa, synthetic construct
MWPLVVLLLLASASCGSAQLILNKTKPVEYTICNKTVVIPCFVSNVEATVLDDFYLKWKFGNENVFTFDGEQRSSTPGKNFSSATIVPEALLNGTASLKMDRSDAVPGNYSCEVTELSREGEIIVELKHRVVLWFSPNENILIIIFPMLAILLFWGQFGVVTLKYNSTVTKEKTILLFVAGLVLTIVMIVGAILFIPGEYSTKKASGLGLIVVPTVILILLQYSVFMIAPAIGMSSFSIAILILQVLGFVLSVVGLRLCISECIALHSPLLISGLGIIALAELLGLVYMKFIASNQRTIQPPRKAVEFKED

SEQ ID NO: 38 - protein, 309 aa, synthetic construct
MWPLVVLLLLGSLRCGSAQLIFNVIKSVEYTVCNQTVVIPCFVNNVETKNITELYVRWKFKGENIFIFDGSQRMSKSSSNFSSAKITPSELLRGIASLKMDKSDAVSGNYTCEVTELSREGETIIELKYRVVSWFSPNENILIVIFPVLAILLFWGQFGIVSLKYKSNYTKEKAIFLLVAGLLLTVLVIVGAFLFIPGEYSTKNASGLGLIVLPTVILILLHYCVFMIAMGMSSITISILILQILGYVLSVVGFSLCVSECVPVHGPLLISGLGIIALAELLGLVYMKCVGNSNHTIQAFASAVCMSEQ

SEQ ID NO: 39 - protein, 277 aa, synthetic construct
MLRVRILLIYLYTLVVITTTKTIEYTACNDTIIIPCTIDNPTKYIRWKLDNHDILTYNKTSKTTILSKWHTSARLHSLSDNDVSLIMEYKDILPGTYTCEDNTGIKSTVKLVQRHTNWFNDYQTMLMFIFTGITLFLLFLEIAYTSISVVFSTNLGILQVFGCIIAMIELCGAFLFYPSMFTLRHIIGLLMMTLPSIFLIITKVFSFWLLCKLSCAVHLIIYYQLAGYILTVLGLGLSLKECVDGTLLLSGLGTIMVSEHFSLLFLVCFPSTQRDYY

SEQ ID NO: 40 - protein, 277 aa, synthetic construct
MTNSKLLLVIYFTLFDIITTKTIEYTACNDTIIIPCTIDNPTKYIRWKLDNHDILIYNKTSKTTILSKWHTSARLHSLSDNDISLIMEYKDILPGNYTCENNKGIKHTVKLIQRHTNWFNDHQTMLMFIFTGITLLLLFSQIAYTSISVIYSTNLGILQVFGCIIAMIEVSGAFLFYPSMFTLRHIIGLLMMTLPSIFLIITKVFSFWILCKLSCAVHLIIYYQLAGYILTVLGLGLSLKECVDGTLLLSGLGTIMVSEHFSLLFLVCFPSTQRDYY

SEQ ID NO: 41 - protein, 298 aa, synthetic construct
MWPLVAVLLLSSLCCGSSQLLFKNINSVTVSSCDKVVVIPCYVSNIETEDIKGLKLNWKFGNTVILIFEGSVNKYSKTDNFQSAWISPLALIKGNASLKLNKNEAKEGNYTCTVRESRREGKHTVELKYRRASWFPLSENILIIISPFMTVLFFWAQFSILIRKYKSNPVNKKIILLSTSGLLLTIIVILGAILFIPGNYSKKKSYGLILFVTPSVIPIVLQYYKFKPAIGRTLHAMIIFTVLQASGFVLAMAGLYLTVNECVPKHGSLLVSGLVIIALVELFGLIYMKWLDPLIGFL

SEQ ID NO: 42 - protein, 327 aa, synthetic construct
MWPLVAALLLGSCCCGSAQLLFSKVKSVEYTKCNETVVIPCIVLNVEAQSTEEMYVKWKLNQTYIFIYDGSKNNSTVETNFSSAKISVSDLLKGDASLKMGRTQAVVGNYTCEVTELSREGKTVIELKHRMDSNSSHGADCSNHTGDEKEASWFSPNEKILIVIFPILAILLFWGKFGILTLKYKSTRTNKRIILLLVAGTVLTVIVIVGAILLIPGENPVKSASGLGLIVISIGILIPLQYNVFMTAFGMASFTIAILITQVLGYVLALVGLCLCIMACEPVHGPLLISGLGIIALAELLGLVYMKFVASNQRTIQPPRKAVAFKE

SEQ ID NO: 43 - protein, 309 aa, synthetic construct
MWPLVATLLLGSWCFGSAQLLLNKTKLVEYTICNKTVVIPCFVINVGAQSTKEMFVKWKFNGTYIFNYDGSANNSTAKKNFSSAKIEVSKLLKGDASLQMSKEDVKVGNYTCEVTELSREGETVVEVKYRMVSWFSPNENILIVIFPILAILLFWGQFGILTLKYKSNCTNMKIILSLIAGLLLTIIVIVGAILFIPGEYSIKNSFGLGFIVISTGVLILLQYNVFMTAFGMTSFTIAILITQVLGYVLAVVGLCLCVTACVPADGPLLISGLGIVALAELLGLVYMKFVASNQRTIQPPRKAVAFKEE

SEQ ID NO: 44 - protein, 321 aa, synthetic construct
MLPLIKKVLTITQGTGSAQLLFDKTRLVEYTSCNETVVIPCFVTNVGAQNPGEMFVKWKFNRTYIFIYDGSRNNSTADSNFSSAKISVSELIKGDASLKLDKADAVVGNYTCEVTELSREGETVVELKYRVVSWFSPNENILIVIFPILAILLFWGQFGLLTLKYKSNRTNRKIILLLIAGVVLTAIVIVGAILFIPGEYSVKNASGLGLIVISTGILILLQYNVFMAAFGMTSFTIAILITQVLGCVLAMVGLCLCMFACVPVHGPLLISGLGIIALAELLGLVYMKFVASNQRTIQPPRKAVEEPLNAFKESKGMMNDE

SEQ ID NO: 45 - protein, 323 aa, synthetic construct
MWPLVAALLLGSACCGSAQLLFNKTKSVEFTFCNDTVVIPCFVTNMEAQNTTEIYVKWKFKGRDIYTFDGALNRSTLPSGFNSAKIEVSQLLKGDASLKLDKSDAASHTGNYTCEVTELTREGETIVELKYRVVSWFSPNENILIVIFPIFAILLFWGQFGIKTLKYRSGGMDEKTIALLVAGLTITVIVIVGAILFVPGEYSLKNATGLGLIVTSTGILILLHYYVFSTAFGLTSFVIAILVIQVIAYVLAVVGLSLCIAACIPMHGPLLISGLSILALAQLLGLVYMKFVASNQKTIQPPRKAVEEPLNAFKESKGMMNDE

SEQ ID NO: 46 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTAPANFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLMITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNEFKEFRGTPTVS

SEQ ID NO: 47 - protein, 323 aa, synthetic construct
MWPLVAALLLGSACCGSAQLLFNKTKSVEFTFCNDTVVIPCFVTNMEAQNTTEVYVKWKFKGRDIYTFDGPLNKSTAPANFSSAKIEVSQLLKGDASLKLDKSDAVSHTGNYTCEVTELTREGETIIELKYRVVSWFSPNENILIVIFPIFAILLFWGQFGIKTLKYRSGGMDEKTIALLVAGLMITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 48 - protein, 320 aa, synthetic construct
MWLLTAGALLAVLGAGSTQLVFNVVDLVEKNACNETVILPCIVTNLKENNDSSMHVSWKRQGQVLFSFNGPEQRIYRHESVPSANFLSKSDLSKGIASLRLKNAEATDGNYSCEVTELNREGETKFELRTRMVDSCDEEKPPTPVDKCERQFDLVQSIVIAALLVFIIVLCWTQLGVVAFKCETVRKKKKHITIPCSIFTVIAVVAVVLFIQDGSISMNQVGLAFTVLPAGMLMVLQYSIFKLILDDLTHKGYALIAFQTVGYIVAVIGFALSVSACPSILLSVVIAGLVVMAVADLLALVYVYSCKIAEAIQTPRKAVD

SEQ ID NO: 49 - protein, 336 aa, synthetic construct
MSLPAACPPLLLLLLLLLGALRHGSAQLVFSPVDFVERDACNTTVVLPCHVVNLKEHNDSNMFVAWKKQGIVLFSFDGAQQRVYRHETVPSANLLSQQDLSKGTASLKLKSSEAEDGNYSCEVTELNREGETKVDLRTVTVDNCGEEKAPTPDNKCGRQFDFMESVVISVLLFLIVVFYWAQIGVIALKCETVHKNKTYVAVGCAIFTVLAVVAVVLFIQGGNITINKVGLGFVVLPAVILMVLQYYIFKIVLVDVTRKGYILIGLQSFGFVVALVGFALCISACPSELLSVVIAGLAVMAFEDLLALLYMYTSCSRMKDHQTPRKAVELKTETES

SEQ ID NO: 50 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVSTNFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLMITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 51 - protein, 277 aa, synthetic construct
MGSAQLMFNVTDVVEKTDCNKTVILPCHVINLKENNADIMFVTWKRQGKRIFSFDGAGRKSLRDPAVPSANLVSEADLPKGVASLRLNSAEAVVGNYSCEVTESNREGETRVELRKYSGSWFLLVERAIIIALLFLVIILCSAQLIVIALKYEIAPQKRMGIIVAGVISTVAAVVGTVLFVQDGYTAQNQAGLGLIVVPAVILVPLQYFMFGMVFDSLPQATFALIGFQILGYIIAVVGFALCVSACPPIHGSVVVAGLAIMAIANLLSLAYVFIMG

SEQ ID NO: 52 - protein, 277 aa, synthetic construct
MGSAQLTLSGTSIIEKNDCNETVVLPCYVTNLKQNNENVMFVTWKKQGVTIFSYHGGNKKFNIDPSFSSARFLSQADLIKGVASLVLDSAQATVGNYSCKVTESNREGEIKMELKNSSGSWFLLVERAVIISLMCLLVILCAAQLSVIGLKYEIESQRKVCIIVALVIFAVVAGVGAALFIQDGYTVQNQAGLGLIVIPAVILVPLQYVMFGIVFDSLLQATLALIGLKLLGFIIAVVGFALCVPACPPLHGSVLIAGLAIMAIASLLSLAYVFIMG

SEQ ID NO: 53 - protein, 293 aa, synthetic construct
MVLLGALGSAQLILSGTSVIEKNGCNKTVVLPCYVTNLRQKNIEAMFVIWKKEGTTIFSYRGATKNFNIDPSFSSAKFLHLENLTNGDASLVLNSKQAIVGNYSCEVTESNREGEFRLELRNGSGSWFLLVERAVIISLVILLVILCAAQLIFIGLKYEIESQKKVCVIIALVIFAIVAGVGAALFIQDGYTVQNQAGLGLMVIPAVILVPLQYVMFGIVFDSQLQATLALIGLKLLGFIIAVVGFALCVPACPPLHGSVLIAGLAIMAIASLLSLAYVFIMGSRMKDEEPLN

SEQ ID NO: 54 - protein, 277 aa, synthetic construct
MGSAQLTLVGTSVIEKNDCNETVVLPCNVTNLEQNNENVMFVTWKKQGDTFFSYRGENKKFNIDPSFSSARFLSQADLVKGVASLVLNSAQATVGNYSCEVTESNREGEIKMELRNSSGSWFLLVERAVIISLVCLLVILCAAQLIVIGLKYEIESQRKVCIIVALVIFAVVAGVGAALFIQDGYTVQNQAGLGLIVIPAVILVPLQYVMFGIVFDSLLQATLALIGLKLLGFIIAVVGFALCVPACPPLHGSVLIAGLAIMAIASLLSLAYVFIMG

SEQ ID NO: 55 - protein, 276 aa, synthetic construct
MGSAQLSLVGTDIIEKNDCNETVVLPCYVTNLKENNENVMFVTWKKQGEIIFSYHGGKKEFNIHPSFSSARLLSQADLIKGVASLVLSSAQATVGNYSCEVTESNREGEIKMELRNSSGSWFLLVERAVIISLMCLLVILCAAQLSVIGLKYEIESQRKVCIIVALVIFAVVAGVGTALFVQDGYTVQSQAGLGLTVIPAVILVPLQYVMFGIVFDSLPQATLALIGLKLLGYIIAVVGFALCVPACPPLHGSVLIAGLAIMAIASLLSLAYVFIM

SEQ ID NO: 56 - protein, 310 aa, synthetic construct
MWPLVVVLLLNSAYCGSAQLILNVTKFVEYTNCNTTVLLPCIVYNIEATGLEELFVKWKFGGKYIFTYDGAKREPKPNSNFSSAKIFPDDLLKGNASLEMDKEHAVPGNYTCEVTELSREGDTVVELKYRLVSWFSPNENILIVIFPMLAILLFWGQFGIVTLKFNSSIMKEKTILLFVAGLVLTIVVIVGAILFIPGEYSTKRASGLGLIVVPTVILILLQYAVFMIAPAIGMSSFSIAILILQVLGFVLSVVGLSLCVSVCAPVHGPLLISGLGIVALAELLGLVYMKFTATNQRTIQPPRKAAFKED

SEQ ID NO: 57 - protein, 272 aa, synthetic construct
MWPLVVLLLLGSLRSGSAQLIFNVIKSVEYTVCNQTVVIPCFVNNVETKNVTELYVRWKFKGENIFIFDGSQRMSKSSSNFSSAKITPSELLRGIASLKMDKSDAVSGNYTCEVTELSREGETIIELKYRVVSWFSPNENILIVIFPVLAILLFWGQFGIVSEYSTKNASGLGLIVLPTVILILLHYCVFMIAMGMSSITISILILQILGYVLSVVGFSLCVSECVPVHGPLLISGLGIIALAELLGLVYMKCVGKSALLLLAYAGNCMSSQ

SEQ ID NO: 58 - protein, 277 aa, synthetic construct
MGSAQLILTATDVVETTNCNKTVVLPCYVTNLQEKNEKVMFVLWKRQGQVIFSFDGANQEVFRDPTVPSANLVSQADLPKGIASLMLNSAEAEVGNYSCQVTESSREGQVKVELRNRSGSWFLLVERAIIIVLLFLIIVLCSAQLSVVAFKYEIVPQKKVSIAVAGFIFTVAAVIGTVLFVQDGFTAQNQAGLGLIVIPAVSMVPLQYFIFRIVFDSLPQATFALIGLHILGYVIAVVGFALCVSACPPLHGSILIAGLAIMAITNLLSLAYVFIMV

SEQ ID NO: 59 - protein, 276 aa, synthetic construct
MGSAQLILTGTDIVEKNDCNETVILPCYVTNLKENNENVMFVTWKKEGKVIFSFHGGRNEFYKDPSFSSAKLVSQADLTKGVASLVLSTAQATVGNYSCEVTESNREGETRMELRQSSGSWFLLVERAIIISLLCIVIILCAAQLTVIALKYEIESQRKVCIIVAVVILAVVAGVGTALFVQDGYTAQNQAGLGLIVVPAVILVPLQYFMFGIVFGDLPQTTLALIGLKILGYIIAVVGFALCVPACPPLHASVLIAGLAIMATASLLSLVYVFIM

SEQ ID NO: 60 - protein, 278 aa, synthetic construct
MGSAQLIFNVTDVVEKTDCNKTVILPCHVINLKENNADIMFVTWKRQGKIIFSFDGARKEFFRDPTVPSANLVSQADLPKGIASLRLNSAEADVGNYSCEVTESNREGETRVELRKYSGSWFLLVERAIIIALLFLVIILCSAQLSVIALKYEIVPQKRMGIIVAGVISTVAAVVGTVLFVQDGYTAQNQAGLGLIVVPAVILVPLQYFMFGMVFDSLPQATFALIGLQILGYIIAVVGFALCVSACPPIHGSVVIAGLAIMAIANLLSLAYVFIMGV

SEQ ID NO: 61 - protein, 314 aa, synthetic construct
MWPLVAALLLGTVCGGSAQLTFNSTRFVEETICNETVILPCIVNNLEIKNIKVMYVKWKHKGKEIFTFDGDQNKPNASDNFKSATIALSLLVEGNASLLMKKSDAVPGNYTCEVTESSREGETTVELRYHVVSWFYPKENILIVIFPILAILLSWGQFGIVTLKYKSNCTSEKTIGLIIAGLALTVVVIVGAVLFVPGEYSVKNASGLGLIVIPTVTLVLLQYCVFMTAFGMTLFAIGILILQLLGYILAVIGLSLCITKCSPMQGPLMISGLGIIALAELLGLVYMKFVASNQKTIPPSRKAVEEPLNAFKED

SEQ ID NO: 62 - protein, 314 aa, synthetic construct
MWPLVVLLLLGSAGCGSAQLIFNITRSVEYTACNETVIIPCYVNNVEATNINEMYVKWKFRGKDIFTFDGAEEKITRDNKFKSTKIIPQKLLNGIASLEMNKEEAVVGNYTCEVTELSREGETIIELKYRIVSWFSPNENILIVIFPILAVLLSWGQFGIVTHKYKSSLMKEKTILLFVGGLVLTIAVIVGAILFVPGEYSTKNSCGLGLIVVPTVILILLQYCVFMIGVWMSSFTIAILILQVLGYVLSVVGLSLCVSECTPVNGPLLISGLGIIALAELLGLVYMKLVASNQKTIQPPRKAVEEPLNAFKED

SEQ ID NO: 63 - protein, 313 aa, synthetic construct
MLPLVVLLLGSACCGSAQLIFNITKSVEYTACNESVIIPCFVNNVEATNINEMYVKWKLKGKDIFTFNGALQKTTHGDKFKSTKIIPQKLLTGIASLEMDKEEAVVGNYTCEVTELSREGETIIELKYRIVSWFSPNENILIVIFPILAVLLSWGQFGIVTIKYKSSLVKEKTILLFVGGLVLTIVVIVGAILFVPGEYSTKNSCGLGLIVIPTVILILLQYCVFIIGVWMSPFTIAILILQVLGYVLSVVGLSLCVSECTPVHGPLLISGLGIIALAELLGLVYMKLVASNQKTIQPPRKAVEEPLNAFKED

SEQ ID NO: 64 - protein, 291 aa, synthetic construct
MGSAQLIFSVTDVVERTDCNKTVILPCYVTNLKENNANVMFVTWKKQGKIIFSFDGARQEFFRDPAVPSANLVSQADLPKGIASLMLNSAEADVGNYSCEVTESNREGETRVELRNHSGSWFLLVERAIIIALLFLVIILCSAQLSVIALKYEIVPQKKMGIIVAGIIFTVAAVVGTVLFVQDGYTAQNQAGLGLIVVPAVILVPLQYFMFGIVFDSLPQATFALIGLQILGYIIAVVGFALCVSACPPLHGSVVIAGLAIMAIANLLSLAYVFIMGSRMKDHPRPGKAVE

SEQ ID NO: 65 - protein, 312 aa, synthetic construct
MWPLVVLLLLGSACCGSAQLIFNITKSVEYTTCNKTVVIPCFVNNVEATSLKEMFVKWKFGGKYIFTYDGDKNESKPNSNFSSASIVPEALLNGIASLKMDKSDAVPGNYTCEVTELSREGETIIELKYRVVSWFSPNENILIVIFPMLAVLLFWGQFGIVTLKYNSSVTKEKTILLFVAGLVLTTFVIVGAILFVPGEYSTKRASGLGLIVVPTVILILLQYSVFMIAPAIGMSSFSIAILILQVLGFVLSVVGLSLCVSECTPVHGPLLISGLGIIALAELLGLVYMKFIASNQRTIQPPRKAVEAFKED

SEQ ID NO: 66 - protein, 312 aa, synthetic construct
MWPLLVLLLLGSASRGSAQLIFNITKFAEYTTCNKTVVIPCLVNNVEAQSLKEMFVKWKFRDKYIFFYDGDKNESSPNSNFSSAFIVPEALLSGIASLKMDKSDAVPGNYTCEVTELTREGETIVELKYRVVSWFSPNENILIVIFPMLAVLLFWGQFGIVTLKYNSNITKEKIIILSVAGLVLSILVIVGAVLFIPGEYSTKRASGLGLIVVPTVILILLQYSVFMIAPAIGMLPISIILLIIQVLGLLISVVGLSLCVSECTPMHGPLLISGLGIIALAELLGLVYMKFVASNQRAIQPPRKAVEAFKED

SEQ ID NO: 67 - protein, 312 aa, synthetic construct
MWPLVVLLLLSSACCGSAQLTFNPVKSVEYTTCNETVVIPCFVNNVEADSLKEMYLKWKFGKNWIFIYDGDRDVSNHSAGYSSASIVKFALLKGIASLKMNKKDAVPGNYTCEVTELSREGETIIELKYREVSWFSTNENILIIIFPMLAVLLFWGQFGIVTLKYSSSVSKEKSIFLFIAGIVLTAFVVIGAILLVPGEYSTKKASGLGLIVVPTVILILLQYSMFMISPAVGLSSFSIAILVIQLLGFVLSVVGLSLCVSACSPVHGPLLISGLGVVALAELLGLVYMKFNASDQRTIQPPRKAVEAFKED

SEQ ID NO: 68 - protein, 310 aa, synthetic construct
MWPLVVVLLLGSVRCGSAQLIFNVIKSVEYTVCNQTVVIPCFVNNVETKNITELYVRWKFKGENIFIFDGSQRMSKPSSNFSSAKITPSELLRGIASLKMDKSDAVVGNYTCEVTELSREGETIIELKYRVVSWFSPNENILIVIFPVLAILLFWGQFGIVTLKYKSNYTKEKAIFLLVAGLLLTVLVIVGAFLFIPGEYSTKNASGLGLIVLPTVILILLHYCVFMIAMGMSSFTISILILQILGYVLSVVGFSLCVSECIPVHGPLLISGLGIIALAELLGLVYMKCVASNHRTIQPPKKAVEAFKED

SEQ ID NO: 69 - protein, 310 aa, synthetic construct
MWPLVAALLLGSACCGSAQLLFNKTKSVEFTFCNETVVIPCFVINVGAQNTKEMYVKWKFKGKYIFIYDGGQNNSTADSNFSSAKIAVSELLKGDASLKMDKRDAVVGNYTCEVTELSREGETVVELKYRVVSWFSPNENILIVIFPILAILLFWGQFGILTLKYKSNRTNKKIILLLIAGLLLTTIVIVGAILFVPGEYSIKNASGLGFIVISTGILILLQYNVFMTAFGMTSFTIAILITQVLGYVLAVVGLCLCIMACVPVHGPLLISGLGIIALAELLGLVYMKFVASNQRTIQPPRKAVEAFKED

SEQ ID NO: 70 - protein, 310 aa, synthetic construct
MWPLVAALLLGSCCCGSAQLLFSKVKSVEFTSCNETVVIPCIVLNVEAQSTEEMYVKWKLNKTYIFIYDGSKNNSTVETNFSSAKISVSDLLKGDASLKMDRQQAVVGNYTCEVTELSREGKTVIELKYRMASWFSPNEKILIVIFPILAILLFWGKFGILTLKYKSTRTNKRIILLLVAGLVLTVIVVVGAILLIPGENPVKSASGLGLIVISIGILILLQYNVFMTAFGMTSFTIAILITQVLGYVLALVGLCLCIMACEPVHGPLLISGLGIIALAELLGLVYMKFVASNQRTIQPPRKAVEAFKED

SEQ ID NO: 71 - protein, 331 aa, synthetic construct
MWPLVAALLLGSCCCGSAQLLFSKVKSVEYTSCNETVVIPCIVLNVEAQSTEEMYVKWKLNKTYIFIYDGSKNNSTVETNFASAKISVSDLLKGDASLKMNKQNAVVGNYTCEVTELSREGKTVIELKYRMDVNCCQGSDCSNHTTPAGDEKEASWFSPNEKILIVIFPILAILLFWGKFGILTLKYKSTRTNKRIILLLIAGTVLTVIVIVGAILLIPGENPVKSASGLGLIVISIGILIPLQYNVFMTAFGMTSFTIPILITQVLGYVLALVGLCLCIMACEPVHGPLLISGLGIIALPELLGLVYMKFVASNQRTIQPPRKAVEAFKE

SEQ ID NO: 72 - protein, 310 aa, synthetic construct
MWPLAAALLLGSCCCGSAQLLFSKVKSVEFTSCNETVVIPCIVLNVEAQSTEEMFVKWKLNKSYIFIYDGNKNSSTVEQNFTSAKISVSDLLKGIASLKMDKHEAVVGNYTCEVTELSREGKTVIELKNRTVSWFSPNEKILIVIFPILAILLFWGKFGILTLKYKSSRTNKRIILLLVAGLVLTVIVVVGAILLIPGEKPVKNASGLGLIVISTGILILLQYNVFMTAFGMTSFTIAILITQVLGYVLAVVGLCLCIMACEPVHGPLLISGLGIIALAELLGLVYMKFVASNQRTIQPPRKAVEAFKED

SEQ ID NO: 73 - protein, 310 aa, synthetic construct
MWPLVATLLLGSWCFGSAQLLLNKTKFVEYTICNETVVIPCFVINVGAQSTKEMFVKWKFNGTYIFIYDGNKNNSTANLNFSSAKIAVSKLLKGDASLKMSKDDVKVGNYTCEVTELSREGETVVEVKYRMVSWFSPNENILIVIFPILAILLFWGQFGILTLKYKSNCTNTKIILLLIAGLLLTIIVIVGAILFIPGEYSIKNSFGLGFIVISTGILILLQYNVFMTAFGMTSFTIAILITQVLGYVLAVVGLCLCVTACVPVHGPLLISGLGIIALAELLGLVYMKFVASNQRTIQPPRKAVEAFKEE

SEQ ID NO: 74 - protein, 321 aa, synthetic construct
MWLLTAGALLAVLGAGSTQLVFNAVDFVEKYACNDTVVLPCIVINLKENNDSSMHVSWKRQGQVIFSFNGPEQRIYRHESVPSANFLSKADLSKGIASLRLKNAEATDGNYSCEVTELNREGETKMELRTHMVDSCDEEKPPTPVDKCERQFDLIQSIVIAVLLVFIIVLCWAQLGVIALKCETVRKKKKHITIACSIFTVIAVVAVVLFIQDGSISMNQIGLAFTVLPAGMLMVLQYSIFKMVLDDLTHKGYALIGFQVVGYIVAVVGFALSVSACPSVLLSVVIAGLVVMAVADLLALVYVYSCSRMKDHQTPRKAVED

SEQ ID NO: 75 - protein, 305 aa, synthetic construct
MWALGAWVLLGAVCAGSAQLQFNAIKSVEQTICNKTVILPCIVTNLAQNNTKVMFVKWKLEKKEFFSFDGSTEKIHRNNNFSSAKLESLQNLSLGIASLSISKTQAVPGNYSCEVTESNREGETVIELIYRIESWFPPVENILIILFLVLAILFYWIQLGVTVSKFETTLQKKIGLIIAGVIITVVAVVGFILLVPDGFKPKNQAGLGLIVLPAVILVPLQYFLLQIVFENNLPPFAILVVVLESLGYLIAVIGFALCITACPPKNGAVVIAGLAIMALTAVVGLIYVILMGSSKKDQPPRKAVD

SEQ ID NO: 76 - protein, 310 aa, synthetic construct
MWPLVAALLLGSVCGGSAQLTFNSTRFVEKTICNETVILPCIVNNLELKNIKAMYVKWKFKGKDIFTFDGNQNKSNASNNFKSANIALSDLLKGNASLKMKKSDAVPGNYTCEVTESNREGETIVELRYHVVSWFSPKENILIVIFPILAILLSWGQFGIVTLKYKSNCTSEKTIGLIIAGLILTIVVIVGAVLFIPGEYSIKNASGLGLIVIPTVILVLLQYCVFMTAFGMTSFAIAILILQLLGYILAVVGLSLCITECAPMQGPLLISGLGIIALAELLGLVYMKFVASNQKTIQPPRKAVEAFKED

SEQ ID NO: 77 - protein, 310 aa, synthetic construct
MWPLVVALLLGSACCGSAQLIFNVTKSVEYTVCNKTVVIPCFVNNVEAKNINEMYVKWKFKGKDIFIFDGAQQKSTPSNNFTSAKISPSELLNGIASLKMDKSDAVVGNYTCEVTELSREGETIIELKYRVVSWFSPNENILIVIFPILAILLSWGQFGIVTLKYKSSHTKEKTILLFVAGLVLTIVVIVGAILFIPGEYSTKNACGLGLIVIPTAILILLQYCVFMIAIGMSSFTIAILILQVLGYVLSVVGLSLCVSECTPVHGPLLISGLGIIALAELLGLVYMKFVASNQKTIQPPRKAVEAFKED

SEQ ID NO: 78 - protein, 314 aa, synthetic construct
MWPLVVLLLLGSARCGSAQLIFNTTKSVEYTVCNQTVVIPCFVNNMEAKSIAELYVRWKFKGKDIFIFDGVQQRSNSSDIFTSAKISPSELLNGIASLTMDKRDAVLGNYTCEVTELSREGETIVELKYRVVSWFSPNENILTVIFPILAILLFWGQFGVVTLKYKSSYTKEKTIFLLVTGLVLTIVVIVGAILFIPGEYSTKNACGLGLIVIPTAILILLQYCVFMIAVGMSFFTIAILILQVLGHVLSVVGFSLCVSECTPVHGPLLISGLGIIALAELLGLVYMKCVASNHKTIQPPRKAVEEPLNAFKED

SEQ ID NO: 79 - protein, 310 aa, synthetic construct
MWPLVAALLLGSWCCGSAQLLLSKVKSVEFTSCNETVIIPCFVLNVEAQSTKEMYVKWKLNRTYIFIYDGNKNNSTVDSNFSSAKISVSDLLKGDASLKMDKRDAVVGNYTCEVTELIREGKTVIELKYRMASWFSPNEKILIVIFPILAILLFWGKFGILTLKYKSTRTNKRIILLLIAGLVLTVIVIVGAILFIPGEYPVKNASGLGLIVISTGILILLQYNVFMTAFGMTSFTIAILITQVLGYVLAVVGLCLCIMACEPVHGPLLISGLGIIALAELLGLVYMKFVASNQRTIQPPRKAVEAFKED

SEQ ID NO: 80 - protein, 291 aa, synthetic construct
MGSAQLTFSGPDVVERTDCNRTVTLPCYVNNLKENNIKVMFVTWKKQGKIIFSFDGAKQEFLRDETVPSANLTSQEDLPKGIASLTLISAEAGVGNYSCEVTESNREGETRVELRHSSGPWFLLVESAIIIVLLLLVIIFCSAQFSFIALKYEIEPQKKTGIIVAGVIFIVAAVVGTVLFAQDGYTAQNQAGLGLIVVPAMIAVPLQYFMFGIVFDSLPPTAFVLIFFQILGYIIAVVGFALCVSACPPLHGSVVIAGLAIMASANLISLAYVFITGSRMKDHPRPGKAVE

SEQ ID NO: 81 - protein, 309 aa, synthetic construct
MWPLVAALLLGSACCGSAQLLFNKTKSVEFTFCNETVVIPCFVTNVEAENTNELYVKWKFKGKDIYTFDGAMNKSTTDSDFKSARIEVSELLKGDASLKMDKSDAVLGNYTCEVTELSREGETIIELKYRVVSWFSPNENILIVIFPIFAILLFWGQFGIKTLKYKSSGMDEKTIALLVAGLVITAIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAVAFKED

SEQ ID NO: 82 - protein, 290 aa, synthetic construct
LGSAQLMFSATDVVEVTKCNKTVILPCYVTNLKENNSSVMFVTWKKQRKTIFSFDGEKQQFFRDPAVPSANLLSQKDLPKGVASLILNSAEADAGNYSCEVTESNREGETTVELRKPSGSWFLLVERAIIIVLLFLVIIFCSAQLSVIALKYGILPQKKMGIVVAGITFTLAAVVGTVLFVQDGYNVQNQAGLGLIVVPALILVPLQYFMFGYVFDSLPQATFVLIGLQILGYIIAVIGFALCVSACPPLHGSVVIAGLAIMAIANLLSLAYVFIMGSSMKDHPRPGKAV

SEQ ID NO: 83 - protein, 309 aa, synthetic construct
MWPLVAALLLGSACCGSAQLLFNKTKSVEFTFCNETVVIPCFVTNVEAENTKEMYVKWKFKGKDIFIFDGAQNKSTAYNNFTSAKIAVSELLKGDASLKMDKSDAVLGNYTCEVTELSREGETIVELKYRVVSWFSPNENILIVIFPILAILLFWGQFGILTLKYKSNRTNEKTILLLVAGLLLTTVVIVGAILFIPGEYSIKNASGLGLIVISTGILILLQYCVFMTAFGMTSFTIAILIIQVLGYVLAVVGLSLCISACVPVHGPLLISGLGIIALAELLGLVYMKFVASNQKTIQPPRKAVAFKED

SEQ ID NO: 84 - protein, 309 aa, synthetic construct
MWPLVAALLLGSACCGSAQLLFNKTKSVEFTFCNETVVIPCFVTNVEAENIKEMYVKWKFKGKDIFIFDGAQNKSTAYNNFTSAKIAVSELLKGDASLKMDKSDAVLGNYTCEVTELSREGETIVELKYRVVSWFSPNENILIVIFPILAILLFWGQFGIVTLKYKSNRTNEKTILLLVAGLLLTIVVIVGAILFIPGEYSIKNASGLGLIVIPTGILILLQYCVFMTAFGMTSFTIAILIIQVLGYVLAVVGLSLCISACVPVHGPLLISGLGIIALAELLGLVYMKFVASNQKTIQPPRKAVAFKED

SEQ ID NO: 85 - protein, 309 aa, synthetic construct
MWPLVAALLLGSACCGSAQLIFNVTKSVEFTVCNETVVIPCFVNNVEAKNIKEMYVKWKFKGKDIFIFDGAQNKSTPSNNFTSAKITPSELLKGIASLKMDKSDAVLGNYTCEVTELSREGETIVELKYRVVSWFSPNENILIVIFPILAILLSWGQFGIVTLKYKSNRTNEKTILLFVAGLLLTIVVIVGAILFIPGEYSIKNASGLGLIVIPTAILILLQYCVFMTAFGMTSFTIAILILQVLGYVLAVVGLSLCISECTPVHGPLLISGLGIIALAELLGLVYMKFVASNQKTIQPPRKAVAFKED

SEQ ID NO: 86 - protein, 309 aa, synthetic construct
MWPLVAALLLGSACCGSAQLTFNATKSVEFTVCNETVVIPCFVNNVETKNIKEMYVKWKFKGKDIFIFDGAQNKSTASSNFTSAKITPSELLKGIASLKMKKSDAVLGNYTCEVTELSREGETIVELKYRVVSWFSPKENILIVIFPILAILLSWGQFGIVTLKYKSNRTNEKTILLFVAGLLLTIVVIVGAILFIPGEYSIKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAVAFKED

SEQ ID NO: 87 - protein, 309 aa, synthetic construct
MWPLVAALLLGSACCGSAQLTFNATRFVEVTICNETVVLPCIVNNVESKNIKDMYVKWKFKGKDIFTFDGNQNKSNASSNFTSANITPSELLKGIASLKMEKSDAVLGNYTCEVTELSREGETIVELKYRVVSWFSPKENILIVIFPILAILLSWGQFGIVTLKYKSNRTNEKTILLIVAGLLLTIVVIVGAILFIPGEYSIKNASGLGLIVIPTVILILLQYCVFMTAFGMTSFAIAILILQVLGYILAVVGLSLCISECAPMQGPLLISGLGIIALAELLGLVYMKFVASNQKTIQPPRKAVAFKED

SEQ ID NO: 88 - protein, 309 aa, synthetic construct
MWPLVAALLLGSVCGGSAQLTFNSTRFVEKTICNETVILPCIVNNLELKNIKAMYVKWKFKGKDIFTFDGNQNKSNASNNFKSANIALSDLLKGNASLKMKKSDAVPGNYTCEVTESNREGETIVELRYHVVSWFSPKENILIVIFPILAILLSWGQFGIVTLKYKSNCTSEKTIGLIIAGLILTIVVIVGAVLFIPGEYSIKNASGLGLIVIPTVILVLLQYCVFMTAFGMTSFAIAILILQLLGYILAVVGLSLCITECAPMQGPLLISGLGIIALAELLGLVYMKFVASNQKTIQPPRKAVAFKED

SEQ ID NO: 89 - protein, 309 aa, synthetic construct
MWPLVAALLLGSVCGGSAQLTFNSTRFVEKTICNETVILPCIVTNLELNNIKAMFVKWKFKGKDIFTFDGNQNKSNASNNFKSANISLSDLLKGNASLKMKKSDAVPGNYTCEVTESNREGETIVELRYHVVSWFSPKENILIVIFPILAILLSWGQFGIVTLKYKSNCTSEKTIGLIIAGLILTIVVIVGAVLFIPGEYSIKNASGLGLIVIPTVILVLLQYCVFMTAFGMTSFAIAILILQLLGYILAVVGLSLCITECAPMQGPLLISGLAIIALAELLGLVYMKFVASNQKTIQPPRKAVAFKED

SEQ ID NO: 90 - protein, 309 aa, synthetic construct
MWPLVAALLLGSACCGSAQLLFNKTKSVEFTFCNDTVVIPCFVTNMEAQNTTEVYVKWKFKGRDIYTFDGALNKSTVPSDFKSAKIEVSQLLKGDASLKMDKSDAVTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAFGLTSFVIAILVIQVIAYVLAVVGLSLCIAACIPMHGPLLISGLSILALAQLLGLVYMKFVASNQKTIQPPRKAVAFKED

SEQ ID NO: 91 - protein, 309 aa, synthetic construct
MWPLVAALLLGSACCGSAQLLFNKTKSVEFTFCNETVVIPCFVTNMEAENTNEVYVKWKFKGKDIYTFDGALNKSTTDSDFKSARIEVSELLKGDASLKMDKSDAVLGNYTCEVTELSREGETIIELKYRVVSWFSPNENILIVIFPIFAILLFWGQFGIKTLKYRSGRMDEKTIALLVAGLVITVIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAVAFKED

SEQ ID NO: 92 - protein, 309 aa, synthetic construct
MWPLVAALLLGSACCGSAQLLFNKTKSVEFTFCNETVVIPCFVTNVEAENTNELYVKWKFKGKDIYTFDGALNKSTTDSDFKSARIEVSELLKGDASLKMDKSDAVLGNYTCEVTELSREGETIIELKYRVVSWFSPNENILIVIFPIFAILLFWGQFGIKTLKYKSSRMDEKTIALLVAGLVITAIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAVAFKED

SEQ ID NO: 93 - protein, 309 aa, synthetic construct
MWPLVAALLLGSACCGSAQLLFNKTKSVEFTFCNETVVIPCFVTNVEAENTNELYVKWKFKGKDIYTFDGALNKSTTYSNFKSARIEVSELLKGDASLKMDKSDAVLGNYTCEVTELSREGETIIELKYRVVSWFSPNENILIVIFPIFAILLFWGQFGIKTLKYKSSHMDEKTIALLVAGLVITAIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAVAFKED

SEQ ID NO: 94 - protein, 309 aa, synthetic construct
MWPLVAALLLASACCGSAQLLFNKTKSVEFTFCNETVVIPCCVNNIEAENLNELYVKWRFKGKDIYTFDGVVNKSNTYGNFKSARMEVPELLKGDASLKIDKSEAVLGNYSCEVTELSREGETFIELKYRVVPWFSPNENILIVIFPIFAVLLFWGQFGIKTLKYKSSHMDEKTIAVLVAGLVTTAVIIVGAILFVPGEYSVKNSTGLGLIVTTTGLLLLLHYYVFKTTIGIPSFVIAILVIQLLAYVLAVVGLSLCISACIPMHGPLLIAGLVIFALIQLLGLVYMKFVASNQKTMNPPRKAVAFKED

SEQ ID NO: 95 - protein, 309 aa, synthetic construct
MWPLVAALLLGSACCGSAQLLFNKTKSVEFTFCNETVVIPCFVTNVEAENTKEMYVKWKFKGKDIFIFDGAQNKSTTYNNFTSARIAVSELLKGDASLKMDKSDAVLGNYTCEVTELSREGETIIELKYRVVSWFSPNENILIVIFPIFAILLFWGQFGIKTLKYKSSHTDEKTILLLVAGLVVTAVVIVGAILFIPGEYSVKNATGLGLIVISTGILILLQYCVFKTAFGMTSFAIAILIIQVLGYVLAVVGLSLCISACIPMHGPLLISGLGIIALAQLLGLVYMKFVASNQKTIQPPRKAVAFKED

SEQ ID NO: 96 - protein, 308 aa, synthetic construct
MWPLVAALLLTSACCGSAQLLFNKTKSVEYTVCNETISIPCFVINMEAENTNEVYVKWKFKGKDIYTFDGSDNRSTHDSAFKTSGIKISELLKGDATLMMHQSNAVPGNYTCEVTVLSREGETTIEVKYRIVSWFSPNENILIVIFPVFALLLFWGQFGIKTLQYRSGRMDEKTIGLLVAGLLVTIIIIVGAILFVPGEYSVKNATGLGLIVTSTGLLIILNYYVFKTAFGITSFVIALLIIQVLVYVLAGVGLSLCISACTPVHGPVLISGLGIFALVQLLGLIYMKFVASNQKTIQPPRKAEFKED

SEQ ID NO: 97 - protein, 308 aa, synthetic construct
MWPLVAALLLGSACCGSAQLLFNKTKSVEFTFCNETVVIPCFVTNVEAENTNELYVKWKFKGKDIYTFDGALNKSTADSNFKSARIEVSELLKGDASLKMDKSDAVLGNYTCEVTELSREGETIIELKYRVVSWFSPNENILIVIFPIFAILLFWGQFGIKTLKYKSSRMDEKTIALLVAGLVVTAIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAAFKED

SEQ ID NO: 98 - protein, 308 aa, synthetic construct
MWPLVAALLLGSACCGSAQLLFNKTKSVEFTFCNETVVIPCFVTNVEAENTNELYVKWKFKGKDIYTFDGAMNKSTTDSNFKSARIEVSELLKGDASLKMDKSDAVLGNYTCEVTELSREGETIIELKYRVVSWFSPNENILIVIFPIFAILLFWGQFGIKTLKYKSSRMDEKTIALLVAGLVTTAIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAAFKED

SEQ ID NO: 99 - protein, 308 aa, synthetic construct
MWPLVAALLLASACCGSAQLLFNKTKSVEFTFCNETVVIPCCVNNIEAENLNELYVKWRFKGKDIYTFDGVVNKSNTYGNFKSARMEVPELLKGDASLKIDKSEAVLGNYSCEVTELSREGETFIELKYRVVPWFSPNENILIVIFPIFAVLLFWGQFGIKTLKYKSSHMDEKTIAVLVAGLVTTAVIIVGAILFVPGEYSVKNSTGLGLIVTTTGLLLLLHYYVFKTTIGIPSFVIAILVIQLLAYVLAVVGLSLCISACIPMHGPLLIAGLVIFALIQLLGLVYMKFVASNQKTMNPPRKAAFKED

SEQ ID NO: 100 - protein, 322 aa, synthetic construct
MWPLVGALLLGSACCGSAQITFNITKSAEFTVCNTTVVIPCFATNVETKLLTDLYVKWKFKGKDIFTYDGAQGQSSTPSSGFKSAKITPTELLHGIASLKMKKSDAELGNYTCEVTELTREGETVIELKYRVVSWFSPKENILIIIFPVLAILLSWGQFGVVTLKYASDPMKGKTILLFVAGLLLTIVVIVGAILFIPGEYSTKNASGLGLIVIPTVILILLQYFVLMTAFGMTSFAIAILILQVLGFVLAVVGLSLCIPECTPVQGPLLISGLGIIALAELLGLVSMKVVASSQGTIQPPRKAVEEPLNAFKESKGMMNDG

SEQ ID NO: 101 - protein, 322 aa, synthetic construct
LLTSCLSVFIEDEVTGSAQITFNITKSAEFTVCNTTVVIPCFATNVETKLLTDLYVKWKFKGKDIFTYDGAQGQSSTPSSGFKSAKITPTELLHGIASLKMKKSDAELGNYTCEVTELTREGETVIELKYRVVSWFSPKENILIIIFPVLAILLSWGQFGVVTLKYASDPMKGKTILLFVAGLLLTIVVIVGAILFIPGEYSTKNASGLGLIVIPTVILILLQYFVLMTAFGMTSFAIAILILQVLGFVLAVVGLSLCIPECTPVQGPLLISGLGIIALAELLGLVSMKVVASSQGTIQPPRKAVEEPLNAFKESKGMMNDG

SEQ ID NO: 102 - protein, 308 aa, synthetic construct
MWPLVAAHLLSSASSGSAQITFNTTKFVEITVCNETVVLPCFANNVETKNLRELYVKWKFKGKDIFIYDGAQDQATPSSNFSSAEITPSELLKGIASLKMKKSDAVLGNYTCEVTELSREGETIVELKYRVVSWFSPKENILIIIFPILAILLSWGQFGIVTLKYKSNRTNEKTILMFVAGLLLTIVVIVGAILFIPGEYSIKNASGLGLIVIPTVILILLQYCAFMTAFGMTSFAIAILILQVLGYVLAVVGLSLCISECTPVQGPLLISGLGIIALAELLGLVYMKFVASNQKTIQPPRKAAFKED

SEQ ID NO: 103 - protein, 308 aa, synthetic construct
MWPLVAALLLGSACCGSAQLLFNKTKSVEFTFCNDTVVIPCFVTNMEAQNTTEVYVKWKFKGRDIYTFDGALNKSTAPANFSSAKIEVSQLLKGDASLKMDKSDAVTGNYTCEVTELTREGETIIELKYRVVSWFSPNENILIVIFPIFAILLFWGQFGIKTLKYRSGGMDEKTIALLVAGLMITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAAFKED

SEQ ID NO: 104 - protein, 308 aa, synthetic construct
MWPLVAALLLGSACCGSAQLLFNKTKSVEFTFCNETVVIPCFVTNMEAENTNEVYVKWKFKGKDIYTFDGALNKSTADSNFKSARIEVSELLKGDASLKMDKSDAVLGNYTCEVTELSREGETIIELKYRVVSWFSPNENILIVIFPIFAILLFWGQFGIKTLKYRSGRMDEKTIALLVAGLVITVIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAAFKED

SEQ ID NO: 105 - protein, 308 aa, synthetic construct
MWPLVAALLLGSACCGSAQLLFNKTKSVEFTFCNETVVIPCFVNNVEAENTKEMYVKWKFKGKDIFIFDGAQNKSTAYNNFTSAKIAVSELLKGDASLKMDKSDAVLGNYTCEVTELSREGETIVELKYRVVSWFSPNENILIVIFPILAILLFWGQFGIVTLKYKSNRTNEKTILLLVAGLLLTTVVIVGAILFIPGEYSIKNASGLGLIVISTGILILLQYCVFMTAFGMTSFTIAILIIQVLGYVLAVVGLSLCISACVPVHGPLLISGLGIIALAELLGLVYMKFVASNQKTIQPPRKAAFKED

SEQ ID NO: 106 - protein, 308 aa, synthetic construct
MWPLVAALLLGSACCGSAQLLFNKTKSVEFTFCNETVVIPCFVNNVEAENIKEMYVKWKFKGKDIFIFDGAQNKSTAYNNFTSAKIAVSELLKGDASLKMDKSDAVLGNYTCEVTELSREGETIVELKYRVVSWFSPNENILIVIFPILAILLFWGQFGIVTLKYKSNRTNEKTILLLVAGLLLTIVVIVGAILFIPGEYSIKNASGLGLIVIPTGILILLQYCVFMTAFGMTSFTIAILIIQVLGYVLAVVGLSLCISACVPVHGPLLISGLGIIALAELLGLVYMKFVASNQKTIQPPRKAAFKED

SEQ ID NO: 107 - protein, 308 aa, synthetic construct
MWPLVAALLLGSACCGSAQLIFNVTKSVEFTVCNETVVIPCFVNNVEAKNIKEMYVKWKFKGKDIFIFDGAQNKSTPSNNFTSAKITPSELLKGIASLKMDKSDAVLGNYTCEVTELSREGETIVELKYRVVSWFSPNENILIVIFPILAILLSWGQFGIVTLKYKSNRTNEKTILLFVAGLLLTIVVIVGAILFIPGEYSIKNASGLGLIVIPTAILILLQYCVFMTAFGMTSFTIAILILQVLGYVLAVVGLSLCISECTPVHGPLLISGLGIIALAELLGLVYMKFVASNQKTIQPPRKAAFKED

SEQ ID NO: 108 - protein, 308 aa, synthetic construct
MWPLVAALLLGSACCGSAQLTFNATKSVEFTVCNETVVIPCFVNNVETKNIKEMYVKWKFKGKDIFIFDGAQNKSTASSNFTSAKITPSELLKGIASLKMKKSDAVLGNYTCEVTELSREGETIVELKYRVVSWFSPKENILIVIFPILAILLSWGQFGIVTLKYKSNRTNEKTILLFVAGLLLTIVVIVGAILFIPGEYSIKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAAFKED

SEQ ID NO: 109 - protein, 308 aa, synthetic construct
MWPLVAALLLGSACCGSAQLTFNATRFVEVTICNETVVLPCIVNNVESKNIKDMYVKWKFKGKDIFTFDGNQNKSNASSNFTSANITPSELLKGIASLKMEKSDAVLGNYTCEVTELSREGETIVELKYRVVSWFSPKENILIVIFPILAILLSWGQFGIVTLKYKSNRTNEKTILLIVAGLLLTIVVIVGAILFIPGEYSIKNASGLGLIVIPTVILILLQYCVFMTAFGMTSFAIAILILQVLGYILAVVGLSLCISECAPMQGPLLISGLGIIALAELLGLVYMKFVASNQKTIQPPRKAAFKED

SEQ ID NO: 110 - protein, 308 aa, synthetic construct
MWPLVAALLLGSVCGGSAQLTFNSTRFVEKTICNETVILPCIVNNLELKNIKAMYVKWKFKGKDIFTFDGNQNKSNASNNFKSANIALSDLLKGNASLKMKKSDAVPGNYTCEVTESNREGETIVELRYHVVSWFSPKENILIVIFPILAILLSWGQFGIVTLKYKSNCTSEKTIGLIIAGLILTIVVIVGAVLFIPGEYSIKNASGLGLIVIPTVILVLLQYCVFMTAFGMTSFAIAILILQLLGYILAVVGLSLCITECAPMQGPLLISGLGIIALAELLGLVYMKFVASNQKTIQPPRKAAFKED

SEQ ID NO: 111 - protein, 308 aa, synthetic construct
MWPLVAALLLGSVCGGSAQLTFNSTRFVEKTICNETVILPCIVTNLELNNIKAMYVKWKYKGKDIFTFDGNQNKSNASNNFKSANIALSDLLKGNASLKMKKSDAVPGNYTCEVTESNREGETIVELRYHVVSWFSPKENILIVIFPILAILLSWGQFGIVTLKYKSNCTSEKTIGLIIAGLILTIVVIVGAVLFIPGEYSIKNASGLGLIVIPTVILVLLQYCVFMTAFGMTSFAIAILILQLLGYILAVVGLSLCITECAPMQGPLLISGLAIIALAELLGLVYMKFVASNQKTIQPPRKAAFKED

SEQ ID NO: 112 - protein, 308 aa, synthetic construct
MWPLVAALLLGSACCGSAQLLFNKTKSVEFTFCNDTVVIPCFVTNMEAQNTTEVYVKWKFKGRDIYTFDGALNKSTVPSDFNSAKIEVSQLLKGDASLKMDKSDAVTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAFGLTSFVIAILVIQVIAYVLAVVGLSLCIAACIPMHGPLLISGLSILALAQLLGLVYMKFVASNQKTIQPPRKAAFKED

SEQ ID NO: 113 - protein, 308 aa, synthetic construct
MWPLVAALLLGSACCGSAQLLFNKTKSVEFTFCNETVVIPCFVTNMEAENTNEVYVKWKFKGKDIYTFDGALNKSTTDSDFKSARIEVSELLKGDASLKMDKSDAVLGNYTCEVTELSREGETIIELKYRVVSWFSPNENILIVIFPIFAILLFWGQFGIKTLKYRSGGMDEKTIALLVAGLVITVIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAAFKED

SEQ ID NO: 114 - protein, 308 aa, synthetic construct
MWPLVAALLLGSACCGSAQLLFNKTKSVEFTFCNETVVIPCFVTNVEAENTNELYVKWKFKGKDIYTFDGALNKSTTDSNFKSARIEVSELLKGDASLKMDKSDAVLGNYTCEVTELSREGETIIELKYRVVSWFSPNENILIVIFPIFAILLFWGQFGIKTLKYKSSGMDEKTIALLVAGLVITAIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAAFKED

SEQ ID NO: 115 - protein, 308 aa, synthetic construct
MWPLVAALLLGSACCGSAQLLFNKTKSVEFTFCNETVVIPCFVNNVEAENTNELYVKWKFKGKDIYTFDGALNKSTTYSNFKSARIEVSELLKGDASLKMDKSDAVLGNYTCEVTELSREGETIIELKYRVVSWFSPNENILIVIFPIFAILLFWGQFGIKTLKYKSSHMDEKTIALLVAGLVITAIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAAFKED

SEQ ID NO: 116 - protein, 308 aa, synthetic construct
MWPLVAALLLGSACCGSAQLLFNKTKSVEFTFCNETVVIPCFVNNVEAENTKEMYVKWKFKGKDIFIFDGAQNKSTTYNNFTSARIAVSELLKGDASLKMDKSDAVLGNYTCEVTELSREGETIIELKYRVVSWFSPNENILIVIFPIFAILLFWGQFGIKTLKYKSSHTDEKTILLLVAGLVVTAVVIVGAILFIPGEYSVKNATGLGLIVISTGILILLQYCVFKTAFGMTSFAIAILIIQVLGYVLAVVGLSLCISACIPMHGPLLISGLGIIALAQLLGLVYMKFVASNQKTIQPPRKAAFKED

SEQ ID NO: 117 - protein, 308 aa, synthetic construct
MWPLVAALLLGSACCGSAQLLFNKTKSVEFTFCNETVVIPCFVTNVEAENTNELYVKWKFKGKDIYTFDGALNKSTTDSNFKSARIEVSELLKGDASLKMDKSDAVLGNYTCEVTELSREGETIIELKYRVVSWFSPNENILIVIFPIFAILLFWGQFGIKTLKYKSSRMDEKTIALLVAGLVITAIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAAFKED

SEQ ID NO: 118 - protein, 308 aa, synthetic construct
MWPLVAALLLGSACCGSAQLLFNKTKSVEFTFCNETVVIPCFVTNVEAENTKEMYVKWKFKGKDIFIFDGAQNKSTTDNNFTSARIAVSELLKGDASLKMDKSDAVLGNYTCEVTELSREGETIIELKYRVVSWFSPNENILIVIFPIFAILLFWGQFGIKTLKYKSSRTDEKTILLLVAGLVVTAIVIVGAILFIPGEYSVKNATGLGLIVISTGILILLQYCVFKTAFGMTSFAIAILIIQVLGYVLAVVGLSLCISACIPMHGPLLISGLGIIALAQLLGLVYMKFVASNQKTIQPPRKAAFKED

SEQ ID NO: 119 - protein, 308 aa, synthetic construct
MWPLVAAQLLSSASSGSAQITFNTTKFVEITVCNETVVLPCFANNVETKNLRELYVKWKFKGKDIFIYDGAQDQATPSSNFSSAEITPSELLKGIASLKMKKSDAVLGNYTCEVTELSREGETIVELKYRVVSWFSPKENILIIIFPILAILLSWGQFGIVTLKYKSNRTNEKTILMFVAGLLLTIVVIVGAILFIPGEYSIKNASGLGLIVIPTVILILLQYCAFMTAFGMTSFAIAILILQVLGYVLAVVGLSLCISECTPVQGPLLISGLGIIALAELLGLVYMKFVASNQKTIQPPRKAAFKED

SEQ ID NO: 120 - protein, 308 aa, synthetic construct
MWPLVAAHLLSSASSGSAQITFNTTKFVEVTVCNETVVLPCFANNVETKNLRELYLKWKFRGKDIFIYDGAQDLATPSSNFSSAEITPSELLKGNASLKMKKSDAVLGNYTCEVTELSREGETVVELKYRVVSWFSPKENILIIIFPILAILLSWGQFGIVTLKYKSNRTNEKTILMFVAGLLLTIVIIVGAILFIPGEYSIKNASGLGLIVIPTVILILLQYCAFMTAFGMTSFAIAILILQVLGYVLAVVGLSLCISECTPVQGPLLISGLGIIALAELLGLVYMKFVASNQKTIQPPRKAAFKED

SEQ ID NO: 121 - protein, 308 aa, synthetic construct
MWPLVAALLLGSACCGSAQLLFNKTKSVEFTFCNDTVVIPCFVTNMEAQNTTEVYVKWKFKGRDIYTFDGALNKSTVPSNFNSAKIEVSQLLKGDASLKMDKSDAVTGNYTCEVTELTREGETIIELKYRVVSWFSPNENILIVIFPIFAILLFWGQFGIKTLKYRSGGMDEKTIALLVAGLMITVIVIVGAILFVPGEYSLKNATGLGLIVTSTGILILLHYYVFSTAFGLTSFVIAILVIQVIAYVLAVVGLSLCIAACIPMHGPLLISGLSILALAQLLGLVYMKFVASNQKTIQPPRKAAFKED

SEQ ID NO: 122 - protein, 308 aa, synthetic construct
MWPLVAALLLGSACCGSAQLLFNKTKSVEFTFCNETVVIPCFVTNMEAENTNEVYVKWKFKGKDIYTFDGALNKSTTDSNFKSARIEVSELLKGDASLKMDKSDAVLGNYTCEVTELSREGETIIELKYRVVSWFSPNENILIVIFPIFAILLFWGQFGIKTLKYRSGRMDEKTIALLVAGLVITVIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAAFKED

SEQ ID NO: 123 - protein, 309 aa, synthetic construct
MWPLVAALLLGSACCGSAQLLFNKTKSVEFTFCNETVVIPCFVTNVEAENTKEMYVKWKFKGKDIFIFDGAQNKSTTYNNFTSAKIAVSELLKGDASLKMDKSDAVLGNYTCEVTELSREGETIVELKYRVVSWFSPNENILIVIFPILAILLFWGQFGIVTLKYKSNRTNEKTILLLVAGLLLTTVVIVGAILFIPGEYSIKNASGLGLIVISTGILILLQYCVFMTAFGMTSFTIAILIIQVLGYVLAVVGLSLCISACVPVHGPLLISGLGIIALAELLGLVYMKFVASNQKTIQPPRKAVAFKED

SEQ ID NO: 124 - protein, 309 aa, synthetic construct
MWPLVAALLLGSACCGSAQLLFNKTKSVEFTFCNETVVIPCFVTNVEAENIKEMYVKWKFKGKDIFIFDGAQNKSTTYNNFTSAKIAVSELLKGDASLKMDKSDAVLGNYTCEVTELSREGETIVELKYRVVSWFSPNENILIVIFPILAILLFWGQFGIVTLKYKSNRTNEKTILLLVAGLLLTIVVIVGAILFIPGEYSIKNASGLGLIVIPTGILILLQYCVFMTAFGMTSFTIAILIIQVLGYVLAVVGLSLCISACVPVHGPLLISGLGIIALAELLGLVYMKFVASNQKTIQPPRKAVAFKED

SEQ ID NO: 125 - protein, 309 aa, synthetic construct
MWPLVAALLLGSACCGSAQLIFNATKSVEFTICNETVVIPCFVNNVEAKNIKEMYVKWKFKGKDIFIFDGAQNKSTTSNNFTSAKITPSELLKGIASLKMDKSDAVLGNYTCEVTELSREGETIVELKYRVVSWFSPNENILIVIFPILAILLSWGQFGIVTLKYKSNRTNEKTILLFVAGLLLTIVVIVGAILFIPGEYSIKNASGLGLIVIPTAILILLQYCVFMTAFGMTSFTIAILILQVLGYVLAVVGLSLCISECTPVHGPLLISGLGIIALAELLGLVYMKFVASNQKTIQPPRKAVAFKED

SEQ ID NO: 126 - protein, 309 aa, synthetic construct
MWPLVAALLLGSACCGSAQLTFNATKFVEFTICNETVVIPCFVNNVETKNIKEMYVKWKFKGKDIFIFDGAQNKSTASSNFTSAKITPSELLKGIASLKMKKSDAVLGNYTCEVTELSREGETIVELKYRVVSWFSPKENILIVIFPILAILLSWGQFGIVTLKYKSNRTNEKTILLFVAGLLLTIVVIVGAILFIPGEYSIKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAVAFKED

SEQ ID NO: 127 - protein, 309 aa, synthetic construct
MWPLVAALLLGSACCGSAQLTFNATRFVEVTICNETVVLPCIVNNVESKNIKDMYVKWKFKGKDIFTFDGNQNKSNASSNFTSANITPSELLNGIASLKMKKSDAVLGNYTCEVTELSREGETIVELKYRVVSWFSPKENILIVIFPILAILLSWGQFGIVTLKYKSNRTNEKTILLIVAGLLLTIVVIVGAILFIPGEYSIKNASGLGLIVIPTVILILLQYCVFMTAFGMTSFAIAILILQVLGYILAVVGLSLCISECAPMQGPLLISGLGIIALAELLGLVYMKFVASNQKTIQPPRKAVAFKED

SEQ ID NO: 128 - protein, 309 aa, synthetic construct
MWPLVAALLLGSVCGGSAQLTFNSTRFVEKTICNETVILPCIVNNLELKNIKAMYVKWKFKGKDIFTFDGNQNKSNASNNFKSANIALSDLLNGNASLKMKKSDAVPGNYTCEVTESNREGETIVELRYHVVSWFSPKENILIVIFPILAILLSWGQFGIVTLKYKSNCTSEKTIGLIIAGLILTIVVIVGAVLFIPGEYSIKNASGLGLIVIPTVILVLLQYCVFMTAFGMTSFAIAILILQLLGYILAVVGLSLCITECAPMQGPLLISGLGIIALAELLGLVYMKFVASNQKTIQPPRKAVAFKED

SEQ ID NO: 129 - protein, 309 aa, synthetic construct
MWPLVAALLLGSVCGGSAQLTFNSTRFVEKTICNETVILPCIVTNLELNNIKAMFVKWKFKGKDIFTFDGNQNKSNASNNFKSANISLSDLLNGNASLKMKKSDAVPGNYTCEVTESNREGETIVELRYHVVSWFSPKENILIVIFPILAILLSWGQFGIVTLKYKSNCTSEKTIGLIIAGLILTIVVIVGAVLFIPGEYSIKNASGLGLIVIPTVILVLLQYCVFMTAFGMTSFAIAILILQLLGYILAVVGLSLCITECAPMQGPLLISGLAIIALAELLGLVYMKFVASNQKTIQPPRKAVAFKED

SEQ ID NO: 130 - protein, 309 aa, synthetic construct
MWPLVAALLLGSACCGSAQLLFNKTKSVEFTFCNDTVVIPCFVTNMEAQNTTEVYVKWKFKGRDIYTFDGALNKSTVPSDFNSAKIEVSQLLKGDASLKMDKSDAVTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAFGLTSFVIAILVIQVIAYVLAVVGLSLCIAACIPMHGPLLISGLSILALAQLLGLVYMKFVASNQKTIQPPRKAVAFKED

SEQ ID NO: 131 - protein, 309 aa, synthetic construct
MWPLVAALLLGSACCGSAQLLFNKTKSVEFTFCNETVVIPCFVTNMEAENTNEVYVKWKFKGKDIYTFDGALNKSTTDSDFKSARIEVSELLKGDASLKMDKSDAVLGNYTCEVTELSREGETIIELKYRVVSWFSPNENILIVIFPIFAILLFWGQFGIKTLKYRSGGMDEKTIALLVAGLVITVIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAVAFKED

SEQ ID NO: 132 - protein, 309 aa, synthetic construct
MWPLVAALLLGSACCGSAQLLFNKTKSVEFTFCNETVVIPCFVTNVEAENTNELYVKWKFKGKDIYTFDGALNKSTTDSNFKSARIEVSELLKGDASLKMDKSDAVLGNYTCEVTELSREGETIIELKYRVVSWFSPNENILIVIFPIFAILLFWGQFGIKTLKYKSSGMDEKTIALLVAGLVITAIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAVAFKED

SEQ ID NO: 133 - protein, 308 aa, synthetic construct
MWPLVAALLLGSACCGSAQLIFNVTKSVEFTICNETVVIPCFVNNVEAKNIKEMYVKWKFKGKDIFIFDGAQNKSTPSNNFTSAKITPSELLNGIASLKMDKSDAVLGNYTCEVTELSREGETIVELKYRVVSWFSPNENILIVIFPILAILLFWGQFGIVTLKYKSNRTNEKTILLFVAGLLLTIVVIVGAILFIPGEYSIKNASGLGLIVIPTVILILLQYCVFMTAFGMTSFTIAILILQVLGYVLAVVGLSLCISECTPVHGPLLISGLGIIALAELLGLVYMKFVASNQKTIQPPRKAAFKED

SEQ ID NO: 134 - protein, 308 aa, synthetic construct
MWPLVAALLLGSACCGSAQLTFNATKSVEFTICNETVVIPCFVNNVETKNIKEMYVKWKFKGKDIFIFDGAQNKSTPSSNFTSAKITPSELLNGIASLKMKKSDAVLGNYTCEVTELSREGETIVELKYRVVSWFSPKENILIVIFPILAILLSWGQFGIVTLKYKSNRTNEKTILLFVAGLLLTIVVIVGAILFIPGEYSIKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAAFKED

SEQ ID NO: 135 - protein, 308 aa, synthetic construct
MWPLVAALLLGSACCGSAQLTFNATRFVEVTICNETVVLPCIVNNVESKNIKDMYVKWKFKGKDIFTFDGNQNKSNASSNFTSANITPSELLNGIASLKMEKSDAVLGNYTCEVTELSREGETIVELKYRVVSWFSPKENILIVIFPILAILLSWGQFGIVTLKYKSNRTNEKTILLIVAGLLLTIVVIVGAILFIPGEYSIKNASGLGLIVIPTVILILLQYCVFMTAFGMTSFAIAILILQVLGYILAVVGLSLCISECAPMQGPLLISGLGIIALAELLGLVYMKFVASNQKTIQPPRKAAFKED

SEQ ID NO: 136 - protein, 308 aa, synthetic construct
MWPLVAALLLGSVCGGSAQLTFNSTRFVEKTICNETVILPCIVNNLELKNIKAMYVKWKFKGKDIFTFDGNQNKSNASNNFKSANIALSDLLNGNASLKMKKSDAVPGNYTCEVTESNREGETIVELRYHVVSWFSPKENILIVIFPILAILLSWGQFGIVTLKYKSNCTSEKTIGLIIAGLILTIVVIVGAVLFIPGEYSIKNASGLGLIVIPTVILVLLQYCVFMTAFGMTSFAIAILILQLLGYILAVVGLSLCITECAPMQGPLLISGLGIIALAELLGLVYMKFVASNQKTIQPPRKAAFKED

SEQ ID NO: 137 - protein, 308 aa, synthetic construct
MWPLVAALLLGSVCGGSAQLTFNSTRFVEKTICNETVILPCIVTNLELNNIKAMFVKWKFKGKDIFTFDGNQNKSNASNNFKSANISLSDLLNGNASLKMKKSDAVPGNYTCEVTESNREGETIVELRYHVVSWFSPKENILIVIFPILAILLSWGQFGIVTLKYKSNCTSEKTIGLIIAGLILTIVVIVGAVLFIPGEYSIKNASGLGLIVIPTVILVLLQYCVFMTAFGMTSFAIAILILQLLGYILAVVGLSLCITECAPMQGPLLISGLAIIALAELLGLVYMKFVASNQKTIQPPRKAAFKED

SEQ ID NO: 138 - protein, 308 aa, synthetic construct
MWPLVAALLLGSACCGSAQLLFNKTKSVEFTFCNETVVIPCFVTNVEAENTNELYVKWKFKGKDIYTFDGALNKSTTDSDFKSARIEVSELLKGDASLKMDKSDAVLGNYTCEVTELSREGETIIELKYRVVSWFSPNENILIVIFPIFAILLFWGQFGIKTLKYKSSGMDEKTIALLVAGLVITAIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAAFKED

SEQ ID NO: 139 - protein, 308 aa, synthetic construct
MWPLVAALLLGSACCGSAQLTFNATRFVEVTICNETVVLPCIVNNVESKNIKDMYVKWKFKGKDIFTFDGNQNKSNASSNFTSANITPSELLKGIASLKMKKSDAVLGNYTCEVTELSREGETIVELKYRVVSWFSPKENILIVIFPILAILLSWGQFGIVTLKYKSNRTNEKTILLIVAGLLLTIVVIVGAILFIPGEYSIKNASGLGLIVIPTVILILLQYCVFMTAFGMTSFAIAILILQVLGYILAVVGLSLCISECAPMQGPLLISGLGIIALAELLGLVYMKFVASNQKTIQPPRKAAFKED

SEQ ID NO: 140 - protein, 308 aa, synthetic construct
MWPLVAALLLGSVCGGSAQLTFNSTRFVEKTICNETVILPCIVTNLELNNIKAMFVKWKFKGKDIFTFDGNQNKSNASNNFKSANISLSDLLKGNASLKMKKSDAVPGNYTCEVTESNREGETIVELRYHVVSWFSPKENILIVIFPILAILLSWGQFGIVTLKYKSNCTSEKTIGLIIAGLILTIVVIVGAVLFIPGEYSIKNASGLGLIVIPTVILVLLQYCVFMTAFGMTSFAIAILILQLLGYILAVVGLSLCITECAPMQGPLLISGLAIIALAELLGLVYMKFVASNQKTIQPPRKAAFKED

SEQ ID NO: 141 - protein, 316 aa, synthetic construct
MWPLVAALLLGSACCGSAQLLFNKTKSVEFTFCNDTVVIPCFVTNMEAQNTTEVYVKWKFKGRDIYTFDGALNKSTVPSDFNSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAFGLTSFVIAILVIQVIAYVLAVVGLSLCIAACIPMHGPLLISGLSILALAQLLGLVYMKFVASNQKTIQPPRKAVEEPLNAFKED

SEQ ID NO: 142 - protein, 309 aa, synthetic construct
MWPLVAALLLGSACCGSAQLLFNKTKSVEFTFCNETVVIPCFVNNVEAENTKEMYVKWKFKGKDIFIFDGAQNKSTTYNNFTSAKIAVSELLKGDASLKMDKSDAVLGNYTCEVTELSREGETIVELKYRVVSWFSPNENILIVIFPILAILLFWGQFGIVTLKYKSNRTNEKTILLLVAGLLLTTVVIVGAILFIPGEYSIKNASGLGLIVISTGILILLQYCVFMTAFGMTSFTIAILIIQVLGYVLAVVGLSLCISACVPVHGPLLISGLGIIALAELLGLVYMKFVASNQKTIQPPRKAVAFKED

SEQ ID NO: 143 - protein, 309 aa, synthetic construct
MWPLVAALLLGSACCGSAQLLFNKTKSVEFTFCNETVVIPCFVNNVEAENIKEMYVKWKFKGKDIFIFDGAQNKSTTYNNFTSAKIAVSELLKGDASLKMDKSDAVLGNYTCEVTELSREGETIVELKYRVVSWFSPNENILIVIFPILAILLFWGQFGIVTLKYKSNRTNEKTILLLVAGLLLTIVVIVGAILFIPGEYSIKNASGLGLIVIPTGILILLQYCVFMTAFGMTSFTIAILIIQVLGYVLAVVGLSLCISACVPVHGPLLISGLGIIALAELLGLVYMKFVASNQKTIQPPRKAVAFKED

SEQ ID NO: 144 - protein, 309 aa, synthetic construct
MWPLVAALLLGSACCGSAQLIFNVTKSVEFTICNETVVIPCFVNNVEAKNIKEMYVKWKFKGKDIFTFDGAQNKSTTSNNFTSAKITPSELLKGIASLKMDKSDAVLGNYTCEVTELSREGETIVELKYRVVSWFSPNENILIVIFPILAILLSWGQFGIVTLKYKSNRTNEKTILLFVAGLLLTIVVIVGAILFIPGEYSIKNASGLGLIVIPTAILILLQYCVFMTAFGMTSFTIAILILQVLGYVLAVVGLSLCISECTPVHGPLLISGLGIIALAELLGLVYMKFVASNQKTIQPPRKAVAFKED

SEQ ID NO: 145 - protein, 309 aa, synthetic construct
MWPLVAALLLGSACCGSAQLTFNATKSVEFTICNETVVIPCFVNNVETKNIKEMYVKWKFKGKDIFTFDGAQNKSTASSNFTSAKITPSELLKGIASLKMKKSDAVLGNYTCEVTELSREGETIVELKYRVVSWFSPKENILIVIFPILAILLSWGQFGIVTLKYKSNRTNEKTILLFVAGLLLTIVVIVGAILFIPGEYSIKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAVAFKED

SEQ ID NO: 146 - protein, 309 aa, synthetic construct
MWPLVAALLLGSACCGSAQLTFNATRFVEVTICNETVVLPCIVNNVESKNIKDMYVKWKFKGKDIFTFDGNQNKSNASSNFTSANITPSELLKGIASLKMKKSDAVLGNYTCEVTELSREGETIVELKYRVVSWFSPKENILIVIFPILAILLSWGQFGIVTLKYKSNRTNEKTILLIVAGLLLTIVVIVGAILFIPGEYSIKNASGLGLIVIPTVILILLQYCVFMTAFGMTSFAIAILILQVLGYILAVVGLSLCISECAPMQGPLLISGLGIIALAELLGLVYMKFVASNQKTIQPPRKAVAFKED

SEQ ID NO: 147 - protein, 309 aa, synthetic construct
MWPLVAALLLGSACCGSAQLLFNKTKSVEFTFCNETVVIPCFVTNVEAENTNELYVKWKFKGKDIYTFDGALNKSTTDSDFKSARIEVSELLKGDASLKMDKSDAVLGNYTCEVTELSREGETIIELKYRVVSWFSPNENILIVIFPIFAILLFWGQFGIKTLKYKSSGMDEKTIALLVAGLVITAIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAVAFKED

SEQ ID NO: 148 - protein, 309 aa, synthetic construct
MWPLVAALLLGSACCGSAQLLFNKTKSVEFTFCNETVVIPCFVNNVEAENTNELYVKWKFKGKDIYTFDGALNKSTTYSNFKSARIEVSELLKGDASLKMDKSDAVLGNYTCEVTELSREGETIIELKYRVVSWFSPNENILIVIFPIFAILLFWGQFGIKTLKYKSSHMDEKTIALLVAGLVITAIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAVAFKED

SEQ ID NO: 149 - protein, 309 aa, synthetic construct
MWPLVAALLLGSACCGSAQLLFNKTKSVEFTFCNETVVIPCFVNNVEAENTKEMYVKWKFKGKDIFIFDGAQNKSTTYNNFTSARIAVSELLKGDASLKMDKSDAVLGNYTCEVTELSREGETIIELKYRVVSWFSPNENILIVIFPIFAILLFWGQFGIKTLKYKSSHTDEKTILLLVAGLVVTAVVIVGAILFIPGEYSVKNATGLGLIVISTGILILLQYCVFKTAFGMTSFAIAILIIQVLGYVLAVVGLSLCISACIPMHGPLLISGLGIIALAQLLGLVYMKFVASNQKTIQPPRKAVAFKED

SEQ ID NO: 150 - protein, 310 aa, synthetic construct
MWPLVAALLLGSACCGSAQLLFNKTKSVEFTFCNETVVIPCFVNNVEAENTKEMYVKWKFKGKDIFIFDGAQNKSTAYNNFTSAKIAVSELLKGDASLKMDKSDAVLGNYTCEVTELSREGETIVELKYRVVSWFSPNENILIVIFPILAILLFWGQFGIVTLKYKSNRTNEKTILLLVAGLLLTTVVIVGAILFIPGEYSIKNASGLGLIVISTGILILLQYCVFMTAFGMTSFTIAILIIQVLGYVLAVVGLSLCISACVPVHGPLLISGLGIIALAELLGLVYMKFVASNQKTIQPPRKAVEAFKED

SEQ ID NO: 151 - protein, 310 aa, synthetic construct
MWPLVAALLLGSACCGSAQLLFNKTKSVEFTFCNETVVIPCFVNNVEAENIKEMYVKWKFKGKDIFIFDGAQNKSTAYNNFTSAKIAVSELLKGDASLKMDKSDAVLGNYTCEVTELSREGETIVELKYRVVSWFSPNENILIVIFPILAILLFWGQFGIVTLKYKSNRTNEKTILLLVAGLLLTIVVIVGAILFIPGEYSIKNASGLGLIVIPTGILILLQYCVFMTAFGMTSFTIAILIIQVLGYVLAVVGLSLCISACVPVHGPLLISGLGIIALAELLGLVYMKFVASNQKTIQPPRKAVEAFKED

SEQ ID NO: 152 - protein, 310 aa, synthetic construct
MWPLVAALLLGSACCGSAQLIFNVTKSVEFTICNETVVIPCFVNNVEAKNIKEMYVKWKFKGKDIFIFDGAQNKSTTSNNFTSAKITPSELLNGIASLKMDKSDAVLGNYTCEVTELSREGETIVELKYRVVSWFSPNENILIVIFPILAILLSWGQFGIVTLKYKSNRTNEKTILLFVAGLLLTIVVIVGAILFIPGEYSIKNASGLGLIVIPTAILILLQYCVFMTAFGMTSFTIAILILQVLGYVLAVVGLSLCISECTPVHGPLLISGLGIIALAELLGLVYMKFVASNQKTIQPPRKAVEAFKED

SEQ ID NO: 153 - protein, 310 aa, synthetic construct
MWPLVAALLLGSACCGSAQLTFNATKFVEFTICNETVVIPCFVNNVETKNIKEMYVKWKFKGKDIFIFDGAQNKSTASSNFTSAKITPSELLNGIASLKMKKSDAVLGNYTCEVTELSREGETIVELKYRVVSWFSPKENILIVIFPILAILLSWGQFGIVTLKYKSNRTNEKTILLFVAGLLLTIVVIVGAILFIPGEYSIKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAVEAFKED

SEQ ID NO: 154 - protein, 310 aa, synthetic construct
MWPLVAALLLGSACCGSAQLTFNATRFVEVTICNETVVLPCIVNNVESKNIKDMYVKWKFKGKDIFTFDGNQNKSNASSNFTSANITPSELLNGIASLKMKKSDAVLGNYTCEVTELSREGETIVELKYRVVSWFSPKENILIVIFPILAILLSWGQFGIVTLKYKSNRTNEKTILLIVAGLLLTIVVIVGAILFIPGEYSIKNASGLGLIVIPTVILILLQYCVFMTAFGMTSFAIAILILQVLGYILAVVGLSLCISECAPMQGPLLISGLGIIALAELLGLVYMKFVASNQKTIQPPRKAVEAFKED

SEQ ID NO: 155 - protein, 310 aa, synthetic construct
MWPLVAALLLGSVCGGSAQLTFNSTRFVEKTICNETVILPCIVNNLELKNIKAMYVKWKFKGKDIFTFDGNQNKSNASNNFKSANIALSDLLNGNASLKMKKSDAVPGNYTCEVTESNREGETIVELRYHVVSWFSPKENILIVIFPILAILLSWGQFGIVTLKYKSNCTSEKTIGLIIAGLILTIVVIVGAVLFIPGEYSIKNASGLGLIVIPTVILVLLQYCVFMTAFGMTSFAIAILILQLLGYILAVVGLSLCITECAPMQGPLLISGLGIIALAELLGLVYMKFVASNQKTIQPPRKAVEAFKED

SEQ ID NO: 156 - protein, 310 aa, synthetic construct
MWPLVAALLLGSVCGGSAQLTFNSTRFVEKTICNETVILPCIVTNLELNNIKAMFVKWKFKGKDIFTFDGNQNKSNASNNFKSANISLSDLLNGNASLKMKKSDAVPGNYTCEVTESNREGETIVELRYHVVSWFSPKENILIVIFPILAILLSWGQFGIVTLKYKSNCTSEKTIGLIIAGLILTIVVIVGAVLFIPGEYSIKNASGLGLIVIPTVILVLLQYCVFMTAFGMTSFAIAILILQLLGYILAVVGLSLCITECAPMQGPLLISGLAIIALAELLGLVYMKFVASNQKTIQPPRKAVEAFKED

SEQ ID NO: 157 - protein, 310 aa, synthetic construct
MWPLVAALLLGSACCGSAQLLFNKTKSVEFTFCNDTVVIPCFVTNMEAQNTTEVYVKWKFKGRDIYTFDGALNKSTVPSDFKSAKIEVSQLLKGDASLKMDKSDAVTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAFGLTSFVIAILVIQVIAYVLAVVGLSLCIAACIPMHGPLLISGLSILALAQLLGLVYMKFVASNQKTIQPPRKAVEAFKED

SEQ ID NO: 158 - protein, 310 aa, synthetic construct
MWPLVAALLLGSACCGSAQLLFNKTKSVEFTFCNETVVIPCFVTNMEAENTNEVYVKWKFKGKDIYTFDGALNKSTTDSDFKSARIEVSELLKGDASLKMDKSDAVLGNYTCEVTELSREGETIIELKYRVVSWFSPNENILIVIFPIFAILLFWGQFGIKTLKYRSGGMDEKTIALLVAGLVITVIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAVEAFKED

SEQ ID NO: 159 - protein, 310 aa, synthetic construct
MWPLVAALLLGSACCGSAQLLFNKTKSVEFTFCNETVVIPCFVTNVEAENTNELYVKWKFKGKDIYTFDGALNKSTTDSDFKSARIEVSELLKGDASLKMDKSDAVLGNYTCEVTELSREGETIIELKYRVVSWFSPNENILIVIFPIFAILLFWGQFGIKTLKYKSSGMDEKTIALLVAGLVITAIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAVEAFKED

SEQ ID NO: 160 - protein, 310 aa, synthetic construct
MWPLVAALLLGSACCGSAQLLFNKTKSVEFTFCNETVVIPCFVNNVEAENTNELYVKWKFKGKDIYTFDGALNKSTTYSNFKSARIEVSELLKGDASLKMDKSDAVLGNYTCEVTELSREGETIIELKYRVVSWFSPNENILIVIFPIFAILLFWGQFGIKTLKYKSSHMDEKTIALLVAGLVITAIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAVEAFKED

SEQ ID NO: 161 - protein, 310 aa, synthetic construct
MWPLVAALLLASACCGSAQLLFNKTKSVEFTFCNETVVIPCCVNNIEAENLNELYVKWRFKGKDIYTFDGVVNKSNTYGNFKSARMEVPELLKGDASLKIDKSEAVLGNYSCEVTELSREGETFIELKYRVVPWFSPNENILIVIFPIFAVLLFWGQFGIKTLKYKSSHMDEKTIAVLVAGLVTTAVIIVGAILFVPGEYSVKNSTGLGLIVTTTGLLLLLHYYVFKTTIGIPSFVIAILVIQLLAYVLAVVGLSLCISACIPMHGPLLIAGLVIFALIQLLGLVYMKFVASNQKTMNPPRKAVEAFKED

SEQ ID NO: 162 - protein, 310 aa, synthetic construct
MWPLVAALLLGSACCGSAQLLFNKTKSVEFTFCNETVVIPCFVNNVEAENTKEMYVKWKFKGKDIFIFDGAQNKSTTYNNFTSARIAVSELLKGDASLKMDKSDAVLGNYTCEVTELSREGETIIELKYRVVSWFSPNENILIVIFPIFAILLFWGQFGIKTLKYKSSHTDEKTILLLVAGLVVTAVVIVGAILFIPGEYSVKNATGLGLIVISTGILILLQYCVFKTAFGMTSFAIAILIIQVLGYVLAVVGLSLCISACIPMHGPLLISGLGIIALAQLLGLVYMKFVASNQKTIQPPRKAVEAFKED

SEQ ID NO: 163 - protein, 309 aa, synthetic construct
MWPLVAALLLGSACCGSAQLLFNKTKSVEFTFCNETVVIPCFVNNVEAENTKEMYVKWKFKGKDIFIFDGAQNKSTAYNNFTSAKIAVSELLKGDASLKMDKSDAVLGNYTCEVTELSREGETIVELKYRVVSWFSPNENILIVIFPILAILLFWGQFGILTLKYKSNRTNEKTILLLVAGLLLTTVVIVGAILFIPGEYSIKNASGLGLIVISTGILILLQYCVFMTAFGMTSFTIAILIIQVLGYVLAVVGLSLCISACVPVHGPLLISGLGIIALAELLGLVYMKFVASNQKTIQPPRKAVAFKED

SEQ ID NO: 164 - protein, 309 aa, synthetic construct
MWPLVAALLLGSACCGSAQLLFNKTKSVEFTFCNETVVIPCFVNNVEAENIKEMYVKWKFKGKDIFIFDGAQNKSTAYNNFTSAKIAVSELLKGDASLKMDKSDAVLGNYTCEVTELSREGETIVELKYRVVSWFSPNENILIVIFPILAILLFWGQFGILTLKYKSNRTNEKTILLLVAGLLLTIVVIVGAILFIPGEYSIKNASGLGLIVISTGILILLQYCVFMTAFGMTSFTIAILIIQVLGYVLAVVGLSLCISECVPVHGPLLISGLGIIALAELLGLVYMKFVASNQKTIQPPRKAVAFKED

SEQ ID NO: 165 - protein, 309 aa, synthetic construct
MWPLVAALLLGSACCGSAQLLFNKTKSVEFTFCNETVVIPCFVNNVEAENIKEMYVKWKFKGKDIFIFDGAQNKSTAYNNFTSAKIAVSELLKGDASLKMDKSDAVLGNYTCEVTELSREGETIVELKYRVVSWFSPNENILIVIFPILAILLFWGQFGILTLKYKSNRTNEKTILLLVAGLLLTIVVIVGAILFIPGEYSIKNASGLGLIVIPTGILILLQYCVFMTAFGMTSFTIAILIIQVLGYVLAVVGLSLCISECVPVHGPLLISGLGIIALAELLGLVYMKFVASNQKTIQPPRKAVAFKED

SEQ ID NO: 166 - protein, 309 aa, synthetic construct
MWPLVAALLLGSACCGSAQLIFNVTKSVEFTICNETVVIPCFVNNVEAKNIKEMYVKWKFKGKDIFIFDGAQNKSTASNNFTSAKITPSELLKGIASLKMDKSDAVLGNYTCEVTELSREGETIVELKYRVVSWFSPNENILIVIFPILAILLFWGQFGIVTLKYKSNRTNEKTILLFVAGLLLTIVVIVGAILFIPGEYSIKNASGLGLIVIPTVILILLQYCVFMTAFGMTSFTIAILILQVLGYVLAVVGLSLCISECTPVHGPLLISGLGIIALAELLGLVYMKFVASNQKTIQPPRKAVAFKED

SEQ ID NO: 167 - protein, 309 aa, synthetic construct
MWPMVAAQVLAMGNMGSAQITFNATKFVEITACNETVVLPCFPNNLEPKSLGDLYVKWKFKGKDIFIYEGNKDQAVVSSNFSSAQITISELLKGVASLKMKKSDAVLGNYTCEVTELSREGETIVELRYRAVSWFSPKENILIIIFPILAILLSWGQFGIVTLKYTSNRTNEKTILMFVTGLILTIIVIVGAILFVPGEYSVKNASGLGLITIPTVVLILLQYCAFMTAFGMTFFAIAILILQVLGFVLAVVGLSLCISECTPVQGPLLISGLGIIALAELLGLVYMKFVASNQKTIQPPRKAVAFKEE

SEQ ID NO: 168 - protein, 309 aa, synthetic construct
MWPLVAALLLGSACCGSAQLLFNKTKSVEFTFCNDTVVIPCFVTNMEAQNTTEVYVKWKFKGRDIYTFDGALNKSTAPANFSSAKIEVSQLLKGDASLKMDKSDAVTGNYTCEVTELTREGETIIELKYRVVSWFSPNENILIVIFPIFAILLFWGQFGIKTLKYRSGGMDEKTIALLVAGLMITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVAFKED

SEQ ID NO: 169 - protein, 309 aa, synthetic construct
MWPLVAALLLGSACCGSAQLLFNKTKSVEFTFCNETVVIPCFVTNMEAENTNEVYVKWKFKGKDIYTFDGALNKSTADSNFKSARIEVSELLKGDASLKMDKSDAVLGNYTCEVTELSREGETIIELKYRVVSWFSPNENILIVIFPIFAILLFWGQFGIKTLKYRSGRMDEKTIALLVAGLVITVIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAVAFKED

SEQ ID NO: 170 - protein, 309 aa, synthetic construct
MWPLVAALLLGSACCGSAQITFNTTKSVEFTVCNETVVIPCFANNVETKNLKELYVKWKFKGKDIFIYDGAQNQSTPSSNFTSAKITPSELLKGIASLKMKKSDAVLGNYTCEVTELSREGETIVELKYRVVSWFSPKENILIIIFPILAILLSWGQFGIVTLKYKSNRTNEKTILLFVAGLLLTIVVIVGAILFIPGEYSIKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAVAFKED

SEQ ID NO: 171 - protein, 309 aa, synthetic construct
MWPLVAALLLGSACCGSAQITFNVTKSVEFTVCNETVVIPCFATNVETKNLTELYVKWKFKGKDIFIYDGAQNQSTPSSNFTSAKITPSELLRGIASLKMKKSDAVLGNYTCEVTELSREGETIIELKYRVVSWFSPKENILIIIFPILAILLSWGQFGVVTLKYKSNRMNEKTILLFVAGLLLTIVVIVGAILFIPGEYSTKNASGLGLIVIPTVILILLQYFVFMTAFGMTSFAIAILILQVLGFVLAVVGLSLCISECTPVQGPLLISGLGIIALAELLGLVYMKFVASNQKTIQPPRKAVAFKED

SEQ ID NO: 172 - protein, 309 aa, synthetic construct
MWPLAVLLILGRVGPGSAQITFNVIKSVEYTVCNETLVIPCFATNVETKQLAELYVKWKFKGKDIFIFDGSRNQSTPSSNFTSAEITPEELLRGIASLKMKKKDAVPGNYTCEVTELSREGETTIQLKYRVVSWFFQKENILIIIFPILAILLFWGQFGVLILNSKSSRMNQETISVLVAGLFLTMVVIVGAILFAPGEYSTKNASGLGLIVIPMVILQVLQYFVFKTSFEMNYFAIAILILQVLGFVLAVVGFSLCVSECVPVQGPLLISGLGIIALAELLGLVYMKFVASNQKTIPPPRKAVAFKED

SEQ ID NO: 173 - protein, 308 aa, synthetic construct
MWPLVAALLLGSACCGSAQLIFNTTKSVEFTICNETVVIPCFVNNVEAKNIKEMYVKWKFKGKDIFTFDGAQNKSTPSNNFTSAKITPSELLKGIASLKMDKSDAVLGNYTCEVTELSREGETIVELKYRVVSWFSPNENILIVIFPILAILLFWGQFGIVTLKYKSNRTNEKTILLFVAGLLLTIVVIVGAILFIPGEYSIKNASGLGLIVIPTVILILLQYCVFMTAFGMTSFTIAILILQVLGYVLAVVGLSLCISECTPVHGPLLISGLGIIALAELLGLVYMKFVASNQKTIQPPRKAAFKED

SEQ ID NO: 174 - protein, 308 aa, synthetic construct
MWPLVAALLLGSACCGSAQLTFNATKFVEFTICNETVVIPCFVNNVETKNIKEMYVKWKFKGKDIFTFDGAQNKSTPSSNFTSAKITPSELLKGIASLKMKKSDAVLGNYTCEVTELSREGETIVELKYRVVSWFSPKENILIVIFPILAILLSWGQFGIVTLKYKSNRTNEKTILLFVAGLLLTIVVIVGAILFIPGEYSIKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAAFKED

SEQ ID NO: 175 - protein, 308 aa, synthetic construct
MWPLVAALLLGSACCGSAQITFNTTKFVEFTVCNETVVIPCFANNVETKNLKELYVKWKFKGKDIFIYDGAQNQSTPSSNFTSAKITPSELLKGIASLKMKKSDAVLGNYTCEVTELSREGETIVELKYRVVSWFSPKENILIIIFPILAILLSWGQFGIVTLKYKSNRTNEKTILLFVAGLLLTIVVIVGAILFIPGEYSIKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAAFKED

SEQ ID NO: 176 - protein, 308 aa, synthetic construct
MWPAMLLLLLSSVCRGSAQLLFDSPRLVEKNACNHSVLLPCSVTNIELNNIRAMFVRWKYKGRDIYTFDGFQDKVTISNDFQSASIFVPDILSGNSSLRLSDDDAVLGNYTCEVTEANREGEIIVELRYHVVSWFNPKENILIVIFPILAILLSWGQFGIVTLKYKANCPSEKTFGLIISGIILTLFVIIGAALFIPGEYSIKNASGLGLIVVPTLMLVLLQYCVFMTAFGMTSFAIAILILQLGGYILAAVGLSLCVMVCAPTQGPLLISGLAIIALAELLGLVYMKFKASNQKPPQPPRKAAFKED

SEQ ID NO: 177 - protein, 308 aa, synthetic construct
MWPLVAALLLGSACCGSAQLLFNKTKSVEFTFCNETVVIPCFVTNMEAENTNEVYVKWKFKGKDIYTFDGALNKSTTDSDFKSARIEVSELLKGDASLKMDKSDAVLGNYTCEVTELSREGETIIELKYRVVSWFSPNENILIVIFPIFAILLFWGQFGIKTLKYRSGRMDEKTIALLVAGLVITVIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAAFKED

SEQ ID NO: 178 - protein, 308 aa, synthetic construct
MWPLVAALLLGSACCGSAQLLFNKTKSVEFTFCNETVVIPCFVNNVEAENTNELYVKWKFKGKDIYTFDGAMNKSTTYSNFKSARIEVSELLKGDASLKMDKSDAVLGNYTCEVTELSREGETIIELKYRVVSWFSPNENILIVIFPIFAILLFWGQFGIKTLKYKSSHMDEKTIALLVAGLVITAIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAAFKED

SEQ ID NO: 179 - protein, 308 aa, synthetic construct
MWPLVAALLLGSACCGSAQLLFNKTKSVEFTFCNETVVIPCFVNNVEAENTKEMYVKWKFKGKDIFIFDGAQNKSTTYNNFTSAKIAVSELLKGDASLKMDKSDAVLGNYTCEVTELSREGETIIELKYRVVSWFSPNENILIVIFPILAILLFWGQFGILTLKYKSNRTNEKTILLLVAGLLLTIVVIVGAILFIPGEYSIKNASGLGLIVISTGILILLQYCVFMTAFGMTSFTIAILIIQVLGYVLAVVGLSLCISACIPVHGPLLISGLGIIALAELLGLVYMKFVASNQKTIQPPRKAAFKED

SEQ ID NO: 180 - protein, 308 aa, synthetic construct
MWPLVAALLLGSACCGSAQLIFNVTKSVEFTICNETVVIPCFVNNVEAKNIKEMYVKWKFKGKDIFTFDGAQNKSTTSNNFTSAKITPSELLKGIASLKMDKSDAVLGNYTCEVTELSREGETIIELKYRVVSWFSPNENILIVIFPILAILLSWGQFGIVTLKYKSNRTNEKTILLFVAGLLLTIVVIVGAILFIPGEYSIKNASGLGLIVIPTAILILLQYCVFMTAFGMTSFTIAILILQVLGYVLAVVGLSLCISECTPVHGPLLISGLGIIALAELLGLVYMKFVASNQKTIQPPRKAAFKED

SEQ ID NO: 181 - protein, 308 aa, synthetic construct
MWPLVAALLLGSACCGSAQLTFNATKSVEFTICNETVVIPCFVNNVETKNIKEMYVKWKFKGKDIFTFDGAQNKSTASSNFTSAKITPSELLKGIASLKMKKSDAVLGNYTCEVTELSREGETIVELKYRVVSWFSPKENILIVIFPILAILLSWGQFGIVTLKYKSNRTNEKTILLFVAGLLLTIVVIVGAILFIPGEYSIKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAAFKED

SEQ ID NO: 182 - protein, 310 aa, synthetic construct
MWPLVAALLLGSACCGSAQLLFNKTKSVEFTFCNETVVIPCFVTNVEAENTNELYVKWKFKGKDIYTFDGALNKSTTDSNFKSARIEVSELLKGDASLKMDKSDAVLGNYTCEVTELSREGETIIELKYRVVSWFSPNENILIVIFPIFAILLFWGQFGIKTLKYKSSRMDEKTIALLVAGLVITAIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAVEAFKED

SEQ ID NO: 183 - protein, 310 aa, synthetic construct
MWPLVAALLLGSACCGSAQLLFNKTKSVEFTFCNETVVIPCFVTNVEAENTNELYVKWKFKGKDIYTFDGAMNKSTTDSNFKSARIEVSELLKGDASLKMDKSDAVLGNYTCEVTELSREGETIIELKYRVVSWFSPNENILIVIFPIFAILLFWGQFGIKTLKYKSSRMDEKTIALLVAGLVTTAIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAVEAFKED

SEQ ID NO: 184 - protein, 310 aa, synthetic construct
MWPLVAALLLGSACCGSAQLLFNKTKSVEFTFCNETVVIPCFVTNVEAENTKEMYVKWKFKGKDIFIFDGAQNKSTTDNNFTSARIAVSELLKGDASLKMDKSDAVLGNYTCEVTELSREGETIIELKYRVVSWFSPNENILIVIFPIFAILLFWGQFGIKTLKYKSSRTDEKTILLLVAGLVVTAIVIVGAILFIPGEYSVKNATGLGLIVISTGILILLQYCVFKTAFGMTSFAIAILIIQVLGYVLAVVGLSLCISACIPMHGPLLISGLGIIALAQLLGLVYMKFVASNQKTIQPPRKAVEAFKED

SEQ ID NO: 185 - protein, 310 aa, synthetic construct
MWPLVAAQLLSSASCGSAQITFNTTKFVEITVCNETVVLPCFANNVETKNLRELYVKWKFKGKDIFIYDGAQDQATPSSNFSSAEITPSELLKGIASLKMKKSDAVLGNYTCEVTELSREGETIVELKYRVVSWFSPKENILIIIFPILAILLSWGQFGIVTLKYKSNRTNEKTILMFVAGLLLTIVVIVGAILFIPGEYSIKNASGLGLIVIPTVILILLQYCAFMTAFGMTSFAIAILILQVLGYVLAVVGLSLCISECTPVQGPLLISGLGIIALAELLGLVYMKFVASNQKTIQPPRKAVEAFKED

SEQ ID NO: 186 - protein, 310 aa, synthetic construct
MWPLVAAHLLSSASSGSAQITFNTTKFVEITVCNETVVLPCFATNVETKNLRELYLKWKFRGKDIFIYDGAQDLATPSSNFSSAEITPSELLKGNASLKMKKSDAVLGNYTCEVTELSREGETVVELKYRVVSWFSPKENILIIIFPILAILLSWGQFGIVTLKYKSNRTNEKTILMFVAGLLLTIVIIVGAILFIPGEYSIKNASGLGLIVIPTVILILLQYCAFMTAFGMTSFAIAILILQVLGYVLAVVGLSLCISECTPVQGPLLISGLGIIALAELLGLVYMKFVASNQKTIQPPRKAVEAFKED

SEQ ID NO: 187 - protein, 310 aa, synthetic construct
MWPLVAALLLGSACCGSAQLLFNKTKSVEFTFCNDTVVIPCFVTNMEAQNTTEVYVKWKFKGRDIYTFDGALNKSTVPSNFNSAKIEVSQLLKGDASLKMDKSDAVTGNYTCEVTELTREGETIIELKYRVVSWFSPNENILIVIFPIFAILLFWGQFGIKTLKYRSGGMDEKTIALLVAGLMITVIVIVGAILFVPGEYSLKNATGLGLIVTSTGILILLHYYVFSTAFGLTSFVIAILVIQVIAYVLAVVGLSLCIAACIPMHGPLLISGLSILALAQLLGLVYMKFVASNQKTIQPPRKAVEAFKED

SEQ ID NO: 188 - protein, 310 aa, synthetic construct
MWPLVAALLLGSACCGSAQLLFNKTKSVEFTFCNETVVIPCFVTNMEAENTNEVYVKWKFKGKDIYTFDGALNKSTTDSNFKSARIEVSELLKGDASLKMDKSDAVLGNYTCEVTELSREGETIIELKYRVVSWFSPNENILIVIFPIFAILLFWGQFGIKTLKYRSGRMDEKTIALLVAGLVITVIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAVEAFKED

SEQ ID NO: 189 - protein, 310 aa, synthetic construct
MWPLVAALLLTSACCGSAQLLFNKTKSVEYTVCNETISIPCFVINMEAENTNEVYVKWKFKGKDIYTFDGSDNRSTHDSAFKTSGIKISELLKGDATLMMHQSNAVPGNYTCEVTVLSREGETTIEVKYRIVSWFSPNENILIVIFPVFALLLFWGQFGIKTLQYRSGRMDEKTIGLLVAGLLVTIIIIVGAILFVPGEYSVKNATGLGLIVTSTGLLIILNYYVFKTAFGITSFVIALLIIQVLVYVLAGVGLSLCISACTPVHGPVLISGLGIFALVQLLGLIYMKFVASNQKTIQPPRKAVEEFKED

SEQ ID NO: 190 - protein, 308 aa, synthetic construct
MWPLVAALLLGSACCGSAQLLFNKTKSVEFTFCNETVVIPCFVNNVEAENTKEMYVKWKFKGKDIFIFDGAQNKSTAYNNFTSAKIAVSELLKGDASLKMDKSDAVLGNYTCEVTELSREGETIVELKYRVVSWFSPNENILIVIFPILAILLFWGQFGILTLKYKSNRTNEKTILLLVAGLLLTTVVIVGAILFIPGEYSIKNASGLGLIVISTGILILLQYCVFMTAFGMTSFTIAILIIQVLGYVLAVVGLSLCISACVPVHGPLLISGLGIIALAELLGLVYMKFVASNQKTIQPPRKAAFKED

SEQ ID NO: 191 - protein, 308 aa, synthetic construct
MWPLVAALLLGSACCGSAQLLFNKTKSVEFTFCNETVVIPCFVNNVEAENIKEMYVKWKFKGKDIFIFDGAQNKSTAYNNFTSAKIAVSELLKGDASLKMDKSDAVLGNYTCEVTELSREGETIVELKYRVVSWFSPNENILIVIFPILAILLFWGQFGILTLKYKSNRTNEKTILLLVAGLLLTIVVIVGAILFIPGEYSIKNASGLGLIVISTGILILLQYCVFMTAFGMTSFTIAILIIQVLGYVLAVVGLSLCISECVPVHGPLLISGLGIIALAELLGLVYMKFVASNQKTIQPPRKAAFKED

SEQ ID NO: 192 - protein, 308 aa, synthetic construct
MWPLVAALLLGSACCGSAQLLFNKTKSVEFTFCNETVVIPCFVNNVEAENIKEMYVKWKFKGKDIFIFDGAQNKSTAYNNFTSAKIAVSELLKGDASLKMDKSDAVLGNYTCEVTELSREGETIVELKYRVVSWFSPNENILIVIFPILAILLFWGQFGILTLKYKSNRTNEKTILLLVAGLLLTIVVIVGAILFIPGEYSIKNASGLGLIVIPTGILILLQYCVFMTAFGMTSFTIAILIIQVLGYVLAVVGLSLCISECVPVHGPLLISGLGIIALAELLGLVYMKFVASNQKTIQPPRKAAFKED

SEQ ID NO: 193 - protein, 308 aa, synthetic construct
MWPLVAALLLGSACCGSAQLIFNVTKSVEFTVCNETVVIPCFVNNVEAKNIKEMYVKWKFKGKDIFIFDGAQNKSTASNNFTSAKITPSELLKGIASLKMDKSDAVLGNYTCEVTELSREGETIVELKYRVVSWFSPNENILIVIFPILAILLSWGQFGIVTLKYKSNRTNEKTILLFVAGLLLTIVVIVGAILFIPGEYSIKNASGLGLIVIPTAILILLQYCVFMTAFGMTSFTIAILILQVLGYVLAVVGLSLCISECTPVHGPLLISGLGIIALAELLGLVYMKFVASNQKTIQPPRKAAFKED

SEQ ID NO: 194 - protein, 308 aa, synthetic construct
MWPMVAAQVLASGNMGSAQITFNATKFVEITACNETVVLPCFPNNLEPKSLGDLYVKWKFKGKDIFIYEGNKDQAVVSSNFSSAQITISELLKGVASLKMKKSDAVLGNYTCEVTELSREGETIVELRYRAVSWFSPKENILIIIFPILAILLSWGQFGIVTLKYTSNRTNEKTILMFVTGLILTIIVIVGAILFVPGEYSVKNASGLGLITIPTVVLILLQYCAFMTAFGMTFFAIAILILQVLGFVLAVVGLSLCISECTPVQGPLLISGLGIIALAELLGLVYMKFVASNQKTIQPPRKAAFKEE

SEQ ID NO: 195 - protein, 308 aa, synthetic construct
MWPLVAALLLGSACCGSAQLLFNKTKSVEFTFCNETVVIPCFVTNVEAENTNELYVKWKFKGKDIYTFDGALNKSTTYSNFKSARIEVSELLKGDASLKMDKSDAVLGNYTCEVTELSREGETIIELKYRVVSWFSPNENILIVIFPIFAILLFWGQFGIKTLKYKSSHMDEKTIALLVAGLVITAIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAAFKED

SEQ ID NO: 196 - protein, 309 aa, synthetic construct
MWPLVAALLLGSACCGSAQLLFNKTKSVEFTFCNETVVIPCFVNNVEAENTNEMYVKWKFKGKDIFIFDGAQNKSTAYNNFTSAKIAVSELLKGDASLKMDKSDAVLGNYTCEVTELSREGETIIELKYRVVSWFSPNENILIVIFPILAILLFWGQFGIVTLKYKSNRTNEKTILLLVAGLLLTTVVIVGAILFIPGEYSVKNASGLGLIVISTGILILLQYCVFMTAFGMTSFTIAILIIQVLGYVLAVVGLSLCISACIPVHGPLLISGLGIIALAELLGLVYMKFVASNQKTIQPPRKAVAFKED

SEQ ID NO: 197 - protein, 309 aa, synthetic construct
MWPLVAALLLGSACCGSAQLLFNKTKSVEFTFCNETVVIPCFVNNVEAENINEMYVKWKFKGKDIFIFDGAQNKSTAYNNFTSAKIAVSELLKGDASLKMDKSDAVLGNYTCEVTELSREGETIIELKYRVVSWFSPNENILIVIFPILAILLFWGQFGIVTLKYKSNRTNEKTILLLVAGLLLTIVVIVGAILFIPGEYSTKNASGLGLIVIPTGILILLQYCVFMTAFGMTSFTIAILIIQVLGYVLAVVGLSLCISACIPVHGPLLISGLGIIALAELLGLVYMKFVASNQKTIQPPRKAVAFKED

SEQ ID NO: 198 - protein, 309 aa, synthetic construct
MWPLVAALLLGSACCGSAQLIFNVTKSVEFTVCNETVVIPCFVNNVEAKNINEMYVKWKFKGKDIFIFDGAQNKSTPSNNFTSAKITPSELLKGIASLKMDKSDAVLGNYTCEVTELSREGETIIELKYRVVSWFSPNENILIVIFPILAILLSWGQFGIVTLKYKSNRTNEKTILLFVAGLLLTIVVIVGAILFIPGEYSTKNASGLGLIVIPTAILILLQYCVFMTAFGMTSFTIAILILQVLGYVLAVVGLSLCISECTPVHGPLLISGLGIIALAELLGLVYMKFVASNQKTIQPPRKAVAFKED

SEQ ID NO: 199 - protein, 309 aa, synthetic construct
MWPLVAALLLGSACCGSAQLTFNVTKSVEFTVCNETVVIPCFVNNVETKNIKEMYVKWKFKGKDIFIFDGAQNKSTPSSNFTSAKITPSELLKGIASLKMKKSDAVLGNYTCEVTELSREGETIIELKYRVVSWFSPKENILIVIFPILAILLSWGQFGIVTLKYKSNRTNEKTILLFVAGLLLTIVVIVGAILFIPGEYSTKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAVAFKED

SEQ ID NO: 200 - protein, 309 aa, synthetic construct
MWPAMLLLLLSSVCRGSAQLLFDSPRLVEKNACNHSVLLPCSVTNIELNNIRAMFVRWKYKGRDIYTFDGFQDKVTISNDFQSASIFVPDILSGNSSLRLSDDDAVLGNYTCEVTEANREGEIIVELRYHVVSWFNPKENILIVIFPILAILLSWGQFGIVTLKYKANCPSEKTFGLIISGIILTLFVIIGAALFIPGEYSIKNASGLGLIVVPTLMLVLLQYCVFMTAFGMTSFAIAILILQLGGYILAAVGLSLCVMVCAPTQGPLLISGLAIIALAELLGLVYMKFKASNQKPPQPPRKAVAFKED

SEQ ID NO: 201 - protein, 309 aa, synthetic construct
MWALGAWVLLGAVCAGSAQLLFNATKSVEQTICNKTVILPCIVTNLELNNTKVMFVKWKFKGKEIFSFDGSKEKFHRNNNFSSANLSLSDLPKGIASLSLSKTEAVPGNYTCEVTESNREGETIVELRYRVGSWFPPVENILIIIFLILAILFYWAQLGVVALKYETNCTLQKTIGLIIAGLIITIVVVVGAILLVPDGYTPKNAAGLGLIVVPAVILVLLQYFLFQIVFELPPFAIVIVVLQLLGYIIAVVGFSLCVTACPPKQGSVVIAGLAIIALAAVIGLVYVKIVGSNQKDHQPPRKAVAFKED

SEQ ID NO: 202 - protein, 305 aa, synthetic construct
MWALGAWVLLGAVCAGSAQLLFNATKSVEQTICNKTVILPCIVTNLELNNTKVMFVKWKLQGKEFFSFDGSKEKFHRNSNFSSANLVSLSDLPKGIASLSLSKTEAVPGNYTCEVTESNREGETVVELRYRVGSWFPPVENILIIVFLILAILFYWAQLGVVASKFETTLQKKIGLIIAGLIITIVAVVGAILLVPDGYTPKNQAGLGLIVVPAVILVPLQYFLFQIVFENLPPFAIVLVVLKSLGYIIAVVGFALCVSACPPKQGSVVIAGLAIMALAAVIGLVYVIIMGSNKKDHQPPRKAVD

SEQ ID NO: 203 - protein, 309 aa, synthetic construct
MWPLVAALLLGSACCGSAQLLFNKTKSVEFTFCNETVVIPCFVNNVEAENTNEMYVKWKFKGKDIFIFDGAQNKSTTYNNFTSARIAVSELLKGDASLKMDKSDAVLGNYTCEVTELSREGETIIELKYRVVSWFSPNENILIVIFPIFAILLFWGQFGIKTLKYKSSHTDEKTILLLVAGLVVTAVVIVGAILFIPGEYSVKNATGLGLIVISTGILILLQYCVFKTAFGMTSFAIAILIIQVLGYVLAVVGLSLCISACIPMHGPLLISGLGIIALAQLLGLVYMKFVASNQKTIQPPRKAVAFKED

SEQ ID NO: 204 - protein, 310 aa, synthetic construct
MWPLVAALLLGSACCGSAQLLFNKTKSVEFTFCNETVVIPCFVNNVEAENTNEMYVKWKFKGKDIFIFDGAQNKSTAYSNFTSAKIAVSELLKGDASLKMDKSDAVLGNYTCEVTELSREGETIIELKYRVVSWFSPNENILIVIFPILAILLFWGQFGIVTLKYKSNRTNEKTILLLVAGLLLTTVVIVGAILFIPGEYSVKNASGLGLIVISTGILILLQYCVFMTAFGMTSFTIAILIIQVLGYVLAVVGLSLCISACIPVHGPLLISGLGIIALAELLGLVYMKFVASNQKTIQPPRKAVEAFKED

SEQ ID NO: 205 - protein, 310 aa, synthetic construct
MWPLVAALLLGSACCGSAQLLFNKTKSVEFTFCNETVVIPCFVNNVEAENINELYVKWKFKGKDIFIFDGAQNKSTAYSNFTSAKIAPSELLKGDASLKMDKSDAVLGNYTCEVTELSREGETIIELKYRVVSWFSPNENILIVIFPILAILLFWGQFGIVTLKYKSNRTNEKTILLLVAGLLLTIVVIVGAILFIPGEYSTKNASGLGLIVIPTGILILLQYCVFMTAFGMTSFTIAILIIQVLGYVLAVVGLSLCISACIPVHGPLLISGLGIIALAELLGLVYMKFVASNQKTIQPPRKAVEAFKED

SEQ ID NO: 206 - protein, 310 aa, synthetic construct
MWPLVAALLLGSACCGSAQLIFNVTKSVEFTVCNETVVIPCFVNNVEAKNINELYVKWKFKGKDIFIFDGAQNKSTPSSNFTSAKITPSELLKGIASLKMDKSDAVLGNYTCEVTELSREGETIIELKYRVVSWFSPNENILIVIFPILAILLFWGQFGIVTLKYKSNRTNEKTILLFVAGLLLTIVVIVGAILFIPGEYSTKNASGLGLIVIPTVILILLQYCVFMTAFGMTSFTIAILILQVLGYVLAVVGLSLCISECTPVHGPLLISGLGIIALAELLGLVYMKFVASNQKTIQPPRKAVEAFKED

SEQ ID NO: 207 - protein, 310 aa, synthetic construct
MWPLVAALLLGSACCGSAQITFNVTKSVEFTVCNETVVIPCFVNNVETKNINELYVKWKFKGKDIFIFDGAQNKSTPSSNFTSAKITPSELLKGIASLKMKKSDAVLGNYTCEVTELSREGETIIELKYRVVSWFSPKENILIVIFPILAILLFWGQFGIVTLKYKSNRTNEKTILLFVAGLLLTIVVIVGAILFIPGEYSTKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAVEAFKED

SEQ ID NO: 208 - protein, 310 aa, synthetic construct
MWPAMLLLLLSSVCRGSAQLLFDSPRLVEKNACNHSVLLPCSVTNIELNNIRAMFVRWKYKGRDIYTFDGFQDKVTISNDFQSASIFVPDILSGNSSLRLSDDDAVLGNYTCEVTEANREGEIIVELRYHVVSWFNPKENILIVIFPILAILLSWGQFGIVTLKYKANCPSEKTFGLIISGIILTLFVIIGAALFIPGEYSIKNASGLGLIVVPTLMLVLLQYCVFMTAFGMTSFAIAILILQLGGYILAAVGLSLCVMVCAPTQGPLLISGLAIIALAELLGLVYMKFKASNQKPPQPPRKAVEAFKED

SEQ ID NO: 209 - protein, 310 aa, synthetic construct
MWALGAWVLLGAVGAGSAQLLFNATKSVEQTICNKTVILPCIVTNLQLNNTKVMFVKWKLQGKEFFSFDGSKEKFHRNSNFSSANLSLSDLPKGIASLSLSKTEAVPGNYTCEVTESNREGETIVELRYSVASWFPPVENILIIVFLILAIIFYWAQLGVVASKFETNCTLQKKIGLIIAGLIITIVAVVGAILLVPDGYTPKNQAGLGLIVVPAVILVPLQYFLFQIVFELPPFAIVLVVLKSLGYIIAVVGFALCVSACPPKQGSVVIAGLAIIALAAVIGLVYVIIIGSNKKDHQPPRKAVEAFKED

SEQ ID NO: 210 - protein, 306 aa, synthetic construct
MWALRVWVLLGTLGAGSAQLLFEKIRSVEMDICNSTVIIPCIVTNLQLNNTKAMFVKWKLQGKEFFSFDGFEDKFHRNSSFQSANLVSLSNLPNGIASLSLSRKEAIPGNYTCEVTESNREGETIVELRYSVASWFQPVENVLIIVTMILAVIFYWAQLGVVAIKFDMTLQKKIGLIIAGLIVTIVAVIGAILLVPDGYTASNQAGLGLIVVPAVILVPLLYFLFQSVFEKPPLFAIILVVLKALGYIIAVVGFALCVSACPPKQGSVVIAGLAIIDLVAVIGLIYVIIIGSNLKDHQPPRKAVED

SEQ ID NO: 211 - protein, 310 aa, synthetic construct
MWPLVAALLLGSACCGSAQLLFNKTKSVEFTFCNDTVVIPCFVTNMEAQNTTEVYVKWKFKGRDIYTFDGALNKSTVPSDFNSAKIEVSQLLKGDASLKMDKSDAVTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAFGLTSFVIAILVIQVIAYVLAVVGLSLCIAACIPMHGPLLISGLSILALAQLLGLVYMKFVASNQKTIQPPRKAVEAFKED

SEQ ID NO: 212 - protein, 310 aa, synthetic construct
MWPLVAALLLGSACCGSAQLLFNKTKSVEFTFCNETVVIPCFVNNVEAENTNEMYVKWKFKGKDIFIFDGAQNKSTTYSNFTSARIAVSELLKGDASLKMDKSDAVLGNYTCEVTELSREGETIIELKYRVVSWFSPNENILIVIFPIFAILLFWGQFGIKTLKYKSSHTDEKTILLLVAGLVVTAVVIVGAILFIPGEYSVKNATGLGLIVISTGILILLQYCVFKTAFGMTSFAIAILIIQVLGYVLAVVGLSLCISACIPMHGPLLISGLGIIALAQLLGLVYMKFVASNQKTIQPPRKAVEAFKED

SEQ ID NO: 213 - protein, 309 aa, synthetic construct
MWPLVAALLLGSACCGSAQLLFNKTKSVEFTFCNETVVIPCFVTNVEAENTNELYVKWKFKGKDIYTFDGALNKSTTDSNFKSARIEVSELLKGDASLKMDKSDAVLGNYTCEVTELSREGETIIELKYRVVSWFSPNENILIVIFPIFAILLFWGQFGIKTLKYKSSRMDEKTIALLVAGLVITAIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAVAFKED

SEQ ID NO: 214 - protein, 309 aa, synthetic construct
MWPLVAALLLGSACCGSAQLLFNKTKSVEFTFCNETVVIPCFVTNVEAENTNELYVKWKFKGKDIYTFDGALNKSTTYSNFKSARIEVSELLKGDASLKMDKSDAVLGNYTCEVTELSREGETIIELKYRVVSWFSPNENILIVIFPIFAILLFWGQFGIKTLKYKSSRMDEKTIALLVAGLVITAIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAVAFKED

SEQ ID NO: 215 - protein, 309 aa, synthetic construct
MWPLVAALLLGSACCGSAQLLFNKTKSVEFTFCNETVVIPCFVNNVEAENTKEMYVKWKFKGKDIFIFDGAQNKSTTYNNFTSARIAVSELLKGDASLKMDKSDAVLGNYTCEVTELSREGETIIELKYRVVSWFSPNENILIVIFPIFAILLFWGQFGIKTLKYKSSRTDEKTILLLVAGLVVTAVVIVGAILFIPGEYSVKNATGLGLIVISTGILILLQYCVFKTAFGMTSFAIAILIIQVLGYVLAVVGLSLCISACIPMHGPLLISGLGIIALAQLLGLVYMKFVASNQKTIQPPRKAVAFKED

SEQ ID NO: 216 - protein, 309 aa, synthetic construct
MWPLVAAQLLSSASCGSAQITFNTTKFVEITVCNETVVLPCFANNVETKNLRELYVKWKFKGKDIFIYDGAQDQATPSSNFSSAEITPSELLKGIASLKMKKSDAVLGNYTCEVTELSREGETIVELKYRVVSWFSPKENILIIIFPILAILLSWGQFGIVTLKYKSNRTNEKTILMFVAGLLLTIVVIVGAILFIPGEYSIKNASGLGLIVIPTVILILLQYCAFMTAFGMTSFAIAILILQVLGYVLAVVGLSLCISECTPVQGPLLISGLGIIALAELLGLVYMKFVASNQKTIQPPRKAVAFKED

SEQ ID NO: 217 - protein, 309 aa, synthetic construct
MWPLVAAHLLSSASSGSAQITFNTTKFVEITVCNETVVLPCFANNVETKNLRELYLKWKFRGKDIFIYDGAQDLATPSSNFSSAEITPSELLKGNASLKMKKSDAVLGNYTCEVTELSREGETVVELKYRVVSWFSPKENILIVIFPILAILLSWGQFGIVTLKYKSNRTNEKTILMFVAGLLLTIVIIVGAILFIPGEYSIKNASGLGLIVIPTVILILLQYCAFMTAFGMTSFAIAILILQVLGYVLAVVGLSLCISECTPVQGPLLISGLGIIALAELLGLVYMKFVASNQKTIQPPRKAVAFKED

SEQ ID NO: 218 - protein, 309 aa, synthetic construct
MWPLVAALLLGSACCGSAQLLFNKTKSVEFTFCNDTVVIPCFVTNMEAQNTTEVYVKWKFKGRDIYTFDGALNKSTVPSNFNSAKIEVSQLLKGDASLKMDKSDAVTGNYTCEVTELTREGETIIELKYRVVSWFSPNENILIVIFPIFAILLFWGQFGIKTLKYRSGGMDEKTIALLVAGLMITVIVIVGAILFVPGEYSLKNATGLGLIVTSTGILILLHYYVFSTAFGLTSFVIAILVIQVIAYVLAVVGLSLCIAACIPMHGPLLISGLSILALAQLLGLVYMKFVASNQKTIQPPRKAVAFKED

SEQ ID NO: 219 - protein, 309 aa, synthetic construct
MWPLVAALLLGSACCGSAQLLFNKTKSVEFTFCNETVVIPCFVTNMEAENTNEVYVKWKFKGKDIYTFDGALNKSTTDSNFKSARIEVSELLKGDASLKMDKSDAVLGNYTCEVTELSREGETIIELKYRVVSWFSPNENILIVIFPIFAILLFWGQFGIKTLKYRSGRMDEKTIALLVAGLVITVIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAVAFKED

SEQ ID NO: 220 - protein, 309 aa, synthetic construct
MWPLVAALLLTSACCGSAQLLFNKTKSVEYTVCNETISIPCFVINMEAENTNEVYVKWKFKGKDIYTFDGSDNRSTHDSAFKTSGIKISELLKGDATLMMHQSNAVPGNYTCEVTVLSREGETTIEVKYRIVSWFSPNENILIVIFPVFALLLFWGQFGIKTLQYRSGRMDEKTIGLLVAGLLVTIIIIVGAILFVPGEYSVKNATGLGLIVTSTGLLIILNYYVFKTAFGITSFVIALLIIQVLVYVLAGVGLSLCISACTPVHGPVLISGLGIFALVQLLGLIYMKFVASNQKTIQPPRKAVEFKED

SEQ ID NO: 221 - protein, 308 aa, synthetic construct
MWPLVAALLLGSACCGSAQITFNTTKSVEFTVCNETVVIPCFATNVETKNLKELYVKWKFKGKDIFIYDGAQNQSTPSSNFTSAKITPSELLKGIASLKMKKSDAVLGNYTCEVTELSREGETIVELKYRVVSWFSPKENILIIIFPILAILLSWGQFGIVTLKYKSNRTNEKTILLFVAGLLLTIVVIVGAILFIPGEYSIKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAAFKED

SEQ ID NO: 222 - protein, 308 aa, synthetic construct
MWPLVAALLLGSACCGSAQITFNTTKSVEFTVCNETVVIPCFANNVETKNLKELYVKWKFKGKDIFIYDGAQNQSTPSSNFTSAKITPSELLKGIASLKMKKSDAVLGNYTCEVTELSREGETIVELKYRVVSWFSPKENILIIIFPILAILLSWGQFGIVTLKYKSNRTNEKTILLFVAGLLLTIVVIVGAILFIPGEYSIKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAAFKED

SEQ ID NO: 223 - protein, 309 aa, synthetic construct
MWPLVAALLLGSACCGSAQLLFNKTKSVEFTFCNETVVIPCFVNNVEAENTKEMYVKWKFKGKDIFIFDGAQNKSTAYNNFTSAKIAVSELLKGDASLKMDKSDAVLGNYTCEVTELSREGETIVELKYRVVSWFSPNENILIVIFPILAILLFWGQFGIVTLKYKSNRTNEKTILLLVAGLLLTTVVIVGAILFIPGEYSIKNASGLGLIVISTGILILLQYCVFMTAFGMTSFTIAILIIQVLGYVLAVVGLSLCISACVPVHGPLLISGLGIIALAELLGLVYMKFVASNQKTIQPPRKAVAFKED

SEQ ID NO: 224 - protein, 309 aa, synthetic construct
MWPLVAALLLGSACCGSAQLLFNKTKSVEFTFCNETVVIPCFVNNVEAENIKEMYVKWKFKGKDIFIFDGAQNKSTAYNNFTSAKIAVSELLKGDASLKMDKSDAVLGNYTCEVTELSREGETIVELKYRVVSWFSPNENILIVIFPILAILLFWGQFGIVTLKYKSNRTNEKTILLLVAGLLLTIVVIVGAILFIPGEYSIKNASGLGLIVIPTGILILLQYCVFMTAFGMTSFTIAILIIQVLGYVLAVVGLSLCISACVPVHGPLLISGLGIIALAELLGLVYMKFVASNQKTIQPPRKAVAFKED

SEQ ID NO: 225 - protein, 308 aa, synthetic construct
MWPLVAALLLGSVCGGSAQLTFNSTRFVEKTICNETVILPCIVNNLELKNIKAMYVKWKFKGKDIFTFDGNQNKSNASNNFKSANITLSDLLKGIASLKMKKSDAVPGNYTCEVTESNREGETIVELRYRVVSWFSPKENILIVIFPILAILLSWGQFGIVTLKYKSNCTSEKTIGLIIAGLILTIVVIVGAILFIPGEYSIKNASGLGLIVIPTVILVLLQYCVFMTAFGMTSFAIAILILQLLGYILAVVGLSLCITECAPMQGPLLISGLGIIALAELLGLVYMKFVASNQKTIQPPRKAAFKED

SEQ ID NO: 226 - protein, 308 aa, synthetic construct
MWPLVAALLLGSVCGGSAQLTFNSTRFVEKTICNETVILPCIVNNLELKNIKAMYVKWKYKGKDIFTFDGNQNKSNASNNFKSANIALSDLLKGNASLKMKKSDAVPGNYTCEVTESNREGETIVELRYHVVSWFSPKENILIVIFPILAILLSWGQFGIVTLKYKSNCTSEKTIGLIIAGLILTIVVIVGAVLFIPGEYSIKNASGLGLIVIPTVILVLLQYCVFMTAFGMTSFAIAILILQLLGYILAVVGLSLCITECAPMQGPLLISGLGIIALAELLGLVYMKFVASNQKTIQPPRKAAFKED

SEQ ID NO: 227 - protein, 308 aa, synthetic construct
MWPLVAALLLGSACCGSAQLLFNKTKSVEFTFCNETVVIPCFVTNVEAENTKEMYVKWKFKGKDIFIFDGAQNKSTTDSNFTSARIAVSELLKGDASLKMDKSDAVLGNYTCEVTELSREGETIIELKYRVVSWFSPNENILIVIFPIFAILLFWGQFGIKTLKYKSSRTDEKTILLLVAGLVVTAIVIVGAILFIPGEYSVKNATGLGLIVISTGILILLQYCVFKTAFGMTSFAIAILIIQVLGYVLAVVGLSLCISACIPMHGPLLISGLGIIALAQLLGLVYMKFVASNQKTIQPPRKAAFKED

SEQ ID NO: 228 - protein, 308 aa, synthetic construct
MWPLVAAQLLSSASCGSAQITFNTTKFVEITVCNETVVLPCFANNVETKNLRELYVKWKFKGKDIFIYDGAQDQATPSSNFSSAEITPSELLKGIASLKMKKSDAVLGNYTCEVTELSREGETIVELKYRVVSWFSPKENILIIIFPILAILLSWGQFGIVTLKYKSNRTNEKTILMFVAGLLLTIVVIVGAILFIPGEYSIKNASGLGLIVIPTVILILLQYCAFMTAFGMTSFAIAILILQVLGYVLAVVGLSLCISECTPVQGPLLISGLGIIALAELLGLVYMKFVASNQKTIQPPRKAAFKED

SEQ ID NO: 229 - protein, 308 aa, synthetic construct
MWPLVAAHLLSSASSGSAQITFNTTKFVEITVCNETVVLPCFATNVETKNLRELYLKWKFRGKDIFIYDGAQDLATPSSNFSSAEITPSELLKGNASLKMKKSDAVLGNYTCEVTELSREGETVVELKYRVVSWFSPKENILIVIFPILAILLSWGQFGIVTLKYKSNRTNEKTILMFVAGLLLTIVIIVGAILFIPGEYSIKNASGLGLIVIPTVILILLQYCAFMTAFGMTSFAIAILILQVLGYVLAVVGLSLCISECTPVQGPLLISGLGIIALAELLGLVYMKFVASNQKTIQPPRKAAFKED

SEQ ID NO: 230 - protein, 308 aa, synthetic construct
MWPLVAALLLGSACCGSAQITFNATKSVEFTVCNETVVIPCFANNVETKNLKELYVKWKFKGKDIFIYDGAQNQSTASSNFTSAKITPSELLKGIASLKMKKSDAVLGNYTCEVTELSREGETIVELKYRVVSWFSPKENILIIIFPILAILLSWGQFGIVTLKYKSNRTNEKTILLFVAGLLLTIVVIVGAILFIPGEYSIKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAAFKED

SEQ ID NO: 231 - protein, 308 aa, synthetic construct
MWPLVAALLLGSACCGSAQITFNVTKSVEFTVCNETVVIPCFATNVETKNLTELYVKWKFKGKDIFIYDGAQNQSTPSSNFTSAKITPSELLRGIASLKMKKSDAVLGNYTCEVTELSREGETIIELKYRVVSWFSPKENILIIIFPILAILLSWGQFGVVTLKYKSNRMNEKTILLFVAGLLLTIVVIVGAILFIPGEYSTKNASGLGLIVIPTVILILLQYFVFMTAFGMTSFAIAILILQVLGFVLAVVGLSLCISECTPVQGPLLISGLGIIALAELLGLVYMKFVASNQKTIQPPRKAAFKED

SEQ ID NO: 232 - protein, 308 aa, synthetic construct
MWPLVAALLLGSACCGSAQLLFNKTKSVEFTFCNETVVIPCFVTNVEAENTNEMYVKWKFKGKDIFIFDGAQNKSTTDSNFTSARIAVSELLKGDASLKMDKSDAVLGNYTCEVTELSREGETIIELKYRVVSWFSPNENILIVIFPIFAILLFWGQFGIKTLKYKSSRTDEKTILLLVAGLVVTAIVIVGAILFIPGEYSVKNATGLGLIVISTGILILLQYCVFKTAFGMTSFAIAILIIQVLGYVLAVVGLSLCISACIPMHGPLLISGLGIIALAQLLGLVYMKFVASNQKTIQPPRKAAFKED

SEQ ID NO: 233 - protein, 308 aa, synthetic construct
MWPLVAAHLLSSASSGSAQITFNTTKFVEITVCNETVVLPCFATNVETKNLRELYLKWKFRGKDIFIYDGAQDLATPSSNFSSAEITPSELLKGNASLKMKKSDAVLGNYTCEVTELSREGETVVELKYRVVSWFSPKENILIIIFPILAILLSWGQFGIVTLKYKSNRTNEKTILMFVAGLLLTIVIIVGAILFIPGEYSIKNASGLGLIVIPTVILILLQYCAFMTAFGMTSFAIAILILQVLGYVLAVVGLSLCISECTPVQGPLLISGLGIIALAELLGLVYMKFVASNQKTIQPPRKAAFKED

SEQ ID NO: 234 - protein, 308 aa, synthetic construct
MWPLVAALLLGSACCGSAQLLFNKTKSVEFTFCNETVVIPCFVTNVEAENTNELYVKWKFKGKDIYTFDGALNKSTTYSNFKSARIEVSELLKGDASLKMDKSDAVLGNYTCEVTELSREGETIIELKYRVVSWFSPNENILIVIFPIFAILLFWGQFGIKTLKYKSSRMDEKTIALLVAGLVITAIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAAFKED

SEQ ID NO: 235 - protein, 309 aa, synthetic construct
MWPLVAALLLGSACCGSAQLIFNVTKSVEFTVCNETVVIPCFVNNVEAKNIKEMYVKWKFKGKDIFIFDGAQNKSTASNNFTSAKITPSELLKGIASLKMDKSDAVLGNYTCEVTELSREGETIVELKYRVVSWFSPNENILIVIFPILAILLSWGQFGIVTLKYKSNRTNEKTILLFVAGLLLTIVVIVGAILFIPGEYSIKNASGLGLIVIPTAILILLQYCVFMTAFGMTSFTIAILILQVLGYVLAVVGLSLCISECTPVHGPLLISGLGIIALAELLGLVYMKFVASNQKTIQPPRKAVAFKED

SEQ ID NO: 236 - protein, 309 aa, synthetic construct
MWPLVAALLLGSACCGSAQITFNATKSVEFTVCNETVVIPCFANNVETKNLKELYVKWKFKGKDIFIYDGAQNQSTASSNFTSAKITPSELLKGIASLKMKKSDAVLGNYTCEVTELSREGETIVELKYRVVSWFSPKENILIIIFPILAILLSWGQFGIVTLKYKSNRTNEKTILLFVAGLLLTIVVIVGAILFIPGEYSIKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAVAFKED

SEQ ID NO: 237 - protein, 309 aa, synthetic construct
MWPLVAALLLGSVCGGSAQLTFNSTRFVEKTICNETVILPCIVTNLELNNIKAMYVKWKFKGKDIFTFDGNQNKSNASNNFKSANIALSDLLKGNASLKMKKSDAVPGNYTCEVTESNREGETIVELRYHVVSWFSPKENILIVIFPILAILLSWGQFGIVTLKYKSNCTSEKTIGLIIAGLILTIVVIVGAVLFIPGEYSIKNASGLGLIVIPTVILVLLQYCVFMTAFGMTSFAIAILILQLLGYILAVVGLSLCITECAPMQGPLLISGLAIIALAELLGLVYMKFVASNQKTIQPPRKAVAFKED

SEQ ID NO: 238 - protein, 309 aa, synthetic construct
MWPLVAALLLGSACCGSAQLLFNKTKSVEFTFCNETVVIPCFVNNVEAENTNELYVKWKFKGKDIYTFDGAMNKSTTYSNFKSARIEVSELLKGDASLKMDKSDAVLGNYTCEVTELSREGETIIELKYRVVSWFSPNENILIVIFPIFAILLFWGQFGIKTLKYKSSHMDEKTIALLVAGLVITAIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAVAFKED

SEQ ID NO: 239 - protein, 308 aa, synthetic construct
MWPLVAALLLGSACCGSAQLLFNKTKSVEFTFCNETVVIPCFVNNVEAENTKEMYVKWKFKGKDIFIFDGAQNKSTTYNNFTSAKIAVSELLKGDASLKMDKSDAVLGNYTCEVTELSREGETIIELKYRVVSWFSPNENILIVIFPILAILLFWGQFGIVTLKYKSNRTNEKTILLLVAGLLLTIVVIVGAILFIPGEYSIKNASGLGLIVISTGILILLQYCVFMTAFGMTSFTIAILIIQVLGYVLAVVGLSLCISACIPVHGPLLISGLGIIALAELLGLVYMKFVASNQKTIQPPRKAAFKED

SEQ ID NO: 240 - protein, 308 aa, synthetic construct
MWPLVAALLLGSVCGGSAQLTFNSTRFVEKTICNETVILPCIVTNLELNNIKAMYVKWKFKGKDIFTFDGNQNKSNASNNFKSANIALSDLLKGNASLKMKKSDAVPGNYTCEVTESNREGETIVELRYHVVSWFSPKENILIVIFPILAILLSWGQFGIVTLKYKSNCTSEKTIGLIIAGLILTIVVIVGAVLFIPGEYSIKNASGLGLIVIPTVILVLLQYCVFMTAFGMTSFAIAILILQLLGYILAVVGLSLCITECAPMQGPLLISGLAIIALAELLGLVYMKFVASNQKTIQPPRKAAFKED

SEQ ID NO: 241 - protein, 308 aa, synthetic construct
MWPLVAALLLGSACCGSAQLLFNKTKSVEFTFCNDTVVIPCFVTNMEAQNTTEVYVKWKFKGRDIYTFDGALNKSTVPSDFKSAKIEVSQLLKGDASLKMDKSDAVTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAFGLTSFVIAILVIQVIAYVLAVVGLSLCIAACIPMHGPLLISGLSILALAQLLGLVYMKFVASNQKTIQPPRKAAFKED

SEQ ID NO: 242 - protein, 309 aa, synthetic construct
MWPLVAALLLGSACCGSAQLLFNKTKSVEFTFCNETVVIPCFVTNVEAENTKEMYVKWKFKGKDIFIFDGAQNKSTADSNFTSAKIAVSELLKGDASLKMDKSDAVLGNYTCEVTELSREGETIVELKYRVVSWFSPNENILIVIFPILAILLFWGQFGILTLKYKSNRTNEKTILLLVAGLLLTTIVIVGAILFIPGEYSIKNASGLGLIVISTGILILLQYCVFMTAFGMTSFTIAILIIQVLGYVLAVVGLSLCISACVPVHGPLLISGLGIIALAELLGLVYMKFVASNQKTIQPPRKAVAFKED

SEQ ID NO: 243 - protein, 309 aa, synthetic construct
MWPLVAALLLGSACCGSAQLLFNKTKSVEFTFCNETVVIPCFVTNVEAENIKEMYVKWKFKGKDIFIFDGAQNKSTASSNFTSAKIAVSELLKGDASLKMDKSDAVLGNYTCEVTELSREGETIVELKYRVVSWFSPNENILIVIFPILAILLFWGQFGIVTLKYKSNRTNEKTILLLVAGLLLTIVVIVGAILFIPGEYSIKNASGLGLIVIPTGILILLQYCVFMTAFGMTSFTIAILIIQVLGYVLAVVGLSLCISACVPVHGPLLISGLGIIALAELLGLVYMKFVASNQKTIQPPRKAVAFKED

SEQ ID NO: 244 - protein, 309 aa, synthetic construct
MWPLVAALLLGSACCGSAQLIFNVTKSVEFTICNETVVIPCFVNNVEAKNIKEMYVKWKFKGKDIFIFDGAQNKSTPSSNFTSAKITPSELLNGIASLKMDKSDAVLGNYTCEVTELSREGETIVELKYRVVSWFSPNENILIVIFPILAILLFWGQFGIVTLKYKSNRTNEKTILLFVAGLLLTIVVIVGAILFIPGEYSIKNASGLGLIVIPTVILILLQYCVFMTAFGMTSFTIAILILQVLGYVLAVVGLSLCISECTPVHGPLLISGLGIIALAELLGLVYMKFVASNQKTIQPPRKAVAFKED

SEQ ID NO: 245 - protein, 309 aa, synthetic construct
MWPLVAALLLGSACCGSAQLTFNATKSVEFTICNETVVIPCFVNNVETKNIKEMYVKWKFKGKDIFIFDGAQNKSTPSSNFTSAKITPSELLNGIASLKMKKSDAVLGNYTCEVTELSREGETIVELKYRVVSWFSPKENILIVIFPILAILLSWGQFGIVTLKYKSNRTNEKTILLFVAGLLLTIVVIVGAILFIPGEYSIKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAVAFKED

SEQ ID NO: 246 - protein, 309 aa, synthetic construct
MWPLVAALLLGSACCGSAQLTFNATRFVEVTICNETVVLPCIVNNVESKNIKDMYVKWKFKGKDIFTFDGNQNKSNASSNFTSANITPSELLNGIASLKMEKSDAVLGNYTCEVTELSREGETIVELKYRVVSWFSPKENILIVIFPILAILLSWGQFGIVTLKYKSNRTNEKTILLIVAGLLLTIVVIVGAILFIPGEYSIKNASGLGLIVIPTVILILLQYCVFMTAFGMTSFAIAILILQVLGYILAVVGLSLCISECAPMQGPLLISGLGIIALAELLGLVYMKFVASNQKTIQPPRKAVAFKED

SEQ ID NO: 247 - protein, 309 aa, synthetic construct
MWPLVAALLLGSACCGSAQLLFNKTKSVEFTFCNETVVIPCFVTNVEAENTNELYVKWKFKGKDIYTFDGAMNKSTTDSNFKSARIEVSELLKGDASLKMDKSDAVLGNYTCEVTELSREGETIIELKYRVVSWFSPNENILIVIFPIFAILLFWGQFGIKTLKYKSSRMDEKTIALLVAGLVTTAIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAVAFKED

SEQ ID NO: 248 - protein, 309 aa, synthetic construct
MWPLVAALLLGSACCGSAQLLFNKTKSVEFTFCNETVVIPCFVTNVEAENTKEMYVKWKFKGKDIFIFDGAQNKSTTDSNFTSARIAVSELLKGDASLKMDKSDAVLGNYTCEVTELSREGETIIELKYRVVSWFSPNENILIVIFPIFAILLFWGQFGIKTLKYKSSRTDEKTILLLVAGLVVTAIVIVGAILFIPGEYSVKNATGLGLIVISTGILILLQYCVFKTAFGMTSFAIAILIIQVLGYVLAVVGLSLCISACIPMHGPLLISGLGIIALAQLLGLVYMKFVASNQKTIQPPRKAVAFKED

SEQ ID NO: 249 - protein, 308 aa, synthetic construct
MWPLVAALLLGSACCGSAQLLFNKTKSVEFTFCNETVVIPCFVTNVEAENTKEMYVKWKFKGKDIFIFDGAQNKSTTYNNFTSARIAVSELLKGDASLKMDKSDAVLGNYTCEVTELSREGETIIELKYRVVSWFSPNENILIVIFPIFAILLFWGQFGIKTLKYKSSHTDEKTILLLVAGLVVTAVVIVGAILFIPGEYSVKNATGLGLIVISTGILILLQYCVFKTAFGMTSFAIAILIIQVLGYVLAVVGLSLCISACIPMHGPLLISGLGIIALAQLLGLVYMKFVASNQKTIQPPRKAAFKED

SEQ ID NO: 250 - protein, 308 aa, synthetic construct
MWPLVAAHLLSSACCGSAQITFNTTKFVEITVCNETVVLPCFANNVETKNLRELYVKWKFKGKDIFIYDGAQDQATASSNFSSAEITPSELLKGIASLKMKKSDAVLGNYTCEVTELSREGETIVELKYRVVSWFSPKENILIIIFPILAILLSWGQFGIVTLKYKSNRTNEKTILMFVAGLLLTIVVIVGAILFIPGEYSIKNASGLGLIVIPTVILILLQYCAFMTAFGMTSFAIAILILQVLGYVLAVVGLSLCISECTPVQGPLLISGLGIIALAELLGLVYMKFVASNQKTIQPPRKAAFKED

SEQ ID NO: 251 - protein, 308 aa, synthetic construct
MWPLVAAHLLSSASSGSAQITFNTTKFVEITVCNETVVLPCFANNVETKNLRELYLKWKFRGKDIFIYDGAQDLATPSSNFSSAEITPSELLKGNASLKMKKSDAVLGNYTCEVTELSREGETVVELKYRVVSWFSPKENILIVIFPILAILLSWGQFGIVTLKYKSNRTNEKTILMFVAGLLLTIVIIVGAILFIPGEYSIKNASGLGLIVIPTVILILLQYCAFMTAFGMTSFAIAILILQVLGYVLAVVGLSLCISECTPVQGPLLISGLGIIALAELLGLVYMKFVASNQKTIQPPRKAAFKED

SEQ ID NO: 252 - protein, 310 aa, synthetic construct
MWPLVAALLLGSACCGSAQITFNVTKSVEFTVCNETVVIPCFATNVETKNLTELYVKWKFKGKDIFIFDGAQNQSTPSSNFTSAKITPSELLRGIASLKMKKSDAVLGNYTCEVTELSREGETIIELKYRVVSWFSPKENILIIIFPILAILLFWGQFGVVTLKYKSNRMNEKTILLFVAGLLLTIVVIVGAILFIPGEYSTKNASGLGLIVIPTVILILLQYFVFMTAFGMTSFAIAILILQVLGFVLAVVGLSLCISECTPVQGPLLISGLGIIALAELLGLVYMKFVASNQKTIQPPRKAVEAFKED

SEQ ID NO: 253 - protein, 310 aa, synthetic construct
MWALGAWVLLGAVCAGSAQLLFNATKSVEQTICNKTVILPCIVTNLELNNTKVMFVKWKFQGKEIFSFDGSQEKFHRNNNFSSANLSLSDLPKGIASLSLSKTEAVPGNYTCEVTESNREGETIVELRYRVGSWFPPVENILIIIFLILAILFYWAQLGVVALKYETNCTLQKTIGLIIAGLIITIVVVVGAILLVPDGYTPKNAAGLGLIVVPAVILVLLQYFLFQIVFELPPFAIVIVVLQLLGYIIAVVGFSLCVTACPPKQGSVVIAGLAIIALAAVIGLVYVKIVGSNQKDHQPPRKAVEAFKED

SEQ ID NO: 254 - protein, 306 aa, synthetic construct
MWALGAWVLLGAVCAGSAQLLFNATKSVEQTICNKTVILPCIVTNLQLNNTKVMFVKWKLQGKEFFSFDGSKEKFHRNSNFSSANLVSLSDLPKGIASLSLSKTEAVPGNYTCEVTESNREGETVVELRYRVGSWFPPVENILIIVFLILAILFYWAQLGVVASKFETTLQKKIGLIIAGLIITIVAVVGAILLVPDGYTPKNQAGLGLIVVPAVILVPLQYFLFQIVFENLPPFAIVLVVLKSLGYIIAVVGFALCVSACPPKQGSVVIAGLAIMALAAVIGLVYVIIIGSNKKDHQPPRKAVED

SEQ ID NO: 255 - protein, 306 aa, synthetic construct
MWALGAWVLLGAVGAGSAQLLFNATKSVEQTICNKTVILPCIVTNLQLNNTKVMFVKWKLQGKEFFSFDGSKEKFHRNSNFSSANLVSLSDLPKGIASLSLSKTEAVPGNYTCEVTESNREGETVVELRYRVGSWFPPVENILIIVFLILAIIFYWAQLGVVASKFETTLQKKIGLIIAGLIITIVAVVGAILLVPDGYTPKNQAGLGLIVVPAVILVPLQYFLFQIVFENLPPFAIVLVVLKSLGYIIAVVGFALCVSACPPKQGSVVIAGLAIMALAAVIGLVYVIIIGSNMKDHQPPRKAVED

SEQ ID NO: 256 - protein, 308 aa, synthetic construct
MWPLVAALLLGSACCGSAQLLFNKTKSVEFTFCNETVVIPCFVNNVEAENIKEMYVKWKFKGKDIFIFDGAQNKSTTYNNFTSAKIAVSELLKGDASLKMDKSDAVLGNYTCEVTELSREGETIVELKYRVVSWFSPNENILIVIFPILAILLFWGQFGIVTLKYKSNRTNEKTILLLVAGLLLTIVVIVGAILFIPGEYSIKNASGLGLIVIPTGILILLQYCVFMTAFGMTSFTIAILIIQVLGYVLAVVGLSLCISACVPVHGPLLISGLGIIALAELLGLVYMKFVASNQKTIQPPRKAAFKED

SEQ ID NO: 257 - protein, 308 aa, synthetic construct
MWPLVAALLLGSACCGSAQLIFNVTKSVEFTICNETVVIPCFVNNVEAKNIKEMYVKWKFKGKDIFTFDGAQNKSTTSNNFTSAKITPSELLKGIASLKMDKSDAVLGNYTCEVTELSREGETIVELKYRVVSWFSPNENILIVIFPILAILLSWGQFGIVTLKYKSNRTNEKTILLFVAGLLLTIVVIVGAILFIPGEYSIKNASGLGLIVIPTAILILLQYCVFMTAFGMTSFTIAILILQVLGYVLAVVGLSLCISECTPVHGPLLISGLGIIALAELLGLVYMKFVASNQKTIQPPRKAAFKED

SEQ ID NO: 258 - protein, 309 aa, synthetic construct
MWPLVAALLLGSACCGSAQLLFNKTKSVEFTFCNETVVIPCFVNNVEAENTKEMYVKWKFKGKDIFIFDGAQNKSTAYNNFTSAKIAVSELLKGDASLKMDKSDAVLGNYTCEVTELSREGETIIELKYRVVSWFSPNENILIVIFPILAILLFWGQFGIVTLKYKSNRTNEKTILLLVAGLLLTIVVIVGAILFIPGEYSIKNASGLGLIVISTGILILLQYCVFMTAFGMTSFTIAILIIQVLGYVLAVVGLSLCISACIPVHGPLLISGLGIIALAELLGLVYMKFVASNQKTIQPPRKAVAFKED

SEQ ID NO: 259 - protein, 309 aa, synthetic construct
MWPLVAALLLGSACCGSAQLIFNVTKSVEFTICNETVVIPCFVNNVEAKNIKEMYVKWKFKGKDIFIFDGAQNKSTASNNFTSAKITPSELLNGIASLKMDKSDAVLGNYTCEVTELSREGETIIELKYRVVSWFSPNENILIVIFPILAILLSWGQFGIVTLKYKSNRTNEKTILLFVAGLLLTIVVIVGAILFIPGEYSIKNASGLGLIVIPTVILILLQYCVFMTAFGMTSFTIAILILQVLGYVLAVVGLSLCISECTPVHGPLLISGLGIIALAELLGLVYMKFVASNQKTIQPPRKAVAFKED

SEQ ID NO: 260 - protein, 309 aa, synthetic construct
MWPLVAALLLGSACCGSAQLTFNATKSVEFTICNETVVIPCFVNNVETKNIKEMYVKWKFKGKDIFIFDGAQNKSTASSNFTSAKITPSELLNGIASLKMKKSDAVLGNYTCEVTELSREGETIVELKYRVVSWFSPKENILIVIFPILAILLSWGQFGIVTLKYKSNRTNEKTILLFVAGLLLTIVVIVGAILFIPGEYSIKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAVAFKED

SEQ ID NO: 261 - protein, 309 aa, synthetic construct
MWPLVAALLLGSACCGSAQITFNATKSVEFTVCNETVVIPCFANNVETKNLKELYVKWKFKGKDIFIYDGAQNQSTPSSNFTSAKITPSELLKGIASLKMKKSDAVLGNYTCEVTELSREGETIVELKYRVVSWFSPKENILIIIFPILAILLSWGQFGIVTLKYKSNRTNEKTILLFVAGLLLTIVVIVGAILFIPGEYSIKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAVAFKED

SEQ ID NO: 262 - protein, 309 aa, synthetic construct
MWPLVAALLLGSACCGSAQLLFNKTKSVEFTFCNETVVIPCFVTNVEAENTNEMYVKWKFKGKDIFIFDGAQNKSTTDSNFTSARIAVSELLKGDASLKMDKSDAVLGNYTCEVTELSREGETIIELKYRVVSWFSPNENILIVIFPIFAILLFWGQFGIKTLKYKSSRTDEKTILLLVAGLVVTAIVIVGAILFIPGEYSVKNATGLGLIVISTGILILLQYCVFKTAFGMTSFAIAILIIQVLGYVLAVVGLSLCISACIPMHGPLLISGLGIIALAQLLGLVYMKFVASNQKTIQPPRKAVAFKED

SEQ ID NO: 263 - protein, 309 aa, synthetic construct
MWPLVAALLLGSACCGSAQITFNATKFVEVTVCNETVVLPCFVNNVETKNIKELYVKWKFKGKDIFIFDGNQNKSNASSNFTSAKITPSELLKGIASLKMKKSDAVLGNYTCEVTELSREGETIVELKYRVVSWFSPKENILIVIFPILAILLSWGQFGIVTLKYKSNRTNEKTILLFVAGLLLTIVVIVGAILFIPGEYSIKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAVAFKED

SEQ ID NO: 264 - protein, 308 aa, synthetic construct
MWPLVAALLLGSACCGSAQLLFNKTKSVEFTFCNETVVIPCFVTNVEAENTKEMYVKWKFKGKDIFIFDGAQNKSTTYNNFTSARIAVSELLKGDASLKMDKSDAVLGNYTCEVTELSREGETIIELKYRVVSWFSPNENILIVIFPIFAILLFWGQFGIKTLKYKSSRTDEKTILLLVAGLVVTAVVIVGAILFIPGEYSVKNATGLGLIVISTGILILLQYCVFKTAFGMTSFAIAILIIQVLGYVLAVVGLSLCISACIPMHGPLLISGLGIIALAQLLGLVYMKFVASNQKTIQPPRKAAFKED

SEQ ID NO: 265 - protein, 308 aa, synthetic construct
MWPLVAALLLGSACCGSAQLLFNKTKSVEFTFCNETVVIPCFVNNVEAENTKEMYVKWKFKGKDIFIFDGAQNKSTTYNNFTSAKIAVSELLKGDASLKMDKSDAVLGNYTCEVTELSREGETIVELKYRVVSWFSPNENILIVIFPILAILLFWGQFGIVTLKYKSNRTNEKTILLLVAGLLLTTVVIVGAILFIPGEYSIKNASGLGLIVISTGILILLQYCVFMTAFGMTSFTIAILIIQVLGYVLAVVGLSLCISACVPVHGPLLISGLGIIALAELLGLVYMKFVASNQKTIQPPRKAAFKED

SEQ ID NO: 266 - protein, 310 aa, synthetic construct
MWPLVAALLLGSACCGSAQLLFNKTKSVEFTFCNETVVIPCFVTNVEAENTKEMYVKWKFKGKDIFIFDGAQNKSTADNNFTSAKIAVSELLKGDASLKMDKSDAVLGNYTCEVTELSREGETIVELKYRVVSWFSPNENILIVIFPILAILLFWGQFGILTLKYKSNRTNEKTILLLVAGLLLTTIVIVGAILFIPGEYSIKNASGLGLIVISTGILILLQYCVFMTAFGMTSFTIAILIIQVLGYVLAVVGLSLCISACVPVHGPLLISGLGIIALAELLGLVYMKFVASNQKTIQPPRKAVEAFKED

SEQ ID NO: 267 - protein, 310 aa, synthetic construct
MWPLVAALLLGSACCGSAQLLFNKTKSVEFTFCNETVVIPCFVTNVEAENIKEMYVKWKFKGKDIFIFDGAQNKSTASNNFTSAKIAVSELLKGDASLKMDKSDAVLGNYTCEVTELSREGETIVELKYRVVSWFSPNENILIVIFPILAILLFWGQFGIVTLKYKSNRTNEKTILLLVAGLLLTIVVIVGAILFIPGEYSIKNASGLGLIVIPTGILILLQYCVFMTAFGMTSFTIAILIIQVLGYVLAVVGLSLCISACVPVHGPLLISGLGIIALAELLGLVYMKFVASNQKTIQPPRKAVEAFKED

SEQ ID NO: 268 - protein, 310 aa, synthetic construct
MWPLVAALLLGSACCGSAQLIFNVTKSVEFTICNETVVIPCFVNNVEAKNIKEMYVKWKFKGKDIFTFDGAQNKSTASNNFTSAKITPSELLKGIASLKMDKSDAVLGNYTCEVTELSREGETIVELKYRVVSWFSPNENILIVIFPILAILLSWGQFGIVTLKYKSNRTNEKTILLFVAGLLLTIVVIVGAILFIPGEYSIKNASGLGLIVIPTAILILLQYCVFMTAFGMTSFTIAILILQVLGYVLAVVGLSLCISECTPVHGPLLISGLGIIALAELLGLVYMKFVASNQKTIQPPRKAVEAFKED

SEQ ID NO: 269 - protein, 310 aa, synthetic construct
MWPLVAALLLGSACCGSAQLTFNATKSVEFTICNETVVIPCFVNNVETKNIKEMYVKWKFKGKDIFTFDGAQNKSTASSNFTSAKITPSELLKGIASLKMKKSDAVLGNYTCEVTELSREGETIVELKYRVVSWFSPKENILIVIFPILAILLSWGQFGIVTLKYKSNRTNEKTILLFVAGLLLTIVVIVGAILFIPGEYSIKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAVEAFKED

SEQ ID NO: 270 - protein, 310 aa, synthetic construct
MWPLVAALLLGSACCGSAQLTFNATRFVEVTICNETVVLPCIVNNVESKNIKDMYVKWKFKGKDIFTFDGNQNKSNASSNFTSANITPSELLKGIASLKMKKSDAVLGNYTCEVTELSREGETIVELKYRVVSWFSPKENILIVIFPILAILLSWGQFGIVTLKYKSNRTNEKTILLIVAGLLLTIVVIVGAILFIPGEYSIKNASGLGLIVIPTVILILLQYCVFMTAFGMTSFAIAILILQVLGYILAVVGLSLCISECAPMQGPLLISGLGIIALAELLGLVYMKFVASNQKTIQPPRKAVEAFKED

SEQ ID NO: 271 - protein, 310 aa, synthetic construct; identical to SEQ ID NO: 76

SEQ ID NO: 272 - protein, 310 aa, synthetic construct
MWPLVAALLLGSVCGGSAQLTFNSTRFVEKTICNETVILPCIVTNLELNNIKAMFVKWKFKGKDIFTFDGNQNKSNASNNFKSANISLSDLLKGNASLKMKKSDAVPGNYTCEVTESNREGETIVELRYHVVSWFSPKENILIVIFPILAILLSWGQFGIVTLKYKSNCTSEKTIGLIIAGLILTIVVIVGAVLFIPGEYSIKNASGLGLIVIPTVILVLLQYCVFMTAFGMTSFAIAILILQLLGYILAVVGLSLCITECAPMQGPLLISGLAIIALAELLGLVYMKFVASNQKTIQPPRKAVEAFKED

SEQ ID NO: 273 - protein, 309 aa, synthetic construct
MWPLVAALLLGSACCGSAQLLFNKTKSVEFTFCNETVVIPCFVNNVEAENTNEMYVKWKFKGKDIFIFDGAQNKSTAYSNFTSAKIAVSELLKGDASLKMDKSDAVLGNYTCEVTELSREGETIIELKYRVVSWFSPNENILIVIFPILAILLFWGQFGIVTLKYKSNRTNEKTILLLVAGLLLTTVVIVGAILFIPGEYSVKNASGLGLIVISTGILILLQYCVFMTAFGMTSFTIAILIIQVLGYVLAVVGLSLCISACIPVHGPLLISGLGIIALAELLGLVYMKFVASNQKTIQPPRKAVAFKED

SEQ ID NO: 274 - protein, 309 aa, synthetic construct
MWPLVAALLLGSACCGSAQLLFNKTKSVEFTFCNETVVIPCFVNNVEAENINELYVKWKFKGKDIFIFDGAQNKSTAYSNFTSAKIAPSELLKGDASLKMDKSDAVLGNYTCEVTELSREGETIIELKYRVVSWFSPNENILIVIFPILAILLFWGQFGIVTLKYKSNRTNEKTILLLVAGLLLTIVVIVGAILFIPGEYSTKNASGLGLIVIPTGILILLQYCVFMTAFGMTSFTIAILIIQVLGYVLAVVGLSLCISACIPVHGPLLISGLGIIALAELLGLVYMKFVASNQKTIQPPRKAVAFKED

SEQ ID NO: 275 - protein, 309 aa, synthetic construct
MWPLVAALLLGSACCGSAQLIFNVTKSVEFTVCNETVVIPCFVNNVEAKNINELYVKWKFKGKDIFIFDGAQNKSTPSSNFTSAKITPSELLKGIASLKMDKSDAVLGNYTCEVTELSREGETIIELKYRVVSWFSPNENILIVIFPILAILLFWGQFGIVTLKYKSNRTNEKTILLFVAGLLLTIVVIVGAILFIPGEYSTKNASGLGLIVIPTVILILLQYCVFMTAFGMTSFTIAILILQVLGYVLAVVGLSLCISECTPVHGPLLISGLGIIALAELLGLVYMKFVASNQKTIQPPRKAVAFKED

SEQ ID NO: 276 - protein, 309 aa, synthetic construct
MWPLVAALLLGSACCGSAQITFNVTKSVEFTVCNETVVIPCFVNNVETKNINELYVKWKFKGKDIFIFDGAQNKSTPSSNFTSAKITPSELLKGIASLKMKKSDAVLGNYTCEVTELSREGETIIELKYRVVSWFSPKENILIVIFPILAILLFWGQFGIVTLKYKSNRTNEKTILLFVAGLLLTIVVIVGAILFIPGEYSTKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAVAFKED

SEQ ID NO: 277 - protein, 309 aa, synthetic construct
MWALGAWVLLGAVCAGSAQLLFNATKSVEQTICNKTVILPCIVTNLELNNTKVMFVKWKFKGKEIFSFDGSQEKFHRNNNFSSANLSLSDLPKGIASLSLSKTEAVPGNYTCEVTESNREGETIVELRYRVGSWFPPVENILIIIFLILAILFYWAQLGVVALKYETNCTLQKTIGLIIAGLIITIVVVVGAILLVPDGYTPKNAAGLGLIVVPAVILVLLQYFLFQIVFELPPFAIVIVVLQLLGYIIAVVGFSLCVTACPPKQGSVVIAGLAIIALAAVIGLVYVKIVGSNQKDHQPPRKAVAFKED

SEQ ID NO: 278 - protein, 305 aa, synthetic construct
MWALGAWVLLGAVCAGSAQLLFNATKSVEQTICNKTVILPCIVTNLELNNTKVMFVKWKLQGKEFFSFDGSKEKFHRNNNFSSANLVSLSDLPKGIASLSLSKTEAVPGNYTCEVTESNREGETIVELRYRVGSWFPPVENILIIVFLILAILFYWAQLGVVASKFETTLQKKIGLIIAGVIITIVAVVGAILLVPDGYTPKNQAGLGLIVVPAVILVPLQYFLFQIVFENLPPFAIVLVVLKSLGYIIAVVGFALCVSACPPKQGSVVIAGLAIMALAAVIGLVYVIIIGSNKKDHQPPRKAVD

SEQ ID NO: 279 - protein, 309 aa, synthetic construct
MWPLVAALLLGSACCGSAQLLFNKTKSVEFTFCNETVVIPCFVNNVEAENTNEMYVKWKFKGKDIFIFDGAQNKSTTYSNFTSARIAVSELLKGDASLKMDKSDAVLGNYTCEVTELSREGETIIELKYRVVSWFSPNENILIVIFPIFAILLFWGQFGIKTLKYKSSHTDEKTILLLVAGLVVTAVVIVGAILFIPGEYSVKNATGLGLIVISTGILILLQYCVFKTAFGMTSFAIAILIIQVLGYVLAVVGLSLCISACIPMHGPLLISGLGIIALAQLLGLVYMKFVASNQKTIQPPRKAVAFKED

SEQ ID NO: 280 - protein, 308 aa, synthetic construct
MWPLVAALLLGSACCGSAQLLFNKTKSVEFTFCNETVVIPCFVTNVEAENTKEMYVKWKFKGKDIFIFDGAQNKSTADNNFTSAKIAVSELLKGDASLKMDKSDAVLGNYTCEVTELSREGETIVELKYRVVSWFSPNENILIVIFPILAILLFWGQFGILTLKYKSNRTNEKTILLLVAGLLLTTIVIVGAILFIPGEYSIKNASGLGLIVISTGILILLQYCVFMTAFGMTSFTIAILIIQVLGYVLAVVGLSLCISACVPVHGPLLISGLGIIALAELLGLVYMKFVASNQKTIQPPRKAAFKED

SEQ ID NO: 281 - protein, 308 aa, synthetic construct
MWPLVAALLLGSACCGSAQLLFNKTKSVEFTFCNETVVIPCFVTNVEAENIKEMYVKWKFKGKDIFIFDGAQNKSTASNNFTSAKIAVSELLKGDASLKMDKSDAVLGNYTCEVTELSREGETIVELKYRVVSWFSPNENILIVIFPILAILLFWGQFGIVTLKYKSNRTNEKTILLLVAGLLLTIVVIVGAILFIPGEYSIKNASGLGLIVIPTGILILLQYCVFMTAFGMTSFTIAILIIQVLGYVLAVVGLSLCISACVPVHGPLLISGLGIIALAELLGLVYMKFVASNQKTIQPPRKAAFKED

SEQ ID NO: 282 - protein, 308 aa, synthetic construct
MWPLVAALLLGSACCGSAQLIFNVTKSVEFTICNETVVIPCFVNNVEAKNIKEMYVKWKFKGKDIFTFDGAQNKSTASNNFTSAKITPSELLKGIASLKMDKSDAVLGNYTCEVTELSREGETIVELKYRVVSWFSPNENILIVIFPILAILLSWGQFGIVTLKYKSNRTNEKTILLFVAGLLLTIVVIVGAILFIPGEYSIKNASGLGLIVIPTAILILLQYCVFMTAFGMTSFTIAILILQVLGYVLAVVGLSLCISECTPVHGPLLISGLGIIALAELLGLVYMKFVASNQKTIQPPRKAAFKED

SEQ ID NO: 283 - protein, 308 aa, synthetic construct
MWPLVAALLLGSACCGSAQLLFNKTKSVEFTFCNETVVIPCFVNNVEAENTNEMYVKWKFKGKDIFIFDGAQNKSTAYSNFTSAKIAVSELLKGDASLKMDKSDAVLGNYTCEVTELSREGETIIELKYRVVSWFSPNENILIVIFPILAILLFWGQFGIVTLKYKSNRTNEKTILLLVAGLLLTTVVIVGAILFIPGEYSVKNASGLGLIVISTGILILLQYCVFMTAFGMTSFTIAILIIQVLGYVLAVVGLSLCISACIPVHGPLLISGLGIIALAELLGLVYMKFVASNQKTIQPPRKAAFKED

SEQ ID NO: 284 - protein, 308 aa, synthetic construct
MWPLVAALLLGSACCGSAQLLFNKTKSVEFTFCNETVVIPCFVNNVEAENINELYVKWKFKGKDIFIFDGAQNKSTAYSNFTSAKIAPSELLKGDASLKMDKSDAVLGNYTCEVTELSREGETIIELKYRVVSWFSPNENILIVIFPILAILLFWGQFGIVTLKYKSNRTNEKTILLLVAGLLLTIVVIVGAILFIPGEYSTKNASGLGLIVIPTGILILLQYCVFMTAFGMTSFTIAILIIQVLGYVLAVVGLSLCISACIPVHGPLLISGLGIIALAELLGLVYMKFVASNQKTIQPPRKAAFKED

SEQ ID NO: 285 - protein, 308 aa, synthetic construct
MWPLVAALLLGSACCGSAQLIFNVTKSVEFTVCNETVVIPCFVNNVEAKNINELYVKWKFKGKDIFIFDGAQNKSTPSSNFTSAKITPSELLKGIASLKMDKSDAVLGNYTCEVTELSREGETIIELKYRVVSWFSPNENILIVIFPILAILLFWGQFGIVTLKYKSNRTNEKTILLFVAGLLLTIVVIVGAILFIPGEYSTKNASGLGLIVIPTVILILLQYCVFMTAFGMTSFTIAILILQVLGYVLAVVGLSLCISECTPVHGPLLISGLGIIALAELLGLVYMKFVASNQKTIQPPRKAAFKED

SEQ ID NO: 286 - protein, 308 aa, synthetic construct
MWPLVAALLLGSACCGSAQITFNVTKSVEFTVCNETVVIPCFVNNVETKNINELYVKWKFKGKDIFIFDGAQNKSTPSSNFTSAKITPSELLKGIASLKMKKSDAVLGNYTCEVTELSREGETIIELKYRVVSWFSPKENILIVIFPILAILLFWGQFGIVTLKYKSNRTNEKTILLFVAGLLLTIVVIVGAILFIPGEYSTKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAAFKED

SEQ ID NO: 287 - protein, 308 aa, synthetic construct
MWALGAWVLLGAVCAGSAQLLFNATKSVEQTICNKTVILPCIVTNLELNNTKVMFVKWKLQGKEFFSFDGSKEKIHRNSNFSSANLSLSDLPKGIASLSLSKTEAVPGNYTCEVTESNREGETVVELRYRVVSWFPPVENILIIVFLILAILFYWAQLGVVASKFETNCTLQKKIGLIIAGLIITIVAVVGAILLVPDGYTPKNQAGLGLIVVPAVILVPLQYFLFQIVFELPPFAIVLVVLKSLGYIIAVVGFALCVSACPPKQGSVVIAGLAIMALAAVIGLVYVIIMGSNKKDHQPPRKAAFKED

SEQ ID NO: 288 - protein, 304 aa, synthetic construct
MWALGAWVLLGAVGAGSAQLLFNATKSVEQTICNKTVILPCIVTNLQLNNTKVMFVKWKLQGKEFFSFDGSKEKIHRNSNFSSANLVSLSDLPKGIASLSLSKTEAVPGNYTCEVTESNREGETVVELRYSVGSWFPPVENILIIVFLILAIIFYWAQLGVVASKFETTLQKKIGLIIAGLIITIVAVVGAILLVPDGYTPKNQAGLGLIVVPAVILVPLQYFLFQIVFENLPPFAIVLVVLKSLGYIIAVVGFALCVSACPPKQGSVVIAGLAIMALAAVIGLVYVIIIGSNMKDHQPPRKAD

SEQ ID NO: 289 - protein, 308 aa, synthetic construct
MWPLVAALLLGSACCGSAQLLFNKTKSVEFTFCNETVVIPCFVNNVEAENTNEMYVKWKFKGKDIFIFDGAQNKSTTYSNFTSARIAVSELLKGDASLKMDKSDAVLGNYTCEVTELSREGETIIELKYRVVSWFSPNENILIVIFPIFAILLFWGQFGIKTLKYKSSHTDEKTILLLVAGLVVTAVVIVGAILFIPGEYSVKNATGLGLIVISTGILILLQYCVFKTAFGMTSFAIAILIIQVLGYVLAVVGLSLCISACIPMHGPLLISGLGIIALAQLLGLVYMKFVASNQKTIQPPRKAAFKED

SEQ ID NO: 290 - protein, 309 aa, synthetic construct
MWPLVAALLLGSACCGSAQITFNATKFVEVTICNETVVLPCFVNNVETKNIKELYVKWKFKGKDIFIFDGNQNKSNASSNFTSAKITPSELLKGIASLKMKKSDAVLGNYTCEVTELSREGETIVELKYRVVSWFSPKENILIVIFPILAILLSWGQFGIVTLKYKSNRTNEKTILLFVAGLLLTIVVIVGAILFIPGEYSIKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAVAFKED

SEQ ID NO: 291 - protein, 308 aa, synthetic construct
MWPLVAALLLGSACCGSAQITFNATKFVEVTVCNETVVLPCFVNNVETKNIKELYVKWKFKGKDIFIFDGNQNKSNASSNFTSAKITPSELLKGIASLKMKKSDAVLGNYTCEVTELSREGETIVELKYRVVSWFSPKENILIVIFPILAILLSWGQFGIVTLKYKSNRTNEKTILLFVAGLLLTIVVIVGAILFIPGEYSIKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAAFKED

SEQ ID NO: 292 - protein, 308 aa, synthetic construct
MWPLVAALLLGSACCGSAQLTFNATRFVEVTICNETVVLPCIVNNVESKNIKDMYVKWKFKGKDIFTFDGNQNKSNASSNFTSANITPSELLNGIASLKMKKSDAVLGNYTCEVTELSREGETIVELKYRVVSWFSPKENILIVIFPILAILLSWGQFGIVTLKYKSNRTNEKTILLIVAGLLLTIVVIVGAILFIPGEYSIKNASGLGLIVIPTVILILLQYCVFMTAFGMTSFAIAILILQVLGYILAVVGLSLCISECAPMQGPLLISGLGIIALAELLGLVYMKFVASNQKTIQPPRKAAFKED

SEQ ID NO: 293 - protein, 310 aa, synthetic construct
MWPLVAALLLGSACCGSAQLLFNKTKSVEFTFCNDTVVIPCFVTNMEAQNTTEVYVKWKFKGRDIYTFDGALNKSTAPANFSSAKIEVSQLLKGDASLKMDKSDAVTGNYTCEVTELTREGETIIELKYRVVSWFSPNENILIVIFPIFAILLFWGQFGIKTLKYRSGGMDEKTIALLVAGLMITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEAFKED

SEQ ID NO: 294 - protein, 310 aa, synthetic construct
MWPLVAALLLGSACCGSAQLTFNATKSVEFTICNETVVIPCFVNNVETKNIKEMYVKWKFKGKDIFIFDGAQNKSTASSNFTSAKITPSELLNGIASLKMKKSDAVLGNYTCEVTELSREGETIVELKYRVVSWFSPKENILIVIFPILAILLSWGQFGIVTLKYKSNRTNEKTILLFVAGLLLTIVVIVGAILFIPGEYSIKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAVEAFKED

SEQ ID NO: 295 - protein, 310 aa, synthetic construct
MWPLVAALLLGSACCGSAQITFNATKSVEFTVCNETVVIPCFANNVETKNLKELYVKWKFKGKDIFIYDGAQNQSTPSSNFTSAKITPSELLKGIASLKMKKSDAVLGNYTCEVTELSREGETIVELKYRVVSWFSPKENILIIIFPILAILLSWGQFGIVTLKYKSNRTNEKTILLFVAGLLLTIVVIVGAILFIPGEYSIKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAVEAFKED

SEQ ID NO: 296 - protein, 310 aa, synthetic construct
MWPLVAALLLGSACCGSAQITFNVTKSVEFTVCNETVVIPCFATNVETKNLTELYVKWKFKGKDIFIYDGAQNQSTPSSNFTSAKITPSELLRGIASLKMKKSDAVLGNYTCEVTELSREGETIIELKYRVVSWFSPKENILIIIFPILAILLSWGQFGVVTLKYKSNRMNEKTILLFVAGLLLTIVVIVGAILFIPGEYSTKNASGLGLIVIPTVILILLQYFVFMTAFGMTSFAIAILILQVLGFVLAVVGLSLCISECTPVQGPLLISGLGIIALAELLGLVYMKFVASNQKTIQPPRKAVEAFKED

SEQ ID NO: 297 - protein, 308 aa, synthetic construct
MWPLVAALLLGSACCGSAQLLFNKTKSVEFTFCNETVVIPCFVNNVEAENTKEMYVKWKFKGKDIFIFDGAQNKSTAYSNFTSAKIAVSELLKGDASLKMDKSDAVLGNYTCEVTELSREGETIVELKYRVVSWFSPNENILIVIFPILAILLFWGQFGIVTLKYKSNRTNEKTILLLVAGLLLTTVVIVGAILFIPGEYSIKNASGLGLIVISTGILILLQYCVFMTAFGMTSFTIAILIIQVLGYVLAVVGLSLCISACVPVHGPLLISGLGIIALAELLGLVYMKFVASNQKTIQPPRKAAFKED

SEQ ID NO: 298 - protein, 308 aa, synthetic construct
MWPLVAALLLGSACCGSAQLIFNVTKSVEFTICNETVVIPCFVNNVEAKNIKEMYVKWKFKGKDIFIFDGAQNKSTASSNFTSAKITPSELLNGIASLKMDKSDAVLGNYTCEVTELSREGETIVELKYRVVSWFSPNENILIVIFPILAILLFWGQFGIVTLKYKSNRTNEKTILLFVAGLLLTIVVIVGAILFIPGEYSIKNASGLGLIVIPTVILILLQYCVFMTAFGMTSFTIAILILQVLGYVLAVVGLSLCISECTPVHGPLLISGLGIIALAELLGLVYMKFVASNQKTIQPPRKAAFKED

SEQ ID NO: 299 - protein, 308 aa, synthetic construct
MWPLVAALLLGSACCGSAQLTFNATKSVEFTICNETVVIPCFVNNVETKNIKEMYVKWKFKGKDIFIFDGAQNKSTASSNFTSAKITPSELLNGIASLKMKKSDAVLGNYTCEVTELSREGETIVELKYRVVSWFSPKENILIVIFPILAILLSWGQFGIVTLKYKSNRTNEKTILLFVAGLLLTIVVIVGAILFIPGEYSIKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAAFKED

SEQ ID NO: 300 - protein, 308 aa, synthetic construct
MWPLVAALLLGSACCGSAQLLFNKTKSVEFTFCNETVVIPCFVNNVEAENTKEMYVKWKFKGKDIFIFDGAQNKSTTYSNFTSARIAVSELLKGDASLKMDKSDAVLGNYTCEVTELSREGETIIELKYRVVSWFSPNENILIVIFPIFAILLFWGQFGIKTLKYKSSHTDEKTILLLVAGLVVTAVVIVGAILFIPGEYSVKNATGLGLIVISTGILILLQYCVFKTAFGMTSFAIAILIIQVLGYVLAVVGLSLCISACIPMHGPLLISGLGIIALAQLLGLVYMKFVASNQKTIQPPRKAAFKED

SEQ ID NO: 301 - protein, 308 aa, synthetic construct
MWPLVAALLLGSACCGSAQLLFNKTKSVEFTFCNETVVIPCFVTNVEAENTNELYVKWKFKGKDIYTFDGALNKSTTDSNFKSARIEVSELLKGDASLKMDKSDAVLGNYTCEVTELSREGETIIELKYRVVSWFSPNENILIVIFPIFAILLFWGQFGIKTLKYKSSRMDEKTIALLVAGLVVTAIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAAFKED

SEQ ID NO: 302 - protein, 308 aa, synthetic construct
MWPLVAALLLGSACCGSAQLLFNKTKSVEFTFCNDTVVIPCFVTNMEAQNTTEVYVKWKFKGRDIYTFDGALNKSTVSTNFSSAKIEVSQLLKGDASLKMDKSDAVTGNYTCEVTELTREGETIIELKYRVVSWFSPNENILIVIFPIFAILLFWGQFGIKTLKYRSGGMDEKTIALLVAGLMITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAAFKED

SEQ ID NO: 303 - protein, 309 aa, synthetic construct
MWPLVAALLLGSACCGSAQLIFNVTKSVEFTICNETVVIPCFVNNVEAKNIKEMYVKWKFKGKDIFIFDGAQNKSTASNNFTSAKITPSELLNGIASLKMDKSDAVLGNYTCEVTELSREGETIVELKYRVVSWFSPNENILIVIFPILAILLFWGQFGIVTLKYKSNRTNEKTILLFVAGLLLTIVVIVGAILFIPGEYSIKNASGLGLIVIPTVILILLQYCVFMTAFGMTSFTIAILILQVLGYVLAVVGLSLCISECTPVHGPLLISGLGIIALAELLGLVYMKFVASNQKTIQPPRKAVAFKED

SEQ ID NO: 304 - protein, 308 aa, synthetic construct
MWPLVAALLLGSACCGSAQLLFNKTKSVEFTFCNETVVIPCFVTNVEAENTKEMYVKWKFKGKDIFIFDGAQNKSTAYNNFTSAKIAVSELLKGDASLKMDKSDAVLGNYTCEVTELSREGETIVELKYRVVSWFSPNENILIVIFPILAILLFWGQFGIVTLKYKSNRTNEKTILLLVAGLLLTTVVIVGAILFIPGEYSIKNASGLGLIVISTGILILLQYCVFMTAFGMTSFTIAILIIQVLGYVLAVVGLSLCISACVPVHGPLLISGLGIIALAELLGLVYMKFVASNQKTIQPPRKAAFKED

SEQ ID NO: 305 - protein, 308 aa, synthetic construct
MWPLVAALLLGSACCGSAQLLFNKTKSVEFTFCNETVVIPCFVTNVEAENIKEMYVKWKFKGKDIFIFDGAQNKSTAYNNFTSAKIAVSELLKGDASLKMDKSDAVLGNYTCEVTELSREGETIVELKYRVVSWFSPNENILIVIFPILAILLFWGQFGIVTLKYKSNRTNEKTILLLVAGLLLTIVVIVGAILFIPGEYSIKNASGLGLIVIPTGILILLQYCVFMTAFGMTSFTIAILIIQVLGYVLAVVGLSLCISACVPVHGPLLISGLGIIALAELLGLVYMKFVASNQKTIQPPRKAAFKED

SEQ ID NO: 306 - protein, 308 aa, synthetic construct
MWPLVAALLLGSVCGGSAQLTFNSTRFVEKTICNETVILPCIVNNLELKNIKAMYVKWKFKGKDIFTFDGNQNKSNASNNFKSANIALSDLLKGNASLKMKKSDAVPGNYTCEVTESNREGETIVELRYHVVSWFSPKENILIVIFPILAILLSWGQFGIVTLKYKSNCTSEKTIGLIIAGLILTIVVIVGAVLFIPGEYSIKNASGLGLIVIPTVILVLLQYCVFMTAFGMTSFAIAILILQLLGYILAVVGLSLCITECAPTQGPLLISGLGIIALAELLGLVYMKFVASNQKTIQPPRKAAFKED

SEQ ID NO: 307 - protein, 308 aa, synthetic construct
MWPLVAALLLGSVCGGSAQLTFNSTRFVEKTICNETVILPCIVTNLELNNIKAMFVKWKFKGKDIFTFDGNQNKSNASNNFKSANISLSDLLKGNASLKMKKSDAVPGNYTCEVTESNREGETIVELRYHVVSWFSPKENILIVIFPILAILLSWGQFGIVTLKYKSNCTSEKTIGLIIAGLILTIVVIVGAVLFIPGEYSIKNASGLGLIVIPTVILVLLQYCVFMTAFGMTSFAIAILILQLLGYILAVVGLSLCITECAPTQGPLLISGLAIIALAELLGLVYMKFVASNQKTIQPPRKAAFKED

SEQ ID NO: 308 - protein, 308 aa, synthetic construct
MWPLVAALLLGSACCGSAQLLFNKTKSVEFTFCNETVVIPCFVTNVEAENTKEMYVKWKFKGKDIFIFDGAQNKSTADSNFTSAKIAVSELLKGDASLKMDKSDAVLGNYTCEVTELSREGETIVELKYRVVSWFSPNENILIVIFPILAILLFWGQFGILTLKYKSNRTNEKTILLLVAGLLLTTIVIVGAILFIPGEYSIKNASGLGLIVISTGILILLQYCVFMTAFGMTSFTIAILIIQVLGYVLAVVGLSLCISACVPVHGPLLISGLGIIALAELLGLVYMKFVASNQKTIQPPRKAAFKED

SEQ ID NO: 309 - protein, 308 aa, synthetic construct
MWPLVAALLLGSACCGSAQLLFNKTKSVEFTFCNETVVIPCFVTNVEAENIKEMYVKWKFKGKDIFIFDGAQNKSTASSNFTSAKIAVSELLKGDASLKMDKSDAVLGNYTCEVTELSREGETIVELKYRVVSWFSPNENILIVIFPILAILLFWGQFGIVTLKYKSNRTNEKTILLLVAGLLLTIVVIVGAILFIPGEYSIKNASGLGLIVIPTGILILLQYCVFMTAFGMTSFTIAILIIQVLGYVLAVVGLSLCISACVPVHGPLLISGLGIIALAELLGLVYMKFVASNQKTIQPPRKAAFKED

SEQ ID NO: 310 - protein, 308 aa, synthetic construct
MWPLVAALLLGSACCGSAQLIFNVTKSVEFTICNETVVIPCFVNNVEAKNIKEMYVKWKFKGKDIFIFDGAQNKSTPSSNFTSAKITPSELLNGIASLKMDKSDAVLGNYTCEVTELSREGETIVELKYRVVSWFSPNENILIVIFPILAILLFWGQFGIVTLKYKSNRTNEKTILLFVAGLLLTIVVIVGAILFIPGEYSIKNASGLGLIVIPTVILILLQYCVFMTAFGMTSFTIAILILQVLGYVLAVVGLSLCISECTPVHGPLLISGLGIIALAELLGLVYMKFVASNQKTIQPPRKAAFKED

SEQ ID NO: 311 - protein, 310 aa, synthetic construct
MWPLVAALLLGSACCGSAQLIFNVTKSVEFTVCNETVVIPCFVNNVEAKNIKEMYVKWKFKGKDIFIFDGAQNKSTPSNNFTSAKITPSELLKGIASLKMDKSDAVLGNYTCEVTELSREGETIVELKYRVVSWFSPNENILIVIFPILAILLSWGQFGIVTLKYKSNRTNEKTILLFVAGLLLTIVVIVGAILFIPGEYSIKNASGLGLIVIPTAILILLQYCVFMTAFGMTSFTIAILILQVLGYVLAVVGLSLCISECTPVHGPLLISGLGIIALAELLGLVYMKFVASNQKTIQPPRKAVEAFKED

SEQ ID NO: 312 - protein, 310 aa, synthetic construct
MWPLVAALLLGSACCGSAQLTFNATKSVEFTVCNETVVIPCFVNNVETKNIKEMYVKWKFKGKDIFIFDGAQNKSTASSNFTSAKITPSELLKGIASLKMKKSDAVLGNYTCEVTELSREGETIVELKYRVVSWFSPKENILIVIFPILAILLSWGQFGIVTLKYKSNRTNEKTILLFVAGLLLTIVVIVGAILFIPGEYSIKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAVEAFKED

SEQ ID NO: 313 - protein, 310 aa, synthetic construct
MWPLVAALLLGSACCGSAQLTFNATRFVEVTICNETVVLPCIVNNVESKNIKDMYVKWKFKGKDIFTFDGNQNKSNASSNFTSANITPSELLKGIASLKMEKSDAVLGNYTCEVTELSREGETIVELKYRVVSWFSPKENILIVIFPILAILLSWGQFGIVTLKYKSNRTNEKTILLIVAGLLLTIVVIVGAILFIPGEYSIKNASGLGLIVIPTVILILLQYCVFMTAFGMTSFAIAILILQVLGYILAVVGLSLCISECAPMQGPLLISGLGIIALAELLGLVYMKFVASNQKTIQPPRKAVEAFKED

SEQ ID NO: 314 - protein, 310 aa, synthetic construct
MWPLVAALLLGSVCGGSAQLTFNSTRFVEKTICNETVILPCIVNNLELKNIKAMYVKWKFKGKDIFTFDGNQNKSNASNNFKSANIALSDLLKGNASLKMKKSDAVLGNYTCEVTESNREGETIVELRYHVVSWFSPKENILIVIFPILAILLSWGQFGIVTLKYKSNCTSEKTIGLIIAGLILTIVVIVGAVLFIPGEYSIKNASGLGLIVIPTVILVLLQYCVFMTAFGMTSFAIAILILQLLGYILAVVGLSLCITECAPMQGPLLISGLGIIALAELLGLVYMKFVASNQKTIQPPRKAVEAFKED

SEQ ID NO: 315 - protein, 310 aa, synthetic construct
MWPLVAALLLGSACCGSAQLLFNKTKSVEFTFCNETVVIPCFVTNMEAENTNEVYVKWKFKGKDIYTFDGALNKSTTDSDFKSARIEVSELLKGDASLKMDKSDAVLGNYTCEVTELSREGETIIELKYRVVSWFSPNENILIVIFPIFAILLFWGQFGIKTLKYRSGRMDEKTIALLVAGLVITVIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAVEAFKED

SEQ ID NO: 316 - protein, 310 aa, synthetic construct
MWPLVAALLLGSACCGSAQLLFNKTKSVEFTFCNETVVIPCFVTNVEAENTNELYVKWKFKGKDIYTFDGALNKSTTDSNFKSARIEVSELLKGDASLKMDKSDAVLGNYTCEVTELSREGETIIELKYRVVSWFSPNENILIVIFPIFAILLFWGQFGIKTLKYKSSGMDEKTIALLVAGLVITAIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAVEAFKED

SEQ ID NO: 317 - protein, 308 aa, synthetic construct
MWPLVAAHLLSSASCGSAQITFNTTKFVEITVCNETVVLPCFATNVETKNLRELYVKWKFKGKDIFIYDGAQDQATPSSNFSSAEITPSELLKGIASLKMKKSDAVLGNYTCEVTELSREGETIVELKYRVVSWFSPKENILIIIFPILAILLSWGQFGIVTLKYKSNRTNEKTILMFVAGLLLTIVVIVGAILFIPGEYSIKNASGLGLIVIPTVILILLQYCAFMTAFGMTSFAIAILILQVLGYVLAVVGLSLCISECTPVQGPLLISGLGIIALAELLGLVYMKFVASNQKTIQPPRKAAFKED

SEQ ID NO: 318 - protein, 308 aa, synthetic construct
MWPLVAALLLGSACCGSAQLTFNATRFVEVTICNETVVLPCIVNNVETKNIKDMYVKWKFKGKDIFTFDGNQNKSNASSNFTSANITPSELLKGIASLKMKKSDAVLGNYTCEVTELSREGETIVELKYRVVSWFSPKENILIVIFPILAILLSWGQFGIVTLKYKSNRTNEKTILLIVAGLLLTIVVIVGAILFIPGEYSIKNASGLGLIVIPTVILILLQYCVFMTAFGMTSFAIAILILQVLGYILAVVGLSLCISECAPMQGPLLISGLGIIALAELLGLVYMKFVASNQKTIQPPRKAAFKED

SEQ ID NO: 319 - protein, 308 aa, synthetic construct
MWPLVAALLLGSVCGGSAQLTFNSTRFVEETICNETVILPCIVNNLEIKNIKVMYVKWKFKGKDIFTFDGNQNKSNASNNFKSANITLSELLKGNASLKMKKSDAVPGNYTCEVTESSREGETIVELRYHVVSWFSPKENILIVIFPILAILLSWGQFGIVTLKYKSNCTSEKTIGLIIAGLVLTIVVIVGAVLFIPGEYSIKNASGLGLIVIPTVILVLLQYCVFMTAFGMTSFAIAILILQLLGYILAVVGLSLCITECAPMQGPLLISGLGIIALAELLGLVYMKFVASNQKTIQPPRKAAFKED

SEQ ID NO: 320 - protein, 308 aa, synthetic construct
MWPLVAALLLGSVCGGSAQLTFNSTRFVEETICNETVILPCIVTNLELNNIKVMFVKWKFKGKDIFTFDGNQNKSNASNNFKSANISLSDLLKGNASLRMSKSDAVPGNYTCEVTESNREGETIVELRYHVVSWFSPKENILIVIFPILAILLSWGQFGIVTLKYKSNCTSEKTIGLIIAGLILTIVVIVGAVLFIPGEYSIKNASGLGLIVIPTVILVLLQYCVFMTAFGMTSFAIAILILQLLGYILAVVGLSLCITECAPMQGPLLISGLAIIALAELLGLVYMKFVASNQKTIQPPRKAAFKED

SEQ ID NO: 321 - protein, 308 aa, synthetic construct
MWPLVAALLLGSACCGSAQLIFNVTKSVEFTICNETVVIPCFVNNVEAKNIKEMYVKWKFKGKDIFIFDGAQNKSTASNNFTSAKITPSELLNGIASLKMDKSDAVLGNYTCEVTELSREGETIVELKYRVVSWFSPNENILIVIFPILAILLFWGQFGIVTLKYKSNRTNEKTILLFVAGLLLTIVVIVGAILFIPGEYSIKNASGLGLIVIPTVILILLQYCVFMTAFGMTSFTIAILILQVLGYVLAVVGLSLCISECTPVHGPLLISGLGIIALAELLGLVYMKFVASNQKTIQPPRKAAFKED

SEQ ID NO: 322 - protein, 308 aa, synthetic construct
MWPLVAALLLGSACCGSAQLTFNATKFVEFTICNETVVIPCFVNNVETKNIKEMYVKWKFKGKDIFIFDGAQNKSTASSNFTSAKITPSELLNGIASLKMKKSDAVLGNYTCEVTELSREGETIVELKYRVVSWFSPKENILIVIFPILAILLSWGQFGIVTLKYKSNRTNEKTILLFVAGLLLTIVVIVGAILFIPGEYSIKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAAFKED

SEQ ID NO: 323 - protein, 308 aa, synthetic construct
MWPLVAALLLGSACCGSAQLTFNATRFVEVTICNETVVLPCIVNNVETKNIKDMYVKWKFKGKDIFTFDGNQNKSNASSNFTSANITPSELLNGIASLKMEKSDAVLGNYTCEVTELSREGETIVELKYRVVSWFSPKENILIVIFPILAILLSWGQFGIVTLKYKSNRTNEKTILLIVAGLLLTIVVIVGAILFIPGEYSIKNASGLGLIVIPTVILILLQYCVFMTAFGMTSFAIAILILQVLGYILAVVGLSLCISECAPMQGPLLISGLGIIALAELLGLVYMKFVASNQKTIQPPRKAAFKED

SEQ ID NO: 324 - protein, 308 aa, synthetic construct
MWPLVAALLLGSVCGGSAQLTFNSTRFVEETICNETVILPCIVNNLEIKNIKVMYVKWKFKGKDIFTFDGNQNKSNASNNFKSANITLSELLNGNASLKMKKSDAVLGNYTCEVTESSREGETIVELRYHVVSWFSPKENILIVIFPILAILLSWGQFGIVTLKYKSNCTSEKTIGLIIAGLVLTIVVIVGAVLFIPGEYSIKNASGLGLIVIPTVILVLLQYCVFMTAFGMTSFAIAILILQLLGYILAVVGLSLCITECAPMQGPLLISGLGIIALAELLGLVYMKFVASNQKTIQPPRKAAFKED

SEQ ID NO: 325 - protein, 308 aa, synthetic construct
MWPLVAALLLGSVCGGSAQLLFNSTRFVEETICNETVILPCIVTNLELNNIKVMFVKWKFKGKDIFTFDGNQNKSNASNNFKSANISLSDLLNGNASLRMSKSDAVLGNYTCEVTESNREGETIVELRYHVVSWFSPKENILIVIFPILAILLSWGQFGIVTLKYKSNCTSEKTIGLIIAGLILTIVVIVGAVLFIPGEYSIKNASGLGLIVIPTVILVLLQYCVFMTAFGMTSFAIAILILQLLGYILAVVGLSLCITECAPMQGPLLISGLAIIALAELLGLVYMKFVASNQKTIQPPRKAAFKED

SEQ ID NO: 326 - protein, 308 aa, synthetic construct
MWPLVAALLLGSACCGSAQLLFNKTKSVEFTFCNETVVIPCFVNNVEAENINELYVKWKFKGKDIFIFDGAQNKSTAYSNFTSAKIAPSELLKGDASLKMDKSDAVLGNYTCEVTELSREGETIIELKYRVVSWFSPNENILIVIFPILAILLFWGQFGIVTLKYKSNRTNEKTILLLVAGLLLTIVVIVGAILFIPGEYSTKNASGLGLIVIPTGILILLQYCVFMTAFGMTSFTIAILIIQVLGYVLAVVGLSLCISACVPVHGPLLISGLGIIALAELLGLVYMKFVASNQKTIQPPRKAAFKED

SEQ ID NO: 327 - protein, 308 aa, synthetic construct
MWPLVAALLLGSACCGSAQITFNVTKSVEFTVCNETVVIPCFVNNVETKNIKELYVKWKFKGKDIFIFDGAQNKSTPSSNFTSAKITPSELLKGIASLKMKKSDAVLGNYTCEVTELSREGETIIELKYRVVSWFSPKENILIVIFPILAILLFWGQFGIVTLKYKSNRTNEKTILLFVAGLLLTIVVIVGAILFIPGEYSTKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAAFKED

SEQ ID NO: 328 - protein, 308 aa, synthetic construct
MWALGAWVLLGAVCAGSAQLLFNATKSVEQTICNKTVILPCIVTNLELNNTKVMFVKWKFKGKEIFSFDGSQEKFHRNNNFSSANLSLSDLPKGIASLSLSKTEAVPGNYTCEVTESNREGETIVELRYRVGSWFPPVENILIIIFLILAILFYWAQLGVVALKYETNCTLQKTIGLIIAGLIITIVVVVGAILLVPDGYTPKNAAGLGLIVVPAVILVLLQYFLFQIVFELPPFAIVIVVLQLLGYIIAVVGFSLCVTACPPKQGSVVIAGLAIMALAAVIGLVYVKIVGSNQKDHQPPRKAAFKED

SEQ ID NO: 329 - protein, 304 aa, synthetic construct
MWALGAWVLLGAVCAGSAQLLFNATKSVEQTICNKTVILPCIVTNLELNNTKVMFVKWKLQGKEFFSFDGSKEKFHRNSNFSSANLVSLQDLPKGIASLSLSKTEAVPGNYTCEVTESNREGETVVELRYRVGSWFPPVENILIIVFLILAILFYWAQLGVVASKFETTLQKKIGLIIAGVIITIVAVVGAILLVPDGYTPKNQAGLGLIVVPAVILVPLQYFLFQIVFENLPPFAIVLVVLKSLGYIIAVVGFALCVTACPPKQGSVVIAGLAIMALAAVIGLVYVIIMGSNKKDHQPPRKAD

SEQ ID NO: 330 - protein, 309 aa, synthetic construct
MWPLVAALLLGSACCGSAQLLFNKTKSVEFTFCNETVVIPCFVTNVEAENTKEMYVKWKFKGKDIFIFDGAQNKSTADNNFTSAKIAVSELLKGDASLKMDKSDAVLGNYTCEVTELSREGETIVELKYRVVSWFSPNENILIVIFPILAILLFWGQFGILTLKYKSNRTNEKTILLLVAGLLLTTIVIVGAILFIPGEYSIKNASGLGLIVISTGILILLQYCVFMTAFGMTSFTIAILIIQVLGYVLAVVGLSLCISACVPVHGPLLISGLGIIALAELLGLVYMKFVASNQKTIQPPRKAVAFKED

SEQ ID NO: 331 - protein, 309 aa, synthetic construct
MWPLVAALLLGSACCGSAQLLFNKTKSVEFTFCNETVVIPCFVTNVEAENIKEMYVKWKFKGKDIFIFDGAQNKSTASNNFTSAKIAVSELLKGDASLKMDKSDAVLGNYTCEVTELSREGETIVELKYRVVSWFSPNENILIVIFPILAILLFWGQFGIVTLKYKSNRTNEKTILLLVAGLLLTIVVIVGAILFIPGEYSIKNASGLGLIVIPTGILILLQYCVFMTAFGMTSFTIAILIIQVLGYVLAVVGLSLCISACVPVHGPLLISGLGIIALAELLGLVYMKFVASNQKTIQPPRKAVAFKED

SEQ ID NO: 332 - protein, 309 aa, synthetic construct
MWPLVAALLLGSACCGSAQLLFNKTKSVEFTFCNETVVIPCFVTNVEAQNTNELYVKWKFKGKDIYTFDGALNKSTTDSNFKSARIEVSELLKGDASLKMDKSDAVLGNYTCEVTELSREGETIIELKYRVVSWFSPNENILIVIFPIFAILLFWGQFGIKTLKYKSSGMDEKTIALLVAGLVITAIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAVAFKED

SEQ ID NO: 333 - protein, 309 aa, synthetic construct
MWPLVAALLLGSACCGSAQLLFNKTKSVEFTFCNETVVIPCFVTNVEAENTNELYVKWKFKGKDIYTFDGAVNKSTTDSNFKSARIEVSELLKGDASLKMDKSDAVLGNYTCEVTELSREGETIIELKYRVVSWFSPNENILIVIFPIFAILLFWGQFGIKTLKYKSSRMDEKTIALLVAGLVITAIIIVGAILFVPGEYSVKNATGLGLIVTSTGLLILLHYYVFKTAFGITSFVIAILIIQVLAYVLAVVGLSLCISACIPMHGPLLISGLGIFALVQLLGLVYMKFVASNQKTIQPPRKAVAFKED

SEQ ID NO: 334 - protein, 309 aa, synthetic construct
MWPLVAALLLGSACCGSAQLLFNKTKSVEFTFCNETVVIPCFVTNVEAENTKEMYVKWKFKGKDIFIFDGAQNKSTTDNNFTSARIAVSELLKGDASLKMDKSDAVLGNYTCEVTELSREGETIIELKYRVVSWFSPNENILIVIFPIFAILLFWGQFGIKTLKYKSSRTDEKTILLLVAGLVVTAIVIVGAILFIPGEYSVKNATGLGLIVISTGILILLQYCVFKTAFGMTSFAIAILIIQVLGYVLAVVGLSLCISACIPMHGPLLISGLGIIALAQLLGLVYMKFVASNQKTIQPPRKAVAFKED

SEQ ID NO: 335 - protein, 308 aa, synthetic construct
MWPLVAALLLGSACCGSAQLLFNKTKSVEFTFCNETVVIPCFVNNVEAENIKEMYVKWKFKGKDIFIFDGAQNKSTAYSNFTSAKIAVSELLKGDASLKMDKSDAVLGNYTCEVTELSREGETIVELKYRVVSWFSPNENILIVIFPILAILLFWGQFGIVTLKYKSNRTNEKTILLLVAGLLLTIVVIVGAILFIPGEYSIKNASGLGLIVIPTGILILLQYCVFMTAFGMTSFTIAILIIQVLGYVLAVVGLSLCISACVPVHGPLLISGLGIIALAELLGLVYMKFVASNQKTIQPPRKAAFKED

SEQ ID NO: 336 - protein, 308 aa, synthetic construct
MWPLVAALLLGSACCGSAQLIFNTTKSVEFTICNETVVIPCFVNNVEAKNIKEMYVKWKFKGKDIFIFDGAQNKSTPSSNFTSAKITPSELLKGIASLKMDKSDAVLGNYTCEVTELSREGETIVELKYRVVSWFSPNENILIVIFPILAILLFWGQFGIVTLKYKSNRTNEKTILLFVAGLLLTIVVIVGAILFIPGEYSIKNASGLGLIVIPTVILILLQYCVFMTAFGMTSFTIAILILQVLGYVLAVVGLSLCISECTPVHGPLLISGLGIIALAELLGLVYMKFVASNQKTIQPPRKAAFKED

SEQ ID NO: 337 - protein, 308 aa, synthetic construct
MWPLVAALLLGSACCGSAQLTFNATKSVEFTICNETVVIPCFVNNVETKNIKEMYVKWKFKGKDIFIFDGAQNKSTPSSNFTSAKITPSELLKGIASLKMKKSDAVLGNYTCEVTELSREGETIVELKYRVVSWFSPKENILIVIFPILAILLSWGQFGIVTLKYKSNRTNEKTILLFVAGLLLTIVVIVGAILFIPGEYSIKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAAFKED

SEQ ID NO: 338 - protein, 308 aa, synthetic construct
MWPLVAAHLLSSASCGSAQITFNTTKFVEITVCNETVVLPCFANNVETKNLRELYVKWKFKGKDIFIYDGAQDQATPSSNFSSAEITPSELLKGIASLKMKKSDAVLGNYTCEVTELSREGETIVELKYRVVSWFSPKENILIIIFPILAILLSWGQFGIVTLKYKSNRTNEKTILMFVAGLLLTIVVIVGAILFIPGEYSIKNASGLGLIVIPTVILILLQYCAFMTAFGMTSFAIAILILQVLGYVLAVVGLSLCISECTPVQGPLLISGLGIIALAELLGLVYMKFVASNQKTIQPPRKAAFKED

SEQ ID NO: 339 - protein, 308 aa, synthetic construct
MWPLVAALLLGSACCGSAQLLFNKTKSVEFTFCNETVVIPCFVNNVEAENTKEMYVKWKFKGKDIFIFDGAQNKSTAYNNFTSAKIAVSELLKGDASLKMDKSDAVLGNYTCEVTELSREGETIIELKYRVVSWFSPNENILIVIFPILAILLFWGQFGIVTLKYKSNRTNEKTILLLVAGLLLTTVVIVGAILFIPGEYSVKNASGLGLIVISTGILILLQYCVFMTAFGMTSFTIAILIIQVLGYVLAVVGLSLCISACIPVHGPLLISGLGIIALAELLGLVYMKFVASNQKTIQPPRKAAFKED

SEQ ID NO: 340 - protein, 308 aa, synthetic construct
MWPLVAALLLGSACCGSAQLLFNKTKSVEFTFCNETVVIPCFVNNVEAENINELYVKWKFKGKDIFIFDGAQNKSTAYNNFTSAKIAPSELLKGDASLKMDKSDAVLGNYTCEVTELSREGETIIELKYRVVSWFSPNENILIVIFPILAILLFWGQFGIVTLKYKSNRTNEKTILLLVAGLLLTIVVIVGAILFIPGEYSTKNASGLGLIVIPTGILILLQYCVFMTAFGMTSFTIAILIIQVLGYVLAVVGLSLCISACIPVHGPLLISGLGIIALAELLGLVYMKFVASNQKTIQPPRKAAFKED

SEQ ID NO: 341 - protein, 308 aa, synthetic construct
MWALGAWVLLGAVCAGSAQLLFNATKSVEQTICNKTVILPCIVTNLELNNTKVMFVKWKFKGKEIFSFDGSKEKFHRNNNFSSANLSLSDLPKGIASLSLSKTEAVPGNYTCEVTESNREGETIVELRYRVGSWFPPVENILIIIFLILAILFYWAQLGVVALKYETNCTLQKTIGLIIAGLIITIVVVVGAILLVPDGYTPKNAAGLGLIVVPAVILVLLQYFLFQIVFELPPFAIVIVVLQLLGYIIAVVGFSLCVTACPPKQGSVVIAGLAIIALAAVIGLVYVKIVGSNQKDHQPPRKAAFKED

SEQ ID NO: 342 - protein, 304 aa, synthetic construct
MWALGAWVLLGAVCAGSAQLLFNATKSVEQTICNKTVILPCIVTNLELNNTKVMFVKWKLQGKEFFSFDGSKEKFHRNSNFSSANLVSLSDLPKGIASLSLSKTEAVPGNYTCEVTESNREGETIVELRYRVGSWFPPVENILIIVFLILAILFYWAQLGVVASKFETTLQKKIGLIIAGLIITIVAVVGAILLVPDGYTPKNQAGLGLIVVPAVILVPLQYFLFQIVFENLPPFAIVLVVLKSLGYIIAVVGFALCVSACPPKQGSVVIAGLAIMALAAVIGLVYVIIIGSNKKDHQPPRKAD

SEQ ID NO: 343 - protein, 308 aa, synthetic construct
MWPLVAALLLGSACCGSAQLLFNKTKSVEFTFCNETVVIPCFVNNVEAENTNEMYVKWKFKGKDIFIFDGAQNKSTTYNNFTSARIAVSELLKGDASLKMDKSDAVLGNYTCEVTELSREGETIIELKYRVVSWFSPNENILIVIFPIFAILLFWGQFGIKTLKYKSSHTDEKTILLLVAGLVVTAVVIVGAILFIPGEYSVKNATGLGLIVISTGILILLQYCVFKTAFGMTSFAIAILIIQVLGYVLAVVGLSLCISACIPMHGPLLISGLGIIALAQLLGLVYMKFVASNQKTIQPPRKAAFKED

SEQ ID NO: 344 - protein, 308 aa, synthetic construct
MWPLVAALLLGSACCGSAQLIFNITKSVEFTVCNETVVIPCFVNNVEAKNINELYVKWKFKGKDIFIFDGAQNKSTPSSNFTSAKITPSELLKGIASLKMDKSDAVLGNYTCEVTELSREGETIIELKYRVVSWFSPNENILIVIFPILAILLFWGQFGIVTLKYKSNRTNEKTILLFVAGLLLTIVVIVGAILFIPGEYSTKNASGLGLIVIPTVILILLQYCVFMTAFGMTSFTIAILILQVLGYVLAVVGLSLCISECTPVHGPLLISGLGIIALAELLGLVYMKFVASNQKTIQPPRKAAFKED

SEQ ID NO: 345 - protein, 308 aa, synthetic construct
MWALGAWVLLGAVCAGSAQLLFNATKSVEQTICNKTVILPCIVTNLELNNTKVMFVKWKFQGKEIFSFDGSKEKFHRNNNFSSANLSLSDLPKGIASLSLSKTEAVPGNYTCEVTESNREGETIVELRYRVGSWFPPVENILIIIFLILAILFYWAQLGVVALKYETNCTLQKTIGLIIAGLIITIVVVVGAILLVPDGYTPKNAAGLGLIVVPAVILVLLQYFLFQIVFELPPFAIVIVVLQLLGYIIAVVGFSLCVTACPPKQGSVVIAGLAIMALAAVIGLVYVKIVGSNQKDHQPPRKAAFKED

SEQ ID NO: 346 - protein, 304 aa, synthetic construct
MWALGAWVLLGAVCAGSAQLLFNATKSVEQTICNKTVILPCIVTNLELNNTKVMFVKWKLQGKEFFSFDGSKEKFHRNNNFSSANLVSLSDLPKGIASLSLSKTEAVPGNYTCEVTESNREGETIVELRYRVGSWFPPVENILIIVFLILAILFYWAQLGVVASKFETTLQKKIGLIIAGLIITIVAVVGAILLVPDGYTPKNQAGLGLIVVPAVILVPLQYFLFQIVFENLPPFAIVLVVLKSLGYIIAVVGFALCVSACPPKQGSVVIAGLAIMALAAVIGLVYVIIIGSNKKDHQPPRKAD

SEQ ID NO: 347 - protein, 304 aa, synthetic construct
MWALGAWVLLGAVCAGSAQLLFNATKSVEQTICNKTVILPCIVTNLELNNTKVMFVKWKLQGKEFFSFDGSKEKFHRNSNFSSANLVSLQDLPKGIASLSLSKTEAVPGNYTCEVTESNREGETVVELRYRVGSWFPPVENILIIVFLILAILFYWAQLGVVASKFETTLQKKIGLIIAGVIITIVAVVGAILLVPDGYTPKNQAGLGLIVVPAVILVPLQYFLFQIVFENLPPFAIVLVVLKSLGYIIAVVGFALCVSACPPKQGSVVIAGLAIMALAAVIGLVYVIIMGSNKKDHQPPRKAD

SEQ ID NO: 348 - protein, 308 aa, synthetic construct
MWPLVAALLLGSACCGSAQLIFNTTKSVEFTICNKTVVIPCFVNNVEAKNIKEMYVKWKFKGKDIFTFDGAQNKSTPSNNFTSAKITPSELLKGIASLKMDKSDAVLGNYTCEVTELSREGETIVELKYRVVSWFSPNENILIVIFPILAILLFWGQFGIVTLKYKSNRTNEKTILLFVAGLLLTIVVIVGAILFIPGEYSIKNASGLGLIVIPTVILILLQYCVFMTAFGMTSFTIAILILQVLGYVLAVVGLSLCISECTPVHGPLLISGLGIIALAELLGLVYMKFVASNQKTIQPPRKAAFKED

SEQ ID NO: 349 - protein, 308 aa, synthetic construct
MWPLVAALLLGSACCGSAQLTFNATRFVEVTICNKTVVLPCIVNNVESKNIKDMYVKWKFKGKDIFTFDGNQNKSNASSNFTSANITPSELLNGIASLKMKKSDAVLGNYTCEVTELSREGETIVELKYRVVSWFSPKENILIVIFPILAILLSWGQFGIVTLKYKSNRTNEKTILLIVAGLLLTIVVIVGAILFIPGEYSIKNASGLGLIVIPTVILILLQYCVFMTAFGMTSFAIAILILQVLGYILAVVGLSLCISECAPMQGPLLISGLGIIALAELLGLVYMKFVASNQKTIQPPRKAAFKED

SEQ ID NO: 350 - protein, 308 aa, synthetic construct
MWPLVAALLLGSVCGGSAQLTFNSTRFVEKTICNKTVILPCIVTNLELNNIKAMFVKWKFKGKDIFTFDGNQNKSNASNNFKSANISLSDLLKGNASLKMKKSDAVPGNYTCEVTESNREGETIVELRYHVVSWFSPKENILIVIFPILAILLSWGQFGIVTLKYKSNCTSEKTIGLIIAGLILTIVVIVGAVLFIPGEYSIKNASGLGLIVIPTVILVLLQYCVFMTAFGMTSFAIAILILQLLGYILAVVGLSLCITECAPMQGPLLISGLAIIALAELLGLVYMKFVASNQKTIQPPRKAAFKED

SEQ ID NO: 351 - protein, 308 aa, synthetic construct
MWPLVAALLLGSACCGSAQLIFNVTKSVEFTICNETVVIPCFVNNVEAKNIKEMYVKWKFKGKDIFIFDGAQNKSTTSNNFTSAKITPSELLNGIASLKMDKSDAVLGNYTCEVTELSREGETIVELKYRVVSWFSPNENILIVIFPILAILLSWGQFGIVTLKYKSNRTNEKTILLFVAGLLLTIVVIVGAILFIPGEYSIKNASGLGLIVIPTAILILLQYCVFMTAFGMTSFTIAILILQVLGYVLAVVGLSLCISECTPVHGPLLISGLGIIALAELLGLVYMKFVASNQKTIQPPRKAAFKED

SEQ ID NO: 352 - protein, 308 aa, synthetic construct
MWPLVAALLLGSACCGSAQLIFNTTKSVEFTICNETVVIPCFVNNVEAKNIKEMYVKWKFKGKDIFIFDGAQNKSTPSNNFTSAKITPSELLKGIASLKMDKSDAVLGNYTCEVTELSREGETIVELKYRVVSWFSPNENILIVIFPILAILLFWGQFGIVTLKYKSNRTNEKTILLFVAGLLLTIVVIVGAILFIPGEYSIKNASGLGLIVIPTVILILLQYCVFMTAFGMTSFTIAILILQVLGYVLAVVGLSLCISECTPVHGPLLISGLGIIALAELLGLVYMKFVASNQKTIQPPRKAAFKED

SEQ ID NO: 353 - protein, 309 aa, synthetic construct
MWPLVAALLLGSACCGSAQITFNVTKSVEFTVCNETVVIPCFATNVETKNLTELYVKWKFKGKDIFIFDGAQNQSTPSSNFTSAKITPSELLRGIASLKMKKSDAVLGNYTCEVTELSREGETIIELKYRVVSWFSPKENILIIIFPILAILLFWGQFGVVTLKYKSNRMNEKTILLFVAGLLLTIVVIVGAILFIPGEYSTKNASGLGLIVIPTVILILLQYFVFMTAFGMTSFAIAILILQVLGFVLAVVGLSLCISECTPVQGPLLISGLGIIALAELLGLVYMKFVASNQKTIQPPRKAVAFKED

SEQ ID NO: 354 - protein, 305 aa, synthetic construct
MWALGAWVLLGAVCAGSAQLLFNATKSVEQTICNKTVILPCIVTNLELNNTKVMFVKWKLQGKEFFSFDGSKEKFHRNSNFSSANLVSLQDLPKGIASLSLSKTEAVPGNYTCEVTESNREGETVVELRYRVGSWFPPVENILIIVFLILAILFYWAQLGVVASKFETTLQKKIGLIIAGVIITIVAVVGAILLVPDGYTPKNQAGLGLIVVPAVILVPLQYFLFQIVFENLPPFAIVLVVLKSLGYIIAVVGFALCVSACPPKQGSVVIAGLAIMALAAVIGLVYVIIMGSNKKDHQPPRKAVD

SEQ ID NO: 355 - protein, 305 aa, synthetic construct
MWALGAWVLLGAVGAGSAQLLFNATKSVEQTICNKTVILPCIVTNLQLNNTKVMFVKWKLQGKEFFSFDGSKEKFHRNSNFSSANLVSLQDLPKGIASLSLSKTEAVPGNYTCEVTESNREGETIVELRYSVGSWFPPVENILIIVFLILAIIFYWAQLGVVASKFETTLQKKIGLIIAGLIITIVAVVGAILLVPDGYTPKNQAGLGLIVVPAVILVPLQYFLFQIVFENLPPFAIVLVVLKSLGYIIAVVGFALCVSACPPKQGSVVIAGLAIMALAAVIGLVYVIIIGSNMKDHQPPRKAVD

SEQ ID NO: 356 - protein, 309 aa, synthetic construct
MWPLVAALLLGSACCGSAQLTFNATKFVEVTVCNETVVLPCFVNNVETKNIKEMYVKWKFKGKDIFIFDGNQNKSNASSNFTSAKITPSELLKGIASLKMKKSDAVLGNYTCEVTELSREGETIVELKYRVVSWFSPKENILIVIFPILAILLSWGQFGIVTLKYKSNRTNEKTILLFVAGLLLTIVVIVGAILFIPGEYSIKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAVAFKED

SEQ ID NO: 357 - protein, 309 aa, synthetic construct
MWPLVAAHLLSSASCGSAQITFNTTKFVEITVCNETVVLPCFANNVETKNLRELYVKWKFKGKDIFIYDGAQDQATPSSNFSSAEITPSELLKGIASLKMKKSDAVLGNYTCEVTELSREGETIVELKYRVVSWFSPKENILIIIFPILAILLSWGQFGIVTLKYKSNRTNEKTILMFVAGLLLTIVVIVGAILFIPGEYSIKNASGLGLIVIPTVILILLQYCAFMTAFGMTSFAIAILILQVLGYVLAVVGLSLCISECTPVQGPLLISGLGIIALAELLGLVYMKFVASNQKTIQPPRKAVAFKED

SEQ ID NO: 358 - protein, 309 aa, synthetic construct; identical to SEQ ID NO: 19

SEQ ID NO: 359 - protein, 308 aa, synthetic construct
MWALGAWVLLGAVCAGSAQLLFNATKSVEQTICNKTVILPCIVTNLELNNTKVMFVKWKFKGKEIFSFDGSKEKFHRNNNFSSANLSLSDLPKGIASLSLSKTEAVPGNYTCEVTESNREGETIVELRYRVGSWFPPVENILIIIFLILAILFYWAQLGVVALKYETNCTLQKTIGLIIAGLIITIVVVVGAILLVPDGYTPKNAAGLGLIVVPAVILVLLQYFLFQIVFELPPFAIVIVVLQLLGYIIAVVGFSLCVTACPPKQGSVVIAGLAIMALAAVIGLVYVKIVGSNQKDHQPPRKAAFKED

SEQ ID NO: 360 - protein, 308 aa, synthetic construct
MWPLVAALLLGSACCGSAQLLFNKTKSVEFTFCNETVVIPCFVTNVEAENTNELYVKWKFKGKDIYTFDGAMNKSTTDSDFKSARIEVSELLKGDASLKMDKSDAVLGNYTCEVTELSREGETIIELKYRVVSWFSPNENILIVIFPIFAILLFWGQFGIKTLKYKSSGMDEKTIALLVAGLVITAIVIVGAILFVPGEYSVKNATGLGLIVTSTGILILLHYYVFKTAFGITSFVIAILIIQVLAYVLAVVGLSLCISACIPMHGPLLISGLGILALAQLLGLVYMKFVASNQKTIQPPRKAAFKED

SEQ ID NO: 361 - protein, 310 aa, synthetic construct
MWPLVAALLLGSACCGSAQLLFNKTKSVEFTFCNETVVIPCFVNNVEAENIKEMYVKWKFKGKDIFIFDGAQNKSTTYNNFTSAKIAVSELLKGDASLKMDKSDAVLGNYTCEVTELSREGETIVELKYRVVSWFSPNENILIVIFPILAILLFWGQFGIVTLKYKSNRTNEKTILLLVAGLLLTIVVIVGAILFIPGEYSIKNASGLGLIVIPTGILILLQYCVFMTAFGMTSFTIAILIIQVLGYVLAVVGLSLCISACVPVHGPLLISGLGIIALAELLGLVYMKFVASNQKTIQPPRKAVEAFKED

SEQ ID NO: 362 - protein, 310 aa, synthetic construct
MWPLVAALLLGSACCGSAQLIFNVTKSVEFTICNETVVIPCFVNNVEAKNIKEMYVKWKFKGKDIFTFDGAQNKSTTSNNFTSAKITPSELLKGIASLKMDKSDAVLGNYTCEVTELSREGETIVELKYRVVSWFSPNENILIVIFPILAILLSWGQFGIVTLKYKSNRTNEKTILLFVAGLLLTIVVIVGAILFIPGEYSIKNASGLGLIVIPTAILILLQYCVFMTAFGMTSFTIAILILQVLGYVLAVVGLSLCISECTPVHGPLLISGLGIIALAELLGLVYMKFVASNQKTIQPPRKAVEAFKED

SEQ ID NO: 363 - protein, 309 aa, synthetic construct
MWPLVAAQLLSSASSGSAQITFNTTKFVEITVCNETVVLPCFATNVETKNLRELYVKWKFKGKDIFIYDGAQDQATPSSNFSSAEITPSELLKGIASLKMKKSDAVLGNYTCEVTELSREGETIVELKYRVVSWFSPKENILIIIFPILAILLSWGQFGIVTLKYKSNRTNEKTILMFVAGLLLTIVVIVGAILFIPGEYSIKNASGLGLIVIPTVILILLQYCAFMTAFGMTSFAIAILILQVLGYVLAVVGLSLCISECTPVQGPLLISGLGIIALAELLGLVYMKFVASNQKTIQPPRKAVAFKED

SEQ ID NO: 364 - protein, 308 aa, synthetic construct
MWPLVAALLLGSACCGSAQITFNITKSVEFTVCNETVVIPCFATNVETKNLTELYVKWKFKGKDIFIFDGAQNQSTPSSNFTSAKITPSELLRGIASLKMKKSDAVLGNYTCEVTELSREGETIIELKYRVVSWFSPKENILIIIFPILAILLFWGQFGVVTLKYKSNRMNEKTILLFVAGLLLTIVVIVGAILFIPGEYSTKNASGLGLIVIPTVILILLQYFVFMTAFGMTSFAIAILILQVLGFVLAVVGLSLCISECTPVQGPLLISGLGIIALAELLGLVYMKFVASNQKTIQPPRKAAFKED

SEQ ID NO: 365 - protein, 308 aa, synthetic construct
MWALGAWVLLGAVCAGSAQLLFNATKSVEQTICNKTVILPCIVTNLELNNTKVMFVKWKFKGKEIFSFDGSQEKFHRNNNFSSANLSLSDLPKGIASLSLSKTEAVPGNYTCEVTESNREGETIVELRYRVGSWFPPVENILIIIFLILAILFYWAQLGVVALKYETNCTLQKTIGLIIAGLIITIVVVVGAILLVPDGYTPKNAAGLGLIVVPAVILVLLQYFLFQIVFELPPFAIVIVVLQLLGYIIAVVGFSLCVTACPPKQGSVVIAGLAIIALAAVIGLVYVKIVGSNQKDHQPPRKAAFKED

SEQ ID NO: 366 - protein, 304 aa, synthetic construct
MWALGAWVLLGAVCAGSAQLLFNATKSVEQTICNKTVILPCIVTNLELNNTKVMFVKWKLQGKEFFSFDGSKEKFHRNSNFSSANLVSLSDLPKGIASLSLSKTEAVPGNYTCEVTESNREGETVVELRYRVGSWFPPVENILIIVFLILAILFYWAQLGVVASKFETTLQKKIGLIIAGLIITIVAVVGAILLVPDGYTPKNQAGLGLIVVPAVILVPLQYFLFQIVFENLPPFAIVLVVLKSLGYIIAVVGFALCVSACPPKQGSVVIAGLAIMALAAVIGLVYVIIMGSNKKDHQPPRKAD

SEQ ID NO: 367 - protein, 304 aa, synthetic construct
MWALGAWVLLGAVGAGSAQLLFNATKSVEQTICNKTVILPCIVTNLQLNNTKVMFVKWKLQGKEFFSFDGSKEKFHRNSNFSSANLVSLSDLPKGIASLSLSKTEAVPGNYTCEVTESNREGETVVELRYSVGSWFPPVENILIIVFLILAIIFYWAQLGVVASKFETTLQKKIGLIIAGLIITIVAVVGAILLVPDGYTPKNQAGLGLIVVPAVILVPLQYFLFQIVFENLPPFAIVLVVLKSLGYIIAVVGFALCVSACPPKQGSVVIAGLAIMALAAVIGLVYVIIIGSNMKDHQPPRKAD

SEQ ID NO: 368 - protein, 309 aa, synthetic construct
MWPLVAALLLGSACCGSAQLLFNKTKSVEFTFCNETVVIPCFVTNVEAENTNEMYVKWKFKGKDIFIFDGAQNKSTAYSNFTSAKIAVSELLKGDASLKMDKSDAVLGNYTCEVTELSREGETIIELKYRVVSWFSPNENILIVIFPILAILLFWGQFGIVTLKYKSNRTNEKTILLLVAGLLLTTVVIVGAILFIPGEYSVKNASGLGLIVISTGILILLQYCVFMTAFGMTSFTIAILIIQVLGYVLAVVGLSLCISACIPVHGPLLISGLGIIALAELLGLVYMKFVASNQKTIQPPRKAVAFKED

SEQ ID NO: 369 - protein, 309 aa, synthetic construct
MWPLVAALLLGSACCGSAQLLFNKTKSVEFTFCNETVVIPCFVTNVEAENINELYVKWKFKGKDIFIFDGAQNKSTAYSNFTSAKIAPSELLKGDASLKMDKSDAVLGNYTCEVTELSREGETIIELKYRVVSWFSPNENILIVIFPILAILLFWGQFGIVTLKYKSNRTNEKTILLLVAGLLLTIVVIVGAILFIPGEYSTKNASGLGLIVIPTGILILLQYCVFMTAFGMTSFTIAILIIQVLGYVLAVVGLSLCISACIPVHGPLLISGLGIIALAELLGLVYMKFVASNQKTIQPPRKAVAFKED

SEQ ID NO: 370 - protein, 309 aa, synthetic construct
MWALGAWVLLGAVCAGSAQLLFNATKSVEQTICNKTVILPCIVTNLELNNTKVMFVKWKFQGKEIFSFDGSQEKFHRNNNFSSANLSLSDLPKGIASLSLSKTEAVPGNYTCEVTESNREGETIVELRYRVGSWFPPVENILIIIFLILAILFYWAQLGVVALKYETNCTLQKTIGLIIAGLIITIVVVVGAILLVPDGYTPKNAAGLGLIVVPAVILVLLQYFLFQIVFELPPFAIVIVVLQLLGYIIAVVGFSLCVTACPPKQGSVVIAGLAIMALAAVIGLVYVKIVGSNQKDHQPPRKAVAFKED

SEQ ID NO: 371 - protein, 305 aa, synthetic construct
MWALGAWVLLGAVCAGSAQLLFNATKSVEQTICNKTVILPCIVTNLELNNTKVMFVKWKLQGKEFFSFDGSKEKFHRNNNFSSANLVSLSDLPKGIASLSLSKTEAVPGNYTCEVTESNREGETIVELRYRVGSWFPPVENILIIVFLILAILFYWAQLGVVASKFETTLQKKIGLIIAGLIITIVAVVGAILLVPDGYTPKNQAGLGLIVVPAVILVPLQYFLFQIVFENLPPFAIVLVVLKSLGYIIAVVGFALCVSACPPKQGSVVIAGLAIMALAAVIGLVYVIIIGSNKKDHQPPRKAVD

SEQ ID NO: 372 - protein, 309 aa, synthetic construct
MWPLVAALLLGSACCGSAQLLFNKTKSVEFTFCNETVVIPCFVTNVEAENTNEMYVKWKFKGKDIFIFDGAQNKSTTYSNFTSARIAVSELLKGDASLKMDKSDAVLGNYTCEVTELSREGETIIELKYRVVSWFSPNENILIVIFPIFAILLFWGQFGIKTLKYKSSHTDEKTILLLVAGLVVTAVVIVGAILFIPGEYSVKNATGLGLIVISTGILILLQYCVFKTAFGMTSFAIAILIIQVLGYVLAVVGLSLCISACIPMHGPLLISGLGIIALAQLLGLVYMKFVASNQKTIQPPRKAVAFKED

SEQ ID NO: 373 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLMITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 374 - protein, 315 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLMITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAEEPLNAFKED

SEQ ID NO: 375 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTNFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLMITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 376 - protein, 315 aa, synthetic construct
MWPLVAALLLGSACCGSAQLLFNKTKSVEFTFCNDTVVIPCFVTNMEAQNTTEVYVKWKFKGRDIYTFDGALNKSTVPTNFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLMITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAEEPLNAFKED

SEQ ID NO: 377 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTAPANFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLMITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 378 - protein, 310 aa, synthetic construct
MWPLVAALLLGSACCGSAQLLFNKTKSVEFTFCNDTVVIPCFVTNMEAQNTTEVYVKWKFKGRDIYTFDGALNKSTVPTNFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLMITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAAFKED

SEQ ID NO: 379 - protein, 316 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLMITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKED

SEQ ID NO: 380 - protein, 316 aa, synthetic construct
MWPLVAALLLGSACCGSAQLLFNKTKSVEFTFCNDTVVIPCFVTNMEAQNTTEVYVKWKFKGRDIYTFDGALNKSTVPTNFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLMITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKED

SEQ ID NO: 381 - protein, 311 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLMITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVAFKED

SEQ ID NO: 382 - protein, 311 aa, synthetic construct
MWPLVAALLLGSACCGSAQLLFNKTKSVEFTFCNDTVVIPCFVTNMEAQNTTEVYVKWKFKGRDIYTFDGALNKSTVPTNFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLMITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVAFKED

SEQ ID NO: 383 - protein, 310 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLMITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAAFKED

SEQ ID NO: 384 - protein, 312 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLMITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEAFKED

SEQ ID NO: 385 - protein, 323 aa, synthetic construct
MCPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 386 - protein, 323 aa, synthetic construct
MWPLVVALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 387 - protein, 323 aa, synthetic construct
MWPLVAALLLVSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 388 - protein, 323 aa, synthetic construct
MWPLVAALLLGSACCGLAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 389 - protein, 323 aa, synthetic construct
MWPLVAALLLGSACCGSAQPLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 390 - protein, 323 aa, synthetic construct
MWPLVAALLLGSACCGSAQLLFNTTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 391 - protein, 323 aa, synthetic construct
MWPLVAALLLGSACCGSAQLLFNKTKSVEFMFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 392 - protein, 323 aa, synthetic construct
MWPLVAALLLGSACCGSAQLLFNKTKSVEFTFCNNTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 393 - protein, 323 aa, synthetic construct
MWPLVAALLLGSACCGSAQLLFNKTKSVEFTFCNDTVVILCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 394 - protein, 323 aa, synthetic construct
MWPLVAALLLGSACCGSAQLLFNKTKSVEFTFCNDTVVIPRFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 395 - protein, 323 aa, synthetic construct
MWPLVAALLLGSACCGSAQLLFNKTKSVEFTFCNDTVVIPCCVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 396 - protein, 323 aa, synthetic construct
MWPLVAALLLGSACCGSAQLLFNKTKSVEFTFCNDTVVIPCFVTNMDAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 397 - protein, 323 aa, synthetic construct
MWPLVAALLLGSACCGSAQLLFNKTKSVEFTFCNDTVVIPCFVTNMEAQNTTDVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 398 - protein, 323 aa, synthetic construct
MWPLVAALLLGSACCGSAQLLFNKTKSVEFTFCNDTVVIPCFVTNMEAQNTTEIYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 399 - protein, 323 aa, synthetic construct
MWPLVAALLLGSACCGSAQLLFNKTKSVEFTFCNDTVVIPCFVTNMEAQNTTEVYIKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 400 - protein, 323 aa, synthetic construct
MWPLVAALLLGSACCGSAQLLFNKTKSVEFTFCNDTVVIPCFVTNMEAQNTTEVYVKWKFKGRHIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 401 - protein, 323 aa, synthetic construct; identical to SEQ ID NO: 5

SEQ ID NO: 402 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTEFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 403 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTGFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 404 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSVKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 405 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQSLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 406 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDAFLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 407 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKIDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 408 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMYKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 409 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDGVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 410 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAFSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 411 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGKYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 412 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTQLTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 413 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTRESETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 414 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGKTIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 415 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETISELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 416 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIVELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 417 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIKLKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 418 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKFRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 419 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYCVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 420 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSCFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 421 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPKENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 422 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNETILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 423 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIGIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 424 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIAIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 425 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAMLLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 426 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFLGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 427 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQLGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 428 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLEYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 429 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSCGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 430 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIDLLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 431 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVFTVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 432 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGTILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 433 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAIVFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 434 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAIIFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 435 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYALKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 436 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKSATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 437 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNSTGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 438 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGVGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 439 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLDLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 440 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLILTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 441 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVSSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 442 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTYTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 443 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTSIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 444 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTVIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 445 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFIIAILVIQVIAYILAVVGLSLCIAACIPMHGPLLISGLSILALAQLLGLVYMKFVASNQKTIQPPRKAVEEPLNAFKESKGMMNDE

SEQ ID NO: 446 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIHVIAYILAVVGLSLCIAACIPMHGPLLISGLSILALAQLLGLVYMKFVASNQKTIQPPRKAVEEPLNAFKESKGMMNDE

SEQ ID NO: 447 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIDYILAVVGLSLCIAACIPMHGPLLISGLSILALAQLLGLVYMKFVASNQKTIQPPRKAVEEPLNAFKESKGMMNDE

SEQ ID NO: 448 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAFILAVVGLSLCIAACIPMHGPLLISGLSILALAQLLGLVYMKFVASNQKTIQPPRKAVEEPLNAFKESKGMMNDE

SEQ ID NO: 449 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAVCIPMHGPLLISGLSILALAQLLGLVYMKFVASNQKTIQPPRKAVEEPLNAFKESKGMMNDE

SEQ ID NO: 450 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACLPMHGPLLISGLSILALAQLLGLVYMKFVASNQKTIQPPRKAVEEPLNAFKESKGMMNDE

SEQ ID NO: 451 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMYGPLLISGLSILALAQLLGLVYMKFVASNQKTIQPPRKAVEEPLNAFKESKGMMNDE

SEQ ID NO: 452 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLRLVYMKFVASNQKTIQPPRKAVEEPLNAFKESKGMMNDE

SEQ ID NO: 453 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYIKFVASNQKTIQPPRKAVEEPLNAFKESKGMMNDE

SEQ ID NO: 454 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKLVASNQKTIQPPRKAVEEPLNAFKESKGMMNDE

SEQ ID NO: 455 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIHPPRKAVEEPLNAFKESKGMMNDE

SEQ ID NO: 456 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQHPRKAVEEPLNAFKESKGMMNDE

SEQ ID NO: 457 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPMKAVEEPLNAFKESKGMMNDE

SEQ ID NO: 458 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKVVEEPLNAFKESKGMMNDE

SEQ ID NO: 459 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVDEPLNAFKESKGMMNDE

SEQ ID NO: 460 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVQEPLNAFKESKGMMNDE

SEQ ID NO: 461 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEKPLNAFKESKGMMNDE

SEQ ID NO: 462 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNVFKESKGMMNDE

SEQ ID NO: 463 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFNESKGMMNDE

SEQ ID NO: 464 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMSDE

SEQ ID NO: 465 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNNE

SEQ ID NO: 466 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDD

SEQ ID NO: 467 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNSTGVGLIVTSTGILILLHYYVFSTAIGLTSFVIAILVIQVIAYILAVVGLSLCIAVCIPMHGPLLISGLSILALAQLLGLVYMKFVASNQKTIQPPRKAVEEPLNAFKESKGMMNDE

SEQ ID NO: 468 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNSTGVDLIVSSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 469 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNSTGVDLILSSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 470 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNSTGVDLILSSTGILILLHYYVFSTAIGLTSFVIAILVIQVIAYILAVVGLSLCIAVCLPMHGPLLISGLSILALAQLLGLVYMKFVASNQKTIQPPRKAVEEPLNAFKESKGMMNDE

SEQ ID NO: 471 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNSTGVDLIVSSTGILILLHYYVFSTAIGLTSFVIAILVIQVIAYILAVVGLSLCIAVCLPMYGPLLISGLSILALAQLLGLVYMKFVASNQKTIQPPRKAVEEPLNAFKESKGMMNDE

SEQ ID NO: 472 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDGFSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 473 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETISKLKFRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 474 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPKETILIGIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 475 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAMLLFLGQLGIKTLEYRSCGMDEKTIDLLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 476 - protein, 323 aa, synthetic construct
MWPLVAALLLGSACCGSAQLLLNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 477 - protein, 323 aa, synthetic construct
MWPLVAALLLGSACCGSAQLLFNKSKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 478 - protein, 323 aa, synthetic construct
MWPLVAALLLGSACCGSAQLLFNKTKFVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 479 - protein, 323 aa, synthetic construct
MWPLVAALLLGSACCGSAQLLFNKTKSVELTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 480 - protein, 323 aa, synthetic construct
MWPLVAALLLGSACCGSAQLLFNKTKSVEFAFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 481 - protein, 323 aa, synthetic construct; identical to SEQ ID NO: 391

SEQ ID NO: 482 - protein, 323 aa, synthetic construct
MWPLVAALLLGSACCGSAQLLFNKTKSVEFTYCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 483 - protein, 323 aa, synthetic construct
MWPLVAALLLGSACCGSAQLLFNKTKSVEFTFYNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 484 - protein, 323 aa, synthetic construct
MWPLVAALLLGSACCGSAQLLFNKTKSVEFTFCNDSVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 485 - protein, 323 aa, synthetic construct
MWPLVAALLLGSACCGSAQLLFNKTKSVEFTFCNDTVLIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 486 - protein, 323 aa, synthetic construct
MWPLVAALLLGSACCGSAQLLFNKTKSVEFTFCNDTVFIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 487 - protein, 323 aa, synthetic construct
MWPLVAALLLGSACCGSAQLLFNKTKSVEFTFCNDTVIIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 488 - protein, 323 aa, synthetic construct
MWPLVAALLLGSACCGSAQLLFNKTKSVEFTFCNDTVVIPCVVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 489 - protein, 323 aa, synthetic construct
MWPLVAALLLGSACCGSAQLLFNKTKSVEFTFCNDTVVIPCFVANMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 490 - protein, 323 aa, synthetic construct
MWPLVAALLLGSACCGSAQLLFNKTKSVEFTFCNDTVVIPCFVTNTEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 491 - protein, 323 aa, synthetic construct
MWPLVAALLLGSACCGSAQLLFNKTKSVEFTFCNDTVVIPCFVTNMEAQSTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 492 - protein, 323 aa, synthetic construct
MWPLVAALLLGSACCGSAQLLFNKTKSVEFTFCNDTVVIPCFVTNMEAQNATEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 493 - protein, 323 aa, synthetic construct
MWPLVAALLLGSACCGSAQLLFNKTKSVEFTFCNDTVVIPCFVTNMEAQNTAEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 494 - protein, 323 aa, synthetic construct
MWPLVAALLLGSACCGSAQLLFNKTKSVEFTFCNDTVVIPCFVTNMEAQNTSEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 495 - protein, 323 aa, synthetic construct; identical to SEQ ID NO: 399

SEQ ID NO: 496 - protein, 323 aa, synthetic construct
MWPLVAALLLGSACCGSAQLLFNKTKSVEFTFCNDTVVIPCFVTNMEAQNTTEVYAKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 497 - protein, 323 aa, synthetic construct
MWPLVAALLLGSACCGSAQLLFNKTKSVEFTFCNDTVVIPCFVTNMEAQNTTEVYVKWKFKGKDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 498 - protein, 323 aa, synthetic construct
MWPLVAALLLGSACCGSAQLLFNKTKSVEFTFCNDTVVIPCFVTNMEAQNTTEVYVKWKFKGRDIYTFDGTLNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 499 - protein, 323 aa, synthetic construct
MWPLVAALLLGSACCGSAQLLFNKTKSVEFTFCNDTVVIPCFVTNMEAQNTTEVYVKWKFKGRDIYTFDGALTKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 500 - protein, 323 aa, synthetic construct
MWPLVAALLLGSACCGSAQLLFNKTKSVEFTFCNDTVVIPCFVTNMEAQNTTEVYVKWKFKGRDIYTFDGALNKYTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 501 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSAVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 502 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVLTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 503 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVATDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 504 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDSSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 505 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFNSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 506 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFRSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 507 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSANIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 508 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEISQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 509 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEASQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 510 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVAQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 511 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSRLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 512 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQFLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 513 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLQKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 514 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLNGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 515 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDATLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 516 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKVDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 517 - protein, 323 aa, synthetic construct; identical to SEQ ID NO: 407

SEQ ID NO: 518 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMGKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 519 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMVKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 520 - protein, 323 aa, synthetic construct; identical to SEQ ID NO: 408

SEQ ID NO: 521 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDRSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 522 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDTSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 523 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSGAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 524 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSNAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 525 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDTVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 526 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVLHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 527 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCKVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 528 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGEMIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 529 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETFIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 530 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETITELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 531 - protein, 323 aa, synthetic construct; identical to SEQ ID NO: 417

SEQ ID NO: 532 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELQYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 533 - protein, 323 aa, synthetic construct; identical to SEQ ID NO: 419

SEQ ID NO: 534 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYHVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 535 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVAWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 536 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFFPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 537 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNVNILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 538 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENVLIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 539 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILTVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 540 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIIIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 541 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIFIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 542 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFSIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 543 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFTIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 544 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPVFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 545 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFVILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 546 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAVLLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 547 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAIFLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 548 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILQFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 549 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFRGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 550 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFDIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 551 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKCRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 552 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRAGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 553 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSRGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 554 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSSGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 555 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMYEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 556 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTVALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 557 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIGLLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 558 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLIAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 559 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVVTVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 560 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITAIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 561 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVTVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 562 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIAIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 563 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVVVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 564 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVMVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 565 - protein, 323 aa, synthetic construct; identical to SEQ ID NO: 432

SEQ ID NO: 566 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGVILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 567 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILSVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 568 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFIPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 569 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNTTGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 570 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIGTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 571 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVISTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 572 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGMLILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 573 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGIFILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 574 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILMLLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 575 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLNYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 576 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLQYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 577 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHHYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 578 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVYSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 579 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTEIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 580 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTATGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 581 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLASFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 582 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTFFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 583 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSLVIAILVIQVIAYILAVVGLSLCIAACIPMHGPLLISGLSILALAQLLGLVYMKFVASNQKTIQPPRKAVEEPLNAFKESKGMMNDE

SEQ ID NO: 584 - protein, 323 aa, synthetic construct; identical to SEQ ID NO: 445

SEQ ID NO: 585 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVNAILVIQVIAYILAVVGLSLCIAACIPMHGPLLISGLSILALAQLLGLVYMKFVASNQKTIQPPRKAVEEPLNAFKESKGMMNDE

SEQ ID NO: 586 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVFAILVIQVIAYILAVVGLSLCIAACIPMHGPLLISGLSILALAQLLGLVYMKFVASNQKTIQPPRKAVEEPLNAFKESKGMMNDE

SEQ ID NO: 587 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVITILVIQVIAYILAVVGLSLCIAACIPMHGPLLISGLSILALAQLLGLVYMKFVASNQKTIQPPRKAVEEPLNAFKESKGMMNDE

SEQ ID NO: 588 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAVLVIQVIAYILAVVGLSLCIAACIPMHGPLLISGLSILALAQLLGLVYMKFVASNQKTIQPPRKAVEEPLNAFKESKGMMNDE

SEQ ID NO: 589 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAMLVIQVIAYILAVVGLSLCIAACIPMHGPLLISGLSILALAQLLGLVYMKFVASNQKTIQPPRKAVEEPLNAFKESKGMMNDE

SEQ ID NO: 590 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILIIQVIAYILAVVGLSLCIAACIPMHGPLLISGLSILALAQLLGLVYMKFVASNQKTIQPPRKAVEEPLNAFKESKGMMNDE

SEQ ID NO: 591 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVTQVIAYILAVVGLSLCIAACIPMHGPLLISGLSILALAQLLGLVYMKFVASNQKTIQPPRKAVEEPLNAFKESKGMMNDE

SEQ ID NO: 592 - protein, 323 aa, synthetic construct; identical to SEQ ID NO: 448

SEQ ID NO: 593 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIASILAVVGLSLCIAACIPMHGPLLISGLSILALAQLLGLVYMKFVASNQKTIQPPRKAVEEPLNAFKESKGMMNDE

SEQ ID NO: 594 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYIVAVVGLSLCIAACIPMHGPLLISGLSILALAQLLGLVYMKFVASNQKTIQPPRKAVEEPLNAFKESKGMMNDE

SEQ ID NO: 595 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILSVVGLSLCIAACIPMHGPLLISGLSILALAQLLGLVYMKFVASNQKTIQPPRKAVEEPLNAFKESKGMMNDE

SEQ ID NO: 596 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVAGLSLCIAACIPMHGPLLISGLSILALAQLLGLVYMKFVASNQKTIQPPRKAVEEPLNAFKESKGMMNDE

SEQ ID NO: 597 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLYIAACIPMHGPLLISGLSILALAQLLGLVYMKFVASNQKTIQPPRKAVEEPLNAFKESKGMMNDE

SEQ ID NO: 598 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIVACIPMHGPLLISGLSILALAQLLGLVYMKFVASNQKTIQPPRKAVEEPLNAFKESKGMMNDE

SEQ ID NO: 599 - protein, 323 aa, synthetic construct; identical to SEQ ID NO: 449

SEQ ID NO: 600 - protein, 323 aa, synthetic construct; identical to SEQ ID NO: 450

SEQ ID NO: 601 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACMPMHGPLLISGLSILALAQLLGLVYMKFVASNQKTIQPPRKAVEEPLNAFKESKGMMNDE

SEQ ID NO: 602 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPTHGPLLISGLSILALAQLLGLVYMKFVASNQKTIQPPRKAVEEPLNAFKESKGMMNDE

SEQ ID NO: 603 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPVHGPLLISGLSILALAQLLGLVYMKFVASNQKTIQPPRKAVEEPLNAFKESKGMMNDE

SEQ ID NO: 604 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPIHGPLLISGLSILALAQLLGLVYMKFVASNQKTIQPPRKAVEEPLNAFKESKGMMNDE

SEQ ID NO: 605 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMRGPLLISGLSILALAQLLGLVYMKFVASNQKTIQPPRKAVEEPLNAFKESKGMMNDE

SEQ ID NO: 606 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHAPLLISGLSILALAQLLGLVYMKFVASNQKTIQPPRKAVEEPLNAFKESKGMMNDE

SEQ ID NO: 607 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGALLISGLSILALAQLLGLVYMKFVASNQKTIQPPRKAVEEPLNAFKESKGMMNDE

SEQ ID NO: 608 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISSLSILALAQLLGLVYMKFVASNQKTIQPPRKAVEEPLNAFKESKGMMNDE

SEQ ID NO: 609 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLNILALAQLLGLVYMKFVASNQKTIQPPRKAVEEPLNAFKESKGMMNDE

SEQ ID NO: 610 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSVLALAQLLGLVYMKFVASNQKTIQPPRKAVEEPLNAFKESKGMMNDE

SEQ ID NO: 611 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILTLAQLLGLVYMKFVASNQKTIQPPRKAVEEPLNAFKESKGMMNDE

SEQ ID NO: 612 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILAVAQLLGLVYMKFVASNQKTIQPPRKAVEEPLNAFKESKGMMNDE

SEQ ID NO: 613 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALTQLLGLVYMKFVASNQKTIQPPRKAVEEPLNAFKESKGMMNDE

SEQ ID NO: 614 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLIYMKFVASNQKTIQPPRKAVEEPLNAFKESKGMMNDE

SEQ ID NO: 615 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVCMKFVASNQKTIQPPRKAVEEPLNAFKESKGMMNDE

SEQ ID NO: 616 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMTFVASNQKTIQPPRKAVEEPLNAFKESKGMMNDE

SEQ ID NO: 617 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFAASNQKTIQPPRKAVEEPLNAFKESKGMMNDE

SEQ ID NO: 618 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFGASNQKTIQPPRKAVEEPLNAFKESKGMMNDE

SEQ ID NO: 619 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASSQKTIQPPRKAVEEPLNAFKESKGMMNDE

SEQ ID NO: 620 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASDQKTIQPPRKAVEEPLNAFKESKGMMNDE

SEQ ID NO: 621 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPSRKAVEEPLNAFKESKGMMNDE

SEQ ID NO: 622 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPGKAVEEPLNAFKESKGMMNDE

SEQ ID NO: 623 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKTVEEPLNAFKESKGMMNDE

SEQ ID NO: 624 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAVKESKGMMNDE

SEQ ID NO: 625 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKAMMNDE

SEQ ID NO: 626 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGVMNDE

SEQ ID NO: 627 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMTNDE

SEQ ID NO: 628 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMINDE

SEQ ID NO: 629 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMVNDE

SEQ ID NO: 630 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMKDE

SEQ ID NO: 631 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNVE

SEQ ID NO: 632 - protein, 323 aa, synthetic construct; identical to SEQ ID NO: 465

SEQ ID NO: 633 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDG

SEQ ID NO: 634 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLSIAASIPMHGPLLISGLSILALAQLLGLVYMKFVASNQKTIQPPRKAVEEPLNAFKESKGMMNDE

SEQ ID NO: 635 - protein, 323 aa, synthetic construct
MWPLVAALLLGSACCGSAQLLFNKTKSVEFMFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKFRVVSWFSPNENILIVIFPIFAILLFWGQFGIKTLKYRSGGMDEKTIALLVAGLVITVIVIVGAILFVPGEYALKNSTGLALIVTSTGILILLHYYVFSTAIGLTSFVIAILVIQVIAYILAVVGLSLCIAACLPMHGPLLISGLSILALAQLLGLVYMKFVASNQKTIQPPRKAVEEPLNAFKESKGMMNDE

SEQ ID NO: 636 - protein, 323 aa, synthetic construct
MWPLVAALLLGSACCGSAQLLFNKTKSVEFMFCNRTVVIPCFVTNMEAQNTTEVYVKWKFKGRDIYTFDGALNKSTVPTDFSSAKIEVSQLLKGDASLKMDKSDAVSHTGNYTCEVTELTREGETIIELKFRVVSWFSPNENILIVIFPIFAILLFWGQFGIKTLKYRSGGMDEKTIALLVAGLVITVIVIVGAILFVPGEYALKNSTGLALIVTSTGILILLHYYVFSTAIGLTSFVIAILVIQVIAYILAVVGLSLCIAECLPMHGPLLISGLSILALAQLLGLVYMKFVASNQKTIQPPRKAVEEPLNAFKESKGMMNDE

SEQ ID NO: 637 - protein, 323 aa, synthetic construct
MWPLVAALLLGSACCGSAQLLFNKTKSVEFMFCNRTVVIPCFVTNMEAQNTTEVYVKWKFKGRDIYTFDGALNKSTVPTDFSSAKIEVSQLLKGDASLKMDKSDAKSHTGNYTCEVTELTREGETIIELKFRVVSWFSPNENILIVIFPIFAILLFWGQFGIKTLKYRSGGMDEKTIALLVAGLVITVIVIVGAILFVPGEYALKNSTGLALIVTSTGILILLHYYVFSTAIGLTSFVIAILVIQVIAYILAVVGLSLCIAECLPMHGPLLISGLSILALAQLLGLVYMKFVASNQKTIQPPRKAVEEPLNAFKESKGMMNDE

SEQ ID NO: 638 - protein, 310 aa, synthetic construct
MWPLVAALLLGSACCGSAQLTFNKTKSVEFTVCNETVVIPCFVNNMEAQNTTEVYVKWKFKGKDIFIFDGAQNKSTVPTDFTSAKITPSELLKGIASLKMKKSDAVSHTGNYTCEVTELTREGETIVELKYRVVSWFSPKENILIVIFPILAILLSWGQFGIVTLKYKSNRTNEKTILLFVAGLLLTIVVIVGAILFIPGEYSIKNASGLGLIVIPTVILILLQYCVFMTAFGMTSFAIAILILQVLGYVLAVVGLSLCISECTPVQGPLLISGLGIIALAELLGLVYMKFVASNQKTIQPPRKAAFKED

SEQ ID NO: 639 - protein, 318 aa, synthetic construct
MWPLVAALLLGSACCGSAQLLFNKTKSVEFYACNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKTHMVDSCDEEKPPTPVDKCERQFDFIQSIVIAVLLFFIIILCWAQLGVIALKCETVRKKKRHITIACSIFTVVAIVAVVLFIQDGSISMNQIGLAFTILPAGLLMVLQYSIFKMVLDDLTHKGYALIGFQVVGYIVAVVGFALSVSACPSVLLSVVIAGLVVMAVADLLALAYVYSCSRMKDHQTPRY

SEQ ID NO: 640 - protein, 319 aa, synthetic construct
MWPLVAALLLGSACCGSAQLLFNKTKSVEFSTCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKISVGNESDCRDRLPCPSGSWFPPVESVFIILFLFLAIIFSWAQLCVVGSKFEIGLGKKVGLIITGSIIIIAGVVGAILLVPDGYTPKNRAGLGLLVVPAVIFLPLQYLLLGIVFENHLSPSAVVLVALKSLGYIIAVVGFALCVSACPPAQGSVVIAGLAIMAFAALISLAYVCIGSNTKDHQPPRNNL

SEQ ID NO: 641 - protein, 19 aa, Gallus gallus
HMVDSCDEEKPPTPVDKCE

SEQ ID NO: 642 - protein, 17 aa, Alligator mississippiensis
SVGNESDCRDRLPCPSG

SEQ ID NO: 643 - protein, 19 aa, Accipiter gentilis
HSVVSCGEENTPTPKEKCG

SEQ ID NO: 644 - protein, 18 aa, Anas platyrhynchos
TVDNCGEEKAPTPDNKCG

SEQ ID NO: 645 - protein, 15 aa, Ficedula albicollis
SVASCGDETPEEKCG

SEQ ID NO: 646 - protein, 19 aa, Phasianus colchicus
MVDSCDEEKPPTPVDKCER

SEQ ID NO: 647 - protein, 16 aa, Erythrura gouldiae
SSVVSCGDGTPEDKCG

SEQ ID NO: 648 - protein, 323 aa, synthetic construct
MWPLVAALLLGSACCGSAQLLFNKTKSVEFTFCNKTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 649 - protein, 323 aa, synthetic construct
MWPLVAALLLGSACCGSAQLLFNKTKSVEFTFCNDTVVIPCFVTNMEAQNTTEVYVKWKFKGRHIYTFDGALNKCTVPTEFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 650 - protein, 323 aa, synthetic construct
MWPLVAALLLGSACCGSAQLLFNTTDSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 651 - protein, 323 aa, synthetic construct
MWPLVAALLLGSACCGSAQLLFNKTKSVEFMFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFFPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 652 - protein, 323 aa, synthetic construct
MWPLVAALLLGSACCGSAQLLFNKTKSVEFMFCNRTVVIPCFVTNMEAQNTTEVYVKWKFKGRDIYTFDGALNKSTVPTDFSSAKIEVSQLLKGDASLKMDKSDAKSHTGNYTCEVTELTREGETIIELKYRVVSWFSPNENILIVIFPIFAILLFWGQFGIKTLKYRSGGMDEKTIALLVAGLVITVIVIVGAILFVPGEYALKNSTGLALIVTSTGILILLHYYVFSTAIGLTSFVIAILVIQVIAYILAVVGLSLCIAECIPMHGPLLISGLSILALAQLLGLVYMKFVASNQKTIQPPRKAVEEPLNAFKESKGMMNDE

SEQ ID NO: 653 - protein, 323 aa, synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSCFSPNENILIVIFPIFAILLFWGQFGIKTLKYRSGGMDEKTIALLVAGLVITVIVIVGAILFVPGEYSLKNCTGVGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

(19) United States
(12) Patent Application Publication
(10) Pub. No.: US 2026/0345676 A1
(43) Pub. Date: Nov. 5, 2026

(54) COMPOSITIONS AND METHODS FOR NON-IMMUNOGENICITY

(71) Applicant: REPLAY HOLDINGS, INC., San Diego, CA (US)

(72) Inventors: Adrian Woolfson, San Diego, CA (US); Herman WALDMANN, San Diego, CA (US); Ashley BUCKLE, San Diego, CA (US); Gustavo FENALTI, San Diego, CA (US)

(21) Appl. No.: 19/099,202

(22) PCT Filed: Jul. 28, 2023
(86) PCT No.: PCT/US2023/071272

§ 371 Date: Jan. 28, 2025

(30) Foreign Application Priority Data
Jul. 29, 2022 (GB) 2211117.3

(51) Int. Cl. A61K 35/12; A61K 38/00; A61K 40/10; A61P 37/06; C07K 14/705; C12N 5/074; C12N 15/86

(52) U.S. Cl. CPC A61K 35/12; A61K 40/10; A61P 37/06; C07K 14/70503; C12N 5/0696; C12N 15/86; A61K 38/00; C07K 2319/03; C12N 2510/00; C12N 2740/15043

(57) ABSTRACT

The present disclosure provides polypeptides comprising a human signal regulatory protein alpha (SIRPα) binding sequence, wherein the polypeptide elicits a substantially similar or decreased immune response when expressed on a surface of a cell as compared to a polypeptide comprising a wild type (WT) CD47 sequence. The polypeptides of the disclosure may display modulated conformational ensembles relative to.

### CROSS-REFERENCE

This application claims the benefit of UK Patent Application No. 2211117.3, filed Jul. 29, 2022, which application is incorporated herein by reference in its entirety.

### SEQUENCE LISTING

The instant application contains a Sequence Listing which has been submitted electronically in SML format and is hereby incorporated by reference in its entirety. Said XML copy, created on Jul. 27, 2023, is named 62600-735_601_SL.xml and is 765,489 bytes in size.

### BACKGROUND

Cellular therapies hold great promise for combating previously intractable diseases. Use of autologous cells can be cost-prohibitive and burdensome. Although allogeneic pluripotent stem cells (PSCs) provide greater scalability and cost savings, their utility is limited by the need to evade the innate immune system (e.g., leukocytes, such as natural killer cells and macrophages).

### SUMMARY

An aspect of the present disclosure provides an engineered polypeptide comprising a signal regulatory protein alpha (SIRPα) binding sequence, wherein the engineered polypeptide is configured to elicit a decreased immune response when expressed on a surface of a cell as compared to a reference polypeptide comprising a sequence of residues 19-290 of any one of SEQ ID NOs: 1-4, wherein the decreased immune response is at least about 5%, 10%, 15%, 20%, 25%, 30%, 35%, 40%, 45%, 50%, 55%, 65%, 70%, 75%, 80%, 85%, 90%, 95%, or 100% decreased relative to the reference polypeptide.

In some embodiments, the immune response comprises natural killer (NK) cell cytotoxicity. In some embodiments, the immune response comprises macrophage cytotoxicity.

An additional aspect of the present disclosure provides for an engineered polypeptide comprising a signal regulatory protein alpha (SIRPα) binding sequence, wherein the polypeptide comprises a conformational ensemble comprising a first metastable state, which first metastable state is configured to bind to SIRPα, and wherein the conformational ensemble comprises a greater proportion of the first metastable state than a conformational ensemble of a reference polypeptide, wherein the reference polypeptide comprises a sequence of residues 19-290 of any one of SEQ ID NOs: 1-4. In some embodiments, the proportion of the conformational ensemble of the engineered polypeptide or the proportion of the conformational ensemble of the reference polypeptide is determined at least in part by hydrogen-deuterium exchange (HDX), small-angle x-ray scattering (SAXS), nuclear magnetic resonance (NMR), or molecular dynamics (MD). In some embodiments, the proportion of the conformational ensemble of the engineered polypeptide or the proportion of the conformational ensemble of the reference polypeptide is determined at least in part by MD. In some embodiments, the first metastable state is characterized by a bending angle between a transmembrane domain (TMD) and an extracellular domain (ECD) of the polypeptide. In some embodiments, the TMD comprises five alpha helices. In some embodiments, the ECD is configured to bind SIRPα. In some embodiments, the bending angle is from about 130 to about 180 degrees. In some embodiments, the first metastable state is characterized by a distance between the TMD and the ECD of the polypeptide. In some embodiments, the distance is from about 10 to about 25 angstroms (Å). In some embodiments, the engineered polypeptide is configured to elicit a decreased immune response when expressed on a surface of a cell as compared to a reference polypeptide comprising a sequence of residue 19 through the final residue of any one of SEQ ID NOs: 1-4, wherein the decreased immune response is at least about 5%, 10%, 15%, 20%, 25%, 30%, 35%, 40%, 45%, 50%, 55%, 65%, 70%, 75%, 80%, 85%, 90%, 95%, or 100% decreased relative to the reference polypeptide. In some embodiments, the immune response comprises natural killer (NK) cell cytotoxicity. In some embodiments, the immune response comprises macrophage cytotoxicity.

An additional aspect of the present disclosure provides an engineered polypeptide comprising a signal regulatory protein alpha (SIRPα) binding sequence, wherein the engineered polypeptide is configured to elicit a decreased immune response when expressed on a surface of a cell as compared to a reference polypeptide comprising a sequence of residues 19-290 of any one of SEQ ID NOs: 1-4, wherein the SIRPα binding sequence comprises at least one mutation relative to any one of SEQ ID NOs: 1-4, wherein the at least one mutation is associated with a cancer.

In some embodiments, the at least one mutation is comprised in a database. In some embodiments, the database comprises the Catalogue of Somatic Mutations in Cancer (COSMIC), the Genome Aggregation Database (gnomAD), or both. In some embodiments, the at least one mutation comprises a mutation selected from the group consisting of: the mutations listed in Table 3, and any combination thereof. In some embodiments, the at least one mutation comprises a mutation selected from the group consisting of: M31, L40, C42, D47, D64, C75, E80, F97, G105, K106, F106, K111, S123, S127, K128, F131, C132, C136, K140, T142, G146, M153, L157, L160, E166, C170, D178, A203, S207, V210, D211, L214, S215, V262, L264, Y267, and any combination thereof. In some embodiments, the polypeptide comprises an additional mutation, wherein the additional mutation is selected from the group consisting of: Y31, A32, R35, K35, P71, A77, A79, N80, L100, K138, L164, M185, A211, S259, E262, and any combination thereof.

An additional aspect of the present disclosure provides an engineered signal regulatory protein alpha (SIRPα) binding polypeptide comprising: an extracellular domain (ECD), a transmembrane domain (TMD), and an extracellular loop region (ECLR), wherein the extracellular loop region comprises a heterologous sequence compared to residues 19 through the final residue of any one of SEQ ID NOs: 1-4.

In some embodiments, the ECD, the TMD, and the ECLR are derived from at least two different organisms. In some embodiments, the ECLR comprises at least about 70%, 80%, 85%, 90%, 91%, 92%, 93%, 94%, 95%, 96%, 97%, 98%, or 99%, or 100% sequence identity to an ECLR of any one of SEQ ID NOs: 639 and 640. In some embodiments, the heterologous sequence comprises a pair of cysteine residues configured to form a disulfide pair. In some embodiments, the heterologous sequence comprises at least about 70%, 80%, 85%, 90%, 91%, 92%, 93%, 94%, 95%, 96%, 97%, 98%, or 99%, or 100% sequence identity to any one of SEQ ID NOs: 641-647. In some embodiments, the engineered polypeptide is configured to elicit a decreased immune response when expressed on a surface of a cell as compared to a reference polypeptide comprising a sequence of any one of SEQ ID NO: 1-4. In some embodiments, the polypeptide and the reference polypeptide are configured to adopt a first metastable state, wherein the first metastable state is configured to bind to SIRPα. In some embodiments, a conformational ensemble of the engineered polypeptide comprises a larger proportion of the first metastable state than a conformational ensemble of the reference polypeptide. In some embodiments, the proportion of the conformational ensemble of the engineered polypeptide or the proportion of the conformational ensemble of the reference polypeptide is determined at least in part by hydrogen-deuterium exchange (HDX), small-angle x-ray scattering (SAXS), nuclear magnetic resonance (NMR), or molecular dynamics (MD). In some embodiments, the proportion of the conformational ensemble of the engineered polypeptide or the proportion of the conformational ensemble of the reference polypeptide is determined at least in part by MD. In some embodiments, the first metastable state is characterized by a bending angle between a transmembrane domain (TMD) and an extracellular domain (ECD) of the polypeptide. In some embodiments, the TMD comprises five alpha helices. In some embodiments, the ECD is configured to bind SIRPα. In some embodiments, the bending angle is from about 130 to about 180 degrees. In some embodiments, the first metastable state is characterized by a distance between the TMD and the ECD of the polypeptide. In some embodiments, the distance is from about 10 to about 25 angstroms (Å). In some embodiments, the SIRPα binding sequence comprises at least about 70%, 80%, 90%, 95%, or 99%, or 100% identity to any one of SEQ ID NOs: 10-640 or 648-653. In some embodiments, the engineered polypeptide comprises a sequence at least about 70%, 80%, 80%, 95%, or 99%, or 100% identical to an extracellular domain (ECD) of any one of SEQ ID NOs: 1-640 and 648-653. In some embodiments, the engineered polypeptide comprises a sequence at least about 70%, 80%, 80%, 95%, or 99%, or 100% identical to a transmembrane domain (TMD) of any one of SEQ ID NOs: 1-640 and 648-653. In some embodiments, the engineered polypeptide comprises a sequence at least about 70%, 80%, 80%, 95%, or 99%, or 100% identical to an extracellular loop region (ECLR) of any one of SEQ ID NOs: 1-640 and 648-653. In some embodiments, the engineered polypeptide comprises at least one amino acid substitution specified in Table 3. In some embodiments, the engineered polypeptide comprises one or more amino acid substitutions selected from the group consisting of: M31, L40, C42, D47, D64, C75, E80, F97, G105, K106, F106, K111, S123, S127, K128, F131, C132, C136, K140, T142, G146, M153, L157, L160, E166, C170, D178, A203, S207, V210, D211, L214, S215, V262, L264, Y267, and any combination thereof. In some embodiments, the engineered polypeptide comprises at least one amnio acid substitution specified in Table 4. In some embodiments, the engineered polypeptide comprises at least one amino acid substitution selected from the group consisting of: Y31, A32, R35, K35, P71, A77, A79, N80, L100, K138, L164, M185, A211, S259, E262, and any combination thereof. In some embodiments, the cell is a stem cell. In some embodiments, the stem cell is an induced pluripotent stem cell (iPSC). In some embodiments, the polypeptide comprises an N-terminal addition. In some embodiments, the TMD, the ECD, and the linker are from more than one organism. In some embodiments, the engineered polypeptide is configured to elicit a reduced integrin response as compared to the reference polypeptide. In some embodiments, the polypeptide is configured to elicit a reduced thrombospondin 1 (TSP-1) response as compared to the reference polypeptide.

An additional aspect of the present disclosure provides an engineered polypeptide comprising a sequence with at least about 70%, 80%, 81, 82, 83, 84, 85, 86, 87, 88, 89, 90, 91, 92, 93, 94, 95, 96, 97, 98, or 99, or 100% identity to any one of SEQ ID NOs: 1-640 and 648-653.

In some embodiments, the engineered polypeptide comprises a sequence at least about 70%, 80%, 80%, 95%, or 99%, or 100% identical to an extracellular domain (ECD) of any one of SEQ ID NOs: 1-640 and 648-653. In some embodiments, the engineered polypeptide comprises a sequence at least about 70%, 80%, 80%, 95%, or 99%, or 100% identical to a transmembrane domain (TMD) of any one of SEQ ID NOs: 1-640 and 648-653. In some embodiments, the engineered polypeptide comprises a sequence at least about 70%, 80%, 80%, 95%, or 99%, or 100% identical to an extracellular loop region (ECLR) of any one of SEQ ID NOs: 1-640 and 648-653. In some embodiments, the engineered polypeptide comprises a sequence at least about 70%, 80%, 80%, 95%, or 99%, or 100% identical to any one of SEQ ID NOs: 641-647.

An additional aspect of the present disclosure provides an engineered polypeptide comprising a signal regulatory protein alpha (SIRPα) binding sequence, wherein the engineered polypeptide comprises a conformational ensemble comprising a first metastable state, wherein the first metastable state is configured to bind SIRPα, and wherein the conformational ensemble comprises a greater proportion of the first metastable state than a conformational ensemble of a reference polypeptide, wherein the reference polypeptide comprises a sequence of residues 19-290 of any one of SEQ ID NOs: 1-4.

In some embodiments, the proportion of the conformational ensemble of the engineered polypeptide or the proportion of the conformational ensemble of the reference polypeptide is determined at least in part by hydrogen-deuterium exchange (HDX), small-angle x-ray scattering (SAXS), nuclear magnetic resonance (NMR), or molecular dynamics (MD). In some embodiments, the proportion of the conformational ensemble of the engineered polypeptide or the proportion of the conformational ensemble of the reference polypeptide is determined at least in part by MD. In some embodiments, the first metastable state is characterized by a bending angle between a transmembrane domain (TMD) and an extracellular domain (ECD) of the polypeptide. In some embodiments, the TMD comprises five alpha helices. In some embodiments, the ECD is configured to bind SIRPα. In some embodiments, the bending angle is from about 130 to about 180 degrees. In some embodiments, the first metastable state is characterized by a distance between the TMD and the ECD of the polypeptide. In some embodiments, the distance is from about 10 to about 25 angstroms (Å). In some embodiments, the engineered polypeptide is configured to elicit a decreased immune response when expressed on a surface of a cell as compared to a reference polypeptide comprising a sequence of residues 19-290 of any one of SEQ ID NOs: 1-4, wherein the decreased immune response is at least about 5%, 10%, 15%, 20%, 25%, 30%, 35%, 40%, 45%, 50%, 55%, 65%, 70%, 75%, 80%, 85%, 90%, 95%, or 100% decreased relative to the reference polypeptide. In some embodiments, the immune response comprises natural killer (NK) cell cytotoxicity. In some embodiments, the immune response comprises macrophage cytotoxicity. In some embodiments, the SIRPα binding sequence comprises at least about 70%, 80%, 90%, 95%, or 99%, or 100% identity to any one of SEQ ID NOs: 10-640 or 684-653. In some embodiments, the engineered polypeptide comprises at least one amino acid substitution specified in Table 3. In some embodiments, the engineered polypeptide comprises one or more amino acid substitutions selected from the group consisting of: M31, L40, C42, D47, D64, C75, E80, F97, G105, K106, F106, K111, S123, S127, K128, F131, C132, C136, K140, T142, G146, M153, L157, L160, E166, C170, D178, A203, S207, V210, D211, L214, S215, V262, L264, Y267, and any combination thereof. In some embodiments, the engineered polypeptide comprises at least one amnio acid substitution specified in Table 4. In some embodiments, the engineered polypeptide comprises at least one amino acid substitution selected from the group consisting of: Y31, A32, R35, K35, P71, A77, A79, N80, L100, K138, L164, M185, A211, S259, E262, and any combination thereof. In some embodiments, the cell is a stem cell. In some embodiments, the stem cell is an induced pluripotent stem cell (iPSC). In some embodiments, the polypeptide comprises an N-terminal addition. In some embodiments, the TMD, the ECD, and the linker are from more than one organism. In some embodiments, the engineered polypeptide is configured to elicit a reduced integrin response as compared to the reference polypeptide. In some embodiments, the polypeptide is configured to elicit a reduced thrombospondin 1 (TSP-1) response as compared to the reference polypeptide.

An additional aspect of the present disclosure provides an engineered cell comprising any of the engineered peptides described herein.

An additional aspect of the present disclosure provides an engineered cell comprising a plurality of SIRPα binding polypeptides, wherein the plurality of SIRPα binding polypeptide comprises any of the engineered peptides described herein.

In some embodiments, the plurality of SIRPα binding polypeptide further comprises a wild type CD47. In some embodiments, the plurality of SIRPα binding polypeptide comprises a plurality of any of the engineered peptides described herein. In some embodiments, the engineered cell is a stem cell. In some embodiments, the stem cell is an embryonic stem cell, a mesenchymal stem cell, an induced pluripotent stem cell, or a hematopoietic stem cell.

An additional aspect of the present disclosure provides a nucleic acid molecule encoding the any of the engineered polypeptides disclosed herein.

An additional aspect of the present disclosure provides a nucleic acid molecule encoding a plurality of SIRPα binding polypeptides, wherein the plurality of SIRPα binding polypeptides comprises a wild type CD47 and any of the engineered polypeptides disclosed herein.

An additional aspect of the present disclosure provides a nucleic acid molecule encoding a plurality of SIRPα binding polypeptides, wherein the plurality of SIRPα binding polypeptides comprises a plurality of any of the engineered polypeptide disclosed herein.

An additional aspect of the present disclosure provides a vector comprising any of the nucleic acids disclosed herein.

In some embodiments, the vector is a plasmid, a minicircle, a CELiD, an adeno-associated virus (AAV) derived virion, a lentivirus, an adenovirus, or a herpes simplex virus (HSV).

An additional aspect of the present disclosure provides a method of generating a hypoimmunogenic cell comprising administering to a cell any of the vectors disclosed herein.

An additional aspect of the present disclosure provides an engineered polypeptide comprising a signal regulatory protein alpha (SIRPα) binding sequence, wherein the polypeptide is configured to elicit a decreased macrophage response when expressed on a surface of a cell as compared to a polypeptide comprising a sequence of any one of SEQ ID NOs: 1-4.

In some embodiments, the SIRPα binding sequence comprises at least about 70%, 80%, 90%, 95%, or 99% identity to any one of the sequences described herein. In some embodiments, the SIRPα binding sequence comprises at least one amino acid substitution specified in Table 3. In some embodiments, the SIRPα binding sequence comprises at least one amino acid substitution specified in Table 3 when optimally aligned to any one of SEQ ID NOs: 1-4. In some embodiments, the SIRPα binding sequence comprises at least one amnio acid substitution specified in Table 4. In some embodiments, the SIRPα binding sequence comprises at least one amino acid substitution specified Table 4 when optimally aligned to any one of SEQ ID NOs: 1-4. In some embodiments, the cell comprises a stem cell. In some embodiments, the stem cell comprises an induced pluripotent stem cell (iPSC). In some embodiments, the polypeptide comprises an N-terminal addition. In some embodiments, the N-terminal addition comprises at least one, two, or three amino acids added to an N-terminus of the polypeptide. In some embodiments, the three amino acids comprise a formula X-3X-2X-1, where X-3 is W; X-2 is selected from Q, A and G; and X-1 is selected from R, P, L, T, F, I, and M. In some embodiments, the three amino are selected from: WQR, WAP, WQL, WQP, WQT, WQF, WQI, WGP, and WQM. In some embodiments, the polypeptide and the reference polypeptide are configured to adopt a first metastable state, wherein the first metastable state is configured to bind to SIRPα. In some embodiments, the reference polypeptide, and optionally the polypeptide, is configured to adopt a second metastable state, wherein the second metastable state does not substantially bind to SIRPα. In some embodiments, a conformational ensemble of the polypeptide comprises a larger proportion of the first metastable state than a conformational ensemble of the reference polypeptide. In some embodiments, the proportion of the conformational ensemble of the polypeptide is determined at least in part by hydrogen-deuterium exchange (HDX), small-angle x-ray scattering (SAXS), nuclear magnetic resonance (NMR), or molecular dynamics (MD). In some embodiments, the first metastable state is characterized by a first bending angle between a transmembrane domain (TMD) and an extracellular domain (ECD) of the polypeptide. In some embodiments, the TMD comprises five alpha helices. In some embodiments, the ECD is configured to bind SIRPα. In some embodiments, the first bending angle is from about 130 to about 180 degrees. In some embodiments, the first bending angle is from about 150 to about 170 degrees. In some embodiments, the second metastable state is characterized by a second bending angle between the TMD and the ECD. In some embodiments, the second bending angle is from about 100 degrees to about 120 degrees. In some embodiments, the first metastable state is characterized by a first distance between the TMD and the ECD of the polypeptide. In some embodiments, the first distance is from about 10 to about 25 angstroms (Å). In some embodiments, the second metastable state is characterized by a second distance between the TMD and the ECD. In some embodiments, the second distance is from about 4 to about 9 angstroms (Å). In some embodiments, the TMD, the ECD, and the linker are all from one organism. In some embodiments, the TMD, the ECD, and the linker are from more than one organism. In some embodiments, at least one of the TMD, the ECD, and the linker is engineered. In some embodiments, the polypeptide is configured to elicit a reduced integrin response as compared to the reference polypeptide. In some embodiments the polypeptide is configured to elicit a reduced TSP-1 response as compared to the reference polypeptide.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows several representative metastates of a SIRPα binding peptide in accordance with some embodiments of the present disclosure.

FIGS. 2A-2B show example free energy surfaces representative of conformational ensembles of SIRPα binding peptides in accordance with some embodiments of the present disclosure.

FIGS. 3A-3B show example plots of reaction coordinates monitoring a transition between metastates in a molecular dynamics simulation of a SIRPα binding polypeptide in accordance with some embodiments of the present disclosure.

FIG. 4 shows a molecular model of a SIRPα binding polypeptide as described herein.

FIGS. 5A-5B show example plots of reaction coordinates monitoring a transition between metastates in a molecular dynamics simulation of a SIRPα binding polypeptide in accordance with some embodiments of the present disclosure.

FIG. 6 illustrates a vector configured for expression of polypeptides as disclosed herein.

FIGS. 7A-7B depict bar plots showing relative proportions of metastates in simulated conformational ensembles of SIRPα binding polypeptides as disclosed herein.

FIG. 7C shows example free energy surfaces representative of conformational ensembles of SIRPα binding peptides in accordance with some embodiments of the present disclosure.

FIGS. 8-18 show graphs quantifying protection from natural killer cell cytotoxicity afforded by SIRPα binding polypeptides of the disclosure.

FIGS. 19-21 depict bar graphs showing measure surface expression of SIRPα binding polypeptides of the present disclosure.

FIG. 22 show a graph quantifying protection from macrophage phagocytosis afforded by SIRPα binding polypeptides of the disclosure.

FIG. 23 depicts a molecular model of a SIRPα binding polypeptide as described herein.

FIG. 24 shows a computer system that is programmed or otherwise configured to implement methods provided herein.

### BRIEF DESCRIPTION OF THE SEQUENCE LISTING

The Sequence Listing filed herewith provides example polynucleotide and polypeptide sequences for use in the methods, compositions, and systems according to the disclosure. Below are representative descriptions of sequences therein.

SEQ ID NOs: 1-4 show representative amino acid constructs of wildtype human CD47 sequences.

SEQ ID NOs: 5-9 show representative cysteine mutations to SEQ ID NO: 1 that may be made to induce oligomerization.

SEQ ID NOs: 10-384 show representative engineered signal regulatory protein alpha (SIRPα) binding sequences in accordance with some embodiments of the disclosure.

SEQ ID NOs: 385-475 show representative SIRPα binding sequences based at least in part on mutations associated with cancer.

SEQ ID NOs: 476-633 and 648 show representative SIRPα binding sequences based at least in part on polymorphisms identified in one or more individuals.

SEQ ID NOs: 634-638 and 648-653 show representative SIRPα binding sequences based at least in part on one or more rational mutations.

SEQ ID NOs: 639-640 show representative SIRPα binding sequences which are chimeric and/or comprise loop insertions as described herein.

SEQ ID NOs: 641-647 show sequences of representative insertions in engineered SIRPα binding sequences of the disclosure.

### DETAILED DESCRIPTION

Cluster of Differentiation 47 (CD47), also known as integrin associated protein (IAP), is a transmembrane protein that in humans is encoded by the cd47 gene. CD47 belongs to the immunoglobulin superfamily and partners with membrane integrins and also binds the ligands thrombospondin 1 (TSP-1) and signal-regulatory protein alpha (SIRPα). It is involved in a range of cellular processes, including apoptosis, proliferation, adhesion, and migration. CD47 is a ~50 kDa heavily glycosylated, ubiquitously expressed membrane protein of the immunoglobulin superfamily with a single IgV-like domain at its N-terminus (its extracellular domain or ECD), a highly hydrophobic stretch with five membrane-spanning segments (also referred to as the transmembrane or™ domain), and an alternatively spliced cytoplasmic C-terminus (C-terminal domain or CTD). Each of the four alternatively spliced cytoplasmic tails (e.g., the C-terminal portions of SEQ ID NOs: 1-4) exists in vivo at different frequencies, but all lack a substantial signaling domain.

CD47 is an integral component of the innate immune system, and binding of the ECD to signal regulatory protein alpha (SIRPα) on immune cells suppresses phagocytosis and NK-cell killing. CD47 is ubiquitously expressed in human cells and has been found to be overexpressed in many different tumor cells.

Certain attempts at engineering hypoimmunogenic cells have centered on evading recognition by the adaptive immune response through abolition or alteration of major histocompatibility complex (MHC) proteins expressed by the cells. Nevertheless, such cells can remain susceptible to killing by actors of the innate immune system, including natural killer (NK) cells and macrophages, which target cells through other cognate binding partners and signaling pathways (e.g., CD47).

Accordingly, disclosed herein are various engineered signal-regulatory protein alpha (SIRPα) binding polypeptides which can overcome immune rejection in cell-based transplantation therapies using universal donor stem cell lines. Some aspects of the present disclosure provide engineered SIRPα binding polypeptides and variants that retain or enhance the native CD47 “don't eat me” signaling (e.g., relative to WT CD47). In some embodiments, the engineered SIRPα agonist constructs described herein display enhanced signaling for the CD47/SIRPα pathway over wild-type (WT) CD47, thus protecting cells expressing such molecules from killing by macrophages and/or NK cells. In some embodiments, the SIRPα binding polypeptides and variants disclosed herein elicit a reduced immune response when expressed on a surface of a cell as compared to WT CD47. In some embodiments, the reduced immune response comprises NK cell cytotoxicity. In some embodiments, the reduced immune response comprises macrophage cytotoxicity. In some embodiments, the engineered SIRPα agonist constructs display “detuning” or decreased signaling through pathways other than SIRPα. In some embodiments, the engineered SIRPα agonist sequences disclosed herein comprise low sequency homology with human CD47 polypeptides and variants. In some embodiments, the engineered SIRPα agonist sequences disclosed herein comprise less than about 90%, about 80%, about 70%, about 60%, about 50%, about 40%, about 30%, about 20%, or less sequence identity with any one of SEQ ID NOs: 1-4. In some embodiments, the engineered molecules may retain basal functions required for cellular homeostasis while displaying enhanced CD47/SIRPα signaling functions; these SIRPα agonist constructs can be expressed as a single entity on the surface of cells (e.g., wherein a human cd47 gene is knocked out or in combination with endogenously expressed wild-type CD47).

Another aspect of the present disclosure comprises rationally designed SIRPα binding sequences that retain binding to human SIRPα and display susceptibility to macrophage phagocytosis and/or NK cell killing comparable to or lower than WT CD47 splice variants 1~4 (corresponding to SEQ ID NOs: 1-4), with the advantage of having “skewed” signaling function towards reduced macrophage phagocytosis and/or NK cell killing signaling and optionally detuned binding of one or more other endogenous binding partners. In some embodiments, the detuned endogenous binding partners comprises integrins. In some embodiments, the detuned endogenous binding partners comprise thrombospondin-1 (TSP-1).

Another aspect of the present disclosure comprises co-expression of different engineered SIRPα binding polypeptides (e.g., 2 or more) comprising co-expression of any of the SIRPα binding polypeptides described herein.

Another aspect of the present disclosure comprises rationally designed SIRPα binding polypeptide sequences that retain binding to human SIRPα but display reduced or abrogated signaling function though other (e.g., non-SIRPα) binding partners, such as reduced or abrogated binding and signaling through TSP-1, integrins and other human CD47 endogenous binding partners. Engineered SIRPα binding sequences or constructs which exhibit detuned CD47 signaling for one or more endogenous binding partners other than CD47 may be referred to as engineered “selective” SIRPα binding sequences or constructs. Another aspect of the present disclosure comprises SIRPα binding mutations based at least in part on any one of WT CD47 splice variants 1~4 that enhance “don't eat me” signaling, thus preventing or reducing phagocytosis (e.g., by macrophages) and/or NK cell killing of cells which express the mutated SIRPα binding polypeptides (e.g., induced pluripotent stem cells (iPSCs)). In some embodiments, the mutations are based at least in part on mutations such as those listed in Table 3 or Table 4, or result from computational in silico calculations (e.g., all-atom molecular dynamics, normal mode analysis, coarse grain simulations, frustration analysis, or protein stability).

Another aspect of the present disclosure comprises engineered SIRPα binding sequences based at least in part on any one of SEQ ID NOs: 1-4 that retain binding of human SIRPα but contain peptide insertions within the wild-type human CD47 RVVSWF peptide linker (e.g., residues 132-137 of any one of SEQ ID NOs: 1-4) connecting the extracellular domain (ECD) and transmembrane domain (TMD) of any one of SEQ ID NOs: 1-4 and can display altered/detuned signaling function (e.g., do not bind or have reduced binding of endogenous protein partners other than SIRPα or do not signal through or have reduced transmembrane signaling through protein partners other than SIRPα). In some embodiments, the peptide insertions are based at least in part on one or more orthologs or homologs of SEQ ID NOs: 1-4. In some embodiments, the peptide insertion comprises at sequence at least about 50%, 60%, 70%, 80%, 85%, 90%, 95%, 99%, or 100% sequence identity to any one of SEQ ID NOs: 641-647. In some embodiments, a peptide insertion as described herein modulates the conformational ensemble of a SIRPα binding polypeptide relative to a reference polypeptide (e.g., comprising a wild-type human CD47 sequence such as comprised in any one of SEQ ID NOs: 1-4).

Another aspect of the present disclosure comprises engineered SIRPα binding sequences based at least in part on any one of SEQ ID NOs: 1-4 that retain binding of human SIRPα but contain peptide insertions that contain two cysteines (e.g., constrained by a disulfide bond) within a region adjacent to or disposed within the RVVSWF peptide linker connecting the ECD and TMD of WT CD47 (e.g., residues 132-137 of any one of SEQ ID NOs: 1-4) and display altered or detuned function (e.g., do not bind or have reduced binding of endogenous protein partners other than SIRPα and/or do not signal through or have decreased signaling functions with protein partners other than SIRPα). In some embodiments, the cysteine residues configured to form a disulfide bond as described herein modulate the conformational ensemble of a SIRPα binding polypeptide relative to a reference polypeptide (e.g., comprising a wild-type human CD47 sequence such as comprised in any one of SEQ ID NOs: 1-4).

Another aspect of the present disclosure comprises engineered SIRPα binding sequences based at least in part on any one of SEQ ID NOs: 1-4 that retain binding of human SIRPα but contain peptide insertions within the RVVSWF peptide linker connecting the ECD and TMD of WT CD47 (e.g., residues 132-137 of any one of SEQ ID NOs: 1-4) and additional mutations (single point or combinations thereof) and display altered/detuned function (e.g., do not bind or have reduced binding of endogenous protein partners other than SIRPα and/or do not signal through or have decreased signaling functions with these other protein partners). In some embodiments, the peptide insertions combined with additional mutations(s) modulates the conformational ensemble of a SIRPα binding polypeptide relative to a reference polypeptide (e.g., a wild-type CD47 sequence, such as comprised in any one of SEQ ID NOs: 1-4). In some embodiments, the mutations(s) modulate the conformational ensemble of a SIRPα binding polypeptide relative to a reference polypeptide (e.g., comprising a wild-type human CD47 sequence such as comprised in any one of SEQ ID NOs: 1-4).

Another aspect of the present disclosure comprises mutations on SIRPα binding polypeptides that enhance dimer or other oligomer (e.g., trimer, tetramer, etc.) formation based at least in part on any one of SEQ ID NOs: 1-4 (such as one or more mutations detailed in Table 2 hereinbelow). In some embodiments, SIRPα binding polypeptides with enhanced oligomer formation display altered or detuned function relative to a reference polypeptide (e.g., do not bind or have reduced binding of endogenous protein partners other than SIRPα and/or do not signal through or have decreased signaling functions with protein partners other than SIRPα).

Another aspect of the present disclosure comprises computationally engineered SIRPα binding sequences based on in silico evolutionary strategy algorithms and/or trained algorithms (such as machine learning algorithms) to enhance SIRPα binding polypeptide stability and retain binding and signaling through SIRPα, while reducing binding to other endogenous protein binding partners and associated signaling. In some embodiments, these constructs have less than about 90%, about 80%, about 70%, about 60%, about 50%, about 40%, about 30%, about 20%, or less sequence identity with any one of SEQ ID NOs: 1-4. In some embodiments, these constructs have a modulated conformational ensemble relative to a reference polypeptide (e.g., comprising a wild-type human CD47 sequence such as comprised in any one of SEQ ID NOs: 1-4).

Another aspect of the present disclosure comprises compositions and methods for dimerization or oligomerization achieved by single or multiple mutations of computationally designed SIRPα binding polypeptide sequences based on in silico evolutionary strategy algorithms and/or trained algorithms (such as machine learning algorithms) to enhance stability relative to any one of SEQ ID NOs: 1-4 and retain binding and signaling of SIRPα, while reducing binding to other endogenous protein binding partners and associated signaling. In some embodiments, these constructs have less than about 90%, about 80%, about 70%, about 60%, about 50%, about 40%, about 30%, about 20%, or less sequence identity with any one of SEQ ID NOs: 1-4. In some embodiments, these constructs have a modulated conformational ensemble relative to a reference polypeptide (e.g., comprising a wild-type human CD47 sequence such as comprised in any one of SEQ ID NOs: 1-4).

Another aspect of the present disclosure comprises computationally engineered SIRPα binding polypeptides comprising peptide insertions within the wild-type human CD47 RVVSWF peptide linker (e.g., residues 132-137 of any one of SEQ ID NOs: 1-4) grafted on computationally designed SIRPα agonist sequences based on in silico evolutionary strategy algorithms. Such insertions may enhance SIRPα agonist stability and retain binding and signaling of SIRPα, while reducing binding to other endogenous protein binding partners and associated signaling. In some embodiments, these constructs have less than about 90%, about 80%, about 70%, about 60%, about 50%, about 40%, about 30%, about 20%, or less sequence identity with any one of SEQ ID NOs: 1-4. In some embodiments, these constructs have a modulated conformational ensemble relative to a reference polypeptide (e.g., comprising a wild-type human CD47 sequence such as comprised in any one of SEQ ID NOs: 1-4).

Another aspect of the present disclosure comprises computationally engineered SIRPα binding sequences comprising mutations (single point mutations or combinations thereof) relative to any one of SEQ ID NOs: 1-4 grafted on computationally designed SIRPα binding sequences based on in silico evolutionary strategy algorithms and/or trained algorithms (such as machine learning algorithms) to enhance SIRPα binding polypeptide stability and retain binding and signaling of SIRPα, while reducing binding to other endogenous protein binding partners and associated signaling. In some embodiments, these constructs have less than about 90%, about 80%, about 70%, about 60%, about 50%, about 40%, about 30%, about 20%, or less sequence identity with any one of SEQ ID NOs: 1-4. In some embodiments, these constructs have a modulated conformational ensemble relative to a reference polypeptide (e.g., comprising a wild-type human CD47 sequence such as comprised in any one of SEQ ID NOs: 1-4).

Another aspect of the present disclosure comprises engineered SIRPα binding polypeptides comprising one or more mutations in CD47 (e.g., one or more of those listed in Table 3 and Table 4) grafted on computationally designed SIRPα binding sequences based on in silico evolutionary strategy algorithms and/or trained algorithms (such as machine learning algorithms) to enhance SIRPα binding polypeptide stability and retain binding and signaling of SIRPα, while reducing binding to other endogenous protein binding partners and associated signaling. In some embodiments, these constructs have less than about 90%, about 80%, about 70%, about 60%, about 50%, about 40%, about 30%, about 20%, or less sequence identity with any one of SEQ ID NOs: 1-4. In some embodiments, a computationally designed SIRPα binding sequence comprising additional mutation(s) is characterized by a conformational ensemble modulated relative to that of a reference polypeptide (e.g., a wild-type CD47 sequence, such as comprised in any one of SEQ ID NOs: 1-4).

Another aspect of the present disclosure comprises computationally engineered SIRPα binding polypeptides comprising chimeras encompassing the ECD domain of residues 19-131 (1-113 following cleavage of the signal peptide) of any one of SEQ ID NOs: 1-4 and the TMD from computationally designed SIRPα agonist sequences based on in silico evolutionary strategy algorithms and/or trained algorithms (such as machine learning algorithms) to enhance SIRPα agonist TMD stability. In some embodiments, the engineered SIRPα agonists comprising chimeras encompassing residues 19-131 (1-113 following cleavage of the signal peptide) of any one of SEQ ID NOs: 1-4 and the TMD from another organism. In some embodiments, the engineered SIRPα agonists comprising chimeras encompassing an engineered (e.g., rationally or computationally) ECD of residues 19-131 (1-113 following cleavage of the signal peptide) of any one of SEQ ID NOs: 1-4 and the TMD from another organism. In some embodiments, a SIRPα binding polypeptide chimera comprising an engineered domain is characterized by a conformational ensemble modulated relative to that of a reference polypeptide (e.g., a wild-type CD47 sequence, such as comprised in any one of SEQ ID NOs: 1-4).

Another aspect of the present disclosure comprises computationally engineered SIRPα binding polypeptides comprising chimeras encompassing residues 19-131 (1-113 following cleavage of the signal peptide) of any one of SEQ ID NOs: 1-4 carrying single or multiple mutations (such as those listed in Table 3, Table 4, and/or rationally/computationally engineered mutations) and the TMD from computationally designed SIRPα agonist sequences based on in silico evolutionary strategy algorithms and/or trained algorithms (such as machine learning algorithms) to enhance SIRPα agonist TMD stability. In some embodiments, the computationally modified chimeras combines with mutation(s) modulates the conformational ensemble of a SIRPα binding polypeptide relative to a reference polypeptide (e.g., a wild-type CD47 sequence, such as comprised in any one of SEQ ID NOs: 1-4).

### DEFINITIONS

The practice of some methods disclosed herein employ, unless otherwise indicated, techniques of immunology, biochemistry, chemistry, molecular biology, microbiology, cell biology, genomics and recombinant DNA. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M.J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)) (which is entirely incorporated by reference herein).

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein, a “cell” generally refers to a biological cell. A cell may be the basic structural, functional and/or biological unit of a living organism. A cell may originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g.,, Botryococcus braunii, Chlamydomonas reinhardtii, Nannochloropsis gaditana, Chlorella pyrenoidosa, Sargassum patens C. Agardh, and the like), seaweeds (e.g., kelp), a fungal cell (e.g.,, a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be synthetically made, sometimes termed an artificial cell).

The term “nucleotide,” as used herein, generally refers to a base-sugar-phosphate combination. A nucleotide may comprise a synthetic nucleotide. A nucleotide may comprise a synthetic nucleotide analog. Nucleotides may be monomeric units of a nucleic acid sequence (e.g., deoxyribonucleic acid (DNA) and ribonucleic acid (RNA)). The term nucleotide may include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives may include, for example, [aS]dATP, 7-deaza-dGTP and 7-deaza-dATP, and nucleotide derivatives that confer nuclease resistance on the nucleic acid molecule containing them. The term nucleotide as used herein may refer to dideoxyribonucleoside triphosphates (ddNTPs) and their derivatives. Illustrative examples of dideoxyribonucleoside triphosphates may include, but are not limited to, ddATP, ddCTP, ddGTP, ddITP, and ddTTP. A nucleotide may be unlabeled or detectably labeled, such as using moieties comprising optically detectable moieties (e.g., fluorophores). Labeling may also be carried out with quantum dots. Detectable labels may include, for example, radioactive isotopes, fluorescent labels, chemiluminescent labels, bioluminescent labels and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl) aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g., biotin-11-dUTP, biotin-16-dUTP, biotin-20-dUTP). A nucleotide may comprise a nucleotide analog. In some embodiments, nucleotide analogs may comprise structures of natural nucleotides that are modified at any position so as to alter certain chemical properties of the nucleotide yet retain the ability of the nucleotide analog to perform its intended function (e.g. hybridization to other nucleotides in RNA or DNA). Examples of positions of the nucleotide which may be derivatized include the 5 position, e.g., 5-(2-amino) propyl uridine, 5-bromo uridine, 5-propyne uridine, 5-propenyl uridine, etc.; the 6 position, e.g., 6-(2-amino) propyl uridine; the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine: O- and N-modified (e.g., alkylated, e.g., N6-methyl adenosine, or as otherwise known in the art) nucleotides; and other heterocyclically modified nucleotide analogs such as those described in Herdewijn, Antisense Nucleic Acid Drug Dev., 2000 Aug. 10 (4): 297-310. Nucleotide analogs may also comprise modifications to the sugar portion of the nucleotides. For example the 2′ OH-group may be replaced by a group selected from H, OR, R, F, Cl, Br, I, SH, SR, NH₂, NHR, NR₂, COOR, or OR, wherein R is substituted or unsubstituted C₁-C₆ alkyl, alkenyl, alkynyl, aryl, etc. Other possible modifications include those described in U.S. Pat. Nos. 5,858,988, and 6,291,438. Examples of positions of the nucleotide which may be derivatized include the 5 position, e.g., 5-(2-amino) propyl uridine, 5-bromo uridine, 5-propyne uridine, 5-propenyl uridine, etc.; the 6 position, e.g., 6-(2-amino) propyl uridine; the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine: O- and N-modified (e.g., alkylated, e.g., N6-methyl adenosine, or as otherwise known in the art) nucleotides; and other heterocyclically modified nucleotide analogs such as those described in Herdewijn, Antisense Nucleic Acid Drug Dev., 2000 Aug. 10 (4): 297-310.

Nucleotide analogs may also comprise modifications to the sugar portion of the nucleotides. For example the 2′ OH-group may be replaced by a group selected from H, OR, R, F, Cl, Br, I, SH, SR, NH₂, NHR, NR₂, COOR, or OR, wherein R is substituted or unsubstituted C₁-C₆ alkyl, alkenyl, alkynyl, aryl, etc. Other possible modifications include those described in U.S. Pat. Nos. 5,858,988, and 6,291,438.

The terms “polynucleotide,” “oligonucleotide,” and “nucleic acid” are used interchangeably to generally refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof. A polynucleotide may be DNA. A polynucleotide may be RNA. A polynucleotide may have any three-dimensional structure and may perform any function. A polynucleotide may comprise one or more analogs (e.g., altered backbone, sugar, or nucleobase). If present, modifications to the nucleotide structure may be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides may be interrupted by non-nucleotide components.

The terms “transfection” or “transfected” generally refer to introduction of a nucleic acid into a cell by non-viral or viral-based methods. The nucleic acid molecules may be gene sequences encoding complete proteins or functional portions thereof. See, e.g., Sambrook et al., 1989, Molecular Cloning: A Laboratory Manual, 18.1-18.88 (which is entirely incorporated by reference herein).

The terms “peptide,” “polypeptide,” and “protein” are used interchangeably herein to generally refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer may be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids may include natural amino acids and non-natural amino acids, which have been chemically modified to include a group or a chemical moiety not naturally present on the amino acid. Amino acid analogues may refer to amino acid derivatives. The term “amino acid” includes both D-amino acids and L-amino acids.

As used herein, the term “non-native” can generally refer to a nucleic acid or polypeptide sequence that is not found in a native nucleic acid or protein. Non-native may refer to affinity tags. Non-native may refer to fusions. Non-native may refer to a naturally occurring nucleic acid or polypeptide sequence that comprises mutations, insertions and/or deletions. A non-native sequence may exhibit and/or encode for an activity (e.g., enzymatic activity, methyltransferase activity, acetyltransferase activity, kinase activity, ubiquitinating activity, etc.) that may also be exhibited by the nucleic acid and/or polypeptide sequence to which the non-native sequence is fused. A non-native nucleic acid or polypeptide sequence may be linked to a naturally-occurring nucleic acid or polypeptide sequence (or a variant thereof) by genetic engineering to generate a chimeric nucleic acid and/or polypeptide sequence encoding a chimeric nucleic acid and/or polypeptide.

The term “promoter,” as used herein, generally refers to the regulatory DNA region which controls transcription or expression of a gene and which may be located adjacent to or overlapping a nucleotide or region of nucleotides at which RNA transcription is initiated. A promoter may contain specific DNA sequences which bind protein factors, often referred to as transcription factors, which facilitate binding of RNA polymerase to the DNA leading to gene transcription. A “basal promoter,” also referred to as a “core promoter,” may generally refer to a promoter that contains all the basic necessary elements to promote transcriptional expression of an operably linked polynucleotide. Eukaryotic basal promoters can contain a TATA-box and/or a CAAT box.

The term “expression,” as used herein, generally refers to the process by which a nucleic acid sequence or a polynucleotide is transcribed from a DNA template (such as into mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, “operably linked,” “operable linkage,” “operatively linked,” or grammatical equivalents thereof generally refer to juxtaposition of genetic elements, e.g., a promoter, an enhancer, a polyadenylation sequence, etc., wherein the elements are in a relationship permitting them to operate in the expected manner. For instance, a regulatory element, which may comprise promoter and/or enhancer sequences, is operatively linked to a coding region if the regulatory element helps initiate transcription of the coding sequence. There may be intervening residues between the regulatory element and coding region so long as this functional relationship is maintained.

A “vector” as used herein, generally refers to a macromolecule or association of macromolecules that comprises or associates with a polynucleotide and which may be used to mediate delivery of the polynucleotide to a cell. Examples of vectors include plasmids, viral vectors, liposomes, and other gene delivery vehicles. The vector generally comprises genetic elements, e.g., regulatory elements, operatively linked to a gene to facilitate expression of the gene in a target.

As used herein, “an expression cassette” and “a nucleic acid cassette” are used interchangeably generally to refer to a combination of nucleic acid sequences or elements that are expressed together or are operably linked for expression. In some cases, an expression cassette refers to the combination of regulatory elements and a gene or genes to which they are operably linked for expression.

A “functional fragment” of a DNA or protein sequence generally refers to a fragment that retains a biological activity (either functional or structural) that is substantially similar to a biological activity of the full-length DNA or protein sequence. A biological activity of a DNA sequence may be its ability to influence expression in a manner known to be attributed to the full-length sequence.

As used herein, an “engineered” or object generally indicates that the object has been modified by human intervention. According to non-limiting examples: a nucleic acid may be modified by changing its sequence to a sequence that does not occur in nature; a nucleic acid may be modified by ligating it to a nucleic acid that it does not associate with in nature such that the ligated product possesses a function not present in the original nucleic acid; an engineered nucleic acid may synthesized in vitro with a sequence that does not exist in nature; a protein may be modified by changing its amino acid sequence to a sequence that does not exist in nature; an engineered protein may acquire a new function or property. An “engineered” system comprises at least one engineered component.

As used herein, the term “optimally aligned” generally refers to an alignment of two amino acid sequences that give the highest percent identity score or maximizes the number of matched residues.

As used herein, “synthetic” and “artificial” are used interchangeably to generally refer to a protein or a domain thereof that has low sequence identity (e.g., less than 80% sequence identity, less than 70% sequence identity, less than 60% sequence identity, less than 50% sequence identity, less than 25% sequence identity, less than 10% sequence identity, less than 5% sequence identity, less than 1% sequence identity) to a naturally occurring human protein. For example, VPR and VP64 domains are synthetic transactivation domains.

The term “sequence identity” or “percent identity” in the context of two or more nucleic acids or polypeptide sequences, generally refers to two (e.g., in a pairwise alignment) or more (e.g., in a multiple sequence alignment) sequences that are the same or have a specified percentage of amino acid residues or nucleotides that are the same, when compared and aligned for maximum correspondence over a local or global comparison window, as measured using a sequence comparison algorithm. Suitable sequence comparison algorithms for polypeptide sequences include, e.g., BLASTP using parameters of a wordlength (W) of 3, an expectation (E) of 10, and the BLOSUM62 scoring matrix setting gap costs at existence of 11, extension of 1, and using a conditional compositional score matrix adjustment for polypeptide sequences longer than 30 residues; BLASTP using parameters of a wordlength (W) of 2, an expectation (E) of 1000000, and the PAM30 scoring matrix setting gap costs at 9 to open gaps and 1 to extend gaps for sequences of less than 30 residues (these are the default parameters for BLASTP in the BLAST suite available at https://blast.ncbi.nlm.nih.gov); or CLUSTALW with parameters of the Smith-Waterman homology search algorithm with parameters of a match of 2, a mismatch of −1, and a gap of −1; MUSCLE with default parameters; MAFFT with parameters retree of 2 and maxiterations of 1000; Novafold with default parameters; HMMER hmmalign with default parameters.

As defined herein, the term “inhibition,” “inhibit,” “inhibiting” and the like in reference to a protein-inhibitor interaction generally means negatively affecting (e.g. decreasing) the activity or function of the protein relative to the activity or function of the protein in the absence of the inhibitor. Inhibition may refer to reduction of a disease or symptoms of disease. Inhibition may refer to a reduction in the activity of a particular protein or nucleic acid target. The protein may be deoxycytidine kinase. Thus, inhibition includes, at least in part, partially or totally blocking stimulation, decreasing, preventing, or delaying activation, or inactivating, desensitizing, or down-regulating signal transduction or enzymatic activity or the amount of a protein.

The term “modulator” generally refers to a composition that increases or decreases the level of a target molecule or the function of a target molecule or the physical state of the target of the molecule.

The term “modulate” generally refers to the act of changing or varying one or more properties. “Modulation” generally to the process of changing or varying one or more properties. For example, a modulator of a target protein can change by increasing or decreasing a property or function of the target molecule or the amount of the target molecule. A modulator of a disease can decrease a symptom, cause, or characteristic of the targeted disease.

As used herein, “immune checkpoint modulator” generally refers to an agent which results in the activation or inhibition of one or more immune checkpoint proteins. For example, immune checkpoint modulators may include, but are not limited to, CD47, PD-L1, A2AR, B7-H3, B7-H4, BTLA, CTLA-4, IDO, KIR, LAG3, NOX2, PD-1, TIM-3, VISTA, and SIGLEC7.

The term “pluripotent cells” generally refers to cells that can self-renew and proliferate while remaining in an undifferentiated state and that can, under the proper conditions, be induced to differentiate into specialized cell types. The term “pluripotent cells,” as used herein, encompass embryonic stem cells and other types of stem cells, including fetal, amniotic, or somatic stem cells. Exemplary human stem cell lines include the H9 human embryonic stem cell line. Additional exemplary stem cell lines include those made available through the National Institutes of Health Human Embryonic Stem Cell Registry and the Howard Hughes Medical Institute HUES collection (as described in Cowan, C. A. et. al, New England J. Med. 350:13. (2004), incorporated by reference herein in its entirety.)

The term “pluripotent stem cells” as used herein generally refers to cell which have the potential to differentiate into any of the three germ layers: endoderm (e.g. the stomach linking, gastrointestinal tract, lungs, etc), mesoderm (e.g. muscle, bone, blood, urogenital tissue, etc) or ectoderm (e.g. epidermal tissues and nervous system tissues). The term “pluripotent stem cells,” as used herein, can also encompass “induced pluripotent stem cells,” or “iPSCs,” a type of pluripotent stem cell derived from a non-pluripotent cell. Examples of parent cells include somatic cells that have been reprogrammed to induce a pluripotent, undifferentiated phenotype by various means. Such “iPS” or “iPSC” cells can be created by inducing the expression of certain regulatory genes or by the exogenous application of certain proteins. Methods for the induction of iPS cells are known in the art and are further described below. (See, e.g., Zhou et al., Stem Cells 27 (11): 2667-74 (2009); Huangfu et al., Nature Biotechnol. 26 (7): 795 (2008); Woltjen et al., Nature 458 (7239): 766-770 (2009); and Zhou et al., Cell Stem Cell 8:381-384 (2009); each of which is incorporated by reference herein in its entirety.)

The term “pluripotent stem cell characteristics” generally refers to characteristics of a cell that distinguish pluripotent stem cells from other cells. The ability to give rise to progeny that can undergo differentiation, under the appropriate conditions, into cell types that collectively demonstrate characteristics associated with cell lineages from all of the three germinal layers (endoderm, mesoderm, and ectoderm) is a pluripotent stem cell characteristic. Expression or non-expression of certain combinations of molecular markers are also pluripotent stem cell characteristics. For example, human pluripotent stem cells express at least several, and in some embodiments, all of the markers from the following non-limiting list: SSEA-3, S SEA-4, TRA-1-60, TRA-1-81, TRA-2-49/6E, ALP, Sox2, E-cadherin, UTF-1, Oct4, Rex1, and Nanog. Cell morphologies associated with pluripotent stem cells are also pluripotent stem cell characteristics. As described herein, cells do not need to pass through pluripotency to be reprogrammed into endodermal progenitor cells and/or hepatocytes.

As used herein, the terms “multipotent” or “multipotent cell” generally refer to a cell type that can give rise to a limited number of other particular cell types. For example, induced multipotent cells are capable of forming endodermal cells. Additionally, multipotent blood stem cells can differentiate itself into several types of blood cells, including lymphocytes, monocytes, neutrophils, etc.

As used herein, the term “oligopotent” generally refers to the ability of an adult stem cell to differentiate into only a few different cell types. For example, lymphoid or myeloid stem cells are capable of forming cells of either the lymphoid or myeloid lineages, respectively.

As used herein, the term “unipotent” generally refers to the ability of a cell to form a single cell type.

As used herein, the term “totipotent” generally refers to the ability of a cell to form an entire organism.

As used herein, the term “non-pluripotent cells” generally refers to mammalian cells that are not pluripotent cells. Examples of such cells include differentiated cells as well as progenitor cells. Examples of differentiated cells include, but are not limited to, cells from a tissue selected from bone marrow, skin, skeletal muscle, fat tissue and peripheral blood. Example cell types include, but are not limited to, fibroblasts, hepatocytes, myoblasts, neurons, osteoblasts, osteoclasts, and T-cells. The starting cells employed for generating the induced multipotent cells, the endodermal progenitor cells, and the hepatocytes can be non-pluripotent cells.

Differentiated cells include, but are not limited to, multipotent cells, oligopotent cells, unipotent cells, progenitor cells, and terminally differentiated cells. In particular embodiments, a less potent cell is considered “differentiated” in reference to a more potent cell.

The term “somatic cell” as used herein generally refers to a cell forming the body of an organism. Somatic cells include cells making up organs, skin, blood, bones and connective tissue in an organism, but not germ cells.

As used herein, the terms “subject” or “patient” generally refer to any animal, such as a domesticated animal, a zoo animal, or a human. The “subject” or “patient” can be a mammal like a dog, cat, bird, livestock, or a human. Specific examples of “subjects” and “patients” include, but are not limited to, individuals (particularly human) with a disease or disorder related to the liver, heart, lung, kidney, pancreas, brain, neural tissue, blood, bone, bone marrow, and the like.

The terms “hypo-immunogenic pluripotent cell” or “HIP cell” as used herein generally refer to a pluripotent cell that retains its pluripotent characteristics and yet gives rise to a reduced immunological rejection response when transferred into an allogeneic host. In some cases, HIP cells do not give rise to an immune response. Thus, “hypo-immunogenic” as used herein generally refers to a significantly reduced or eliminated immune response when compared to the immune response of a parental (e.g., wild-type) cell as outlined herein. In some cases, the immune response comprises natural killer (NK) cell cytotoxicity. In some cases, the immune response comprises macrophage cytotoxicity. In some cases, HIP cells as described herein are immunologically silent and yet retain pluripotent capabilities.

The term “allogeneic” as used herein generally refers to the genetic dissimilarity of a host organism and a cellular transplant.

“Inhibitors,” “activators,” and “modulators” generally refer to substances or conditions which impact a function or expression of a biologically-relevant molecule. The term “modulator” generally includes both inhibitors and activators. They may be identified using in vitro and in vivo assays for expression or activity of a target molecule.

The term “Inhibitors” generally refers to agents that, e.g., inhibit expression or bind to target molecules or proteins. They may partially or totally block stimulation or have protease inhibitor activity. They may reduce, decrease, prevent, or delay activation, including inactivation, desensitization, or down regulation of the activity of the described target protein. Modulators may be antagonists of the target molecule or protein.

“Activators” generally refers to agents that, e.g., induce or activate the function or expression of a target molecule or protein. They may bind to, stimulate, increase, open, activate, or facilitate the target molecule activity. Activators may be agonists of the target molecule or protein.

As used herein the term “modification” generally to an alteration that physically differentiates the modified molecule from the parent molecule. In one embodiment, an amino acid change in a CD47, HSVtk, EC-CD, or iCasp9 variant polypeptide prepared according to the methods described herein differentiates it from the corresponding parent that has not been modified according to the methods described herein, such as wild-type proteins, a naturally occurring mutant proteins or another engineered protein that does not include the modifications of such variant polypeptide. In another embodiment, a variant polypeptide includes one or more modifications that differentiates the function of the variant polypeptide from the unmodified polypeptide. For example, an amino acid change in a variant polypeptide affects its receptor binding profile. In other embodiments, a variant polypeptide comprises substitution, deletion, or insertion modifications, or combinations thereof. In another embodiment, a variant polypeptide includes one or more modifications that increases its affinity for a receptor compared to the affinity of the unmodified polypeptide.

In some cases, a variant polypeptide includes one or more substitutions, insertions, or deletions relative to a corresponding native or parent sequence. In certain embodiments, a variant polypeptide comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more modifications.

As used herein and in the appended claims, the singular forms “a,” “an,” and “the” include plural reference unless the context clearly indicates otherwise.

It is understood that aspect and variations of the invention described herein include “consisting” and/or “consisting essentially of” aspects and variations.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

### SIPRα Binding Polypeptides

In one aspect, the present disclosure provides for a signal regulatory protein alpha (SIRPα) binding polypeptide. In some cases, a SIRPα binding peptide comprises a sequence of a wild-type (WT) CD47 variant. Example WT CD47 splice variants include Q08722-1 (SEQ ID NO: 1), Q08722-2 (SEQ ID NO: 2), Q08722-3 (SEQ ID NO: 3), and Q08722-4 (SEQ ID NO: 4), which are shown in Table 1. In some cases, a SIRPα binding peptide comprises one or more modifications (e.g., mutations) of a WT CD47 variant. In some cases, a SIRPα binding peptide is an engineered SIRPα binding peptide. Engineered SIRPα binding peptides can be generated by one or more of the strategies disclosed herein. Relative to a reference polypeptide (e.g., a WT CD47 variant, such as any one of SEQ ID NOs: 1-4), an engineered SIRPα binding peptide may comprise different structural and/or functional properties as described herein. In some embodiments, the reference polypeptide comprises from residue 19 to the final residue of any one of SEQ ID NOs: 1-4. In some embodiments, the reference polypeptide comprises residues 19-290 of any one of SEQ ID Nos: 1-4.

The engineered SIRPα binding peptide may comprise an extracellular domain (ECD). The ECD can comprise a sequence with at least about 50%, at least about 55%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to an ECD of any one of SEQ ID NOs: 1-4, 10-640, or 648-653. The domain boundaries of the ECD can be identified, for example, by optimal alignment to any one of SEQ ID NOs: 1-4. In an example, an ECD of SEQ ID NO: 1 comprises residues 19-131 of SEQ ID NO: 1 (residues 1-113 following cleavage of the leader peptide).

The engineered SIRPα binding polypeptide may comprise an extracellular loop region (ECLR). The ECLR may comprise portions of the ECD and TMD which are on the extracellular side of a cell membrane when expressed by a cell. The ECLR can comprise, at least in part, an extracellular loop 1 (ECL1) region. The ECL1 region can comprise any of the extracellular residues of the loop region connecting helices II and III of a TMD as described herein (e.g., as comprised in wild type CD47 or an engineered SIRPα binding polypeptide of the disclosure). In an example, the ECL1 comprises residues 198-207 of SEQ ID NO: 1. The ECLR can comprise, at least in part, an extracellular loop 2 (ECL2) region. The ECL2 region can comprise any of the extracellular residues of the loop region connecting helices VI and V of a TMD as described herein (e.g., as comprised in wild type CD47 or an engineered SIRPα binding polypeptide of the disclosure). In an example, the ECL2 comprises residues 258-267 of SEQ ID NO: 1. The ECLR can comprise, at least in part, a peptide linker connecting the C-terminal end of the ECD and the N-terminal extracellular tip of the TMD. In an example, the peptide linker comprises residues 132-137 of SEQ ID NO: 1. The ECLR can comprise a sequence with at least about 50%, at least about 55%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to an ECLR of any one of SEQ ID NOs: 1-4, 10-640, or 648-653. The domain boundaries of the ECLR can be identified, for example, by optimal alignment to any one of SEQ ID NOs: 1-4. In an example, an ECLR of SEQ ID NO: 1 comprises residues 132-137, 198-207, and 258-267 of SEQ ID NO: 1 (residues 114-119, 180-189, and 240-249 following cleavage of the leader peptide).

The engineered SIRPα binding peptide may comprise a transmembrane domain (TMD). The TMD can comprise five transmembrane helices which are configured to span the lipid membrane of a cell expressing the SIRPα polypeptide. The TMD can comprise a first transmembrane helix (helix I). In an example, the helix I comprises residues 142-162 of SEQ ID NO: 1. The TMD can comprise a second transmembrane helix (helix II). In an example, the helix II comprises residues 177-197 of SEQ ID NO: 1. The TMD can comprise a third transmembrane helix (helix III). In an example, the helix III comprises residues 208-228 of SEQ ID NO: 1. The TMD can comprise a fourth transmembrane helix (helix IV). In an example, the helix IV comprises residues 236-256 of SEQ ID NO: 1. The TMD can comprise a fifth transmembrane helix (helix V). In an example, the helix V comprises residues 269-289 of SEQ ID NO: 1. The TMD can comprise a sequence with at least about 50%, at least about 55%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to an TMD of any one of SEQ ID NOs: 1-4, 10-640, or 648-653. The domain boundaries of the TMD can be identified, for example, by optimal alignment to any one of SEQ ID NOs: 1-4. In an example, an TMD of SEQ ID NO: 1 comprises residues 142-162, 177-197, 208-228, 236-256, and 269-289 SEQ ID NO: 1 (residues 124-144, 159-179, 190-210, 218-238, and 251-271 following cleavage of the leader peptide).

The SIRPα binding polypeptide may comprise a signal peptide. The signal peptide may comprise the first 18 residues of any one of SEQ ID NOs: 1-4. The signal peptide may target the protein to the endoplasmic reticulum for further processing and ultimate cell-surface expression. In some embodiments, the signal peptide is cleaved to give a mature polypeptide. In some embodiments, the engineered SIRPα binding polypeptide does not comprise a signal peptide.

The α SIRPα binding polypeptide may comprise a C-terminal domain (CTD). The CTD may be present on the cytoplasmic side of a membrane of a cell expressing the SIRPα binding polypeptide. The CTD may comprise residue 290 through the final residue of any one of SEQ ID NOs: 1-4. In some embodiments, the engineered SIRPα binding peptide does not comprise a CTD.

TABLE 1
WT CD47 Splice Variants

| | |
|---|---|
| | SEQ ID |
| Sequence | NO |

>sp/Q08722-1|CD47_HUMAN Leukocyte surface antigen CD47 OS = Homo sapiens

| | |
|---|---|
| OX = 9606 GN = CD47 PE = 1 SV = 1 | |
| MWPLVAALLLGSACCGSAQLLFNKTKSVEFTFCNDTVVIPCFVTNMEAQNTTEVYVKWK | 1 |
| FKGRDIYTFDGALNKSTVPTDFSSAKIEVSQLLKGDASLKMDKSDAVSHTGNYTCEVTELT | |
| REGETIIELKYRVVSWFSPNENILIVIFPIFAILLFWGQFGIKTLKYRSGGMDEKTIALLVAGL | |
| VITVIVIVGAILFVPGEYSLKNATGLGLIVTSTGILILLHYYVFSTAIGLTSFVIAILVIQVIAYI | |
| LAVVGLSLCIAACIPMHGPLLISGLSILALAQLLGLVYMKFVASNQKTIQPPRKAVEEPLNA | |
| FKESKGMMNDE | |

>sp/Q08722-2|CD47_HUMAN Isoform OA3-293 of Leukocyte surface antigen CD47
OS = Homo sapiens OX = 9606 GN = CD47

| | |
|---|---|
| MWPLVAALLLGSACCGSAQLLFNKTKSVEFTFCNDTVVIPCFVTNMEAQNTTEVYVKWK | 2 |
| FKGRDIYTFDGALNKSTVPTDFSSAKIEVSQLLKGDASLKMDKSDAVSHTGNYTCEVTELT | |
| REGETIIELKYRVVSWFSPNENILIVIFPIFAILLFWGQFGIKTLKYRSGGMDEKTIALLVAGL | |
| VITVIVIVGAILFVPGEYSLKNATGLGLIVTSTGILILLHYYVFSTAIGLTSFVIAILVIQVIAYI | |
| LAVVGLSLCIAACIPMHGPLLISGLSILALAQLLGLVYMKFV | |

>sp/Q08722-3|CD47_HUMAN Isoform OA3-305 of Leukocyte surface antigen CD47

| | |
|---|---|
| OS = Homo sapiens OX = 9606 GN = CD47 | |
| MWPLVAALLLGSACCGSAQLLFNKTKSVEFTFCNDTVVIPCFVTNMEAQNTTEVYVKWK | 3 |
| FKGRDIYTFDGALNKSTVPTDFSSAKIEVSQLLKGDASLKMDKSDAVSHTGNYTCEVTELT | |
| REGETIIELKYRVVSWFSPNENILIVIFPIFAILLFWGQFGIKTLKYRSGGMDEKTIALLVAGL | |
| VITVIVIVGAILFVPGEYSLKNATGLGLIVTSTGILILLHYYVFSTAIGLTSFVIAILVIQVIAYI | |
| LAVVGLSLCIAACIPMHGPLLISGLSILALAQLLGLVYMKFVASNQKTIQPPRNN | |

>sp|Q08722-4|CD47_HUMAN Isoform OA3-312 of Leukocyte surface antigen CD47

| | |
|---|---|
| OS = Homo sapiens OX = 9606 GN = CD47 | |
| MWPLVAALLLGSACCGSAQLLFNKTKSVEFTFCNDTVVIPCFVTNMEAQNTTEVYVKWK | 4 |
| FKGRDIYTFDGALNKSTVPTDFSSAKIEVSQLLKGDASLKMDKSDAVSHTGNYTCEVTELT | |
| REGETIIELKYRVVSWFSPNENILIVIFPIFAILLFWGQFGIKTLKYRSGGMDEKTIALLVAGL | |
| VITVIVIVGAILFVPGEYSLKNATGLGLIVTSTGILILLHYYVFSTAIGLTSFVIAILVIQVIAYI | |
| LAVVGLSLCIAACIPMHGPLLISGLSILALAQLLGLVYMKFVASNQKTIQPPRKAVEEPLN | |

In some embodiments, a polypeptide (e.g., engineered SIRPα binding polypeptide) as described herein comprises one or more cysteine mutations (e.g., relative to a reference polypeptide comprising a sequence of any one of SEQ ID NOs: 1-4). Cysteine residues may be introduced into Examples of cysteine mutations to induce oligomerization (e.g., relative to a reference sequence, such as SEQ ID NO: 1) are shown below in Table 2.

TABLE 2
Example cysteine mutations to induce oligomerization

| Sequence (mutated cysteines in bold) | SEQ ID NO |
|---|---|
| MWPLVAALLLGSACCGSAQLLFNKTKSVEFTFCNDTVVIPCFVTNMEAQNTTEVYVKWK | 5 |
| FKGRDIYTFDGALNKCTVPTDFSSAKIEVSQLLKGDASLKMDKSDAVSHTGNYTCEVTEL | |
| TREGETIIELKYRVVSWFSPNENILIVIFPIFAILLFWGQFGIKTLKYRSGGMDEKTIALLVAG | |
| LVITVIVIVGAILFVPGEYSLKNATGLGLIVTSTGILILLHYYVFSTAIGLTSFVIAILVIQVIAY | |
| ILAVVGLSLCIAACIPMHGPLLISGLSILALAQLLGLVYMKFVASNQKTIQPPRKAVEEPLN | |
| AFKESKGMMNDE | |
| MWPLVAALLLGSACCGSAQLLFNKTKSVEFTFCNDTVVIPCFVTNMEAQNTTEVYVKWK | 6 |
| FKGRDIYTFDGALNKSTVPTDFSSAKIEVSQLLKGDASLKMDKSDAVSHTGNYTCEVTELT | |
| REGETIIELKYRVVSWFSPNENILIVIFPIFAILLFWGQFGIKTLKYRSGGMDEKTIALLVAGL | |
| VITVIVIVGAILFVPGEYSLKNACGLGLIVTSTGILILLHYYVFSTAIGLTSFVIAILVIQVIAYI | |
| LAVVGLSLCIAACIPMHGPLLISGLSILALAQLLGLVYMKFVASNQKTIQPPRKAVEEPLNA | |
| FKESKGMMNDE | |
| MWPLVAALLLGSACCGSAQLLFNKTKSVEFTFCNDTVVIPCFVTNMEAQNTTEVYVKWK | 7 |
| FKGRDIYTFDGALNKSTVPTDFSSAKIEVSQLLKGDASLKMDKSDAVSHTGNYTCEVTELT | |
| REGETIIELKYRVVSWFSPNENILIVIFPIFAILLFWGQFGIKTLKYRSGGMDEKTIALLVAGL | |
| VITVIVIVGAILFVPGEYSLKNATGLGLIVTSTGILILLHYCVFSTAIGLTSFVIAILVIQVIAYI | |
| LAVVGLSLCIAACIPMHGPLLISGLSILALAQLLGL VYMKFVASNQKTIQPPRKAVEEPLNA | |
| FKESKGMMNDE | |
| MWPLVAALLLGSACCGSAQLLFNKTKSVEFTFCNDTVVIPCFVTNMEAQNTTEVYVKWK | 8 |
| FKGRDIYTFDGALNKCTVPTDFSSAKIEVSQLLKGDASLKMDKSDAVSHTGNYTCEVTEL | |
| TREGETIIELKYRVVSWFSPNENILIVIFPIFAILLFWGQFGIKTLKYRSGGMDEKTIALLVAG | |
| LVITVIVIVGAILFVPGEYSLKNACGLGLIVTSTGILILLHYCVFSTAIGLTSFVIAILVIQVIA | |
| YILAVVGLSLCIAACIPMHGPLLISGLSILALAQLLGLVYMKFVASNQKTIQPPRKAVEEPL | |
| NAFKESKGMMNDE | |
| MWPLVAALLLGSACCGSAQLLFNKTKSVEFTFCNDTVVIPCFVTNMEAQNTTEVYVKWK | 9 |
| FKGRDIYTFDGALNKCTVPTDFSSAKIEVSQLLKGDASLKMDKSDAVSHTGNYTCEVTEL | |
| TREGETIIELKYRVVSWFSPNENILIVIFPIFAILLFWGQFGIKTLKYRSGGMDEKTIALLVAG | |
| LVITVIVIVGAILFVPGEYSLKNACGLGLIVTSTGILILLHYCVFSTAIGLTSFVIAILVIQVIAY | |
| ILAVVGLSLCIAACIPMHGPLLISGLSILALAQLLGLVYMKFVASNQKTIQPPRKAVEEPLN | |
| AFKESKGMMNDE | |

### CD47 Cancer Mutants

In some embodiments, a polypeptide (e.g., engineered SIRPα binding polypeptide) as described herein comprises one or more mutations that have been identified in a cancer or associated with a cancer. (e.g., relative to a reference polypeptide comprising a sequence of any one of SEQ ID NOs: 1-4).

In some aspects, the mutation may be a single CD47 point mutations. In some embodiments, the mutation may comprise a plurality of CD47 mutations. In some embodiments, the mutation has been identified in a cancer sample. In some embodiments, the mutation may be available in a public database (e.g., COSMIC, TCGA, cBioPortal, OncoDB). In some embodiments, the mutation may be present in a very low percentage of cancer samples or may be overexpressed in some cancer samples/types. Some of these mutations have been mapped onto the crystal structure of CD47 and found to cluster on critical functional regions of the receptor. For example, CD47 mutations associated with cancer as disclosed herein have been mapped to the extra cellular loop region (ECLR) region, transmembrane helix III, and the extracellular domain (ECD).

In some embodiments, the mutation is mapped on a distinct region of the molecule. In some embodiments, a SIRPα binding polypeptide comprising a cancer mutation as described herein may have functional effects that depend on its location on the crystal structure of the polypeptide. Accordingly, these mutations may be subdivided based on where they occur in the molecule, such as in the extracellular domain (ECD), in the transmembrane domain (TMD), in the extracellular loop region (ECLR), in the C-terminal domain (CTD) or between any two of these domains. Based on the relative position of these mutations within the molecule, they may impact secondary, tertiary, or quaternary structure by creating opportunities for new non-covalent and/or covalent interactions or abrogating others. These structural changes may in turn affect functional aspects of the SIRPα binding polypeptide transmembrane signaling. For example, cancer mutant W136C can be configured to form a cysteine residue with C259, resulting in an additional disulfide bond between the ECD and TMD interdomain linker and the extracellular portion of helix V. As discussed elsewhere herein, such changes in the interactions between domains can alter the conformational ensembles of polypeptides, leading to various functional consequences, such as enhanced binding to SIRPα. In another example, mutations may influence or alter the quaternary assembly of SIRPα binding polypeptides by encouraging or disrupting formation of dimers or oligomers. In some embodiments, mutations may increase stability of the polypeptide (alone or in combination), and mutations that may disrupt binding of endogenous protein partners (other than SIRPα). Accordingly, engineered polypeptides of the disclosure may comprise one or more of the cancer mutations disclosed herein to impart a certain functional consequence (e.g., enhanced SIPRα binding) relative to a reference polypeptide (e.g., wild-type human CD47 molecule). In another example, mutations associated with cancer as discussed herein were found to map to the ECD. In some embodiments, a mutation in the ECD may alter/disrupt binding to TSP-1, integrins or increase affinity for SIRPα. In some embodiments, CD47 ECD cancer mutation sites represent regions of instability on CD47, and a mutation may increase receptor stability which may enhance function.

Non-limiting examples of CD47 mutations associated with cancer which may appear in polypeptides of the disclosure are listed in Table 3.

TABLE 3
CD47 mutations associated with cancer

| | SEQ ID NO: 1 | | |
|---|---|---|---|
| | Position (including | | PDB 7MYZ Position |
| Wild | peptide leader | | (excluding peptide |
| type AA | sequence) | Mutant AA | leader sequence) |
| W | 2 | C | NA |
| A | 6 | V | NA |
| G | 11 | V | NA |
| S | 17 | L | NA |
| L | 20 | P | 2 |
| K | 24 | T | 6 |
| T | 31 | M | 13 |
| D | 35 | N | 17 |
| P | 40 | L | 22 |
| C | 41 | R | 23 |
| F | 42 | C | 24 |
| E | 47 | D | 29 |
| E | 53 | D | 35 |
| V | 54 | I | 36 |
| V | 56 | I | 38 |
| D | 64 | H | 46 |
| S | 75 | C | 57 |
| D | 80 | E | 62 |
| D | 80 | G | 62 |
| A | 84 | V | 66 |
| L | 91 | S | 73 |
| S | 97 | F | 79 |
| M | 100 | I | 82 |
| D | 101 | Y | 83 |
| A | 105 | G | 87 |
| V | 106 | F | 88 |
| N | 111 | K | 93 |
| E | 118 | Q | 100 |
| G | 123 | S | 105 |
| E | 124 | K | 106 |
| I | 127 | S | 109 |
| I | 127 | V | 109 |
| E | 128 | K | 110 |
| Y | 131 | F | 113 |
| R | 132 | C | 114 |
| W | 136 | C | 118 |
| N | 140 | K | 122 |
| N | 142 | T | 124 |
| V | 146 | G | 128 |
| V | 146 | A | 128 |
| I | 153 | M | 135 |
| W | 157 | L | 139 |
| F | 160 | L | 142 |
| K | 166 | E | 148 |
| G | 170 | C | 152 |
| A | 178 | D | 160 |
| I | 186 | F | 168 |
| A | 194 | T | 176 |
| L | 196 | V | 178 |
| L | 196 | I | 178 |
| S | 203 | A | 185 |
| N | 206 | S | 188 |
| A | 207 | S | 189 |
| L | 210 | V | 192 |
| G | 211 | D | 193 |
| V | 214 | L | 196 |
| T | 215 | S | 197 |
| S | 216 | Y | 198 |
| A | 231 | S | 213 |
| A | 231 | V | 213 |
| V | 238 | I | 220 |
| Q | 245 | H | 227 |
| A | 248 | D | 230 |
| Y | 249 | F | 231 |
| A | 262 | V | 244 |
| I | 264 | L | 246 |
| H | 267 | Y | 249 |
| G | 285 | R | 267 |
| M | 289 | I | 271 |
| F | 291 | L | 273 |
| Q | 300 | H | 282 |
| P | 301 | H | 283 |
| R | 303 | M | 285 |
| A | 305 | V | 287 |
| E | 307 | D | 289 |
| E | 307 | Q | 289 |
| E | 308 | K | 290 |
| A | 312 | V | 294 |
| K | 314 | N | 296 |
| N | 321 | S | 303 |
| D | 322 | N | 304 |
| E | 323 | D | 305 |

In some cases, a SIRPα binding polypeptide comprises at least one mutant listed in Table 3. In some cases, the SIRPα binding polypeptide comprises any 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 76 mutations listed in Table 3. In some cases, the SIRPα binding polypeptide comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 76 mutations listed in Table 3. In some cases, the SIRPα binding polypeptide comprises at most 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 mutation listed in Table 3.

In some embodiments, a SIRPα binding polypeptide comprises at least one mutation of: M31, L40, C42, D47, D64, C75, E80, F97, G105, K106, F106, K111, S123, S127, K128, F131, C132, C136, K140, T142, G146, M153, L157, L160, E166, C170, D178, A203, S207, V210, D211, L214, S215, V262, L264, Y267, or any combination thereof, where the mutation position is relative to any one of SEQ ID NOs: 1-4. In some embodiments, a SIRPα binding polypeptide comprises an M31 residue. In some embodiments, a SIRPα binding polypeptide comprises an L40 residue. In some embodiments, a SIRPα binding polypeptide comprises a C42 residue. In some embodiments, a SIRPα binding polypeptide comprises a D47 residue. In some embodiments, a SIRPα binding polypeptide comprises a D64 residue. In some embodiments, a SIRPα binding polypeptide comprises a C75 residue. In some embodiments, a SIRPα binding polypeptide comprises an E80 residue. In some embodiments, a SIRPα binding polypeptide comprises an F97 residue. In some embodiments, a SIRPα binding polypeptide comprises a G105 residue. In some embodiments, a SIRPα binding polypeptide comprises a K106 residue. In some embodiments, a SIRPα binding polypeptide comprises an F106 residue. In some embodiments, a SIRPα binding polypeptide comprises a K111 residue. In some embodiments, a SIRPα binding polypeptide comprises an S123 residue. In some embodiments, a SIRPα binding polypeptide comprises an S127 residue. In some embodiments, a SIRPα binding polypeptide comprises a K128 residue. In some embodiments, a SIRPα binding polypeptide comprises an F131 residue. In some embodiments, a SIRPα binding polypeptide comprises a C132 residue. In some embodiments, a SIRPα binding polypeptide comprises a C136 residue. In some embodiments, a SIRPα binding polypeptide comprises a K140 residue. In some embodiments, a SIRPα binding polypeptide comprises a T142 residue. In some embodiments, a SIRPα binding polypeptide comprises a G146 residue. In some embodiments, a SIRPα binding polypeptide comprises an M153 residue. In some embodiments, a SIRPα binding polypeptide comprises an L157 residue. In some embodiments, a SIRPα binding polypeptide comprises an L160 residue. In some embodiments, a SIRPα binding polypeptide comprises an E166 residue. In some embodiments, a SIRPα binding polypeptide comprises a C170 residue. In some embodiments, a SIRPα binding polypeptide comprises a D178 residue. In some embodiments, a SIRPα binding polypeptide comprises a A203 residue. In some embodiments, a SIRPα binding polypeptide comprises an S207 residue. In some embodiments, a SIRPα binding polypeptide comprises a V210 residue. In some embodiments, a SIRPα binding polypeptide comprises a D211 residue. In some embodiments, a SIRPα binding polypeptide comprises an L214 residue. In some embodiments, a SIRPα binding polypeptide comprises an S215 residue. In some embodiments, a SIRPα binding polypeptide comprises a V262 residue. In some embodiments, a SIRPα binding polypeptide comprises an L264 residue. In some embodiments, a SIRPα binding polypeptide comprises a Y267 residue.

In some cases, a SIRPα binding polypeptide comprises a sequence at least about 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, or 100% identical to any one of SEQ ID NOs: 385-475. In some cases, a SIRPα binding polypeptide comprises a sequence at least about 80%, 81%, 82%, 83%, 84%, 85%, 86%, 87%, 88%, 89%, 90%, 91%, 92%, 93%, 94%, 95%, 96%, 97%, 98%, or 99%, or 100% identical to SEQ ID NO: 389. In some cases, a SIRPα binding polypeptide comprises a sequence at least about 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, or 100% identical to SEQ ID NO: 391. In some cases, a SIRPα binding polypeptide comprises a sequence at least about 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, or 100% identical to SEQ ID NO: 419. In some cases, a SIRPα binding polypeptide comprises a sequence at least about 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, or 100% identical to SEQ ID NO: 393. In some cases, a SIRPα binding polypeptide comprises a sequence at least about 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, or 100% identical to SEQ ID NO: 395. In some cases, a SIRPα binding polypeptide comprises a sequence at least about 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, or 100% identical to SEQ ID NO: 397. In some cases, a SIRPα binding polypeptide comprises a sequence at least about 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, or 100% identical to SEQ ID NO: 406. In some cases, a SIRPα binding polypeptide comprises a sequence at least about 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, or 100% identical to SEQ ID NO: 411. In some cases, a SIRPα binding polypeptide comprises a sequence at least about 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, or 100% identical to SEQ ID NO: 413. In some cases, a SIRPα binding polypeptide comprises a sequence at least about 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, or 100% identical to SEQ ID NO: 467. In some cases, a SIRPα binding polypeptide comprises a sequence at least about 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, or 100% identical to SEQ ID NO: 468. In some cases, a SIRPα binding polypeptide comprises a sequence at least about 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, or 100% identical to SEQ ID NO: 469. In some cases, a SIRPα binding polypeptide comprises a sequence at least about 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, or 100% identical to SEQ ID NO: 469. In some cases, a SIRPα binding polypeptide comprises a sequence at least about 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, or 100% identical to SEQ ID NO: 470. In some cases, a SIRPα binding polypeptide comprises a sequence at least about 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, or 100% identical to SEQ ID NO: 471. In some cases, a SIRPα binding polypeptide comprises a sequence at least about 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, or 100% identical to SEQ ID NO: 473. In some cases, a SIRPα binding polypeptide comprises a sequence at least about 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, or 100% identical to SEQ ID NO: 474. In some cases, a SIRPα binding polypeptide comprises a sequence at least about 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, or 100% identical to SEQ ID NO: 475. In some cases, a SIRPα binding polypeptide comprises a sequence at least about 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, or 100% identical to SEQ ID NO: 472.

### CD47 Polymorphisms

In some embodiments, a polypeptide (e.g., engineered SIRPα binding polypeptide) as described herein comprises one or more mutations that have been identified as a naturally occurring polymorphism (e.g., relative to a reference polypeptide comprising a sequence of any one of SEQ ID NOs: 1-4). Some of these mutations have been mapped onto the crystal structure of human CD47 and found to cluster on critical functional regions of the receptor. For example, CD47 polymorphisms as disclosed herein have been mapped to the extra cellular loop region (ECLR) region, transmembrane domain (TMD), and the extracellular domain (ECD).

In some embodiments, the polymorphism is mapped on a distinct region of the molecule. In some embodiments, a polymorphism described herein may have functional effects that depend on its location on the crystal structure of the molecule (e.g., SIRPα binding polypeptide). Accordingly, these polymorphisms may be subdivided based on where they occur in the molecule, such as in the extracellular domain (ECD), in the transmembrane domain (TMD), in the extracellular loop region (ECLR), or between any two of these molecules. Based on the relative position of these polymorphisms within the molecule, they may impact secondary, tertiary, or quaternary structure by creating opportunities for new non-covalent and/or covalent interactions or abrogating others. These structural changes may in turn affect functional aspects of the SIRPα binding polypeptide transmembrane signaling. For example, polymorphism C33Y can disrupt an interdomain disulfide bond, thus altering the conformational ensemble of the molecule. In another example, polymorphisms may influence or alter the quaternary assembly of SIRPα binding polypeptides by encouraging or disrupting formation of dimers or oligomers. In some embodiments, polymorphisms may increase stability of the receptor (alone or in combination), and polymorphisms that may disrupt binding of endogenous protein partners (other than SIRPα). Accordingly, engineered polypeptides of the disclosure may comprise one or more of the polymorphisms disclosed herein to impart a certain functional consequence (e.g., enhanced SIPRα binding) relative to a wild type CD47 molecule.

Non-limiting examples of CD47 polymorphisms which may appear in polypeptides of the disclosure are listed in Table 4.

TABLE 4
CD47 polymorphisms

| | SEQ ID NO: 1 | | |
|---|---|---|---|
| | Position (including | | PDB 7 MYZ Position |
| Wild | peptide leader | | (Excluding peptide |
| type AA | sequence) | Mutant AA | leader sequence) |
| A | 6 | P | NA |
| G | 11 | C | NA |
| C | 15 | R | NA |
| A | 18 | T | NA |
| L | 20 | I | 2 |
| F | 22 | L | 4 |
| T | 25 | S | 7 |
| S | 27 | F | 9 |
| F | 30 | L | 12 |
| T | 31 | A | 13 |
| T | 31 | M | 13 |
| F | 32 | Y | 14 |
| C | 33 | Y | 15 |
| T | 36 | S | 18 |
| V | 38 | L | 20 |
| V | 38 | F | 20 |
| V | 38 | I | 20 |
| F | 42 | V | 24 |
| T | 44 | A | 26 |
| M | 46 | T | 28 |
| N | 50 | S | 32 |
| T | 51 | A | 33 |
| T | 52 | A | 34 |
| T | 52 | S | 34 |
| V | 56 | I | 38 |
| V | 56 | A | 38 |
| R | 63 | K | 45 |
| A | 71 | T | 53 |
| N | 73 | T | 55 |
| S | 75 | Y | 57 |
| T | 76 | A | 58 |
| P | 78 | L | 60 |
| P | 78 | A | 60 |
| F | 81 | S | 63 |
| S | 82 | N | 64 |
| S | 82 | R | 64 |
| K | 85 | N | 67 |
| V | 88 | I | 70 |
| V | 88 | A | 70 |
| S | 89 | A | 71 |
| Q | 90 | R | 72 |
| L | 91 | F | 73 |
| L | 92 | Q | 74 |
| K | 93 | N | 75 |
| S | 97 | T | 79 |
| M | 100 | V | 82 |
| M | 100 | I | 82 |
| D | 101 | G | 83 |
| D | 101 | V | 83 |
| D | 101 | Y | 83 |
| K | 102 | R | 84 |
| K | 102 | T | 84 |
| D | 104 | G | 86 |
| D | 104 | N | 86 |
| A | 105 | T | 87 |
| S | 107 | L | 89 |
| E | 115 | K | 97 |
| T | 125 | M | 107 |
| I | 126 | F | 108 |
| I | 127 | T | 109 |
| E | 128 | K | 110 |
| K | 130 | Q | 112 |
| R | 132 | C | 114 |
| R | 132 | H | 114 |
| S | 135 | A | 117 |
| S | 138 | F | 120 |
| E | 141 | V | 123 |
| I | 143 | V | 125 |
| I | 145 | T | 127 |
| V | 146 | I | 128 |
| V | 146 | F | 128 |
| P | 149 | S | 131 |
| P | 149 | T | 131 |
| I | 150 | V | 132 |
| A | 152 | V | 134 |
| I | 153 | V | 135 |
| L | 154 | F | 136 |
| L | 155 | Q | 137 |
| W | 157 | R | 139 |
| G | 161 | D | 143 |
| Y | 167 | C | 149 |
| S | 169 | A | 151 |
| G | 170 | R | 152 |
| G | 170 | S | 152 |
| D | 173 | Y | 155 |
| I | 177 | V | 159 |
| A | 178 | G | 160 |
| V | 181 | I | 163 |
| I | 186 | V | 168 |
| V | 188 | A | 170 |
| I | 189 | T | 171 |
| V | 190 | A | 172 |
| I | 191 | V | 173 |
| I | 191 | M | 173 |
| A | 194 | T | 176 |
| A | 194 | V | 176 |
| F | 197 | S | 179 |
| V | 198 | I | 180 |
| A | 207 | T | 189 |
| V | 214 | G | 196 |
| T | 215 | I | 197 |
| I | 219 | M | 201 |
| L | 220 | F | 202 |
| I | 221 | M | 203 |
| H | 224 | N | 206 |
| H | 224 | Q | 206 |
| Y | 225 | H | 207 |
| F | 228 | Y | 210 |
| A | 231 | E | 213 |
| I | 232 | T | 214 |
| T | 235 | A | 217 |
| S | 236 | F | 218 |
| F | 237 | L | 219 |
| V | 238 | I | 220 |
| I | 239 | N | 221 |
| I | 239 | F | 221 |
| A | 240 | T | 222 |
| I | 241 | V | 223 |
| I | 241 | M | 223 |
| V | 243 | I | 225 |
| I | 244 | T | 226 |
| Y | 249 | F | 231 |
| Y | 249 | S | 231 |
| L | 251 | V | 233 |
| A | 252 | S | 234 |
| V | 254 | A | 236 |
| C | 259 | Y | 241 |
| A | 261 | V | 243 |
| A | 262 | V | 244 |
| I | 264 | L | 246 |
| I | 264 | M | 246 |
| M | 266 | T | 248 |
| M | 266 | V | 248 |
| M | 266 | I | 248 |
| H | 267 | R | 249 |
| G | 268 | A | 250 |
| P | 269 | A | 251 |
| G | 274 | S | 256 |
| S | 276 | N | 258 |
| I | 277 | V | 259 |
| A | 279 | T | 261 |
| L | 280 | V | 262 |
| A | 281 | T | 263 |
| V | 287 | I | 269 |
| Y | 288 | C | 270 |
| K | 290 | T | 272 |
| V | 292 | A | 274 |
| V | 292 | G | 274 |
| N | 295 | S | 277 |
| N | 295 | D | 277 |
| P | 302 | S | 284 |
| R | 303 | G | 285 |
| A | 305 | T | 287 |
| F | 313 | V | 295 |
| G | 318 | A | 300 |
| M | 319 | V | 301 |
| M | 320 | T | 302 |
| M | 320 | I | 302 |
| M | 320 | V | 302 |
| N | 321 | K | 303 |
| D | 322 | V | 304 |
| D | 322 | N | 304 |
| E | 323 | G | 305 |

In some cases, a SIRPα binding polypeptide comprises at least one polymorphism listed in Table 4. In some cases, the SIRPα binding polypeptide comprises any 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, or 132 polymorphisms listed in Table 4. In some cases, the SIRPα binding polypeptide comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, or 132 polymorphisms listed in Table 4. In some cases, the SIRPα binding polypeptide comprises at most 132, 131, 130, 129, 128, 127, 126, 125, 124, 123, 122, 121, 120, 119, 118, 117, 116, 115, 114, 113, 112, 111, 110, 109, 108, 107, 106, 105, 104, 103, 102, 101, 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 polymorphism listed in Table 4.

In some cases, a SIRPα binding polypeptide comprises a sequence at least about 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, or 100% identical to any one of SEQ ID NOs: 476-633 and 648. In some cases, a SIRPα binding polypeptide comprises a sequence at least about 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, or 100% identical to SEQ ID NO: 648.

### Rational Mutations

In some embodiments, a polypeptide (e.g., engineered SIRPα binding polypeptide) as described herein comprises one or more mutations that have been developed or determined based on knowledge of the peptide functionality (e.g., relative to a reference polypeptide comprising a sequence of any one of SEQ ID NOs: 1-4). In some embodiments, determination of the potentially useful mutations may use tools and data that may include bioinformatic models, protein structure data, crystallographic information, protein functional data, and protein conformation models. Analysis may include algorithmic calculations, expert determinations, or both.

In some embodiments, a polypeptide comprises one or more mutations configured to generate a new covalent or non-covalent interaction or to abrogate an existing covalent or non-covalent interaction. In an example, a SIRPα binding sequence comprises a D35R mutation (e.g., relative to any one of SEQ ID NOs: 1-4) and an A262E mutation. The arginine residue may form an inter-domain ionic interaction with the glutamate and thus alter the conformational ensemble and function of the polypeptide relative to a wild-type CD47. In another example, a polypeptide comprises a S138F mutation configured to interact with a T31M cancer mutation. The bulky aromatic sidechain of the phenylalanine residue was selected to compensate for the loss of steric bulk near the methionine and further restrict movement between the TMD and ECD. As a result, the mutated polypeptide can display an altered conformational ensemble relative to the wild type sequence.

In some embodiments, the SIRPα binding polypeptide comprises at least one residue of: Y31, A32, R35, K35, P71, A77, A79, N80, L100, K138, L164, M185, A211, S259, E262, and any combination thereof, wherein the mutation is relative to any one of SEQ ID NOs: 1-4. In some embodiments, the SIRPα binding polypeptide comprises a Y31 residue. In some embodiments, the SIRPα binding polypeptide comprises a A32 residue. In some embodiments, the SIRPα binding polypeptide comprises a R35 residue. In some embodiments, the SIRPα binding polypeptide comprises a K35 residue. In some embodiments, the SIRPα binding polypeptide comprises a P71 residue. In some embodiments, the SIRPα binding polypeptide comprises a A77 residue. In some embodiments, the SIRPα binding polypeptide comprises a A79 residue. In some embodiments, the SIRPα binding polypeptide comprises a N80 residue. In some embodiments, the SIRPα binding polypeptide comprises a L100 residue. In some embodiments, the SIRPα binding polypeptide comprises a K138 residue. In some embodiments, the SIRPα binding polypeptide comprises a L164 residue. In some embodiments, the SIRPα binding polypeptide comprises a M185 residue. In some embodiments, the SIRPα binding polypeptide comprises a A211 residue. In some embodiments, the SIRPα binding polypeptide comprises a S259 residue. In some embodiments, the SIRPα binding polypeptide comprises a E262 residue.

In some embodiments, a SIRPα binding polypeptide comprises a sequence at least about 80%, 81%, 82%, 83%, 84%, 85%, 86%, 87%, 88%, 89%, 90%, 91%, 92%, 93%, 94%, 95%, 96%, 97%, 98%, or 99%, or 100% identical to any one of SEQ ID NOs: 634-638 and 648-653. In some embodiments, a SIRPα binding polypeptide comprises a sequence at least about 80%, 81%, 82%, 83%, 84%, 85%, 86%, 87%, 88%, 89%, 90%, 91%, 92%, 93%, 94%, 95%, 96%, 97%, 98%, or 99%, or 100% identical to SEQ ID NO: 634. In some embodiments, a SIRPα binding polypeptide comprises a sequence at least about 80%, 81%, 82%, 83%, 84%, 85%, 86%, 87%, 88%, 89%, 90%, 91%, 92%, 93%, 94%, 95%, 96%, 97%, 98%, or 99%, or 100% identical to SEQ ID NO: 635. In some embodiments, a SIRPα binding polypeptide comprises a sequence at least about 80%, 81%, 82%, 83%, 84%, 85%, 86%, 87%, 88%, 89%, 90%, 91%, 92%, 93%, 94%, 95%, 96%, 97%, 98%, or 99%, or 100% identical to SEQ ID NO: 636. In some embodiments, a SIRPα binding polypeptide comprises a sequence at least about 80%, 81%, 82%, 83%, 84%, 85%, 86%, 87%, 88%, 89%, 90%, 91%, 92%, 93%, 94%, 95%, 96%, 97%, 98%, or 99%, or 100% identical to SEQ ID NO: 637. In some embodiments, a SIRPα binding polypeptide comprises a sequence at least about 80%, 81%, 82%, 83%, 84%, 85%, 86%, 87%, 88%, 89%, 90%, 91%, 92%, 93%, 94%, 95%, 96%, 97%, 98%, or 99%, or 100% identical to SEQ ID NO: 638. In some embodiments, a SIRPα binding polypeptide comprises a sequence at least about 80%, 81%, 82%, 83%, 84%, 85%, 86%, 87%, 88%, 89%, 90%, 91%, 92%, 93%, 94%, 95%, 96%, 97%, 98%, or 99%, or 100% identical to SEQ ID NO: 648. In some embodiments, a SIRPα binding polypeptide comprises a sequence at least about 80%, 81%, 82%, 83%, 84%, 85%, 86%, 87%, 88%, 89%, 90%, 91%, 92%, 93%, 94%, 95%, 96%, 97%, 98%, or 99%, or 100% identical to SEQ ID NO: 649. In some embodiments, a SIRPα binding polypeptide comprises a sequence at least about 80%, 81%, 82%, 83%, 84%, 85%, 86%, 87%, 88%, 89%, 90%, 91%, 92%, 93%, 94%, 95%, 96%, 97%, 98%, or 99%, or 100% identical to SEQ ID NO: 650. In some embodiments, a SIRPα binding polypeptide comprises a sequence at least about 80%, 81%, 82%, 83%, 84%, 85%, 86%, 87%, 88%, 89%, 90%, 91%, 92%, 93%, 94%, 95%, 96%, 97%, 98%, or 99%, or 100% identical to SEQ ID NO: 651. In some embodiments, a SIRPα binding polypeptide comprises a sequence at least about 80%, 81%, 82%, 83%, 84%, 85%, 86%, 87%, 88%, 89%, 90%, 91%, 92%, 93%, 94%, 95%, 96%, 97%, 98%, or 99%, or 100% identical to SEQ ID NO: 652. In some embodiments, a SIRPα binding polypeptide comprises a sequence at least about 80%, 81%, 82%, 83%, 84%, 85%, 86%, 87%, 88%, 89%, 90%, 91%, 92%, 93%, 94%, 95%, 96%, 97%, 98%, or 99%, or 100% identical to SEQ ID NO: 653.

### Chimeras

In some embodiments, a polypeptide (e.g., engineered SIRPα binding polypeptide) as described herein comprises one or more regions, or portions thereof, derived from non-human proteins (e.g., relative to a reference polypeptide comprising a sequence of any one of SEQ ID NOs: 1-4). In some embodiments, the region is an extracellular domain (ECD). In some embodiments, the region is a transmembrane domain (TMD), or portion thereof. In some embodiments, the region is an extracellular loop region (ECLD). In some embodiments, the region is a C-terminal intracellular domain. In some embodiments, the region comprises any two of the ECD, TMD, ECLR, or C-terminal intracellular domain, or potion thereof. In some embodiments, the region comprises any three of the ECD, TMD, ECLR, or C-terminal intracellular domain, or portion thereof. In some embodiments, the region comprises all of the ECD, TMD, ECLR, or C-terminal domain, or portion thereof.

In some embodiments, to avoid signaling transduction of certain (e.g., non-SIRPα) binding partners upon binding, engineered SIRPα binding polypeptides may comprise hybrid molecules with detuned human function. In some embodiments, the TMD and C-terminal domains may be based on a domain from a non-human species. In some embodiments, the ECD domains may contain specific residues that may have been replaced with a residue from a different species. In some embodiments, the TMD and C-terminal domains may contain specific residues that may have been replaced with a residue from a different species. A polypeptide of such a composition may be prevented from binding to human TSP-1 and integrins and abrogate transmembrane signaling. In some embodiments, chimeras may also contain a modified TMD domain.

In some embodiments, the non-human sequence may be derived from any other non-human that expresses CD47. Representative, non-limiting species from which sequences may be derived include Gallus gallus, Alligator mississippiensis, Accipiter gentilis, Anas platyrhynchos, Ficedula albicollis, Phasianus colchicus, and Chloebia gouldiae (Erythrura gouldiae). In some embodiments, the non-human sequence may comprise a sequence that has at least about 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or more identity to any one of SEQ ID NOs: 641-647. In some embodiments, a polypeptide as described herein (e.g., hybrid SIRPα binding polypeptide) comprises a sequence that has at least about 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or more identity to SEQ ID NO: 648.

### Loop Insertions

In some embodiments, a polypeptide (e.g., engineered SIRPα binding polypeptide) as described herein comprises a modified peptide sequence such that a peptide linker is inserted into a loop or linker (e.g. the RVVSWF linker comprised in residues 132-137 of any one of SEQ ID NOs: 1-4) of the polypeptide structure (e.g., relative to a reference polypeptide comprising a sequence of any one of SEQ ID NOs: 1-4). In some embodiments, the loop is a polypeptide sequence between the ECD domain and the TMD domain of the polypeptide. In some embodiments, the loop comprises residues 132-137 of any one of SEQ ID NOs: 1-4. The loop insertion may be inserted between any two residues in the range of 132-137, prior to residue 132, or subsequent to residue 137, relative to a reference polypeptide sequence of any one of SEQ ID NOs: 1-4. In some embodiments, the linker is added between the ECD domain of the reference peptide with a IgV-like protein fold and the first helix of the TMD comprised by a protein fold containing 5 transmembrane helices. In some embodiments, the linker sequence may be between 3, 4, 5, 6, 7, 8, 9, 10, 11, 12, 13, 14, 15, 16, or more residues in length. The linker may comprise a pair of cysteine residues configured to form a disulfide bond. The pair of cysteine residues may be separated by any suitable number of residues. In some embodiments, the pair of cysteine residues are separated by 5, 6, 7, 8, 9, 10, 11, 12, 13, 14, 15, 16, 17, 18, 19, 20, or more residues. In some embodiments, the pair of cysteine residues are separated by at least 5, 6, 7, 8, 9, 10, 11, 12, 13, 14, 15, 16, 17, 18, 19, 20, or more residues. In some embodiments, the pair of cysteine residues are separated by no more than 20, 19, 18, 17, 16, 15, 14, 13, 12, 11, 10, 9, 8, 7, 6, 5, or fewer residues.

In some embodiments, a polypeptide as described herein comprises a sequence that has at least about 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or more identity to SEQ ID NO: 640. In some embodiments, a polypeptide as described herein comprises a sequence that has at least about 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or more identity to SEQ ID NO: 639.

In some embodiments, a polypeptide as described herein (e.g., synthetic SIRPα binding polypeptide) comprises a sequence that has at least about 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or more identity to any one of SEQ ID NOs: 10-384. In some embodiments, the percent identity is between any two of these values. In some embodiments, the polypeptide has no more than about 99%, 98%, 97%, 95%, 94%, 93%, 92%, 91%, 90%, 89%, 88%, 87%, 86%, 85%, 84%, 83%, 82%, 81%, 80%, 70%, 60%, 50%, 40%, 30%, 20%, or less identity to any one of the sequences in SEQ IS NOs: 10-384. In some embodiments, the percent identity is between any two of these values.

In some embodiments, a polypeptide as described herein (e.g., synthetic SIRPα binding polypeptide) comprises a sequence that has at least about 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or more identity to any one of SEQ ID NOs: 47, 50, 81, 108, 141, 215, 286, 377, 381, or 638. In some embodiments, the percent identity is between any two of these values. In some embodiments, the polypeptide has no more than about 99%, 98%, 97%, 95%, 94%, 93%, 92%, 91%, 90%, 89%, 88%, 87%, 86%, 85%, 84%, 83%, 82%, 81%, 80%, 70%, 60%, 50%, 40%, 30%, 20%, or less identity to any one of the sequences in SEQ ID NOs: 47, 50, 81, 108, 141, 215, 286, 377, 381, or 638. In some embodiments, the percent identity is between any two of these values.

### Conformational Ensembles of SIRPα Binding Polypeptide Sequences

In some embodiments, a polypeptide as described herein (e.g., synthetic SIRPα binding polypeptide) comprises a conformational ensemble which has been modulated relative to a conformational ensemble of a reference polypeptide. In some cases, the reference polypeptide is a WT CD47 sequence (e.g., any one of SEQ ID NOs: 1-4).

Conformational ensembles of polypeptides as described herein (e.g., engineered SIRPα binding polypeptide, reference polypeptide) can be determined by any suitable operation. In some cases, a conformational ensemble may be observed or predicted by techniques of biophysics and molecular simulation such as x-ray crystallography (XRC), nuclear magnetic resonance (NMR), hydrogen-deuterium exchange (HDX), small-angle e-ray scattering (SAXS), neutron diffraction, electron paramagnetic resonance (EPR), cryogenic electron microscopy (cyroEM), molecular dynamics (MD), Monte Carlo (MC) methods, artificial intelligence and machine learning algorithms, and any combination thereof.

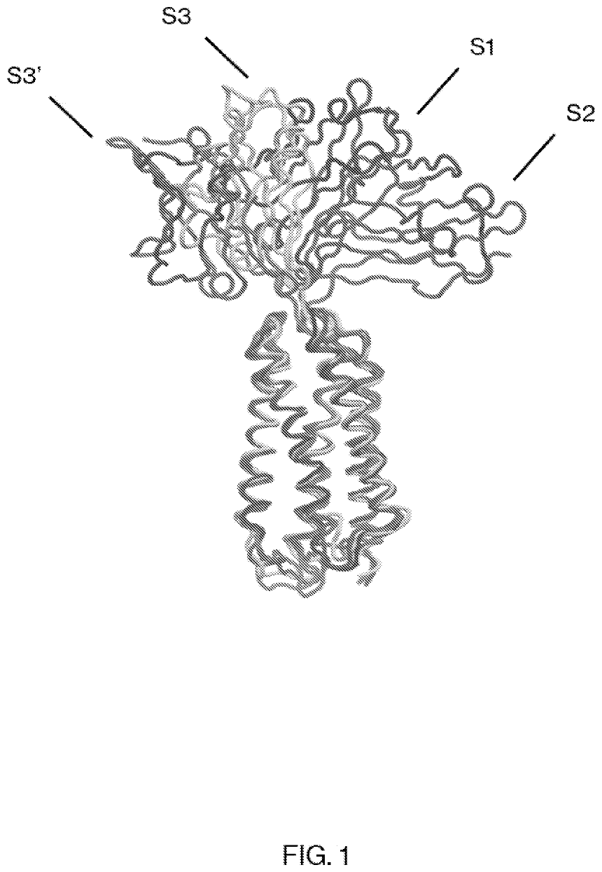

As described hereinbelow, computational analysis of molecular dynamics (MD) simulations of WT CD47 resulted in the identification of four primary metastable states (“metastates”) that characterize the vast majority of the conformational ensemble of CD47. A superposition of these metastates is illustrated in FIG. 1. Of these, the S1 and S2 metastates were found to comprise a majority of the conformational ensemble and the primary motion of the molecule at equilibrium was a transition between these two metastates.

The S1 and S2 metastates (as well as other metastates of polypeptides disclosed herein) may be characterized in terms of a reaction coordinate. In some cases, the reaction coordinate comprises an atomic coordinate, set of atomic coordinates, order parameter, or a combination or transformation thereof. In general, any atomic coordinate, set of atomic coordinates, order parameters, or transformations thereof may be used as a reaction coordinate if the coordinate, set of coordinates, order parameter, or transformation thereof adopts differing values that readily distinguish the metastable states from one another. In an example, an interatomic distance between two atoms, one on the ECD and one on the TMD, of a SIRPα binding protein as described herein can be used as a reaction coordinate to identify the metastate (or potential set of metastates) to which a particular conformation of the SIRPα binding protein may be assigned. In an example, interatomic distance between the CQ atoms of residues R132 and E151 is used as a reaction coordinate. In another example, a bending angle between an ECD and TMD can be used as a reaction coordinate to identify the metastate (or potential set of metastates) to which a particular conformation of the SIRPα binding protein may be assigned. The angle between the domains for a given configuration can be determined as the angle of the vector between an x-y plane set at an origin (e.g., center of mass of one domain) and a vector between the origin and the center of mass of a subset of residues of the other domain. In a particular example, the first domain was the TMD and the second vector was the vector between the origin and the center of mass of residues N50 through V54 of an ECD.

Without wishing to be bound by a particular theory, certain SIRPα agonists as described herein may elicit a reduced immune response when expressed on the surface of a cell due to comprising a conformational ensemble which comprises a different proportion of these metastable states as compared to a reference polypeptide (e.g., comprising a sequence of any one of SEQ ID NOs; 1-4). In some embodiments, a SIRPα agonist of the present disclosure comprises a conformational ensemble with a greater proportion of the “S1” metastate as discussed above. In some embodiments, a cell product would contain one or more SIRPα agonist of the present disclosure containing different distributions of conformational ensembles (e.g. some may display greater S1 proportion, some may display greater S2 proportion). Various strategies as discussed herein (cancer mutations, polymorphisms, oligomerization, chimera, loop insertions, rational mutations) may be employed singly or in combination to engineer SIRPα agonist polypeptide sequences with a conformational ensembles that comprise a larger proportion of the S1 metastate and thus enhance SIRPα signaling by having a larger proportion of the molecule(s) available to interact with SIRPα. Alternatively, (e.g., engineered) SIRPα binding polypeptides of the disclosure may enhance signaling through SIRPα by a mechanism other than increasing the proportion of the S1 metatstate in their conformational ensemble (e.g., relative to WT CD47), such as by reducing affinity for other endogenous binding partners and thus enhancing the amount of “free” SIRPα binding molecules or sites available.

### N-Terminal Modifications of Engineered Selective SIRPα Binding Peptides

In some embodiments, the engineered SIRPα binding peptides described herein further comprise N-terminal modifications that further enhance binding to SIRPα. In some embodiments, these N-terminal modifications are made in addition to any one of the modifications (e.g., sequence modifications, such as mutations or computational design strategies) described herein.

In some embodiments, the engineered SIRPα binding peptide comprises at least 1 amino acid added to the N-terminus of the mature protein. In some embodiments, the engineered SIRPα binding peptide comprises at least 2 amino acids added to the N-terminus of the mature protein. In some embodiments the engineered SIRPα binding peptide comprises at least 3 amino acids added to the N-terminus of the mature protein. In some embodiments, the 3 amino acids added have the formula X-3X-2X-1, where X-3 is W; X-2 is selected from Q, A and G; and X-1 is selected from R, P, L, T, F, I, and M. In some embodiments, the three amino acids added are selected from: WQR, WAP, WQL, WQP, WQT, WQF, WQI, WGP, and WQM.

### Nucleic Acid Molecules

Also provided herein are nucleic acids that encode any of the peptides, polypeptides, fusion proteins, and compositions described herein.

In some embodiments, nucleic acid molecules may include polymeric forms of nucleotides of any length, either ribonucleotides or deoxyribonucleotides. In some embodiments, nucleic acid molecules may only to the primary structure of the molecule. In some embodiments, nucleic acid molecules may be triple-, double- and single-stranded DNA, as well as triple-, double- and single-stranded RNA. In some embodiments, nucleic acid molecules may be modified by methylation and/or by capping. In some embodiments, nucleic acid molecules may be natural, synthetic, or a combination of both.

### Methods of Generating Hypoimmunogenic Cells

Also provided herein are methods of making any of the peptides, polypeptides, fusion proteins, and compositions of the disclosure. Such method may comprise introducing into a cell a nucleic acid sequence encoding the peptide to produce a recombinant cell; and culturing the recombinant cell under conditions sufficient for the expression of the peptide. In some embodiments, the introducing step includes introducing into a cell an expression vector including a nucleic acid sequence encoding the peptide. In some embodiments,

Also provided herein are vectors that include any of the nucleic acids provided herein. The vector may refer to a polynucleotide capable of inducing the expression of a recombinant peptide in a host cell. In some embodiments, the vector further comprises a promoter and/or enhancer operably linked to any of the nucleic acids described herein. In some embodiments,

In certain embodiments, vectors could be constructed to comprise exogenous nucleic acid sequences for genetic modification of any cells used herein, particularly the starting cells, such as stroma cells or stem or progenitor cells in the culturing methods or compositions. One of skill in the art would be well equipped to construct a vector through standard recombinant techniques.

Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide.

Such components also might include markers, such as detectable and/or selection markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. A large variety of such vectors are known in the art and are generally available. When a vector is maintained in a host cell, the vector can either be stably replicated by the cells during mitosis as an autonomous structure, incorporated within the genome of the host cell, or maintained in the host cell's nucleus or cytoplasm.

Genetic modification or introduction of exogenous nucleic acids into starting cells of the culturing composition or methods may use any suitable methods for nucleic acid delivery for transformation of a cell, as described herein or as would be known to one of ordinary skill in the art. Such methods include, but are not limited to, direct delivery of DNA such as by ex vivo transfection, by injection, including microinjection; by calcium phosphate precipitation; by using DEAE-dextran followed by polyethylene glycol; by direct sonic loading; by liposome mediated transfection and receptor-mediated transfection; by microprojectile bombardment; by agitation with silicon carbide fibers; by Agrobacterium-mediated transformation; by PEG-mediated transformation of protoplasts; by desiccation/inhibition-mediated DNA uptake, and any combination of such methods. Through the application of techniques such as these, organelle(s), cell(s), tissue(s) or organism(s) may be stably or transiently transformed.

The cells in certain embodiments can be made to contain one or more genetic alterations by genetic engineering of the cells either before or after differentiation. A cell is said to be “genetically altered”, “genetically modified” or “transgenic” when an exogenous nucleic acid or polynucleotide has been transferred into the cell by any suitable means of artificial manipulation, or where the cell is a progeny of the originally altered cell that has inherited the polynucleotide. For example, the cells can be processed to increase their replication potential by genetically altering the cells to express telomerase reverse transcriptase, either before or after they progress to restricted developmental lineage cells or terminally differentiated cells.

In certain embodiments, cells containing an exogenous nucleic acid construct may be identified in vitro or in vivo by including a marker in the expression vector, such as a selectable or screenable marker. Such markers would confer an identifiable change to the cell permitting easy identification of cells containing the expression vector, or help enrich or identify differentiated cells by using a tissue-specific promoter. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Cells of the present disclosure may comprise one or a plurality of SIRPα binding polypeptide as described herein. In some embodiments, the plurality of SIRPα binding polypeptides comprises a wild type CD47 (e.g., comprising any one of the sequences of SEQ ID NOs: 1-4). In some embodiments, the plurality of SIRPα binding polypeptides does not comprise a wildtype CD47 sequence. In some embodiments, the plurality of SIRPα binding polypeptides comprises a plurality of engineered SIRPα binding polypeptides. The plurality of engineered SIRPα binding polypeptides may comprise any engineered SIRPα binding polypeptides disclosed herein.

### Computer Systems

The present disclosure provides computer systems that are programmed to implement methods of the disclosure. FIG. 24 shows a computer system 2401 that is programmed or otherwise configured to design the SIRPα agonists described herein (e.g. any one of the methods described herein). The computer system 2401 can regulate various aspects of sequence design of the present SIRPα agonists, such as, for example, stability and an enhanced “don't eat me” signal. The computer system 2401 can be an electronic device of a user or a computer system that is remotely located with respect to the electronic device. The electronic device can be a mobile electronic device.

The computer system 2401 includes a central processing unit (CPU, also “processor” and “computer processor” herein) 2405, which can be a single core or multi core processor, or a plurality of processors for parallel processing. The computer system 2401 also includes memory or memory location 2410 (e.g., random-access memory, read-only memory, flash memory), electronic storage unit 2415 (e.g., hard disk), communication interface 2420 (e.g., network adapter) for communicating with one or more other systems, and peripheral devices 2425, such as cache, other memory, data storage and/or electronic display adapters. The memory 2410, storage unit 2415, interface 2420 and peripheral devices 2425 are in communication with the CPU 2405 through a communication bus (solid lines), such as a motherboard. The storage unit 2415 can be a data storage unit (or data repository) for storing data. The computer system 2401 can be operatively coupled to a computer network (“network”) 2430 with the aid of the communication interface 2420. The network 2430 can be the Internet, an internet and/or extranet, or an intranet and/or extranet that is in communication with the Internet. The network 2430 in some cases is a telecommunication and/or data network. The network 2430 can include one or more computer servers, which can enable distributed computing, such as cloud computing. The network 2430, in some cases with the aid of the computer system 2401, can implement a peer-to-peer network, which may enable devices coupled to the computer system 2401 to behave as a client or a server.

The CPU 2405 can execute a sequence of machine-readable instructions, which can be embodied in a program or software. The instructions may be stored in a memory location, such as the memory 2410. The instructions can be directed to the CPU 2405, which can subsequently program or otherwise configure the CPU 2405 to implement methods of the present disclosure. Examples of operations performed by the CPU 2405 can include fetch, decode, execute, and writeback.

The CPU 2405 can be part of a circuit, such as an integrated circuit. One or more other components of the system 2401 can be included in the circuit. In some cases, the circuit is an application specific integrated circuit (ASIC).

The storage unit 2415 can store files, such as drivers, libraries and saved programs. The storage unit 2415 can store user data, e.g., user preferences and user programs. The computer system 2401 in some cases can include one or more additional data storage units that are external to the computer system 2401, such as located on a remote server that is in communication with the computer system 2401 through an intranet or the Internet.

The computer system 2401 can communicate with one or more remote computer systems through the network 2430. For instance, the computer system 2401 can communicate with a remote computer system of a user. Examples of remote computer systems include personal computers (e.g., portable PC), slate or tablet PC's (e.g., Apple®iPad, Samsung® Galaxy Tab), telephones, Smart phones (e.g., Apple® iPhone, Android-enabled device, Blackberry®), or personal digital assistants. The user can access the computer system 2401 via the network 2430.

Methods as described herein can be implemented by way of machine (e.g., computer processor) executable code stored on an electronic storage location of the computer system 2401, such as, for example, on the memory 2410 or electronic storage unit 2415. The machine executable or machine readable code can be provided in the form of software. During use, the code can be executed by the processor 2405. In some cases, the code can be retrieved from the storage unit 2415 and stored on the memory 2410 for ready access by the processor 2405. In some situations, the electronic storage unit 2415 can be precluded, and machine-executable instructions are stored on memory 2410.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 2401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 2401 can include or be in communication with an electronic display 2435 that comprises a user interface (UI) 2440 for providing, for example, SIRPα agonist sequences described herein. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 2405. The algorithm can, for example, design the sequences for the SIRPα agonists described herein.

### Examples

The following examples are provided to further illustrate some embodiments of the present disclosure but are not intended to limit the scope of the disclosure; it will be understood by their exemplary nature that other procedures, methodologies, or techniques known to those skilled in the art may alternatively be used.

### Example 1. Molecular Dynamics Simulation of SIRPα Binding Polypeptides

To investigate the thermodynamics and kinetics of signal regulatory protein alpha (SIRPα) binding to cluster of differentiation 47 (CD47), molecular dynamics (MD) simulations of the wild type (WT) CD47 alone and in complex with SIRPα were performed and analyzed. MD simulations were performed generally in accordance with the protocols of Fenalti, G; et al. Nat. Commun. 2021, 12, 5218. (doi.org/10.1038/s41467-021-25475-w; also referred to herein as “Fenalti”), which is incorporated herein by this reference in its entirety. Three independent simulations, respectively, were performed and concatenated. The concatenated trajectories were analyzed using principal component analysis (PCA) to identify the dominant metastable states present at equilibrium.

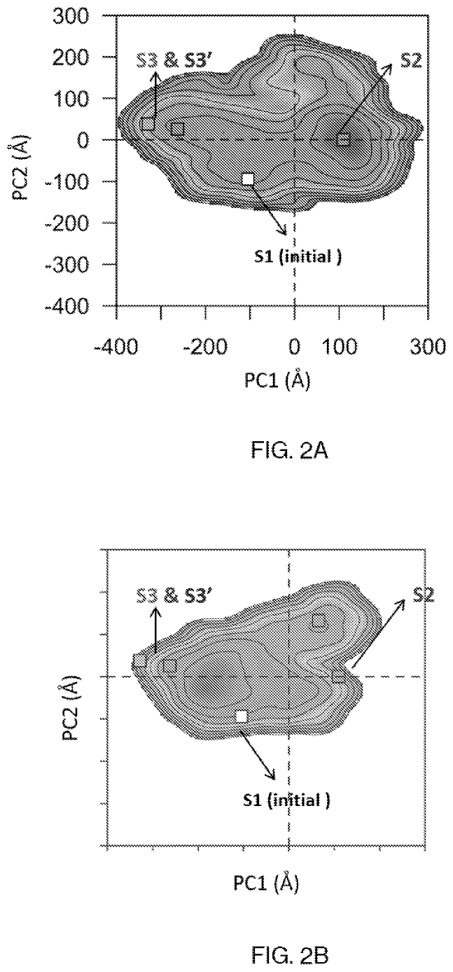

The equilibrium conformational ensemble of WT CD47 without SIRPα was found to comprise several dominant metastable states, referred to herein as S1, S2, S3, and S3′. Representative microstates (individual conformations) of each metastate are depicted in FIG. 1. FIG. 2A depicts a PCA plot showing the relative distribution of conformations along the first two principal components of WT CD47 without SIRPα. FIG. 2B depicts a PCA plot showing the relative distribution of conformations along the first two principal components for WT CD47 in complex with SIRPα. As indicated by the relative density (saturation) in each plot, binding of SIRPα shifts the conformational ensemble from predominantly the S2 metastate to predominantly the S1 metastate. Based on this observation, it was hypothesized that SIRPα preferentially binds the S1 metastate of CD47.

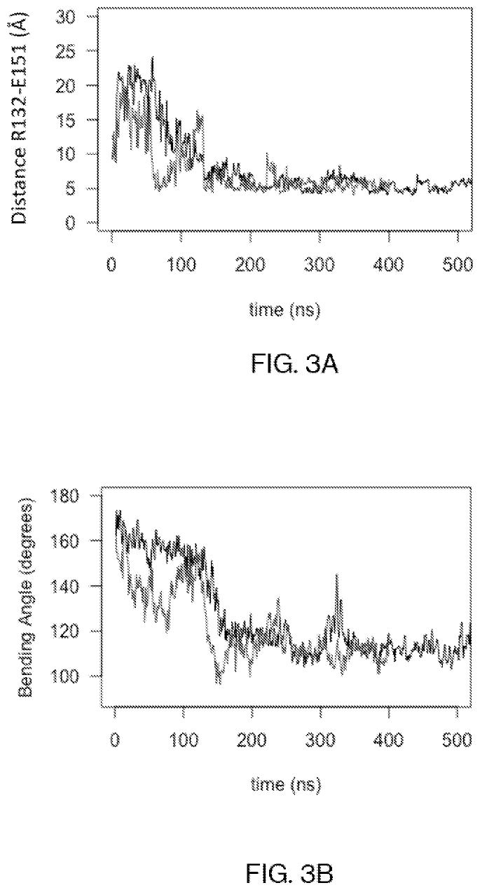

To further describe the S1 and S2 metastates, as well as the interconversion between them, several reaction coordinates were selected and investigated. A qualitative investigation of representative confirmations from the S1 and S2 metastates suggested that the states could be distinguished by the relative distance and angle between the extracellular domain (ECD) and transmembrane domain (TMD) of the molecule. Accordingly, each of these was selected as a potential reaction coordinate. The distance between the two domains was calculated by taking the distance between the C_{α} atoms of residues R132 and E141. This distance was found to range from about 4-9 Å in the S2 state to about 10-25 Å in the S1 state (FIG. 3A). The angle between the domains was also calculated and found to range from about 100-120 degrees in the S2 state to about 130-180 degrees in the S1 state (FIG. 3B). The angle between the domains was calculated by determining the angle of the vector between an x-y plane set at the origin (center of mass of the TMD bundle) and a vector between the origin and the center of mass of residues N50 through V54 in the ECD.

### Example 2: Investigation of mutations in CD47

Documented mutations found in human CD47 and associated with cancer samples (e.g., as comprised in Table 3), as well as other polymorphisms (e.g., as comprised in Table 4), were mapped on distinct regions of a crystal structure of the molecule in complex with an antibody (PDB ID: 7MYZ). Certain mutations were found to cluster on critical functional regions of the molecule, including the extracellular domain (ECD), transmembrane domain (TMD) and extracellular loop region (ECLR).

### Cancer Mutations Located in the Transmembrane Domain (TMD) of CD47

Some cancer mutations sites (e.g., as comprised in Table 3) were mapped to the TMD of CD47 (e.g., corresponding to any of positions 142-162, 177-197, 208-228, 236-256, or 269-289 of any of SEQ ID NOs: 1-4). These mutations were hypothesized to impact protein packing of the TMD, and thus transmembrane signaling, through modulation of noncovalent interactions including hydrophobic interactions (e.g., hydrophobic interactions in the core of the TMD, between the transmembrane helices, or hydrophobic interactions pointing out to the lipidic membrane environment), hydrogen bond interactions, and covalent (e.g., disulfide bonds, such as between cancer mutant W136C and C259).

### Cancer Mutations Located in the Extracellular Domain (ECD) of WT CD47

Some cancer mutation sites (e.g., as comprised in Table 3) were mapped to the ECD of CD47 (e.g., corresponding to any of positions 19-141 of any of SEQ ID NOs: 1-4). Computational analysis revealed that the ECD of CD47 comprises regions predicted to be regions of instability on CD47. Accordingly, it was hypothesized that cancer mutations in the ECD of CD47 may act to increase stability of the receptor and thus enhance signaling through the SIRPα pathway, affect distribution of metastable states, or affect interactions with the lipidic membrane environment.

### Other CD47 Polymorphisms

Analysis of the CD47 polymorphisms (e.g., as comprised in Table 4), showed these mutations are spread across the different domains of CD47 when mapped onto the CD47 crystal structure. It was hypothesized that this region maps to the dimer surface and to regions that bind other CD47 endogenous binders such as thrombospondin 1 (TSP-1), integrins or other endogenous protein partners.

### Example 3. Chimeric Polypeptides and Loop Insertions

SIRPα Agonist Peptide Insertions Identified from Analysis of the Receptor Evolution Across Multiple Species.

SIRPα agonist peptide insertions were identified in the RVVSWF peptide linker (positions 132-137 of any of SEQ ID NOs; 1-4) linking the ECD and the TMD of human CD47.

Several species were identified to have peptides that vary in amino acid length and are constrained by a disulfide bond (e.g., SEQ ID NOs: 641-647). These peptides are predicted to influence ECD domain dynamics and can be tested for function (e.g., protection from NK-cell cytotoxicity, protection from macrophage cytotoxicity) according to the procedures disclosed herein. They may also affect binding kinetics to other endogenous protein partners or affect oligomerization of SIRPα polypeptides.

### Example 4. Conformational Ensemble of a SIRPα Binding Polypeptide

A synthetic SIRPα binding polypeptide is generated in accordance with the operations described herein. The conformational ensemble of the polypeptide is characterized (e.g., at least in part by one or more of hydrogen-deuterium exchange (HDX), small angle x-ray scattering (SAXS), nuclear magnetic resonance (NMR), or molecular dynamics (MD)). The conformational ensemble of a reference polypeptide (e.g., comprising the sequence of any one of SEQ ID NOs: 1-4) is examined similarly.

In each case, the conformational ensemble is found to comprise a first metastable state which is configured to bind to SIRPα. The first metastable state comprises conformations characterized by one or more of the following features:
A bending angle between a transmembrane domain (TMD) and an extracellular domain (TMD) of about 130 to about 180 degrees, such as about 150 to about 170 degrees; and
A distance between the TMD and the ECD of from about 10 angstroms (Å) to about 25 Å, or more than about 25 Å.

A metastable state characterized by these features may be referred to as an “S1” or “S1-like” metastate herein.

The conformational ensemble of the synthetic SIRPα binding polypeptide is found to comprise a higher proportion (e.g., as determined, estimated, or predicted by one of more of HDX, SAXS, NMR, or MD) of the first metastable state than the conformational ensemble of the reference polypeptide.

The conformational ensemble of the reference polypeptide is also found to comprise a second metastable state which does not substantially bind SIRPα. The second metastable state is characterized by one or more of the following features:
A bending angle between a transmembrane domain (TMD) and an extracellular domain (TMD) of about 100 degrees to about 120 degrees, or less than 100 degrees; and
A distance between the TMD and the ECD of from about 4 angstroms (Å) to about 9 Å, or less than about 4 Å.

The conformational ensemble of the polypeptide may also be found to comprise the second metastable state but at a smaller proportion than the conformational ensemble of the reference polypeptide.

A metastable state characterized by these features may be referred to as an “S2” or “S2-like” metastate herein.

### Example 5: Enhanced S1 Sampling through Cancer Mutations and Polymorphisms

To test the hypothesis that CD47 mutations associated with cancer (e.g., as comprised in Table 3) and certain CD47 polymorphisms (e.g., as comprised in Table 4) enhance the SIRPα binding activity of CD47 by shifting the conformational ensemble of the molecule toward a higher proportion of the S1 metastate, constructs comprising some of these mutations were simulated using MD in accordance with the protocols outlined in Fenalti and the resulting conformational ensembles sampled and compared to wild type CD47 (e.g., SEQ ID NO: 1).

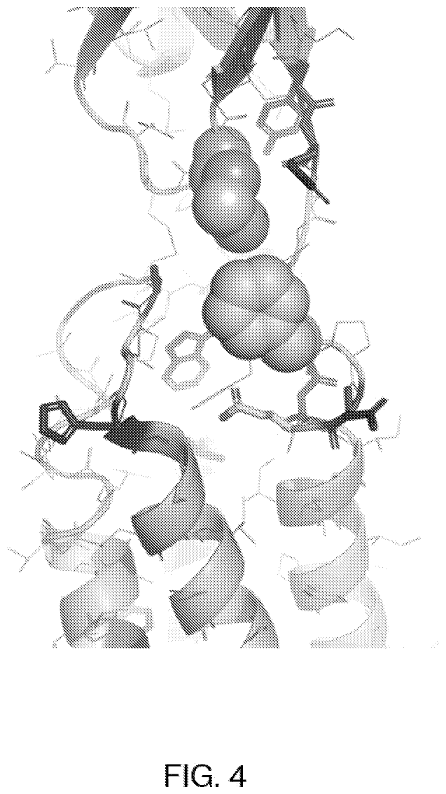

A sequence comprising an R132C mutation (SEQ ID NO: 419), which is associated with cancer samples, was simulated using MD. Residue 132 occurs in the ECLR of CD47, and thus the R132C mutant was hypothesized to reduce relative motion between the ECD and TMD, thus increasing the relative proportion of the S1 metastate in its conformational ensemble. Another sequence comprising T31M and S138F (SEQ ID NO: 648) was also simulated using MD. Residues 31 and 138 also occur in the ECLR. Accordingly, it was hypothesized that these two hydrophobic substitutions would fill the ECLR and restrict hinge motion between the ECD and TMD, thus increasing the relative proportion of the S1 metastate. A representative model of the T31M/S138F mutant showing the mutated residues in the ECLR is depicted in FIG. 4.

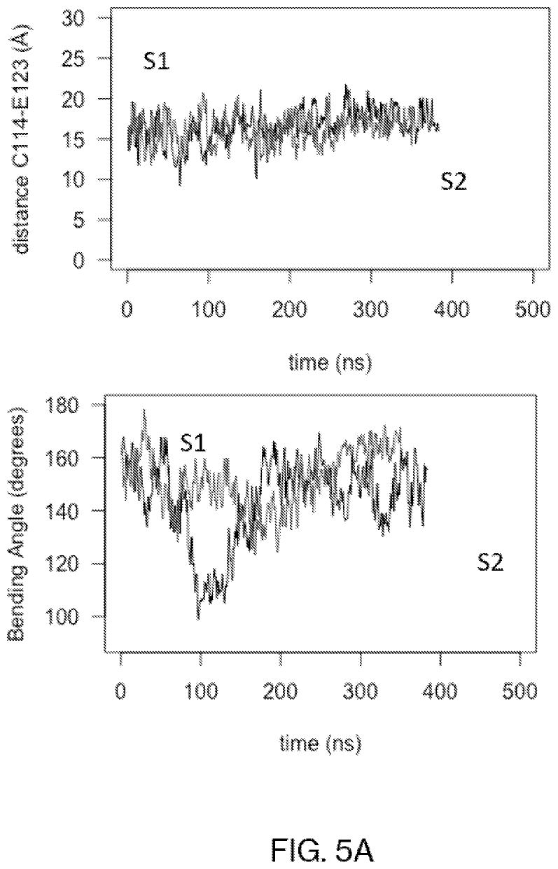

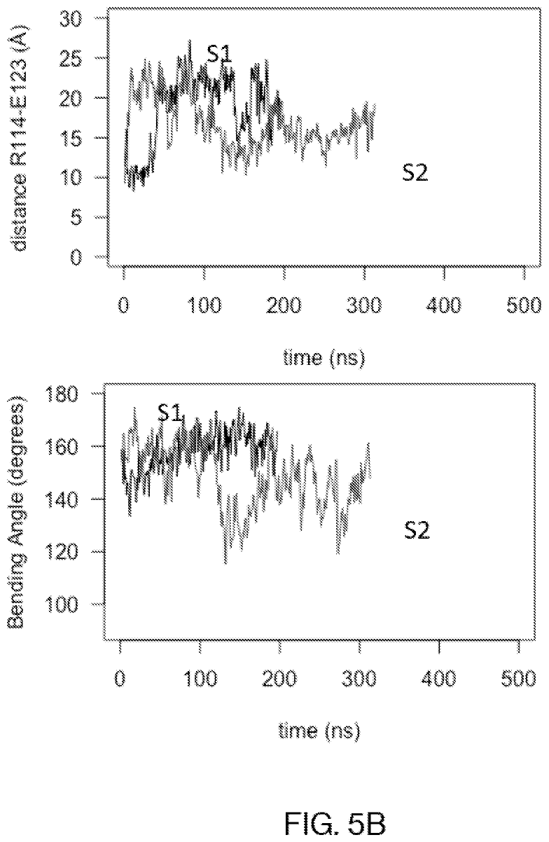

Results of two separate MD for each construct are shown in FIG. 5A (R132C) and FIG. 5B (T31M, S138F). These Figures depict the distance between the ECD and TMD domains (top panels; calculated as described in Example 2) and the angle of the domains (bottom panels; calculated as described in Example 2). As shown in FIGS. 5A and 5B, these mutation in each case increase the proportion of time the simulated molecule (the conformational ensemble) spends in the S1 metastate as compared to WT CD47 (FIGS. 3A and 3B).

### Example 6. Macrophage Assay

This example outlines a macrophage killing assay to measure protection from macrophage cytotoxicity conferred by the engineered SIRPα binding polypeptides of the present disclosure.

### Construct Generation

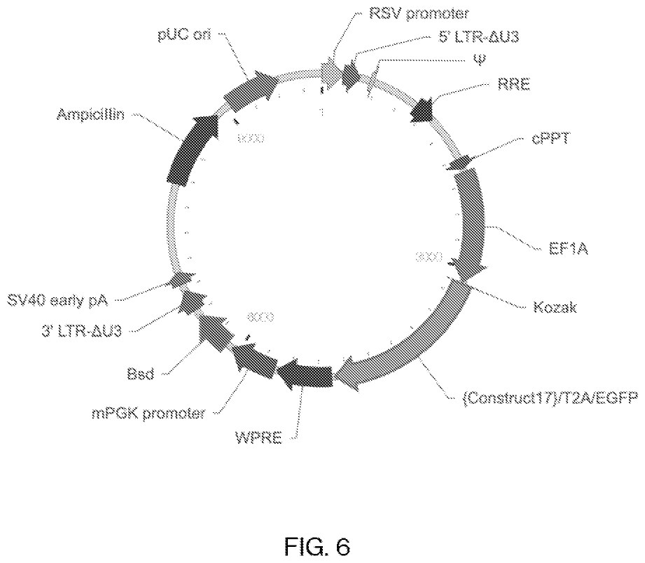

DNA encoding engineered SIRPα binding peptides as described herein are synthesized by VectorBuilder Inc. and cloned into a mammalian gene expression lentiviral vector (pLV) under the EF1α promotor and blasticidin antibiotic selection. A representative diagram for a polypeptide as disclosed herein is illustrated in FIG. 6. The constructs comprise a T2A ribosomal skipping sequence so the engineered polypeptide to GFP expression ratio is 1:1 and the expression product comprises two unlinked proteins, engineered SIRPα binding polypeptides that traffic to the membrane and cytosolic GFP.

### Target Cell Line Development

A K562 CD47^{-/-} cell line is thawed and allowed to recover for 1 week before transductions. The cell line is seeded into a T75 flask at 0.2×10⁶ VC/mL in 20 mL working volume for maintenance in RPMI+10% FBS+1% Pen-Strep. Cells are typically passaged every 2-3 days or when they reach a concentration of 1×10⁶ VC/mL. Cell counts and viability are recorded with a cell counter (e.g., NucloeCounter® NC-202TM).

Lentiviral transductions are conducted for the engineered SIRPα binding polypeptide constructs as described herein using an MOI=1, such as by adding 0.25×10⁶ VC/mL in 2 mL cell suspension (0.5×10⁶ total cells) of CD47 KO K562 per well of a 6 well plate, followed by addition of 5 μL and 10 μL of LV and polybrene.

Cells are kept in culture for 3 days in 6 well plate and scaled into T25 (0.2× 106 VC/mL in 5 mL) before applying antibiotic selection pressure (e.g. about 6 μg blasticidin). After antibiotic addition cells are maintained in selective media for 3-5 passages (generally 3) prior to cell sorting.

### Cell Sorting and Analysis for Cell Line Characterization

Cell sorting is performed in a Sony MA900 cell sorter. Double positive GFP-CD47 cells are sorted. Cells are expanded post-sorting in RPMI+10% FBS+1% PS+6 μg BLAST media. For FACS analysis, cells transduced with Lentivirus on early passages (2-4) are assessed for GFP and CD47 or engineered SIRPα binding protein expression.

### Characterization of Cell Lines: SIRPα Binding Protein Expression

For each cell line generated, the amount of engineered SIRPα binding polypeptide expressed on the cell surface is determined using the monoclonal antibodies B6H12 and CC2C6 dyed with Brilliant Violet™ 650 Dye (BV605). An isotype control dyed with PerCP-Cyanine5.5 is used for background subtraction, and surface expression levels are generally normalized using human WT CD47 transduced cells for comparison and analysis.

### Functional Assessment: Macrophage Cytotoxicity

To assess protection, THP-1 derived macrophages are prepared according to documented procedures and are co-cultured with K562-CD47 KO BFluc cell lines generated as described above displaying different engineered SIRPα binding polypeptides (target cells). Macrophages (effector cells) are titrated and at high ratios of macrophage cells, target cells are 100% susceptible to cytotoxic killing. Negative control CD47 knockout (K562-CD47-KO BFluc) target cells will be substantially all killed, and no luminescence signal will be observed from Luciferase containing target cells. Titration of effector to target cells (E: T) results in less killing of target cells and thus a luciferase signal. The data can be expressed as a percentage of cell killing for each construct to enable comparison between different constructs.

### Example 7. Natural Killer (NK) Cell Assay

This example outlines an NK cell killing assay to measure protection from macrophage cytotoxicity conferred by the engineered SIRPα binding polypeptides of the present disclosure.

### Construct Generation

DNA encoding engineered SIRPα binding peptides as described herein are synthesized by VectorBuilder Inc. and cloned into a mammalian gene expression lentiviral vector (pLV) under the EF1α promotor and blasticidin antibiotic selection. A representative diagram for a polypeptide as disclosed herein is illustrated in FIG. 6. The constructs comprise a T2A ribosomal skipping sequence so the engineered polypeptide to GFP expression ratio is 1:1 and the expression product comprises two unlinked proteins, engineered SIRPα binding polypeptides that traffic to the membrane and cytosolic GFP.

### Target Cell Line Development

K562 CD47 KO cell lines are thawed and allowed to recover for 1 week before transductions. The cell line is seeded into a T75 flask at 0.2×10⁶ VC/mL in 20 mL working volume for maintenance in RPMI+10% FBS+1% Pen-Strep. Cells are typically passaged every 2-3 days or when they reach a concentration of 1×10⁶ VC/mL. Cell counts and viability are recorded with a cell counter (e.g., NucloeCounter® NC-202TM).

The first round of transductions are performed with a commercial vector to integrate a FLuc transgene, followed by a second round of transductions to integrate polypeptides of the disclosure. KO and K562 cell lines containing engineered polypeptides are typically seeded into a T75 flask at 0.2×10⁶ VC/mL in 20 mL working volume for maintenance in RPMI+10% FBS +1% Pen-Strep. Cells are typically passaged every 2-3 days or when they reach a concentration of 106 VC/mL.

### Cell Sorting and Analysis for Cell Line Characterization

Cell sorting is performed in a Sony MA900 cell sorter. Double positive GFP-CD47 cells are sorted and expanded post-sort in RPMI+10% FBS+1% PS+6 μg BLAST media. Cell counts and viability are recorded with an automated cell counter (e.g., NucloeCounter® NC-202TM) prior to each assay.

### Characterization of Cell Lines: SIRPα Binding Protein Expression

For each cell line generated, the amount of engineered SIRPα binding polypeptide expressed on the cell surface is determined using the monoclonal antibodies B6H12 and CC2C6 dyed with Brilliant Violet™ 650 Dye (BV605). Mean fluorescence intensity (MFI) is quantified for cells expressing each construct. An isotype control dyed with PercCP-Cyanine5.5 is used for background subtraction and surface expression levels are generally normalized using human WT CD47 transduced cells for comparison and analysis.

### Functional Assessment: NK Cell Cytotoxicity

To assess protection, IL-2 activated NK cells (about 20 nM) from healthy donors are co-cultured with K562-CD47 KO BFluc cell lines generated as described above displaying different engineered SIRPα binding polypeptides (target cells). A parental K562 cell line is used as control as it is representative of the relative endogenous levels of CD47. Activated NK cells (effector cells) are titrated and at high ratios of NK cells, target cells are 100% susceptible to cytotoxic killing. Negative control cells with wild type CD47 knocked out (K562-CD47-KO BFluc target cells) will be substantially all killed, and no luminescence signal will be observed from Luciferase containing target cells. Titration of effector to target cells (E: T) results in less killing of target cells and thus a luciferase signal. The data can be expressed as a percentage of cell killing for each construct to enable comparison between different K562-CD47-KOBFluc constructs. Additional positive controls can include K562 cells engineered to overexpress WT CD47 (K562 CD47 T2A GFP) and human leukocyte antigen E (HLA-E) constructs (K562 HLA-E).

### Example 8. Conformational Ensembles of Engineered SIRPα Binding Proteins

In accordance with the operations described herein, several engineered SIRPα binding polypeptide sequences comprising mutations associated with cancer (e.g., one or more mutations listed in Table 3) were generated. A brief summary of the constructs are listed in Table 5. The conformational ensembles of the sequences were simulated by molecular dynamics (MD) in accordance with the molecular dynamics procedures outlined in Fenalti and Examples 1 and 4 herein above. Control constructs used as a basis of comparison were wild type human CD47 (WT CD47), WT CD47 in complex with signal regulatory peptide alpha (SIRPα), and WT CD47 in complex with the fragment antigen binding (Fab) region of a CD47-specific antibody (WT+Fab).

TABLE 5
Representative Engineered SIRPα Binding Sequences

| Construct | Sequence |
|---|---|
| Construct 4 | SEQ ID NO: 653 |
| Construct 11 | SEQ ID NO: 635 |
| Construct 20 | SEQ ID NO: 650 |
| Construct 21 | SEQ ID NO: 391 |
| Construct 22 | SEQ ID NO: 419 |
| Construct 23 | SEQ ID NO: 648 |
| Construct 25 | SEQ ID NO: 393 |
| Construct 26 | SEQ ID NO: 395 |
| Construct 27 | SEQ ID NO: 396 |
| Construct 28 | SEQ ID NO: 397 |
| Construct 29 | SEQ ID NO: 649 |
| Construct 30 | SEQ ID NO: 406 |
| Construct 32 | SEQ ID NO: 472 |
| Construct 33 | SEQ ID NO: 411 |
| Construct 35 | SEQ ID NO: 473 |
| Construct 37 | SEQ ID NO: 474 |
| Construct 39 | SEQ ID NO: 475 |
| Construct D | SEQ ID NO: 651 |
| Construct E | SEQ ID NO: 652 |

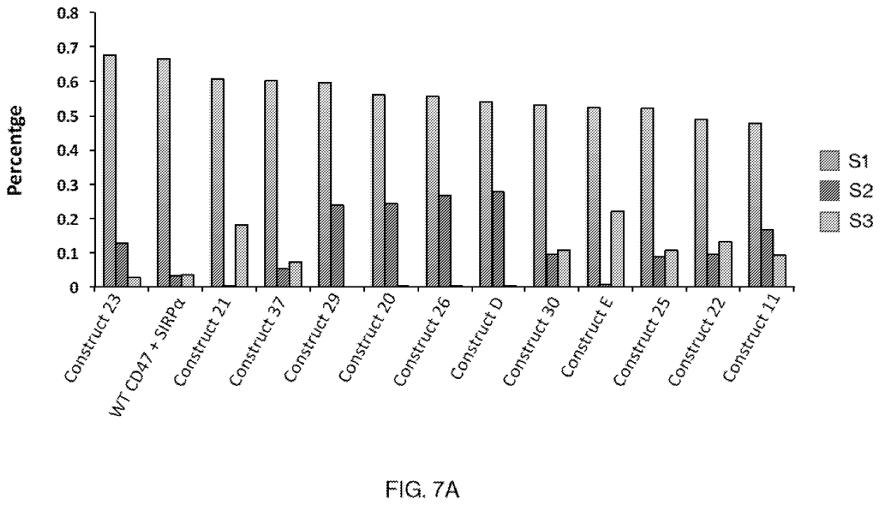

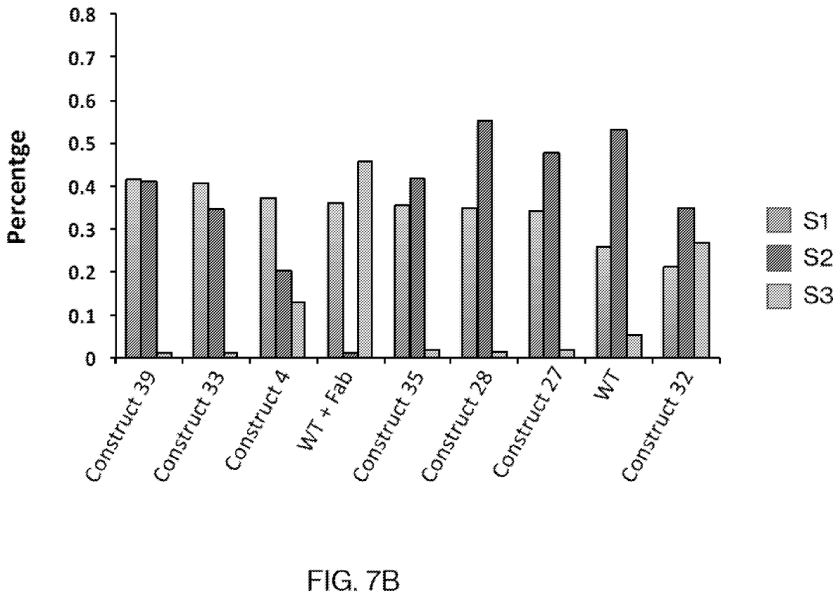

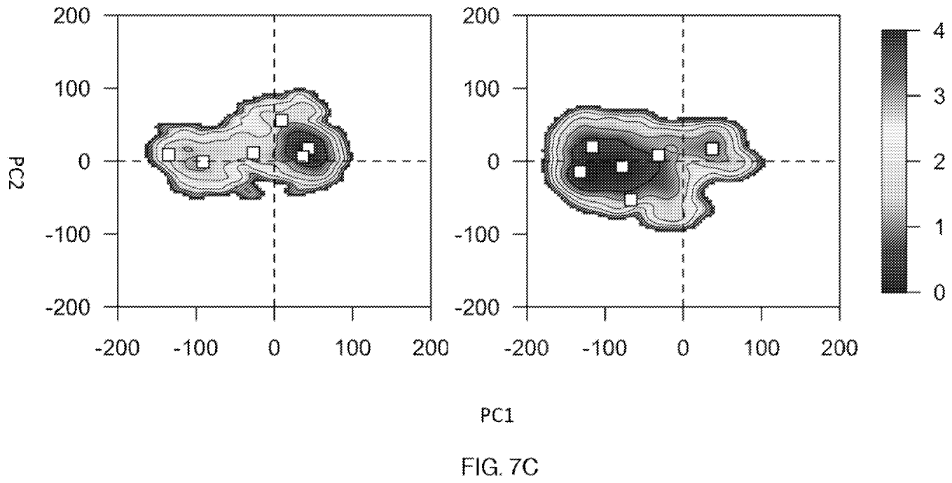

The results of each simulation were sampled and analyzed as discussed in Examples 1 and 4. FIG. 7A and FIG. 7B summarize the conformational ensembles of each construct tested as well as the controls. As illustrated in FIGS. 7A-7B, almost every construct shifted the conformational ensemble toward a greater proportion of the S1 metastate relative to wild type, with the exception of Construct 32 (SEQ ID NO: 472).

For Construct 22 (SEQ ID NO: 419), which comprises an R132C mutation relative to SEQ ID NO: 1, the free energy landscape was investigated by performing principal component analysis (PCA) on the downsampled trajectory and compared to that of the wild type. As illustrated in FIG. 7C, the introduction of the R132C mutation associated with cancer shifts the conformational ensemble from predominantly “S2-like” to predominantly “S1-like.”

### Example 9. Reduced Immunogenicity of Engineered SIRPα Binding Proteins (Cancer Mutants)

Engineered SIRPα binding sequences comprising one or more mutations associated with cancer (e.g., comprised in Table 3) were generated and tested for NK-cell killing and surface expression in accordance with the procedures of Example 7. The constructs tested are summarized below. Control constructs included endogenous expression of wild type CD47 (WT), CD47 knockout (CD47 KO), overexpression of wildtype CD47 (CD47 T2A GFP), and expression of HLA-E (HLA-E), which were similarly tested.

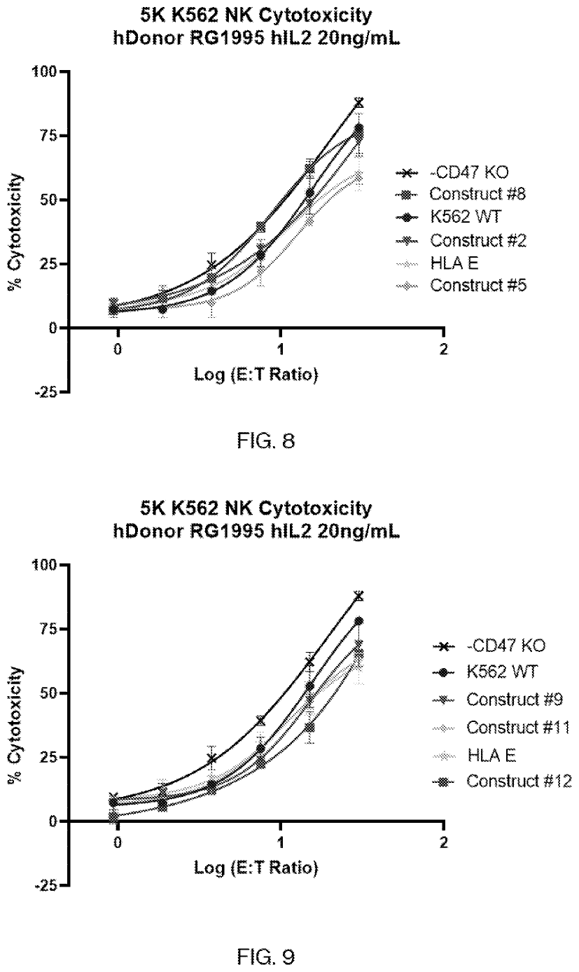

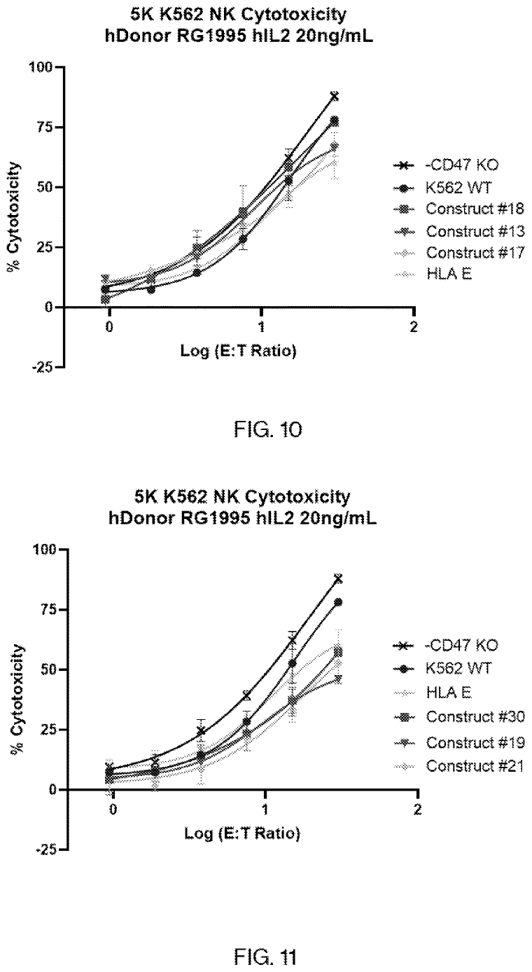

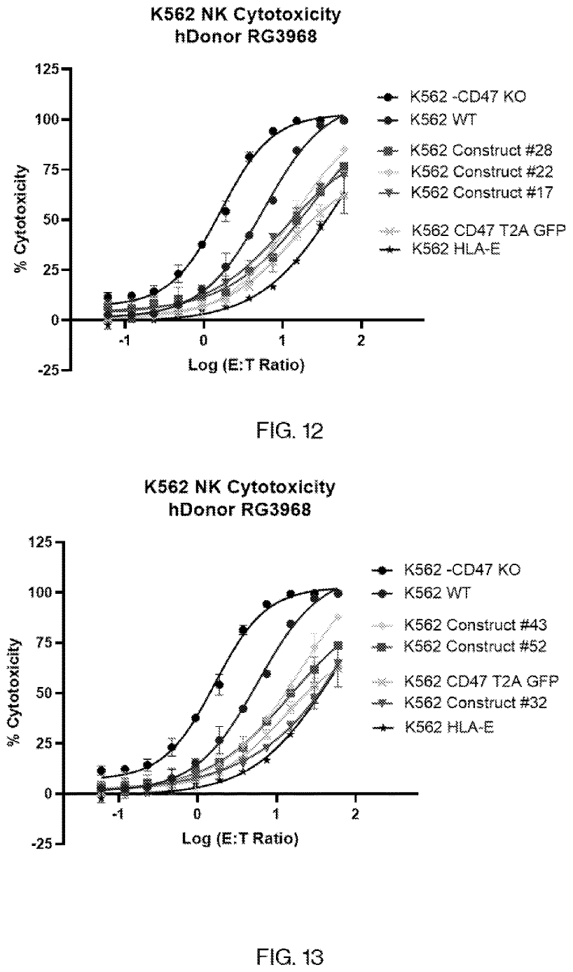

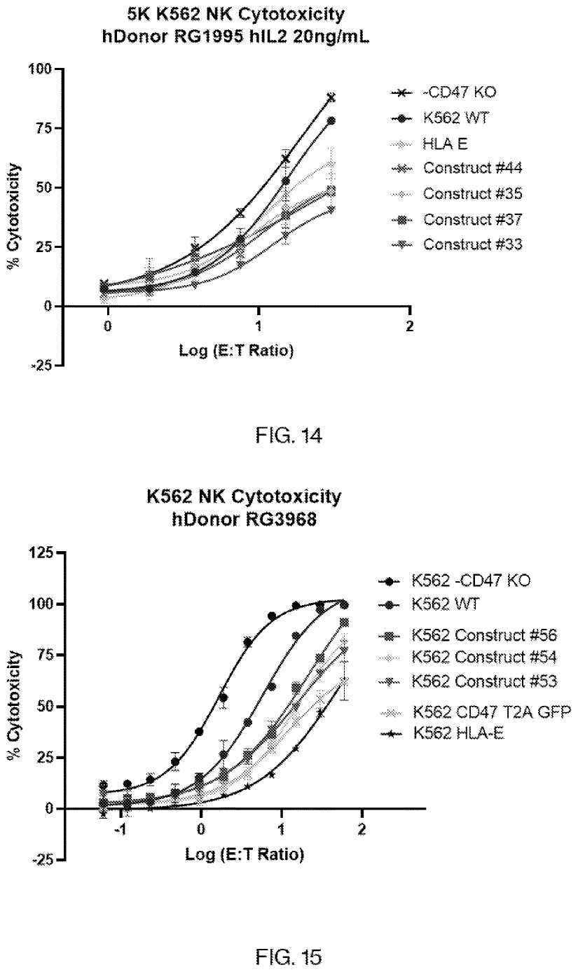

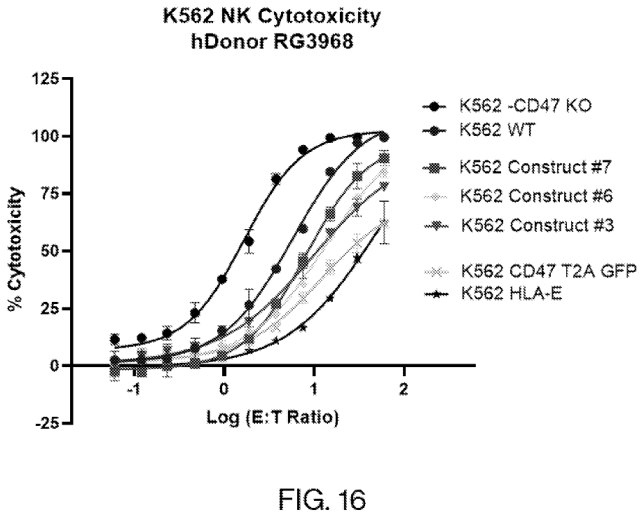

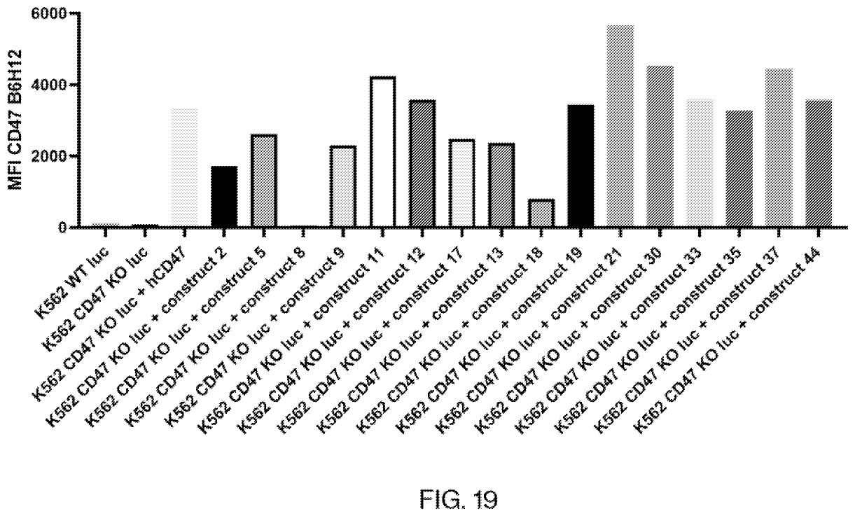

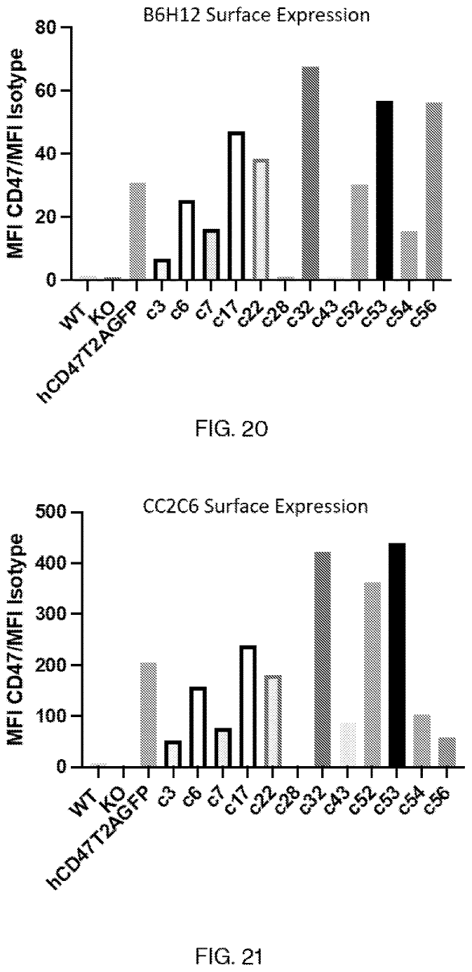

TABLE 6
Engineered SIRPα constructs
comprising cancer-associated mutations

| | | NK Cell | |
|---|---|---|---|
| Construct | Sequence | Protection | Surface Expression |
| Construct 6 | SEQ ID NO: 467 | FIG. 16 | FIGS. 20-21 |
| Construct 7 | SEQ ID NO: 468 | FIG. 16 | FIGS. 20-21 |
| Construct 8 | SEQ ID NO: 469 | FIG. 8 | FIG. 19 |
| Construct 9 | SEQ ID NO: 470 | FIG. 9 | FIG. 19 |
| Construct 11 | SEQ ID NO: 635 | FIG. 9 | FIG. 19 |
| Construct 12 | SEQ ID NO: 636 | FIG. 9 | FIG. 19 |
| Construct 13 | SEQ ID NO: 637 | FIG. 10 | FIG. 19 |
| Construct 19 | SEQ ID NO: 389 | FIG. 11 | FIG. 19 |
| Construct 21 | SEQ ID NO: 391 | FIG. 11 | FIG. 19 |
| Construct 22 | SEQ ID NO: 419 | FIG. 12 | FIGS. 20-21 |
| Construct 28 | SEQ ID NO: 397 | FIG. 12 | FIGS. 20-21 |
| Construct 30 | SEQ ID NO: 406 | FIG. 11 | FIG. 19 |
| Construct 32 | SEQ ID NO: 472 | FIG. 13 | FIGS. 20-21 |
| Construct 33 | SEQ ID NO: 411 | FIG. 14 | FIG. 19 |
| Construct 35 | SEQ ID NO: 473 | FIG. 14 | FIG. 19 |
| Construct 37 | SEQ ID NO: 474 | FIG. 14 | FIG. 19 |

FIGS. 8-14, and 16 illustrate the protection from NK-cell cytotoxicity conferred by the tested constructs. As illustrated in the Figures, each of the constructs provided protection from NK-cell killing, showing reduced NK-cell cytotoxicity relative to endogenous expression of WT CD47. Additionally, Construct 32 (SEQ ID NO: 472) showed protection from NK-cell killing enhanced beyond even that of overexpression of WT CD47 (FIG. 13).

FIGS. 19-21 illustrate the measured surface expression for the constructs. Surface expression was measured in accordance with the procedures of Example 7 using monoclonal antibodies for CD47: B6H12 (FIGS. 19 and 20) and CC2C6 (FIG. 21). Control constructs included endogenous expression of wild type CD47 (WT), CD47 knockout (KO), and overexpression of wild type CD47 (hCD47 T2A GFP). FIG. 19 illustrates measured mean fluorescence intensity (MFI) of a B6H12 monoclonal antibody specific for human CD47. FIG. 20 illustrates MFI of B6H12 except that each value has been normalized relative to the endogenously expressed wild type value. FIG. 21 illustrates the MFI of another CD47-specific monoclonal antibody, CC2C6, again with each value normalized relative to endogenous expression. In general, constructs for which surface expression could be measured showed surface expression greater than that of endogenously expressed. For some constructs (e.g., Construct 8 and Construct 28), expression could not be measured, presumably because the mutations altered the epitope such that monoclonal antibody binding could no longer be observed.

### Example 10. Reduced Immunogenicity of Engineered SIRPα Binding Proteins

Sequences predicted to bind to SIRPα and also optionally display one or more of (i) detuned binding of other CD47 cognate binding molecules (e.g., integrins, TSP-1); (ii) enhanced SIRPα signal transductions, (iii) enhanced binding to SIRPα, and (iv) increased stability were generated by rational mutational approaches. These sequences were then tested for NK cell killing and cell surface expression in accordance with the procedures of Example 7. Control constructs including endogenous expression of wild type CD47 (WT), CD47 knockout (CD47 KO), overexpression of wildtype CD47 (CD47 T2A GFP), and expression of HLA-E (HLA-E) were similarly tested.

TABLE 7
SIRPα binding polypeptide sequences

| | | NK Cell | |
|---|---|---|---|
| Construct | Sequence | Protection | Surface Expression |
| Construct 5 | SEQ ID NO: 634 | FIG. 8 | FIGS. 20-21 |
| Construct 17 | SEQ ID NO: 638 | FIG. 10 | FIG. 19 |
| Construct 18 | SEQ ID NO: 108 | FIG. 10 | FIG. 19 |
| Construct 43 | SEQ ID NO: 286 | FIG. 13 | FIGS. 20-21 |
| Construct 44 | SEQ ID NO: 377 | FIG. 14 | FIG. 19 |
| Construct 52 | SEQ ID NO: 47 | FIG. 13 | FIGS. 20-21 |
| Construct 53 | SEQ ID NO: 50 | FIG. 15 | FIGS. 20-21 |
| Construct 54 | SEQ ID NO: 141 | FIG. 15 | FIGS. 20-21 |
| Construct 56 | SEQ ID NO: 81 | FIG. 15 | FIGS. 20-21 |

FIGS. 8, 10, and 13-15 illustrate the protection from NK-cell cytotoxicity conferred by the tested constructs. As illustrated in the Figures, constructs generally conferred protection from NK cell cytotoxicity, showing reduced NK-cell cytotoxicity relative to endogenous expression of wild type CD47.

FIGS. 19-21 illustrate the measured surface expression for the constructs. Surface expression was measured in accordance with the procedures of Example 7 using monoclonal antibodies for CD47: B6H12 (FIGS. 19 and 20) and CC2C6 (FIG. 21). Control constructs included endogenous expression of wild type CD47 (WT), CD47 knockout (KO), and overexpression of wild type CD47 (hCD47 T2A GFP). FIG. 19 illustrates measured mean fluorescence intensity (MFI) of a B6H12 monoclonal antibody specific for human CD47. FIG. 20 illustrates MFI of B6H12 except that each value has been normalized relative to the endogenously expressed wild type value. FIG. 21 illustrates the MFI of another CD47-specific monoclonal antibody, CC2C6, again with each value normalized relative to endogenous expression. In general, constructs for which surface expression could be measured showed surface expression greater than that of endogenously expressed.

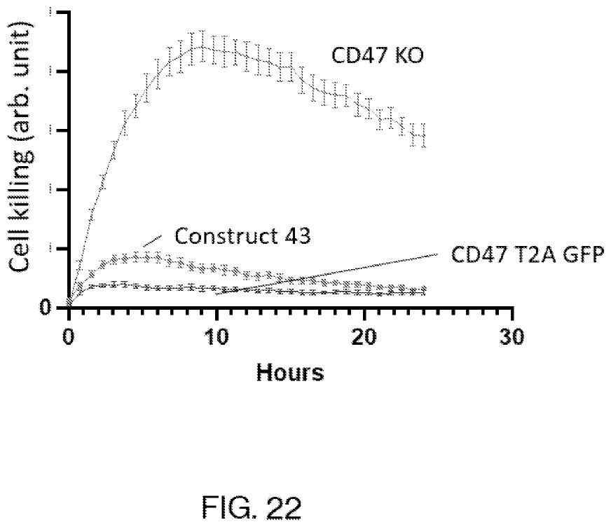

For Construct 43 (SEQ ID NO: 286), protection from macrophage cytotoxicity was additionally investigated according to the procedures of Example 6. FIG. 22 illustrates protection from macrophage phagocytosis (macrophage cytotoxicity) conferred by CD47 and Construct 43. Relative to CD47 knockout cells (CD47), cells expressing Construct 43 or overexpressing WT CD47 (CD47 T2A GFP) showed reduced macrophage susceptibility, suggesting protection from macrophage cytotoxicity was conferred by Construct 43 expression.

### Example 11. Reduced Immunogenicity of Engineered SIRPα Binding Proteins (Chimeras)

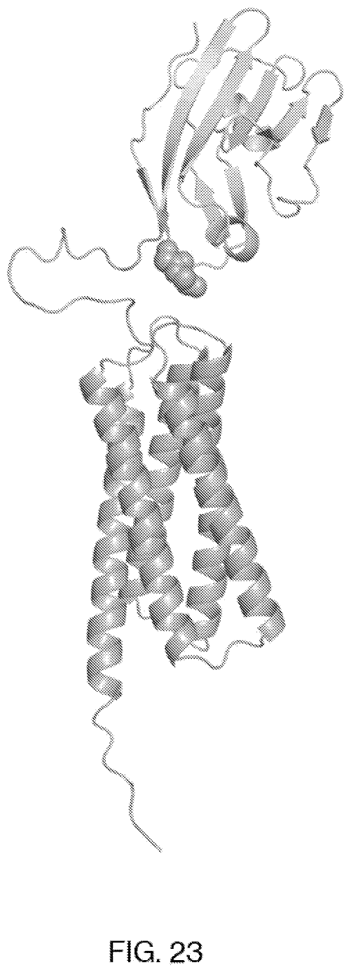

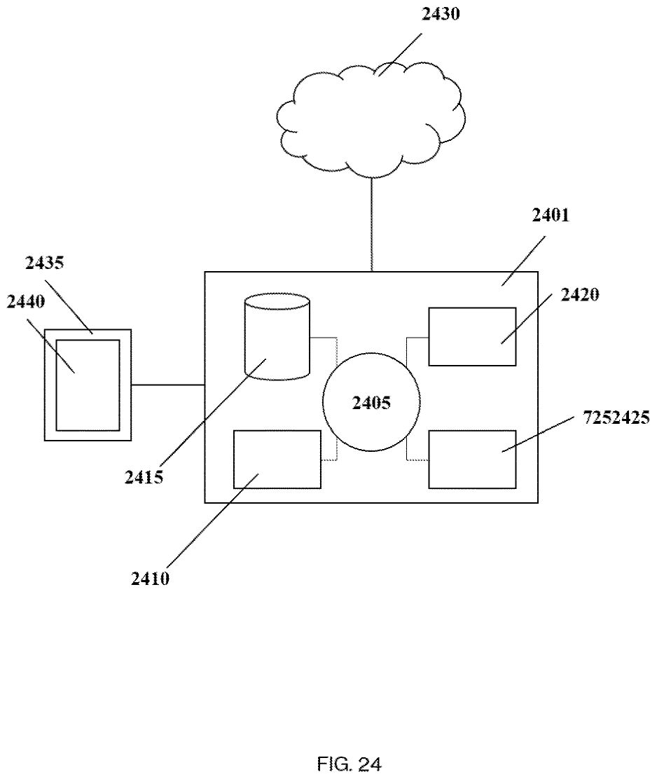

Chimeric SIRPα binding sequence comprising peptide loop insertions and in some cases domains from non-human species were generated and tested for construct-cell killing and surface expression in accordance with the procedures of Example 7. A model of Construct 3 is shown in FIG. 23. The constructs tested are summarized below. Control constructs included endogenous expression of wild type CD47 (WT), CD47 knockout (CD47 KO), overexpression of wildtype CD47 (CD47 T2A GFP), and expression of HLA-E (HLA-E), which were similarly tested.

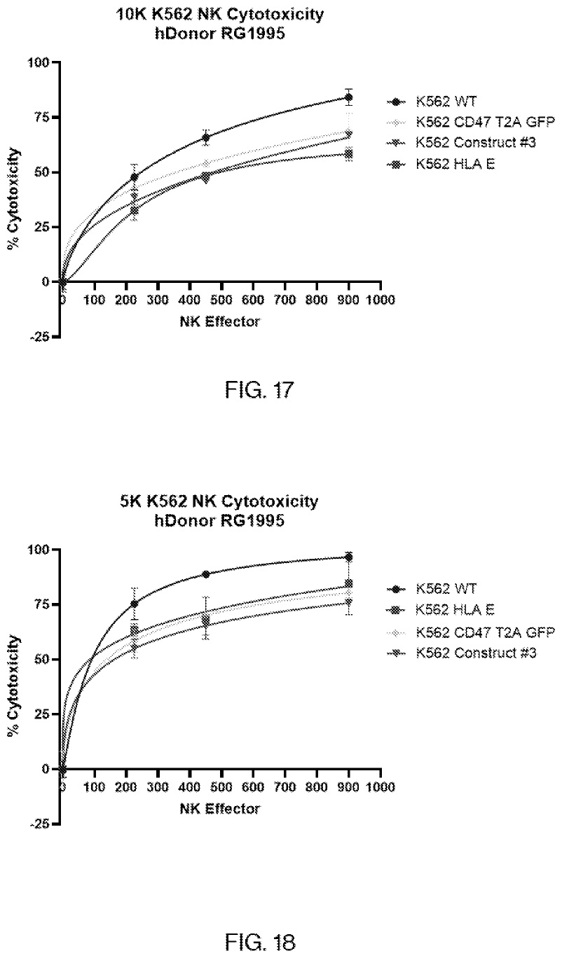

TABLE 8
Chimeric engineered SIRPα constructs

| | | NK Cell | |
|---|---|---|---|
| Construct | Sequence | Protection | Surface Expression |
| Construct 2 | SEQ ID NO: 639 | FIG. 8 | FIGS. 20-21 |
| Construct 3 | SEQ ID NO: 640 | FIGS. 16-18 | FIG. 19 |

FIGS. 8 and 16-18 illustrate the protection from NK-cell cytotoxicity conferred by the tested constructs. As illustrated in the Figures, each of the constructs provided protection from NK-cell killing, showing reduced NK-cell cytotoxicity relative to endogenous expression of WT CD47. Additionally, in certain donors, Construct 3 (SEQ ID NO: 640) showed protection from NK-cell killing enhanced beyond even that of overexpression of WT CD47 (FIG. 8).

FIGS. 19-21 illustrate the measured surface expression for the constructs. Surface expression was measured in accordance with the procedures of Example 7 using monoclonal antibodies for CD47: B6H12 (FIGS. 19 and 20) and CC2C6 (FIG. 21). Control constructs included endogenous expression of wild type CD47 (WT), CD47 knockout (KO), and overexpression of wild type CD47 (hCD47 T2A GFP). FIG. 19 illustrates measured mean fluorescence intensity (MFI) of a B6H12 monoclonal antibody specific for human CD47. FIG. 20 illustrates MFI of B6H12 except that each value has been normalized relative to the endogenously expressed wild type value. FIG. 21 illustrates the MFI of another CD47-specific monoclonal antibody, CC2C6, again with each value normalized relative to endogenous expression. In general, constructs for which surface expression could be measured showed surface expression greater than that of endogenously expressed. Notably, Construct 3, which in some donors showed better protection from NK cell killing than overexpression of CD47, also showed lower surface expression than overexpressed CD47.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. An engineered polypeptide comprising a signal regulatory protein alpha (SIRPα) binding sequence, wherein said engineered polypeptide is configured to elicit a decreased immune response when expressed on a surface of a cell as compared to a reference polypeptide comprising a sequence of residues 19-290 of any one of SEQ ID NOs: 1-4, wherein said decreased immune response is at least about 5%, 10%, 15%, 20%, 25%, 30%, 35%, 40%, 45%, 50%, 55%, 65%, 70%, 75%, 80%, 85%, 90%, 95%, or 100% decreased relative to said reference polypeptide.

2. The engineered polypeptide of claim 1, wherein said immune response comprises natural killer (NK) cell cytotoxicity.

3. The engineered polypeptide of claim 1 or 2, wherein said immune response comprises macrophage cytotoxicity.

4. An engineered polypeptide comprising a signal regulatory protein alpha (SIRPα) binding sequence, wherein said polypeptide comprises a conformational ensemble comprising a first metastable state, which first metastable state is configured to bind to SIRPα, and wherein said conformational ensemble comprises a greater proportion of said first metastable state than a conformational ensemble of a reference polypeptide, wherein said reference polypeptide comprises a sequence of residues 19-290 of any one of SEQ ID NOs: 1-4.

5. The engineered polypeptide of claim 4, wherein said proportion of said conformational ensemble of said engineered polypeptide or said proportion of said conformational ensemble of said reference polypeptide is determined at least in part by hydrogen-deuterium exchange (HDX), small-angle x-ray scattering (SAXS), nuclear magnetic resonance (NMR), or molecular dynamics (MD).

6. The engineered polypeptide of claim 5, wherein said proportion of said conformational ensemble of said engineered polypeptide or said proportion of said conformational ensemble of said reference polypeptide is determined at least in part by MD.

7. The engineered polypeptide of any one of claims 4-6, wherein said first metastable state is characterized by a bending angle between a transmembrane domain (TMD) and an extracellular domain (ECD) of said polypeptide.

8. The engineered polypeptide of claim 7, wherein said TMD comprises five alpha helices.

9. The engineered polypeptide of claim 7 or 8, wherein said ECD is configured to bind SIRPα.

10. The engineered polypeptide of any one of claims 7-9, wherein said bending angle is from about 130 to about 180 degrees.

11. The engineered polypeptide of any one of claims 4-10, wherein said first metastable state is characterized by a distance between said TMD and said ECD of said polypeptide.

12. The engineered polypeptide of claim 11, wherein said distance is from about 10 to about 25 angstroms (Å).

13. The engineered polypeptide of any one of claims 4-12, wherein said engineered polypeptide is configured to elicit a decreased immune response when expressed on a surface of a cell as compared to a reference polypeptide comprising a sequence of residue 19 through the final residue of any one of SEQ ID NOs: 1-4, wherein said decreased immune response is at least about 5%, 10%, 15%, 20%, 25%, 30%, 35%, 40%, 45%, 50%, 55%, 65%, 70%, 75%, 80%, 85%, 90%, 95%, or 100% decreased relative to said reference polypeptide.

14. The engineered polypeptide of claim 13, wherein said immune response comprises natural killer (NK) cell cytotoxicity.

15. The engineered polypeptide of claim 13 or 14, wherein said immune response comprises macrophage cytotoxicity.

16. An engineered polypeptide comprising a signal regulatory protein alpha (SIRPα) binding sequence, wherein said engineered polypeptide is configured to elicit a decreased immune response when expressed on a surface of a cell as compared to a reference polypeptide comprising a sequence of residues 19-290 of any one of SEQ ID NOs: 1-4, wherein said SIRPα binding sequence comprises at least one mutation relative to any one of SEQ ID NOs: 1-4, wherein said at least one mutation is associated with a cancer.

17. The engineered polypeptide of claim 16, wherein said at least one mutation is comprised in a database.

18. The engineered polypeptide of claim 17, wherein said database comprises the Catalogue of Somatic Mutations in Cancer (COSMIC), the Genome Aggregation Database (gnomAD), or both.

19. The engineered polypeptide of any one of claims 16-18, wherein said at least one mutation comprises a mutation selected from the group consisting of: the mutations listed in Table 3, and any combination thereof.

20. The engineered polypeptide of any one of claims 16-18, wherein said at least one mutation comprises a mutation selected from the group consisting of: M31, L40, C42, D47, D64, C75, E80, F97, G105, K106, F106, K111, S123, S127, K128, F131, C132, C136, K140, T142, G146, M153, L157, L160, E166, C170, D178, A203, S207, V210, D211, L214, S215, V262, L264, Y267, and any combination thereof.

21. The engineered polypeptide of any one of claims 16-20, wherein said polypeptide comprises an additional mutation, wherein said additional mutation is selected from the group consisting of:, Y31, A32, R35, K35, P71, A77, A79, N80, L100, K138, L164, M185, A211, S259, E262, and any combination thereof.

22. An engineered signal regulatory protein alpha (SIRPα) binding polypeptide comprising: an extracellular domain (ECD), a transmembrane domain (TMD), and an extracellular loop region (ECLR), wherein said extracellular loop region comprises a heterologous sequence compared to residues 19 through the final residue of any one of SEQ ID NOs: 1-4.

23. The engineered polypeptide of claim 22, wherein said ECD, said TMD, and said ECLR are derived from at least two different organisms.

24. The engineered polypeptide of claim 22 or 23, wherein said ECLR comprises at least about 70%, 80%, 85%, 90%, 91%, 92%, 93%, 94%, 95%, 96%, 97%, 98%, or 99%, or 100% sequence identity to an ECLR of any one of SEQ ID NOs: 639 and 640.

25. The engineered polypeptide of any one of claims 22-24, wherein said heterologous sequence comprises a pair of cysteine residues configured to form a disulfide pair.

26. The engineered polypeptide of claim 25, wherein said heterologous sequence comprises at least about 70%, 80%, 85%, 90%, 91%, 92%, 93%, 94%, 95%, 96%, 97%, 98%, or 99%, or 100% sequence identity to any one of SEQ ID NOs: 641-647.

27. The engineered polypeptide of any one of claims 22-26, wherein said engineered polypeptide is configured to elicit a decreased immune response when expressed on a surface of a cell as compared to a reference polypeptide comprising a sequence of any one of SEQ ID NO: 1-4.

28. The engineered polypeptide of any one of claim 1-3 or 27, wherein said polypeptide and said reference polypeptide are configured to adopt a first metastable state, wherein said first metastable state is configured to bind to SIRPα.

29. The engineered polypeptide of claim 28, wherein a conformational ensemble of said engineered polypeptide comprises a larger proportion of said first metastable state than a conformational ensemble of said reference polypeptide.

30. The engineered polypeptide of claim 29, wherein said proportion of said conformational ensemble of said engineered polypeptide or said proportion of said conformational ensemble of said reference polypeptide is determined at least in part by hydrogen-deuterium exchange (HDX), small-angle x-ray scattering (SAXS), nuclear magnetic resonance (NMR), or molecular dynamics (MD).

31. The engineered polypeptide of claim 30, wherein said proportion of said conformational ensemble of said engineered polypeptide or said proportion of said conformational ensemble of said reference polypeptide is determined at least in part by MD.

32. The engineered polypeptide of any one of claims 28-30, wherein said first metastable state is characterized by a bending angle between a transmembrane domain (TMD) and an extracellular domain (ECD) of said polypeptide.

33. The engineered polypeptide of claim 32, wherein said TMD comprises five alpha helices.

34. The engineered polypeptide of claim 32 or 33, wherein said ECD is configured to bind SIRPα.

35. The engineered polypeptide of any one of claims 32-34, wherein said bending angle is from about 130 to about 180 degrees.

36. The engineered polypeptide of any one of claims 28-35, wherein said first metastable state is characterized by a distance between said TMD and said ECD of said polypeptide.

37. The engineered polypeptide of claim 36, wherein said distance is from about 10 to about 25 angstroms (Å).

38. The engineered polypeptide of any one of claims 1-37, wherein said SIRPα binding sequence comprises at least about 70%, 80%, 90%, 95%, or 99%, or 100% identity to any one of SEQ ID NOs: 10-640 or 648-653.

39. The engineered polypeptide of any one of claims 1-38, wherein said engineered polypeptide comprises a sequence at least about 70%, 80%, 80%, 95%, or 99%, or 100% identical to an extracellular domain (ECD) of any one of SEQ ID NOs: 1-640 and 648-653.

40. The engineered polypeptide any one of claims 1-39, wherein said engineered polypeptide comprises a sequence at least about 70%, 80%, 80%, 95%, or 99%, or 100% identical to a transmembrane domain (TMD) of any one of SEQ ID NOs: 1-640 and 648-653.

41. The engineered polypeptide of any one of claims 1-40, wherein said engineered polypeptide comprises a sequence at least about 70%, 80%, 80%, 95%, or 99%, or 100% identical to an extracellular loop region (ECLR) of any one of SEQ ID NOs: 1-640 and 648-653.

42. The engineered polypeptide of any one of claims 1-41, wherein said engineered polypeptide comprises at least one amino acid substitution specified in Table 3.

43. The engineered polypeptide of claim 42, wherein said engineered polypeptide comprises one or more amino acid substitutions selected from the group consisting of: M31, L40, C42, D47, D64, C75, E80, F97, G105, K106, F106, K111, S123, S127, K128, F131, C132, C136, K140, T142, G146, M153, L157, L160, E166, C170, D178, A203, S207, V210, D211, L214, S215, V262, L264, Y267, and any combination thereof.

44. The engineered polypeptide of any one of claims 1-43, wherein said engineered polypeptide comprises at least one amnio acid substitution specified in Table 4.

45. The engineered polypeptide of any one of claims 1-44, wherein said engineered polypeptide comprises at least one amino acid substitution selected from the group consisting of: Y31, A32, R35, K35, P71, A77, A79, N80, L100, K138, L164, M185, A211, S259, E262, and any combination thereof.

46. The engineered polypeptide of any one of claims 1-45, wherein said cell is a stem cell.

47. The engineered polypeptide of claim 46, wherein said stem cell is an induced pluripotent stem cell (iPSC).

48. The engineered polypeptide of any one of claims 1-47, wherein said polypeptide comprises an N-terminal addition.

49. The engineered polypeptide of any one of claims 1-48, wherein said TMD, said ECD, and said linker are from more than one organism.

50. The engineered polypeptide of any one of claims 1-49, wherein said engineered polypeptide is configured to elicit a reduced integrin response as compared to said reference polypeptide.

51. The engineered polypeptide of any one of claims 1-50, wherein said polypeptide is configured to elicit a reduced thrombospondin 1 (TSP-1) response as compared to said reference polypeptide.

52. An engineered polypeptide comprising a sequence with at least about 70%, 80%, 81, 82, 83, 84, 85, 86, 87, 88, 89, 90, 91, 92, 93, 94, 95, 96, 97, 98, or 99, or 100% identity to any one of SEQ ID NOs: 1-640 and 648-653.

53. The engineered polypeptide of claim 52, wherein said engineered polypeptide comprises a sequence at least about 70%, 80%, 80%, 95%, or 99%, or 100% identical to an extracellular domain (ECD) of any one of SEQ ID NOs: 1-640 and 648-653.

54. The engineered polypeptide of claim 52 or 53, wherein said engineered polypeptide comprises a sequence at least about 70%, 80%, 80%, 95%, or 99%, or 100% identical to a transmembrane domain (TMD) of any one of SEQ ID NOs: 1-640 and 648-653.

55. The engineered polypeptide of any one of claims 52-54, wherein said engineered polypeptide comprises a sequence at least about 70%, 80%, 80%, 95%, or 99%, or 100% identical to an extracellular loop region (ECLR) of any one of SEQ ID NOs: 1-640 and 648-653.

56. The engineered polypeptide of any one of claims 52-55, wherein said engineered polypeptide comprises a sequence at least about 70%, 80%, 80%, 95%, or 99%, or 100% identical to any one of SEQ ID NOs: 641-647.

57. An engineered polypeptide comprising a signal regulatory protein alpha (SIRPα) binding sequence, wherein said engineered polypeptide comprises a conformational ensemble comprising a first metastable state, wherein said first metastable state is configured to bind SIRPα, and wherein said conformational ensemble comprises a greater proportion of said first metastable state than a conformational ensemble of a reference polypeptide, wherein said reference polypeptide comprises a sequence of residues 19-290 of any one of SEQ ID NOs: 1-4.

58. The engineered polypeptide of claim 57, wherein said proportion of said conformational ensemble of said engineered polypeptide or said proportion of said conformational ensemble of said reference polypeptide is determined at least in part by hydrogen-deuterium exchange (HDX), small-angle x-ray scattering (SAXS), nuclear magnetic resonance (NMR), or molecular dynamics (MD).

59. The engineered polypeptide of claim 58, wherein said proportion of said conformational ensemble of said engineered polypeptide or said proportion of said conformational ensemble of said reference polypeptide is determined at least in part by MD.

60. The engineered polypeptide of any one of claims 57-59, wherein said first metastable state is characterized by a bending angle between a transmembrane domain (TMD) and an extracellular domain (ECD) of said polypeptide.

61. The engineered polypeptide of claim 60, wherein said TMD comprises five alpha helices.

62. The engineered polypeptide of claim 60 or 61, wherein said ECD is configured to bind SIRPα.

63. The engineered polypeptide of any one of claims 60-62, wherein said bending angle is from about 130 to about 180 degrees.

64. The engineered polypeptide of any one of claims 57-63, wherein said first metastable state is characterized by a distance between said TMD and said ECD of said polypeptide.

65. The engineered polypeptide of claim 64, wherein said distance is from about 10 to about 25 angstroms (Å).

66. The engineered polypeptide of any one of claims 57-65, wherein said engineered polypeptide is configured to elicit a decreased immune response when expressed on a surface of a cell as compared to a reference polypeptide comprising a sequence of residues 19-290 of any one of SEQ ID NOs: 1-4, wherein said decreased immune response is at least about 5%, 10%, 15%, 20%, 25%, 30%, 35%, 40%, 45%, 50%, 55%, 65%, 70%, 75%, 80%, 85%, 90%, 95%, or 100% decreased relative to said reference polypeptide.

67. The engineered polypeptide of claim 66, wherein said immune response comprises natural killer (NK) cell cytotoxicity.

68. The engineered polypeptide of claim 66 or 67, wherein said immune response comprises macrophage cytotoxicity.

69. The engineered polypeptide of any one of claims 57-68, wherein said SIRPα binding sequence comprises at least about 70%, 80%, 90%, 95%, or 99%, or 100% identity to any one of SEQ ID NOs: 10-640 or 684-653.

70. The engineered polypeptide of any one of claims 57-69, wherein said engineered polypeptide comprises at least one amino acid substitution specified in Table 3.

71. The engineered polypeptide of claim 70, wherein said engineered polypeptide comprises one or more amino acid substitutions selected from the group consisting of: M31, L40, C42, D47, D64, C75, E80, F97, G105, K106, F106, K111, S123, S127, K128, F131, C132, C136, K140, T142, G146, M153, L157, L160, E166, C170, D178, A203, S207, V210, D211, L214, S215, V262, L264, Y267, and any combination thereof.

72. The engineered polypeptide of any one of claims 57-71, wherein said engineered polypeptide comprises at least one amnio acid substitution specified in Table 4.

73. The engineered polypeptide of any one of claims 57-72, wherein said engineered polypeptide comprises at least one amino acid substitution selected from the group consisting of: Y31, A32, R35, K35, P71, A77, A79, N80, L100, K138, L164, M185, A211, S259, E262, and any combination thereof.

74. The engineered polypeptide of any one of claims 57-73, wherein said cell is a stem cell.

75. The engineered polypeptide of claim 74, wherein said stem cell is an induced pluripotent stem cell (iPSC).

76. The engineered polypeptide of any one of claims 57-75, wherein said polypeptide comprises an N-terminal addition.

77. The engineered polypeptide of any one of claims 57-76, wherein said TMD, said ECD, and said linker are from more than one organism.

78. The engineered polypeptide of any one of claims 57-77, wherein said engineered polypeptide is configured to elicit a reduced integrin response as compared to said reference polypeptide.

79. The engineered polypeptide of any one of claims 57-78, wherein said polypeptide is configured to elicit a reduced thrombospondin 1 (TSP-1) response as compared to said reference polypeptide.

80. An engineered cell comprising the engineered polypeptide of any one of claims 1-79.

81. An engineered cell comprising a plurality of SIRPα binding polypeptides, wherein the plurality of SIRPα binding polypeptide comprises an engineered polypeptide of claims 1-79.

82. The engineered cell of claim 81, wherein said plurality of SIRPα binding polypeptide further comprises a wild type CD47.

83. The engineered cell of claim 81 or 82, wherein said plurality of SIRPα binding polypeptide comprises a plurality of engineered polypeptides of claims 1-79.

84. The engineered cell of any one of claims 81-83, wherein said engineered cell is a stem cell.

85. The engineered cell of claim 84, wherein said stem cell is an embryonic stem cell, a mesenchymal stem cell, an induced pluripotent stem cell, or a hematopoietic stem cell.

86. A nucleic acid molecule encoding the engineered polypeptide of any one of claims 1-79.

87. A nucleic acid molecule encoding a plurality of SIRPα binding polypeptides, wherein said plurality of SIRPα binding polypeptides comprises a wild type CD47 and the engineered polypeptide of any one of claims 1-79.

88. A nucleic acid molecule encoding a plurality of SIRPα binding polypeptides, wherein said plurality of SIRPα binding polypeptides comprises a plurality of engineered polypeptides of any one of claims 1-79.

89. A vector comprising the nucleic acid of any one of claims 86-88.

90. The vector of claim 89, wherein said vector is a plasmid, a minicircle, a CELiD, an adeno-associated virus (AAV) derived virion, a lentivirus, an adenovirus, or a herpes simplex virus (HSV).

91. A method of generating a hypoimmunogenic cell comprising administering to a cell the vector of claim 89 or 90.
